(19) United States
(12) Patent Application Publication
(10) Pub. No.: US 2024/0392394 A1
(43) Pub. Date: Nov. 28, 2024

(54) COMPOSITIONS, KITS AND METHODS FOR DETECTION OF VIRAL VARIANT SEQUENCES

(71) Applicant: LIFE TECHNOLOGIES CORPORATION, Carlsbad, CA (US)

(72) Inventors: Pius BRZOSKA, Woodside, CA (US); Joyce WILDE, Moss Beach, CA (US); Manohar FURTADO, San Ramon, CA (US); Sara KENDRICK, Zionsville, IN (US); Camilla ULEKLEIV, Fetsund (NO); Junko STEVENS, Menlo Park, CA (US); Kamini VARMA, Saratoga, CA (US); Kathleen HAYASHIBARA, Campbell, CA (US)

(21) Appl. No.: 18/262,672

(22) PCT Filed: Jan. 25, 2022
(86) PCT No.: PCT/US2022/013665

§ 371 Date: Jul. 24, 2023

Related U.S. Application Data
(60) Provisional application No. 63/199,792, filed on Jan. 25, 2021.
(60) Provisional application No. 63/199,922, filed on Feb. 3, 2021.
(60) Provisional application No. 63/200,014, filed on Feb. 9, 2021.
(60) Provisional application No. 63/200,384, filed on Mar. 3, 2021.
(60) Provisional application No. 63/201,037, filed on Apr. 9, 2021.
(60) Provisional application No. 63/179,159, filed on Apr. 23, 2021.
(60) Provisional application No. 63/184,919, filed on May 6, 2021.
(60) Provisional application No. 63/251,407, filed on Oct. 1, 2021.
(60) Provisional application No. 63/282,830, filed on Nov. 24, 2021.
(60) Provisional application No. 63/286,712, filed on Dec. 7, 2021.
(60) Provisional application No. 63/286,965, filed on Dec. 7, 2021.
(60) Provisional application No. 63/289,733, filed on Dec. 15, 2021.
(60) Provisional application No. 63/298,156, filed on Jan. 10, 2022.

(51) Int. Cl. C12Q 1/70

(52) U.S. Cl. CPC C12Q 1/701; C12Q 2600/156

(57) ABSTRACT

Disclosed are compositions, assays, methods, diagnostic methods, kits and diagnostic kits for the specific and differential detection of SARS-CoV-2, including SARS-CoV-2 variants, or other coronaviruses from samples including veterinary samples, clinical samples, food samples, forensic sample, an environmental sample (e.g., soil, dirt, garbage, sewage, air, or water), including food processing and manufacturing surfaces, or a biological sample.

### CROSS-REFERENCE TO RELATED APPLICATIONS

This application claims priority to and the benefit of United States Provisional Patent Application Ser. Nos:
63/199,792 filed Jan. 25, 2021;
63/199,922 filed Feb. 3, 2021;
63/200,014 filed Feb. 9, 2021;
63/200,384 filed Mar. 3, 2021;
63/201,037 filed Apr. 9, 2021;
63/179,159 filed Apr. 23, 2021;
63/184,919 filed May 6, 2021;
63/251,407 filed Oct. 1, 2021;
63/282,830 filed Nov. 24, 2021;
63/286,712 filed Dec. 7, 2021;
63/286,965 filed Dec. 7, 2021;
63/289,733 filed Dec. 15, 2021; and
63/298,156 filed Jan. 10, 2022.
   Each of the foregoing applications is incorporated herein in its entirety by this reference.

### INCORPORATION BY REFERENCE OF SEQUENCE LISTING

This application includes a Sequence Listing submitted electronically in ASCII format. The ASCII copy of the Sequence Listing, created on Jan. 15, 2022, is named 25 LT01605PCT-11398.267a-SL.txt and is 1,328,052 bytes in size. The ASCII copy of the Sequence Listing is expressly incorporated herein by this reference.

### FIELD

The present teachings relate to compositions, methods, systems and kits for specific detection, diagnosis and differentiation of viruses involved in infectious diseases. 30 Differential detection of specific viral agents allows accurate diagnosis so that appropriate treatment and infection control measures can be provided in a timely manner.

### BACKGROUND

Coronaviruses are a family of viruses having a positive-sense single stranded RNA genome of about 30 kilobases in length. Human coronaviruses were first identified in the mid 1960's as being one of the many etiologic agents of the common cold. People around the world commonly get infected with human coronavirus strains 229E (an alpha coronavirus), NL63 (an alpha coronavirus), OC43 (a beta coronavirus), and HKU1 (a beta coronavirus). These infections present with mild clinical symptoms and are associated with an extremely low mortality rate.

Some coronaviruses infect non-human animals where they can evolve and undergo zoonosis, expanding their tropism to humans. Such crossover events have proven devastating in years past. For example, the Middle East Respiratory Syndrome (MERS) was caused by MERS-CoV, a beta coronavirus that crossed over from dromedary camels to humans. MERS-CoV was associated with a high mortality rate of approximately 35%, but its low transmissibility rate helped to limit its spread and potential for devastation. As another example, Severe Acute Respiratory Syndrome (SARS), which was caused by SARS-CoV, another beta coronavirus, was believed to have been transmitted from bats to civet cats who then transmitted the virus to humans. Although not as deadly as MERS-CoV, SARS-CoV was nevertheless associated with a moderately high mortality rate of approximately 9.6%. Likely due, at least in part, to the lifecycle of SARS-CoV within humans, the spread of this virus was limited mostly to Southeast Asian countries. Human infected with SARS-CoV often became symptomatic prior to shedding infectious virions, making quarantining a particularly useful tool for limiting exposure and spread of the infection.

More recently, a new beta coronavirus, SARS-CoV-2 (also known as 2019-nCoV), has emerged, potentially from a crossover event between animals and humans in Wuhan, China. While the epidemiological data are incomplete, reports so far indicate that nearly 317 million people worldwide are believed to have been infected by SARS-CoV-2. However, unlike MERS-CoV and SARS-CoV before it, SARS-CoV-2 appears to be significantly less lethal on average. Due to its increased transmissibility, the seemingly small percentage of deaths associated with SARS-CoV-2 belies its worldwide impact, having caused an estimated 5.51 million deaths, at the time of this writing, in the worldwide pandemic. The raw number of humans impacted by SARS-CoV-2 dwarfs the total number of deaths caused by MERS-CoV and SARS-CoV combined-reportedly around 1,600.

Further, because SARS-CoV-2 is an RNA virus, it can mutate with relatively high frequency, with some estimating that SARS-CoV-2 undergoes about 1-2 mutations per month. Some variants, however, have acquired mutations more rapidly than expected. Indeed, as the pandemic has progressed, multiple new mutations and variants have been identified. The term “variant” is used to describe a subtype of a microorganism that is genetically distinct from a major “reference” form. SARS-CoV-2 variants are designated according to the Pango lineage nomenclature system, and more recently have also been identified using a World Health Organization (WHO) label. For example, for much of 2021 the dominant variant of SARS-CoV-2 in the United States and most of the world was the B.1.617.2 variant (under the Pango lineage nomenclature), more commonly referred to as “the Delta variant” (under the corresponding WHO label). At the time of this writing, the dominant variant is the B.1.1.529 variant (under the Pango lineage nomenclature), more commonly referred to as “the Omicron variant”.

The U.S. Centers for Disease Control and Prevention (CDC) and the WHO categorize variants as Variants Being Monitored (VBM) or Variants Under Monitoring (VUM), Variants of Interest (VOI), and Variants of Concern (VOC). A VBM is a variant for which there are data indicating an impact on medical countermeasures, or that has been associated with more severe disease or increased transmission but are no longer detected or are circulating at very low levels. A VOI is a variant with specific genetic markers that are predicted to affect transmission, diagnostics, therapeutics, or immune escape, but currently has limited prevalence or expansion. A VOC is a variant for which there is evidence of an increase in transmissibility, more severe disease (e.g., increased hospitalizations or deaths), significant reduction in neutralization by antibodies generated during previous infection or vaccination, reduced effectiveness of treatments or vaccines, or diagnostic detection failures. At the time of this writing, the CDC and the WHO each classify the Delta variant and the B.1.1.529 variant (“the Omicron variant”) as VOCs, and the WHO additionally includes the B.1.1.7 variant (“the Alpha variant”, previously referred to as “the UK variant”), the B.1.351 variant (“the Beta variant”, previously referred to as “the South African variant”), and the P.1 variant (“the Gamma variant”) as VOCs.

The Omicron variant includes approximately 30 genomic changes, including the 69-70del S gene mutation and 15 mutations in the receptor binding domain. Concerns associated with the Omicron variant include its increased transmissibility, apparent reduction in vaccine effectiveness, and increased risk for reinfections. Through much of 2021, the Delta variant was the dominant form of the virus in the United States and in many other parts of the world. Compared to the reference form of SARS-CoV-2, the Delta variant attributes include increased transmissibility and, in some cases, reduced neutralization by monoclonal antibody treatments and post-vaccination sera. The Alpha variant is estimated to be 70% more transmissible than the original SARS-CoV-2, and early studies indicate the possibility of increased risk of death in patients infected with this variant. The Beta variant is reportedly more contagious than the original SARS-CoV-2 and may be associated with poor response to antibody-based therapies.

Assays designed for earlier variants of SARS-CoV-2 may have decreased efficacy in detecting such newly emerging variants. For example, the Omicron, Delta, and Alpha variants have several mutations associated with the S protein region, which is a common target for detection assays. These mutations are substantial enough that some test components and protocols designed for earlier SARS-CoV-2 forms may show a negative result for the S protein region. This phenomenon is often referred to as “S gene dropout.” Although these and other new variants may still be detectable with some of the assays designed for earlier variants, their emergence highlights the continued risk that further mutations will render earlier assays less effective or even ineffective.

Given the present and continuing emergence of new and/or variant coronaviruses, there is an urgent need to develop methods for the rapid detection and characterization of existing and future coronavirus strains so that appropriate treatment and infection control measures can be properly instituted in a timely manner. In particular, given that SARS-CoV-2 is expected to continue to mutate and develop new variants as the pandemic progresses, there is an urgent need to develop assays capable of effectively detecting new variants and/or capable of distinguishing between different variants and/or mutations of SARS-CoV-2.

Accordingly, there are several disadvantages with current methods, systems, compositions, and kits for detecting SARS-CoV-2, particularly as new mutations and variants continue to emerge, that are addressed by the compositions, methods, and kits disclosed herein.

### BRIEF DESCRIPTION OF THE DRAWINGS

FIGS. 1A and 1B illustrate the sequence identity between SARS-CoV-2 and three closely related coronaviruses, namely, Bat-SL-CoVZC45, Bat-SL-CoVZXC21 and SARS-CoVGZO2. (Taken from Lu et al. “Genomic characterization and epidemiology of 2019 novel coronavirus: implications for virus origins and receptor binding.” Roujian Lu et al., The Lancet, Elsevier, Available online 30 Jan. 2020).

FIG. 1C is a schematic diagram of the RNA genome of SARS-CoV-2, illustrating potential target regions that assays described herein may be targeted toward.

FIG. 1D is a schematic illustration of the SARS-CoV-2 virion structure.

FIGS. 2A and 2B provide an overview of an exemplary assay design for detecting SARS-CoV-2 and distinguishing between a reference form and one or more variant forms.

FIGS. 2C and 2D illustrate primers and probes that may be utilized for the identification of SARS-CoV-2 variants and/or mutations and/or for distinguishing such SARS-CoV-2 variants from other SARS-CoV-2 variants, including distinguishing from a reference SARS-CoV-2 associated with the originally described SARS-CoV-2.

FIGS. 3A-3E illustrate allelic discrimination plots resulting from the use of assays targeting del69V70, N501Y, P681H, K417N, and E484K mutations, showing that assays as described herein are able to effectively discriminate between SARS-CoV-2 mutant sequences and a co-mixed SARS-CoV-2 reference sequence.

### DETAILED DESCRIPTION

All publications and patent applications cited herein, as well as the Appendices attached hereto, are incorporated by reference in their entirety for all purposes to the same extent as if each individual Appendix, publication or patent application were specifically and individually indicated to be so incorporated by reference. Although the Appendices attached hereto may include particular examples that reference specific target nucleic acids, formulations, and process steps, it will be understood that these examples may be modified by using any of the formulations, components, and/or process steps described elsewhere herein, including by using any of the primers and/or probes described herein. Further, although the present invention has been described in some detail by way of illustration and example for purposes of clarity and understanding, it will be apparent that certain changes and modifications may be practiced within the scope of the spirit and substance of this disclosure and of the appended claims.

### Overview of Compositions, Systems, and Kits for Detection of Viral Sequences

Given the present and continuing emergence of new genetic mutations and variants of interest and the importance of understanding the biological impact of such mutations in various contexts (such as, for example, tracking and diagnosis of the presence of infectious organisms, cancer-associated mutations, genealogy, and the like), there is an urgent need to develop compositions, kits, methods, and the like for the accurate and rapid detection and characterization of genetically variable targets. In the case of SARS-CoV-2, for example, appropriate variant tracking can be implemented so that treatment and infection control measures can be properly instituted in a timely manner. Each misidentified or misdiagnosed instance of SARS-CoV-2 infection further convolutes the epidemiological data and prevents the implementation of appropriate, informed solutions that may help reign in the pandemic. For example, missed diagnoses may be related to the failure of present detection assays to properly detect new and emerging variants of SARS-CoV-2.

In some embodiments, the present disclosure relates to compositions, kits and methods for detection of coronaviruses, in particular the coronavirus SARS-CoV-2. Also disclosed herein are compositions, kits, and methods for detecting one or more mutations and/or variants of SARS-CoV-2. Also disclosed herein are compositions, kits, and methods for determining whether detected SARS-CoV-2 one or more mutations associated with a “variant” form of SARS-CoV-2 or one or more alleles associated with the “reference” SARS-CoV-2 genome (as those terms are defined herein). For example, some embodiments relate to assays capable of detecting the presence of reference SARS-CoV-2, one or more variants, or combinations thereof. When an example “embodiment” or a particular “assay” is described herein, it will be understood that the features of the embodiment may be applicable to a composition (e.g., the particular physical components of an assay such as primers and/or probes), a kit (e.g., primers and/or probes and additional buffers, reagents, etc.), or a method (e.g., a process for detecting target nucleic acids) as appropriate. For simplicity, many embodiments are presented by describing “assays”, but it will be understood that the associated methods of using the assays are also intended to form part of this disclosure.

The SARS-CoV-2 virus, also known as 2019-nCoV, is associated with the human respiratory disease COVID-19. The virus isolated from early cases of COVID-19 was provisionally named 2019-nCoV. The Coronavirus Study Group of the International Committee on Taxonomy of Viruses has subsequently given the official designation of SARS-CoV-2. For the purposes of this disclosure SARS-CoV-2 and 2019-nCoV are considered to refer to the same virus.

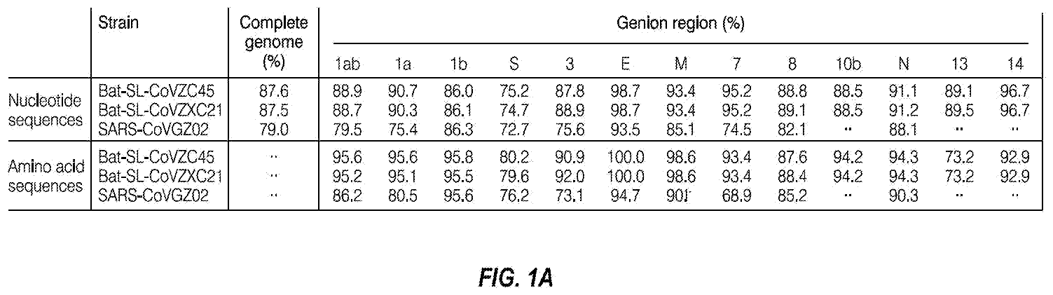

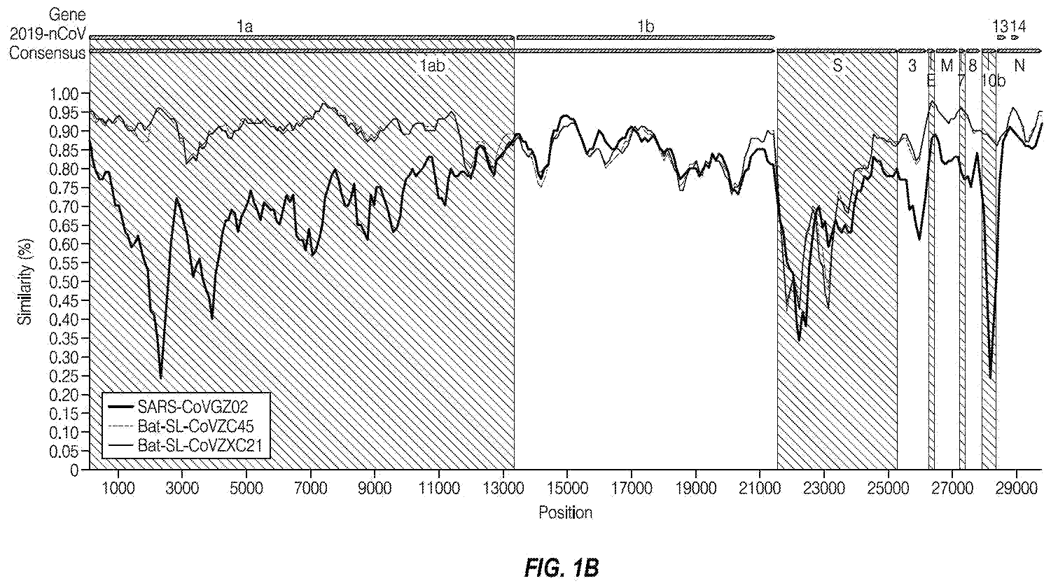

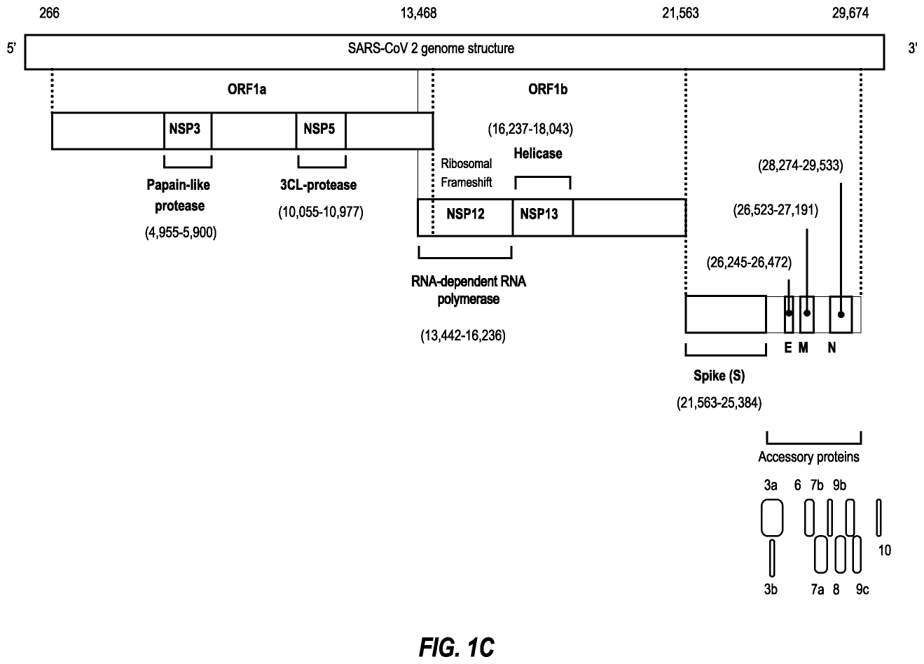

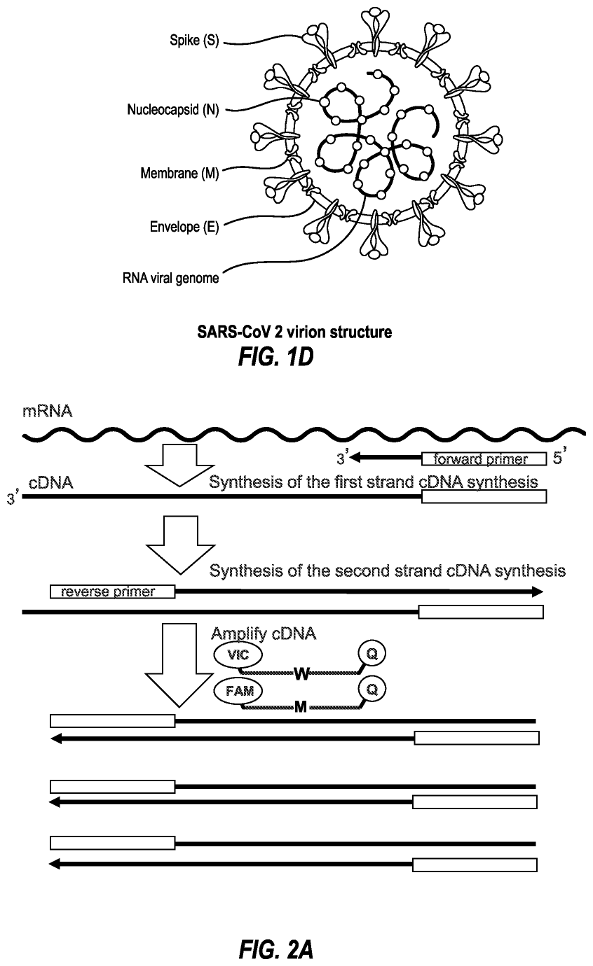

Initial genetic characterization SARS-CoV-2 was reported by Lu et al. (“Genomic characterization and epidemiology of 2019 novel coronavirus: implications for virus origins and receptor binding.” Roujian Lu et al., The Lancet, Elsevier, Available online 30 Jan. 2020). Lu identified three coronavirus that show close homology to SARS-CoV-2: Bat-SL-CoVZC45, Bat-SL-CoVZXC21 and SARS-CoVGZO2. The sequence identity between these strains is depicted in FIGS. 1A and 1B. This analysis identified three genetic regions with significant variability between SARS-CoV-2 and the other viruses, specifically in the viral genes encoding the viral proteins for an Orf protein (e.g., Orf1a, Orf1b, Orf1ab, Orf8), the S protein and the N protein.

The genetic sequence of this “reference” form of SARS-CoV-2 is based on the sequence associated with NCBI accession no. NC_045512.2 (see GenBank: MN908947.3) which describes a genome of 29,903 base pairs. As an example of certain regions of the “reference” genome, the region of bp 1000 to bp 3000 associated with Orf1ab, is shown in Table 1.

TABLE 1
(bp 1 corresponds to bp 1000 of MN908947) SEQ ID NO: 7311.

```
      1 tgaaaagagc tatgaattgc agacaccttt tgaaattaaa ttggcaaaga aatttgacac
    61 cttcaatggg gaatgtccaa attttgtatt tcccttaaat tccataatca agactattca
  121 accaagggtt gaaaagaaaa agcttgatgg ctttatgggt agaattcgat ctgtctatcc
  181 agttgcgtca ccaaatgaat gcaaccaaat gtgcctttca actctcatga agtgtgatca
  241 ttgtggtgaa acttcatggc agacgggcga ttttgttaaa gccacttgcg aattttgtgg
  301 cactgagaat ttgactaaag aaggtgccac tacttgtggt tacttacccc aaaatgctgt
  361 tgttaaaatt tattgtccag catgtcacaa ttcagaagta ggacctgagc atagtcttgc
  421 cgaataccat aatgaatctg gcttgaaaac cattcttcgt aagggtggtc gcactattgc
  481 ctttggaggc tgtgtgttct cttatgttgg ttgccataac aagtgtgcct attgggttcc
  541 acgtgctagc gctaacatag gttgtaacca tacaggtgtt gttggagaag gttccgaagg
  601 tcttaatgac aaccttcttg aaatactcca aaaagagaaa gtcaacatca atattgttgg
  661 tgactttaaa cttaatgaag agatcgccat tattttggca tctttttctg cttccacaag
  721 tgcttttgtg gaaactgtga aaggtttgga ttataaagca ttcaaacaaa ttgttgaatc
  781 ctgtggtaat tttaaagtta caaaaggaaa agctaaaaaa ggtgcctgga atattggtga
  841 acagaaatca atactgagtc ctctttatgc atttgcatca gaggctgctc gtgttgtacg
  901 atcaattttc tcccgcactc ttgaaactgc tcaaaattct gtgcgtgttt tacagaaggc
  961 cgctataaca atactagatg gaatttcaca gtattcactg agactcattg atgctatgat
1021 gttcacatct gatttggcta ctaacaatct agttgtaatg gcctacatta caggtggtgt
1081 tgttcagttg acttcgcagt ggctaactaa catctttggc actgtttatg aaaaactcaa
1141 acccgtcctt gattggcttg aagagaagtt taaggaaggt gtagagtttc ttagagacgg
1201 ttgggaaatt gttaaattta tctcaacctg tgcttgtgaa attgtcggtg gacaaattgt
1261 cacctgtgca aaggaaatta aggagagtgt tcagacattc tttaagcttg taaataaatt
1321 tttggctttg tgtgctgact ctatcattat tggtggagct aaacttaaag ccttgaattt
1381 aggtgaaaca tttgtcacgc actcaaaggg attgtacaga aagtgtgtta aatccagaga
1441 agaaactggc ctactcatgc ctctaaaagc cccaaaagaa attatottct tagagggaga
1501 aacacttccc acagaagtgt taacagagga agttgtcttg aaaactggtg atttacaacc
1561 attagaacaa cctactagtg aagctgttga agctccattg gttggtacac cagtttgtat
1621 taacgggctt atgttgctcg aaatcaaaga cacagaaaag tactgtgccc ttgcacctaa
1681 tatgatggta acaaacaata ccttcacact caaaggcggt gcaccaacaa aggttacttt
1741 tggtgatgac actgtgatag aagtgcaagg ttacaagagt gtgaatatca cttttgaact
1801 tgatgaaagg attgataaag tacttaatga gaagtgctct gcctatacag ttgaactcgg
1861 tacagaagta aatgagttcg cctgtgttgt ggcagatgct gtcataaaaa ctttgcaacc
1921 agtatctgaa ttacttacac cactgggcat tgatttagat gagtggagta tggctacata
1981 ctacttattt gatgagtctg g
```

For the S gene region, bp 21564 thru 23564 is shown in Table 2.

TABLE 2
(bp 1 corresponds to bp 21564 of MN908947) SEQ ID NO: 7312.

```
      1 tgtttgtttt tcttgtttta ttgccactag tctctagtca gtgtgttaat cttacaacca
    61 gaactcaatt accccctgca tacactaatt ctttcacacg tggtgtttat taccctgaca
  121 aagttttcag atcctcagtt ttacattcaa ctcaggactt gttcttacct ttcttttcca
  181 atgttacttg gttccatgct atacatgtct ctgggaccaa tggtactaag aggtttgata
  241 accctgtcct accatttaat gatggtgttt attttgcttc cactgagaag tctaacataa
  301 taagaggctg gatttttggt actactttag attcgaagac ccagtcccta cttattgtta
  361 ataacgctac taatgttgtt attaaagtct gtgaatttca attttgtaat gatccatttt
  421 tgggtgttta ttaccacaaa aacaacaaaa gttggatgga aagtgagttc agagtttatt
  481 ctagtgcgaa taattgcact tttgaatatg tctctcagcc ttttcttatg gaccttgaag
  541 gaaaacaggg taatttcaaa aatcttaggg aatttgtgtt taagaatatt gatggttatt
  601 ttaaaatata ttctaagcac acgcctatta atttagtgcg tgatctccct cagggttttt
  661 cggctttaga accattggta gatttgccaa taggtattaa catcactagg tttcaaactt
  721 tacttgcttt acatagaagt tatttgactc ctggtgattc ttcttcaggt tggacagctg
  781 gtgctgcagc ttattatgtg ggttatcttc aacctaggac ttttctatta aaatataatg
  841 aaaatggaac cattacagat gctgtagact gtgcacttga ccctctctca gaaacaaagt
  901 gtacgttgaa atccttcact gtagaaaaag gaatctatca aacttctaac tttagagtcc
  961 aaccaacaga atctattgtt agatttccta atattacaaa cttgtgccct tttggtgaag
1021 tttttaacgc caccagattt gcatctgttt atgcttggaa caggaagaga atcagcaact
1081 gtgttgctga ttattctgtc ctatataatt ccgcatcatt ttccactttt aagtgttatg
1141 gagtgtctcc tactaaatta aatgatctct gctttactaa tgtctatgca gattcatttg
1201 taattagagg tgatgaagtc agacaaatcg ctccagggca aactggaaag attgctgatt
1261 ataattataa attaccagat gattttacag gctgcgttat agcttggaat tctaacaatc
1321 ttgattctaa ggttggtggt aattataatt acctgtatag attgtttagg aagtctaatc
1381 tcaaaccttt tgagagagat atttcaactg aaatctatca ggccggtagc acaccttgta
1441 atggtgttga aggttttaat tgttactttc ctttacaatc atatggtttc caacccacta
1501 atggtgttgg ttaccaacca tacagagtag tagtactttc ttttgaactt ctacatgcac
1561 cagcaactgt ttgtggacct aaaaagtcta ctaatttggt taaaaacaaa tgtgtcaatt
1621 tcaacttcaa tggtttaaca ggcacaggtg ttcttactga gtctaacaaa aagtttctgc
1681 ctttccaaca atttggcaga gacattgctg acactactga tgctgtccgt gatccacaga
1741 cacttgagat tcttgacatt acaccatgtt cttttggtgg tgtcagtgtt ataacaccag
1801 gaacaaatac ttctaaccag gttgctgttc tttatcagga tgttaactgc acagaagtcc
1861 ctgttgctat tcatgcagat caacttactc ctacttggcg tgtttattct acaggttcta
1921 atgtttttca aacacgtgca ggctgtttaa taggggctga acatgtcaac aactcatatg
1981 agtgtgacat acccattggt g
```

For the N gene region, bp 28275 thru 29558 is shown in Table 3.

TABLE 3
(bp 1 corresponds to bp 28275 of MN908947) SEQ ID NO: 7313.

```
      1 tgtctgataa tggaccccaa aatcagcgaa atgcaccccg cattacgttt ggtggaccct
    61 cagattcaac tggcagtaac cagaatggag aacgcagtgg ggcgcgatca aaacaacgtc
  121 ggccccaagg tttacccaat aatactgcgt cttggttcac cgctctcact caacatggca
  181 aggaagacct taaattccct cgaggacaag gcgttccaat taacaccaat agcagtccag
  241 atgaccaaat tggctactac cgaagagcta ccagacgaat tcgtggtggt gacggtaaaa
  301 tgaaagatct cagtccaaga tggtatttct actacctagg aactgggcca gaagctggac
  361 ttccctatgg tgctaacaaa gacggcatca tatgggttgc aactgaggga gccttgaata
  421 caccaaaaga tcacattggc acccgcaatc ctgctaacaa tgctgcaatc gtgctacaac
  481 ttcctcaagg aacaacattg ccaaaaggct tctacgcaga agggagcaga ggcggcagtc
  541 aagcctcttc tcgttcctca tcacgtagtc gcaacagttc aagaaattca actccaggca
  601 gcagtagggg aacttctcct gctagaatgg ctggcaatgg cggtgatgct gctcttgctt
  661 tgctgctgct tgacagattg aaccagcttg agagcaaaat gtctggtaaa ggccaacaac
  721 aacaaggcca aactgtcact aagaaatctg ctgctgaggc ttctaagaag cctcggcaaa
  781 aacgtactgc cactaaagca tacaatgtaa cacaagcttt cggcagacgt ggtccagaac
  841 aaacccaagg aaattttggg gaccaggaac taatcagaca aggaactgat tacaaacatt
  901 ggccgcaaat tgcacaattt gcccccagcg cttcagcgtt cttcggaatg tcgcgcattg
  961 gcatggaagt cacaccttcg ggaacgtggt tgacctacac aggtgccatc aaattggatg
1021 acaaagatcc aaatttcaaa gatcaagtca ttttgctgaa taagcatatt gacgcataca
1081 aaacattccc accaacagag cctaaaaagg acaaaaagaa gaaggctgat gaaactcaag
1141 ccttaccgca gagacagaag aaacagcaaa ctgtgactct tcttcctgct gcagatttgg
1201 atgatttctc caaacaattg caacaatcca tgagcagtgc tgactcaact caggcctaaa
1261 ctcatgcaga ccacacaagg caga
```

As used herein, in the context of SARS-CoV-2 as a target organism, a “mutant” or “variant” has one or more mutations (e.g., SNP or deletion mutations) in one or more of the above regions and/or other sites of the genomic sequence as compared to the “reference” SARS-CoV-2.

Several SARS-CoV-2 qPCR based tests currently on the market are designed to target one or more regions shown in Tables 1-3. Examples include the kit developed by the CDC containing probes targeting the N protein; the kit developed by the Chinese CDC targeting the N and Orf proteins, as well as the WHO kit targeting the N protein, the E protein, and the closely related RdRp SARS/Wuhan coronavirus. However, as discussed above, new mutations and variants of SARS-CoV-2 have emerged and continue to emerge, and the currently available assays are not optimized for such newly emerging variants. The currently available assays cannot be utilized to identify new SARS-CoV-2 variants or distinguish between different variants and may even fail to detect the presence of certain SARS-CoV-2 variants and thus lead to false negative test results. In contrast, embodiments described herein can be beneficially utilized to detect and identify various emerging mutations and variants of SARS-CoV-2, to distinguish variant forms from the original reference form, and/or to distinguish variant forms from one another.

Further, because SARS-CoV-2 is an RNA virus, it can mutate with relatively high frequency, meaning additional mutations and variants will continue to emerge over time. Specific detection of SARS-CoV-2 can be enhanced even in the case of such future variants by targeting multiple regions of the SARS-CoV-2 genome (e.g., by combining assays specific for the N gene, S gene, Orf1 regions, and/or other genomic regions) thereby compensating for possible virus mutations and/or SARS-CoV-2 variants. FIG. 1C is a diagram of the SARS-CoV-2 RNA genome showing particular regions that may be targeted. As shown, potential target genes include the Orf1a, Orf1b, S, E, M, and N genes, among several other accessory proteins. The SARS-CoV-2 genome encodes two large genes Orf1a and Orf1b, which encode 16 non-structural proteins (NSP1-NSP16). These NSPs are processed to form a replication-transcription complex (RTC) that is involved in genome transcription and replication. With additional reference to FIG. 1D, which illustrates the SARS-CoV-2 virion structure, the structural genes encode the structural proteins, spike (S), envelope (E), membrane (M), and nucleocapsid (N). The accessory proteins are unique to SARS-CoV-2 in terms of number, genomic organization, sequence, and function.

For example, in some embodiments, positive identification of SARS-CoV-2 is determined by detection of N gene and S gene targets. In some embodiments, positive identification of SARS-CoV-2 is determined by detection of N gene and Orf1 region targets. In some embodiments, positive identification of SARS-CoV-2 is determined by detection of S gene and Orf1 targets. In some embodiments, positive identification of SARS-CoV-2 is determined by detection of N gene, S gene, and Orf1 region targets. In some embodiments, positive identification of a SARS-CoV-2 variant is determined by detection of at least one of an N gene, S gene and Orf1 region target combined with non-detection of at least one of an N gene, S gene and Orf1 region target. This may include, for example, non-detection of the S gene due to S gene dropout common to several variants along with positive detection of one or more other targets. In some embodiments, such as where only 1 of 2 or 3 targets is detected (e.g., N gene or Orf1 region target(s) detected and S gene target not detected) or 2 of 3 targets are detected (e.g., N gene and Orf1 region target(s) detected and S gene target not detected), the methods as described herein can further include confirmation by Sanger sequencing for determination of a positive diagnosis of SARS-CoV-2 and/or specifying the variant involved.

Table 4 illustrates some of the mutations that have occurred in the SARS-CoV-2 genome, as well as some of their associated variants, where known. The numbering system used to designate these mutations is based on the “reference” sequence as defined above. For example, the mutation “S.N501Y.AAT.TAT” refers to a mutant form of the spike (S) protein wherein amino acid residue no. 501 is changed from asparagine (A) to tyrosine (Y). The latter portion of the label may recite the mutation according to standard nucleotide variation, or as in the example “AAT.TAT” compares the reference codon to the mutant codon and illustrates that the mutation is associated with a change from an adenine (A) to a thymine (T) (i.e., the AAT of the reference codon is changed to a TAT in the mutant codon). Mutations may be listed according to nucleotide variation and/or according to amino acid variation. Not all mutations are necessarily within a gene region and thus some labels may omit a gene prefix. Note that RNA comprises uracil (U), but notation included herein may sometimes simply refer to the corresponding DNA base pair thymine (T). The initial part of the label specific to the gene or protein involved and/or the latter portion of the label specific to the nucleotide mutation may occasionally be dropped from the label for convenience. The latter portion of the label may also be shortened to simply show the single reference nucleotide and mutant nucleotide, rather than the entire reference and mutant codon. Those with skill in the art will readily recognize the mutation nomenclature used herein.

TABLE 4
SARS-CoV-2 Mutations

| | | | Earliest Documented |
|---|---|---|---|
| Mutation | WHO Label | Associated Variants | Samples |
| S.D215G.GAT.GGT | Beta | B.1.351 | South Africa |
| S.D614G.GAT.GGT | Various | B.1.1.207, P.1, | Nigeria, United |
| | | B.1.1.33, B.1.1.7, | Kingdom, South Africa, |
| | | B.1.177, B.1.258, | Brazil/Amazon |
| | | B.1.351, B.1.525, | |
| | | Mink variant | |
| S.delH69V70 | Alpha | B.1.1.7, B.1.258, | United Kingdom |
| | | B.1.525 | |
| S.delY144 | Alpha | B.1.1.7 | United Kingdom |
| S.E484K.GAA.AAA | Beta, | P.1, B.1.1.33, | South Africa, Brazil |
| | Gamma, | B.1.351, B.1.525 | |
| | Eta | | |
| S.E484Q.GAA.CAA | Delta | B.1.617, B.1.617.1, | India |
| | (absence | B.1.617.3 | |
| | of this | | |
| | mutation) | | |
| S.F888L.TTT.CTT | Eta | B.1.525 | 20A/S: 484K United |
| | | | Kingdom |
| S.K417N.AAG.AAT | Beta | B.1.351 | South Africa |
| S.K417T.AAG.ACG | Gamma | P.1 | Brazil/Amazon |
| S.L18F.CTT.TTT | Gamma | B.1.351, P.1, P.1.15, | South Africa, |
| | | P.1.7, P.1.17, P.1.2, | Brazil/Amazon |
| | | P.1.12, P.1.6, P.1.14 | |
| S.L452R.CTG.CGG | Delta, | B.1.617, B.1.617.1, | India, California |
| | Epsilon | B.1.617.2, B.1.617.3, | |
| | | B.1.429 | |
| S.N439K.AAC.AAA | | B.1.258, B.1.466.2 | |
| S.N501Y.AAT.TAT | Alpha, | P.1, B.1.1.7, B.1.351 | United Kingdom, South |
| | Beta, | | African, Brazil/ |
| | Gamma | | Amazon |
| S.P681H.CCT.CAT | Alpha | B.1.1.207, B.1.1.7 | Nigeria, United |
| | | | Kingdom |
| S.P681R.CCT.CGT | Delta | B.1.617.1, B.1.617.2, | India |
| | | B.1.617.3 | |
| S.S13I.AGT.ATT | Epsilon | B.1.427, B.1.429 | California |
| S.S477N.AGC.AAC | Iota | B.1.526, B.1.1.397 | New York |
| S.T20N.ACC.AAC | Gamma | P.1, P.1.12, P.1.14, | Brazil/Amazon |
| | | P.1.2, P.1.6, P.1.15, | |
| | | P.1.17, P.1.7 | |
| S.V1176F.GTT.TTT | | P.2 | Brazil |
| Orflab.A13057T | Mu | B.1.621 | Colombia |
| Orf1.A1708D.GCT.GAT | Alpha | B.1.1.7, Q.7 | United Kingdom |
| S.A222V.GCT.GTT | | B.1.177 | |
| Orflab.A2710T | Omicron | B.1.1.529 | Various |
| CoV1.A28272T | Mu, Epsilon | B.1.621, A.2.5.2, | Colombia |
| | | B.1.429 | |
| S.A570D.GCT.GAT | Alpha | B.1.1.7, Q.7 | United Kingdom |
| S.A701V.GCA.GTA | Beta | B.1.351 | South Africa |
| N.D3L.GAT.CTA | Alpha | B.1.1.7, Q.7 | United Kingdom |
| S.D80A.GAT.GCT | Beta | B.1.351 | South Africa |
| S.delL242.244L | Beta | B.1.351 | South Africa |
| S.E583D.GAG.GAT | | B.177 | |
| S.E583D.GAG.GAC | | B.177 | |
| S.EFR156-158G | Delta | B.1.617.2 | India |
| S.F157L.TTC.TTA | | A.23 | |
| S.F157S.TTC_TCC | Iota | B.1.526, B.1.637 | United States |
| S.G339D.GGT_GAG | Omicron | B.1.1.529 | Various countries |
| S.G446V.GGT.GTT | | | |
| S.G769V.GGA.GTA | | | |
| S.H66D.CAT.GAT | | | |
| Orf1.K1795Q.AAA.CAA | Gamma | P.1, P.1.12, P.1.2, | |
| | | P.1.7, P.1.6, P.1.15 | |
| S.L452Q.CTG.CAG | Lambda | C.37 | Mink |
| S.N450K.AAT.AAG | | B.1.214 | |
| S.N460K.AAT.AAA | | | |
| S.N460K.AAT.AAG | | Q.7 | |
| S.N460T.AAT.ACT | | | |
| P5743S.CCA_TCA | Mu | B.1.621 | Colombia |
| Orf8.Q27ST.CAA.TAA | Alpha | B.1.1.7 | United Kingdom |
| S.Q493R.CAA_CGA | Omicron | B.1.1.529 | Various countries |
| S.Q498R.CAA.CGA | | | |
| S.Q52R.CAG.CGG | Eta | 1.525 | |
| S.Q613H.CAG.CAT | | A.23 | |
| S.Q677H.CAG.CAT | Eta | 1.525 | |
| S.Q677H.CAG.CAC | Eta | 1.525 | |
| S.R246I.AGA.ATA | Beta | B.1.351 | South Africa |
| S.R346K.AGA_AAA | Mu | B.1.621 | Colombia |
| S.S982A.TCA.GCA | Alpha | B.1.1.7, Q.7 | United Kingdom |
| S.Y145H.TAC.CAC | Delta+ | AY.4.2 | India |
| T1055A.ACA_GCA | Mu | B.1.621 | Colombia |
| Orflab.T13195C | Omicron | B.1.1.529 | Various |
| S.T19R.ACA.AGA | Delta | B.1.617.2 | India |
| S.T478K.ACA.AAA | Delta | B.1.617.2 | |
| S.T547K.ACA.AAA | Omicron | B.1.1.529 | Various |
| S.T716I.ACA.ATA | Alpha | B.1.1.7 | United Kingdom |
| S.T95I.ACT_ATT | Iota, Mu | B.1.526, B.1.621.1 | United States, |
| | | | Colombia |
| S.V367F.GTC.TTC | | A.23 | |
| S.W152L.TGG.TTG | | R.1 | |
| S.Y453F.TAT.TTT | | B.1.1.298, B.1.438.1 | Mink |
| S.D1118H.G_C | | B.1.1.7, Q.7 | United Kingdom |
| Orflab.G514G.A1807G | Eta | B.1.525 | |
| Orflab. F2387V.T7424G | Lambda | C.37 | |
| S.W152C.G22018T | Epsilon | B.1.429 | |
| S.T478R.C22995G | | B.1.637 | |
| S.N679K.T23599A | | C.1.2 | |
| S.N764K.C23854A | Omicron | BA.3, P.1.17, BA.1, | |
| | | BA.2 | |
| S.Q1071H.A24775T | | B.1.617.1 | |
| A28273T | | B.1.1.397 | |
| N.P142P.A28699G | Eta | B.1.525 | |
| N.G214C.G28913T | Lambda | C.37 | |

As explained above, these mutant variants, as well as others that may emerge in the future, may not be detected with the same efficacy using conventional diagnostic assays. Moreover, even if such variants are generally detected by conventional assays, the conventional assays are likely unable to determine whether the detected SARS-CoV-2 nucleic acid is associated with the reference form or with a particular mutation sequence in a specific region or gene (e.g., an S gene variant or mutation), such as for a particular single nucleotide polymorphism (SNP). This lack of resolution can prove problematic in attempts to track the spread and progression of such variants and/or requires more expensive and lengthy sequencing testing to identify particular variants.

### Example Assays & Associated Components

Embodiments disclosed herein include primers and optionally probes useful for the detection of SARS-CoV-2 and/or for the identification of variants thereof, in a sample (e.g., a biological or environmental sample). Such primers, oligonucleotides, and probes can be used in a nucleic acid assay (singleplex or multiplex) for detection and identification one or more nucleic acid targets in a sample. The singleplex and multiplex assays described herein demonstrate a high level of sensitivity, specificity, and accuracy. In some embodiments, an assay is designed to detect and differentiate between different forms of SARS-CoV-2. For example, an assay may be configured to detect the presence of SARS-CoV-2 nucleic acid within a biological sample and to identify whether the detected SARS-CoV-2 is from reference SARS-CoV-2 or from a variant. In some embodiments, for example, an assay includes differentially labeled probes such that at least one probe is designed for association with reference allele amplicons while at least one, different probe is designed for association with amplicons of a mutant/variant.

In some embodiments, an assay includes differentially labelled probes such that at least one probe is designed for association with amplicons of a first mutant/variant while at least one, different probe is designed for association with amplicons of a second mutant/variant. Additional labelled probes for additional mutants/variants and/or for the reference form may be further included. Thus, even though some embodiments may be “singleplex” in the sense that they include a single forward primer and single corresponding reverse primer for a single target genomic region, they are nevertheless “multiplex” in that they are capable of detecting one or more SARS-CoV-2 variants and/or distinguishing between forms of SARS-CoV-2 (e.g., distinguishing between reference SARS-CoV-2 and mutants/variants and/or distinguishing between different variants) due to the inclusion of different probes that associate with different SARS-CoV-2 variant forms.

In some embodiments, assays are configured to detect an amplification product of a particular target region by detecting a signal from a label (i.e., a detectable label) or other signal-generating process, where the signal indicates formation of the amplification product. In some embodiments, the label is attached to, or otherwise associated with, the corresponding forward primer and/or reverse primer used to generate the amplification product. Additionally, or alternatively, the label is attached to, or otherwise associated with, a probe configured to associate with a probe binding sequence within the target region. In some embodiments, the label is an optically detectable label. Alternatively, the label may be detectable via non-optical means including electronically, electrically, or using NMR, sound, radioactivity, and the like.

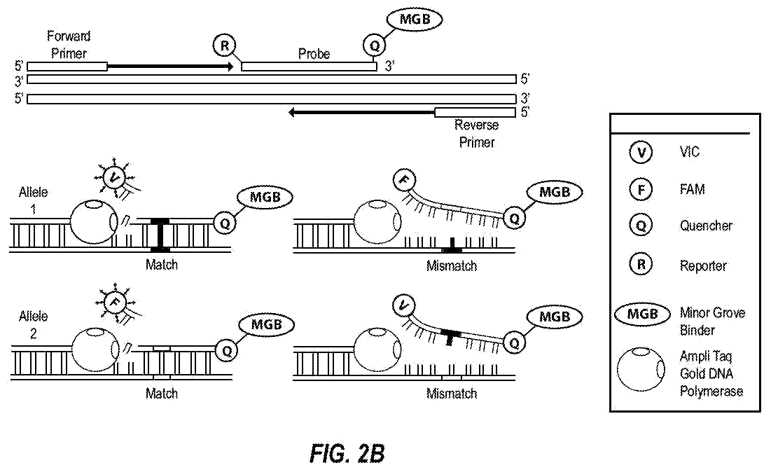

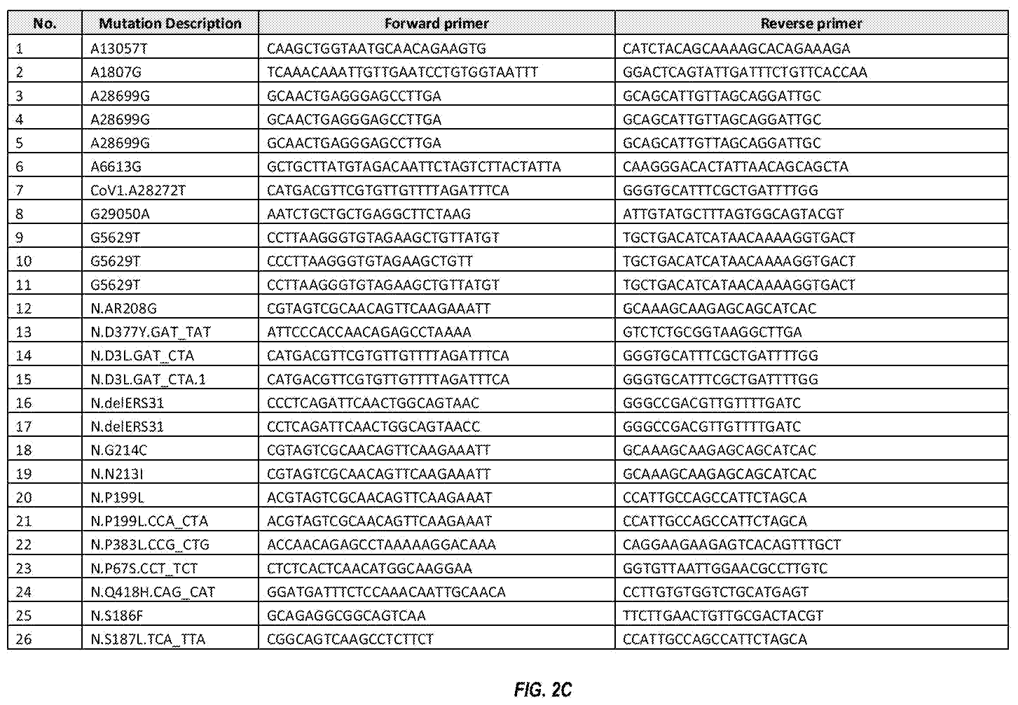

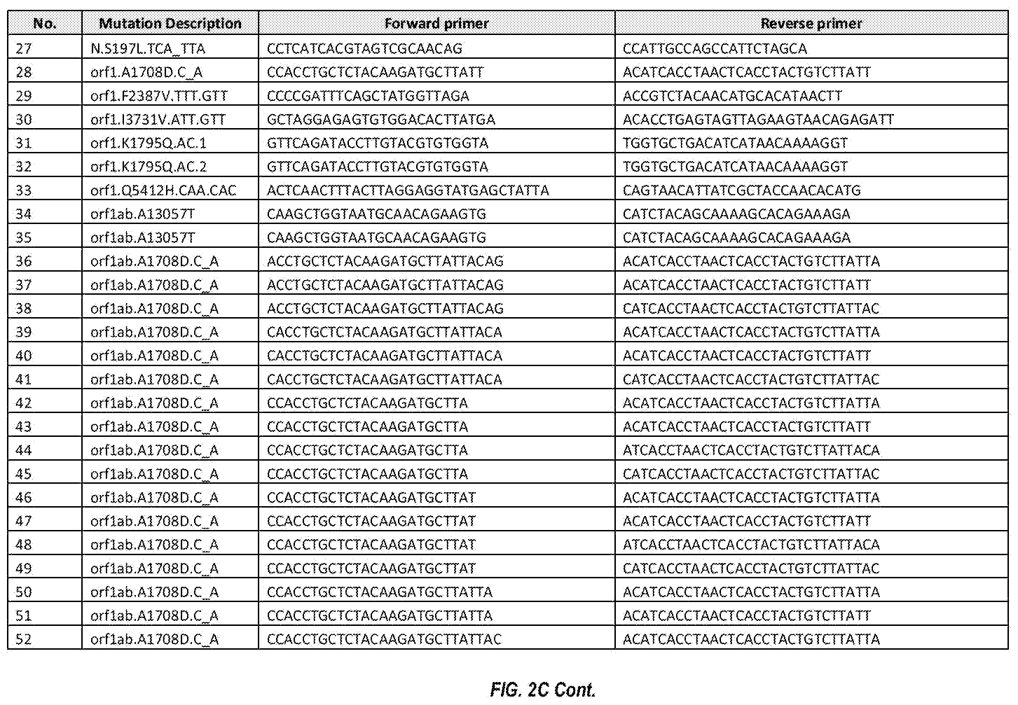

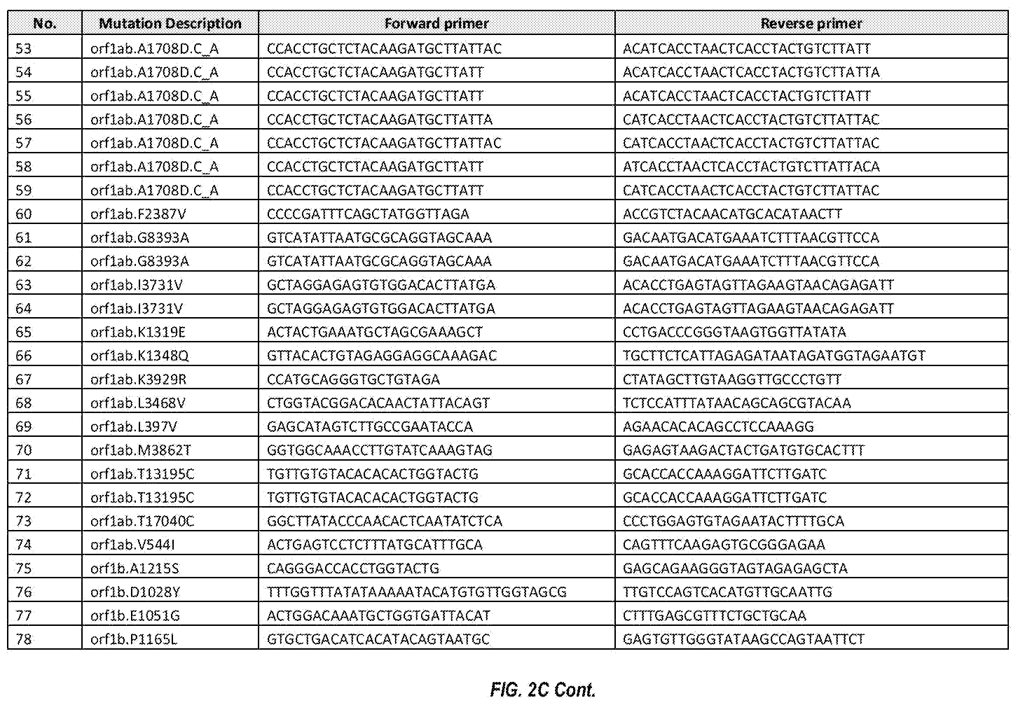

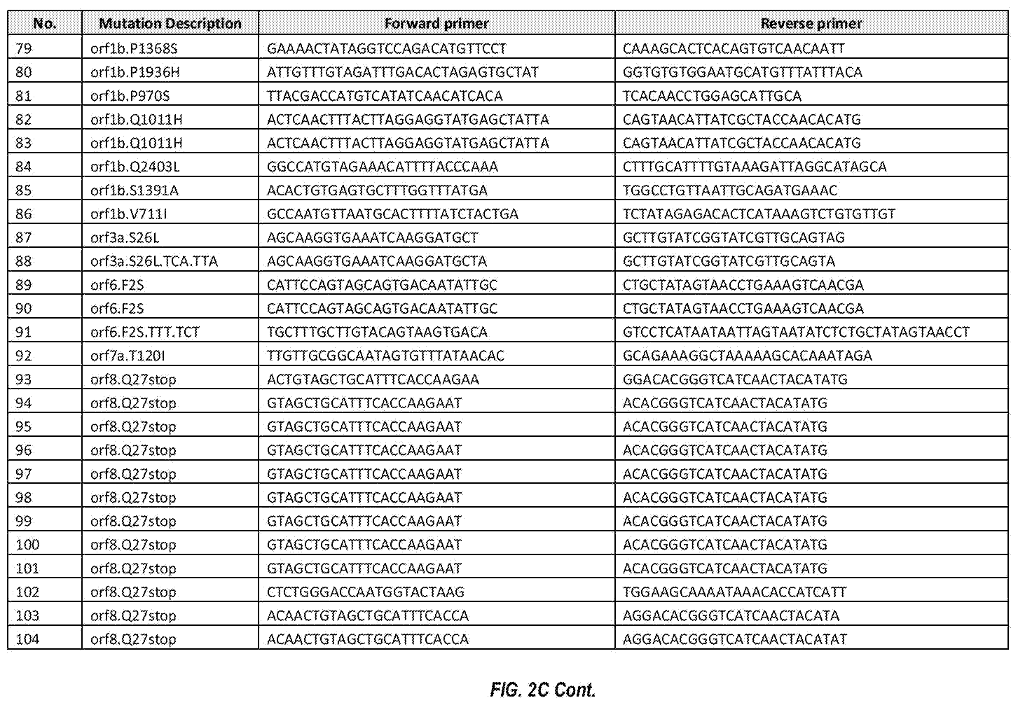

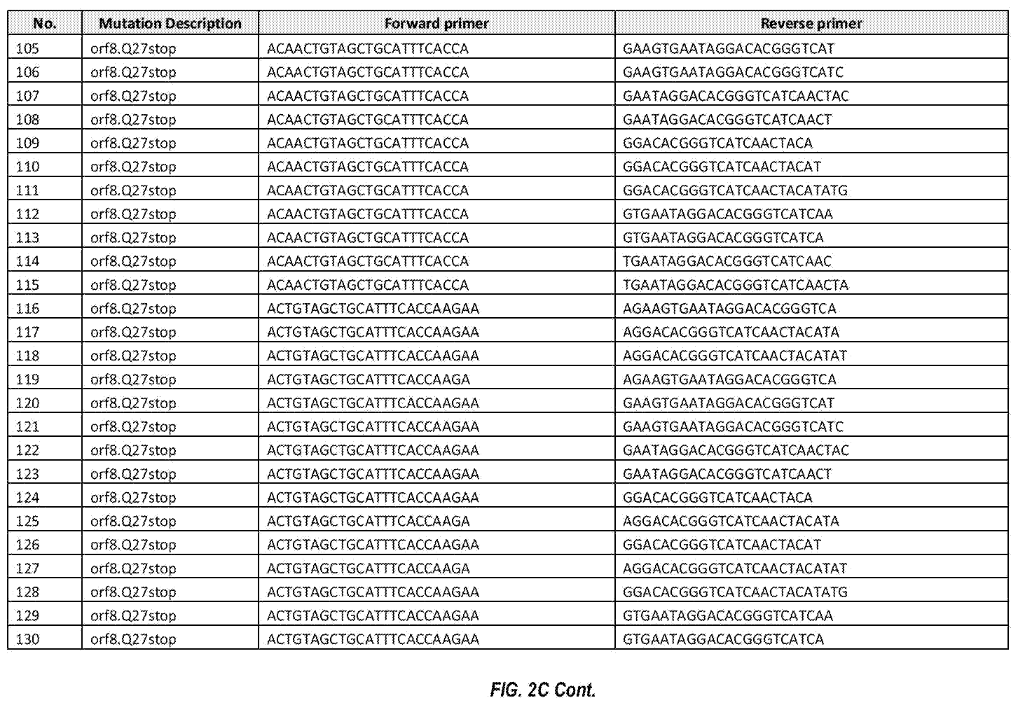

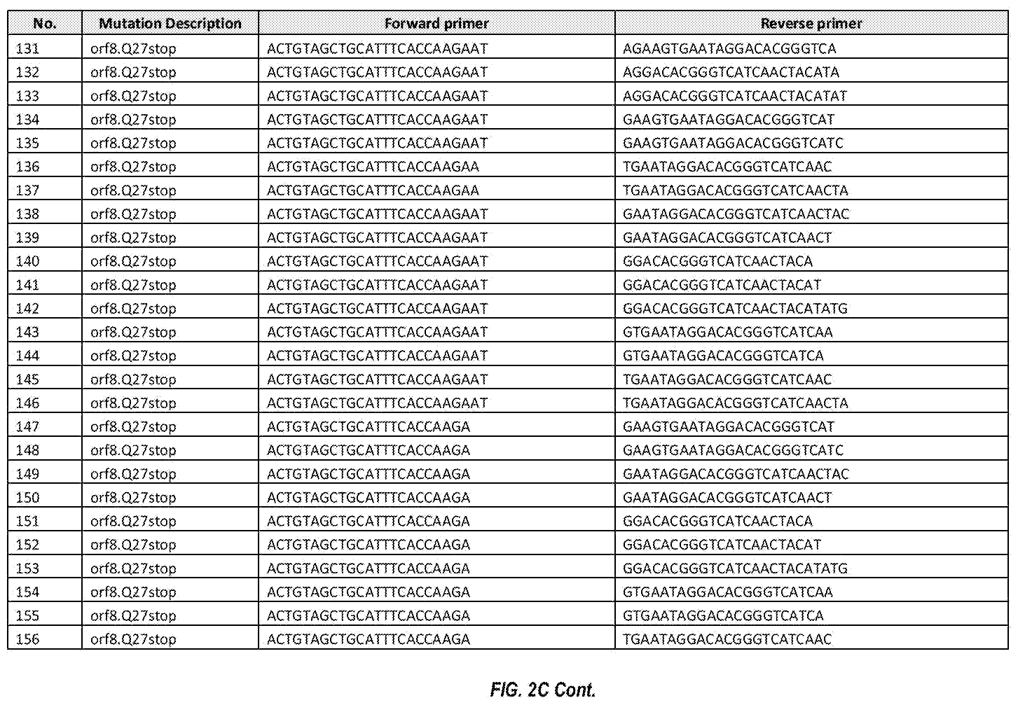

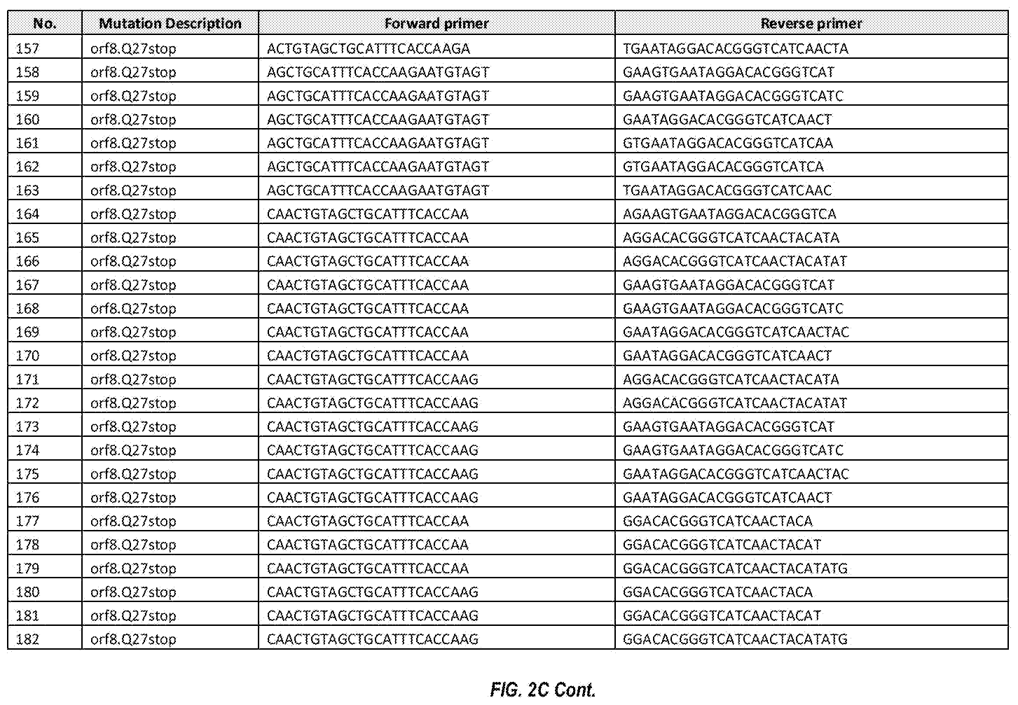

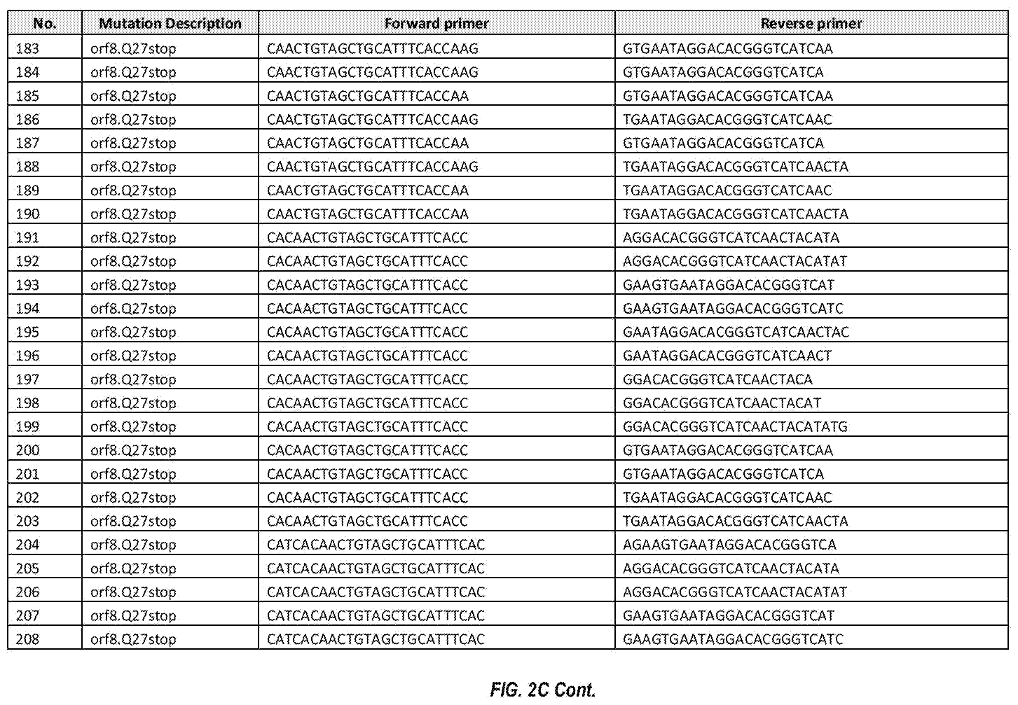

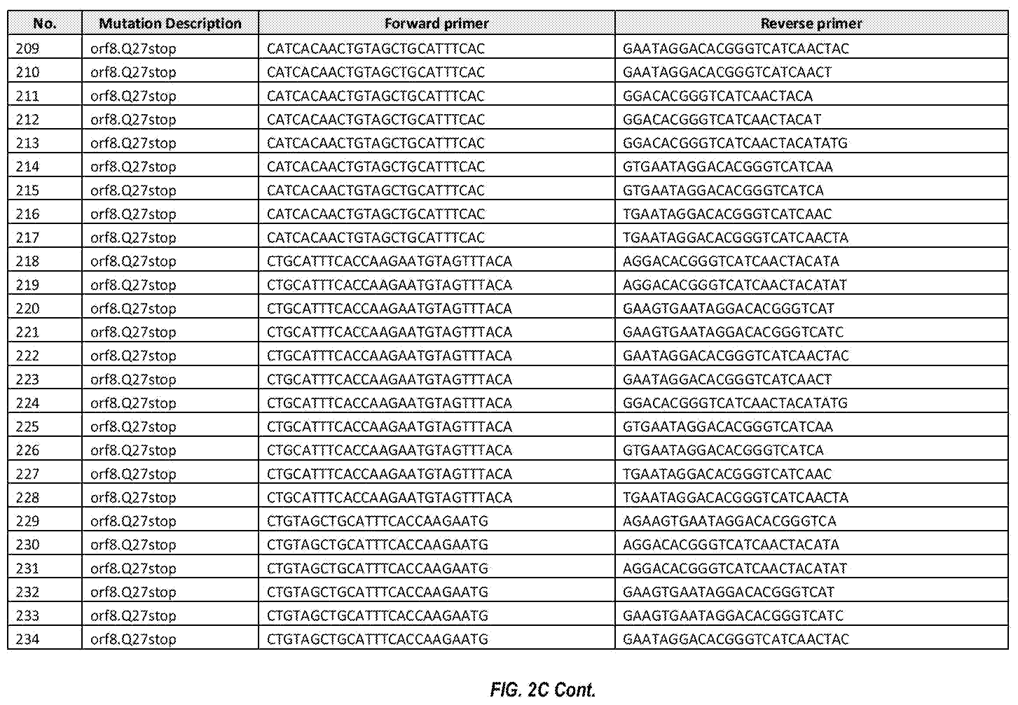

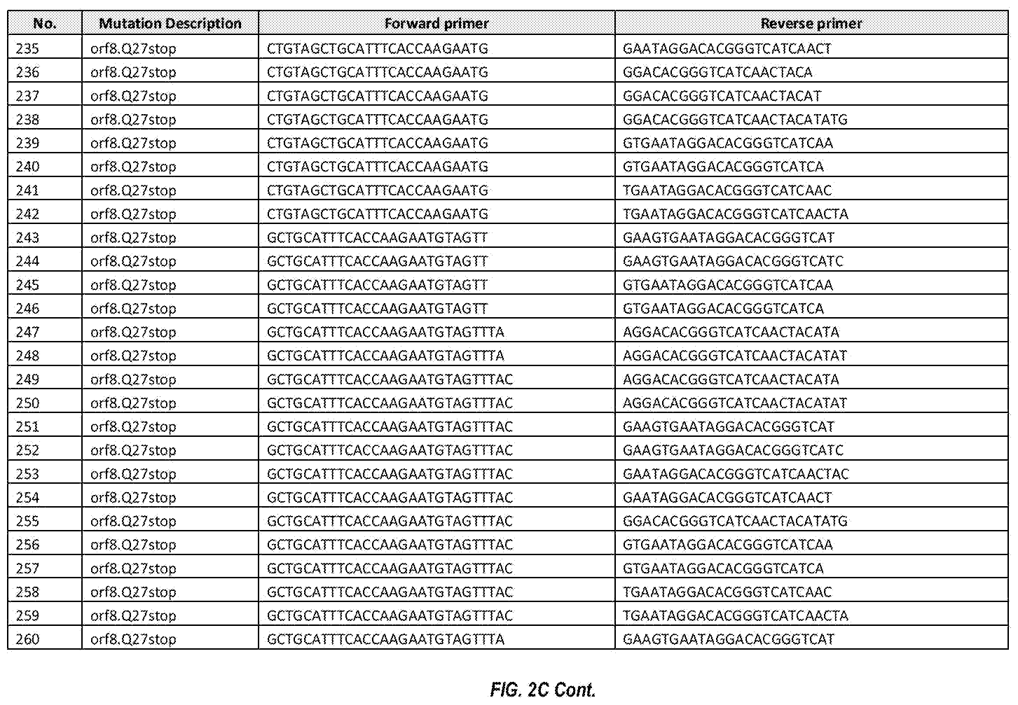

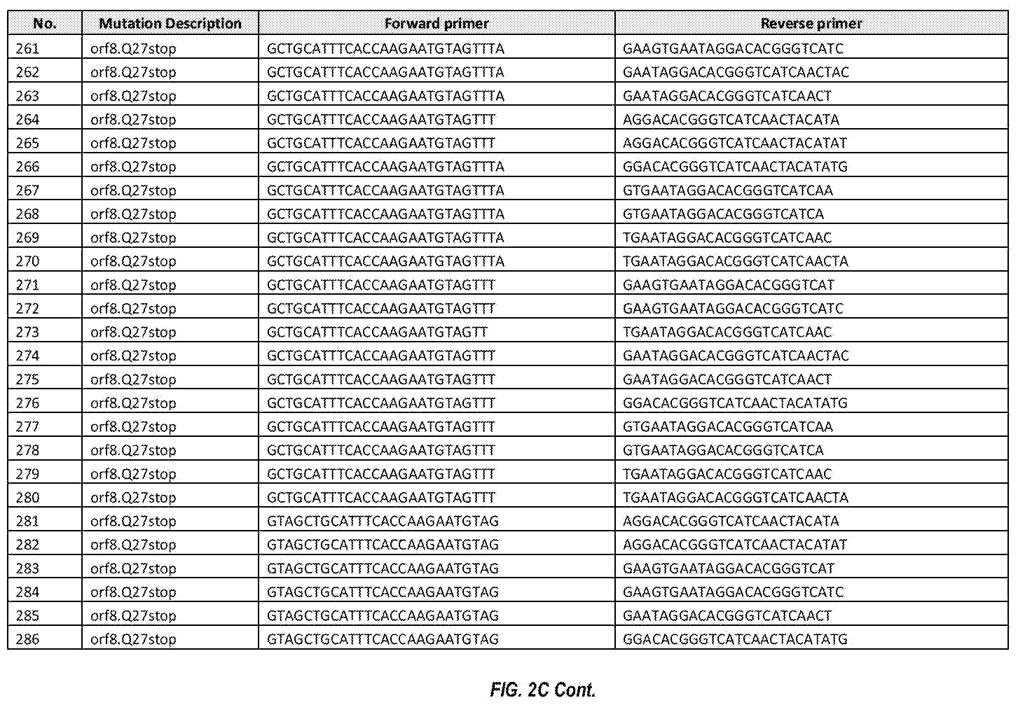

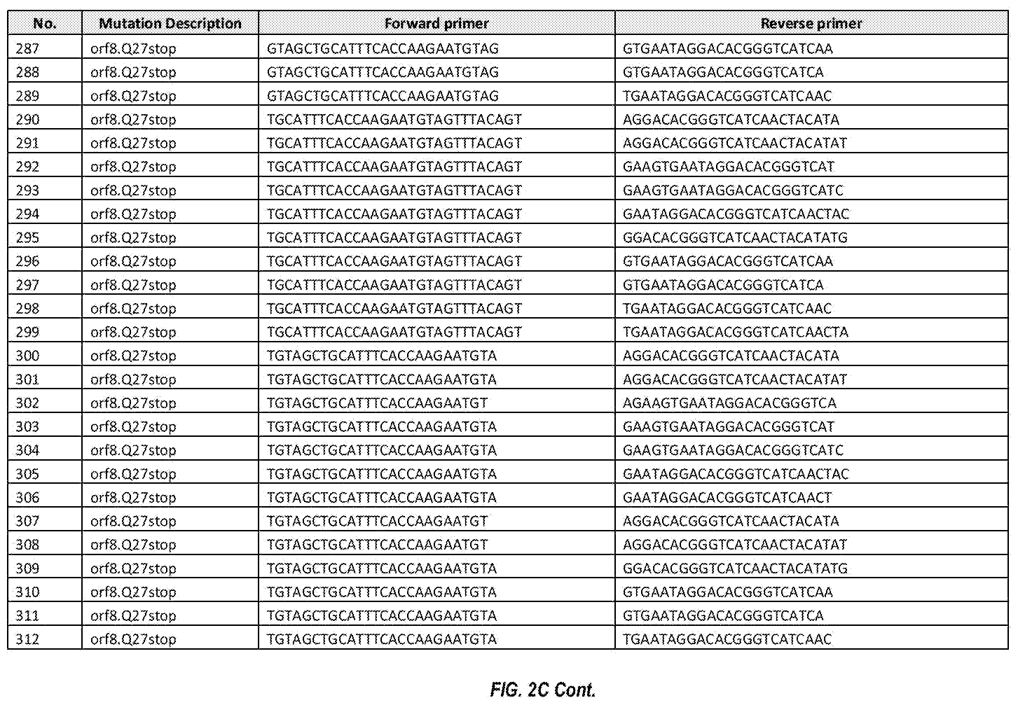

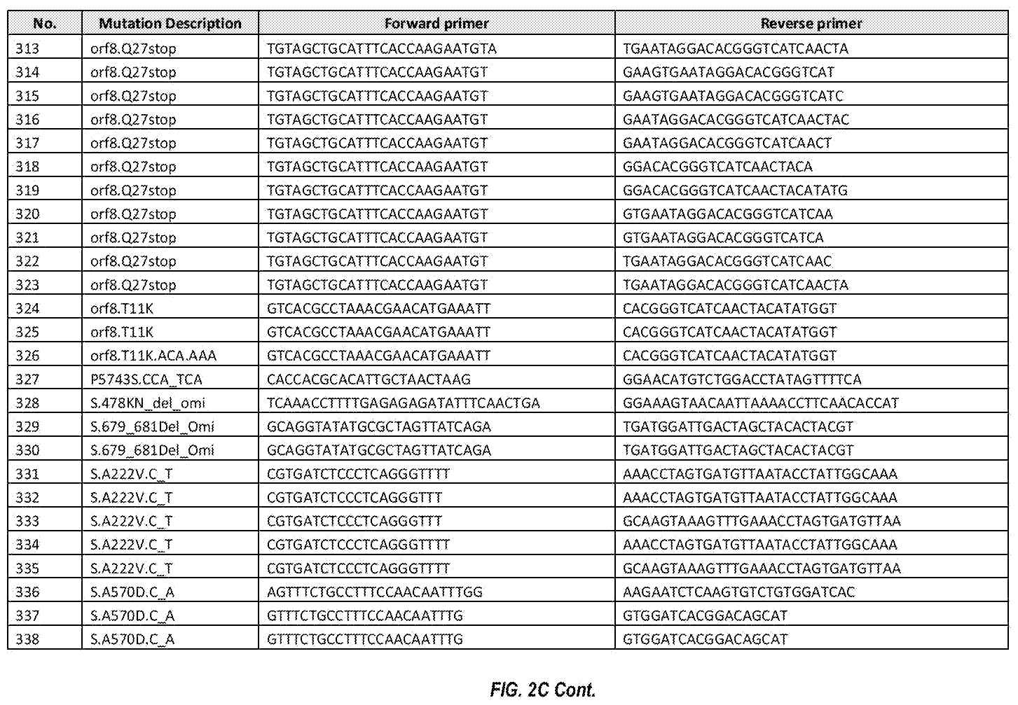

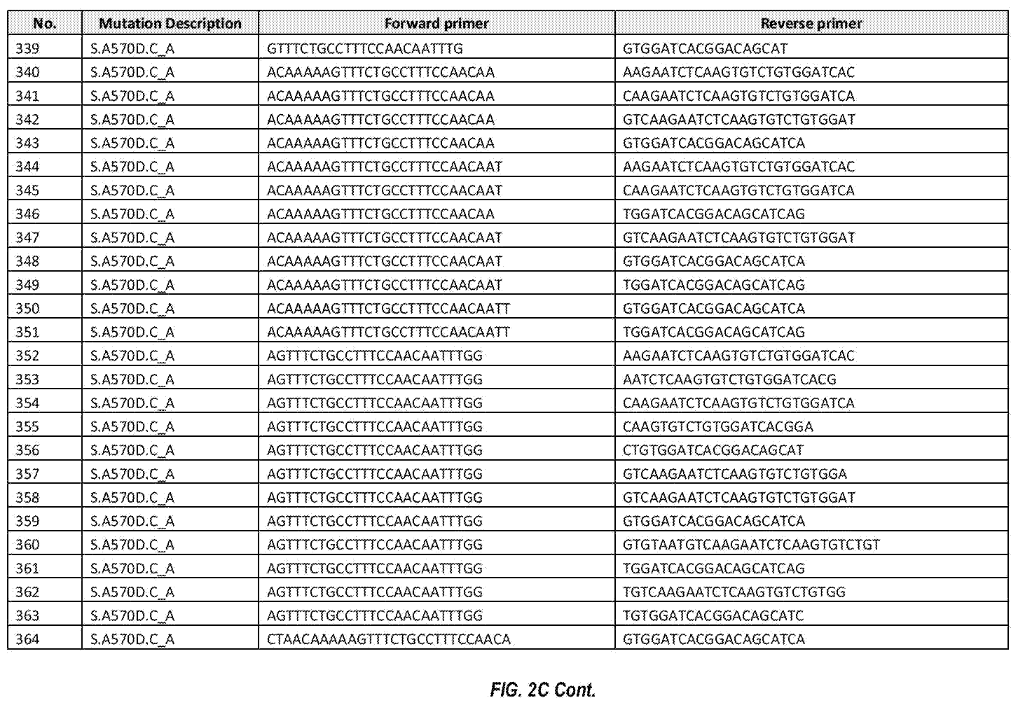

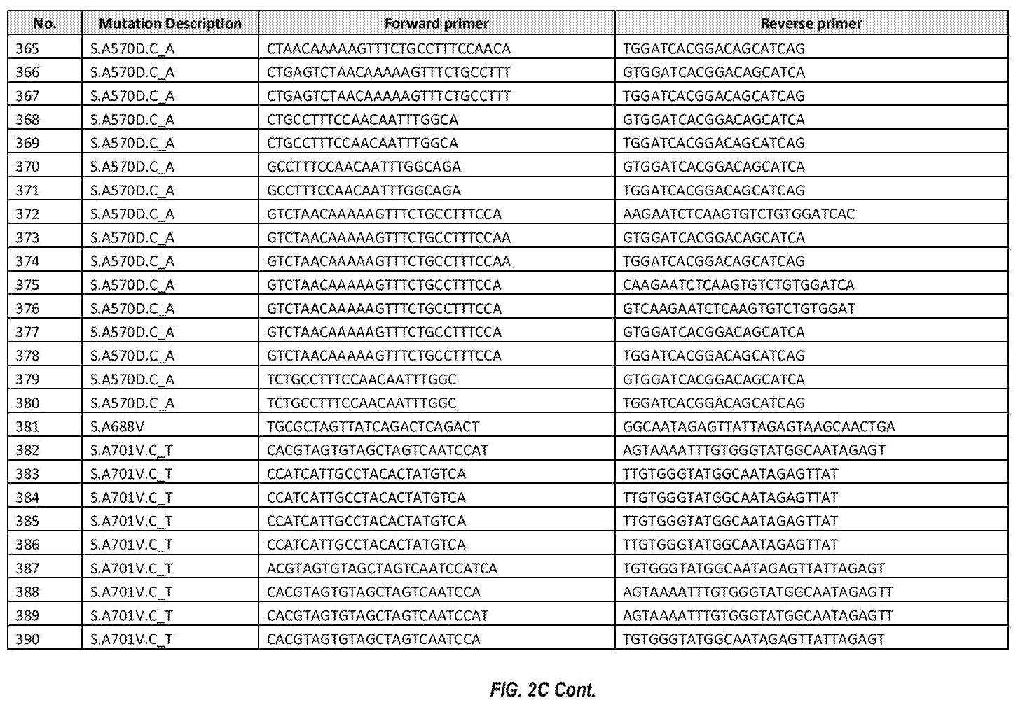

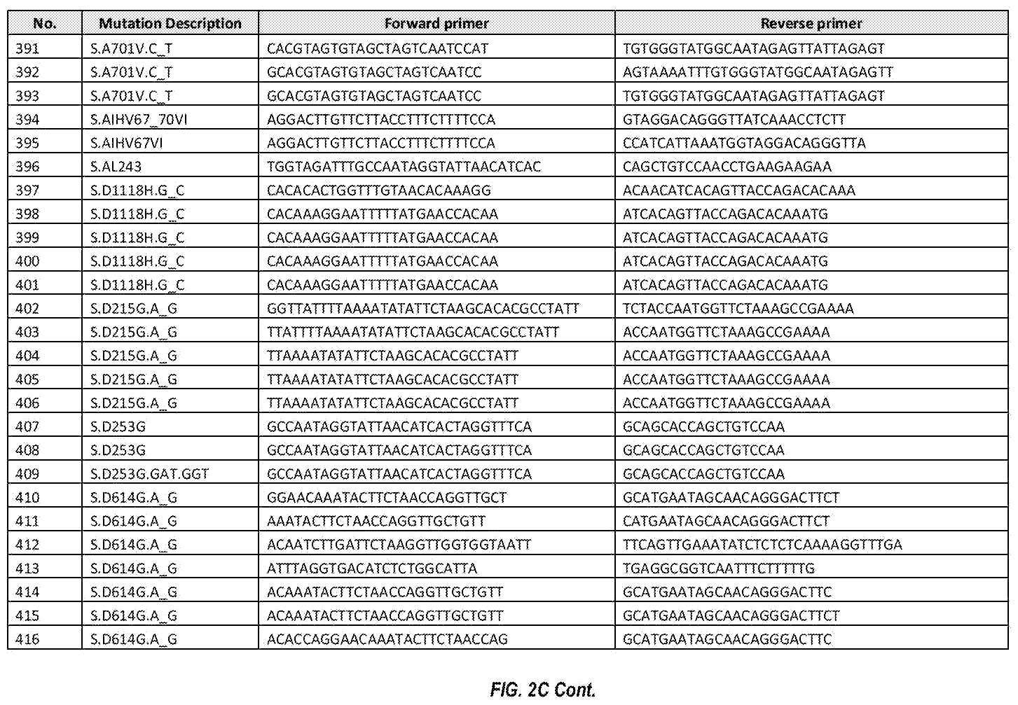

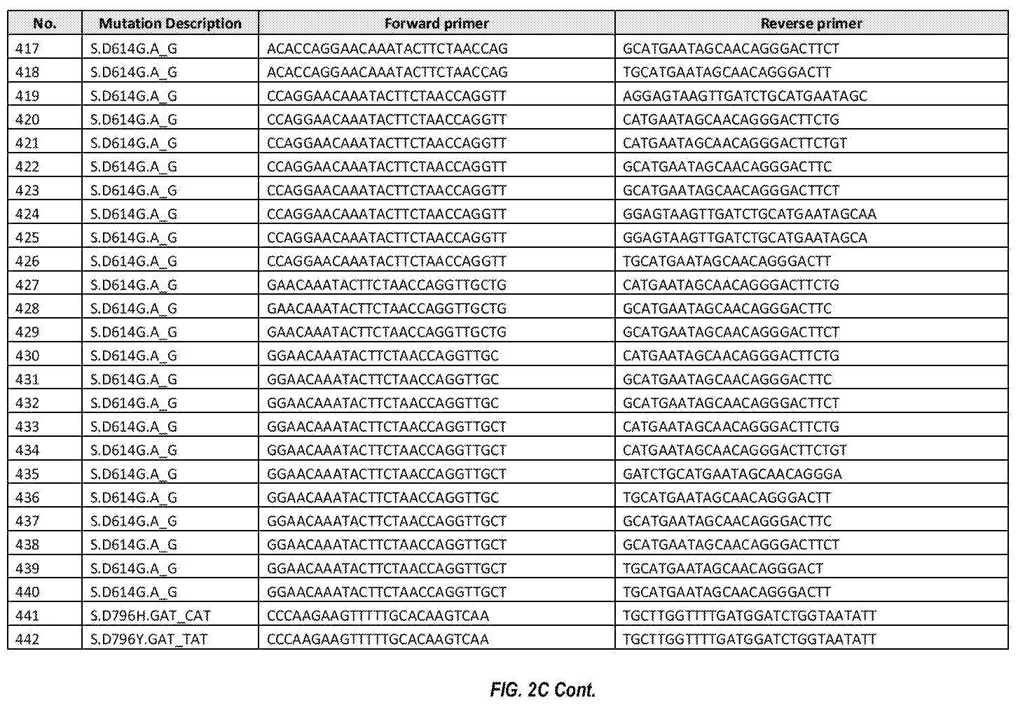

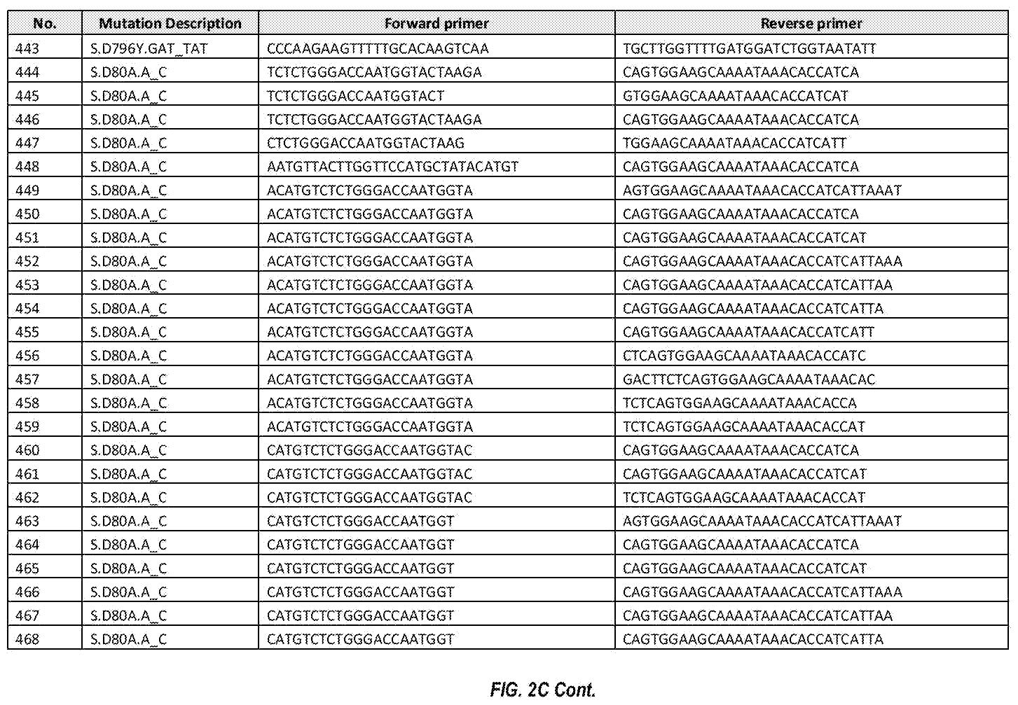

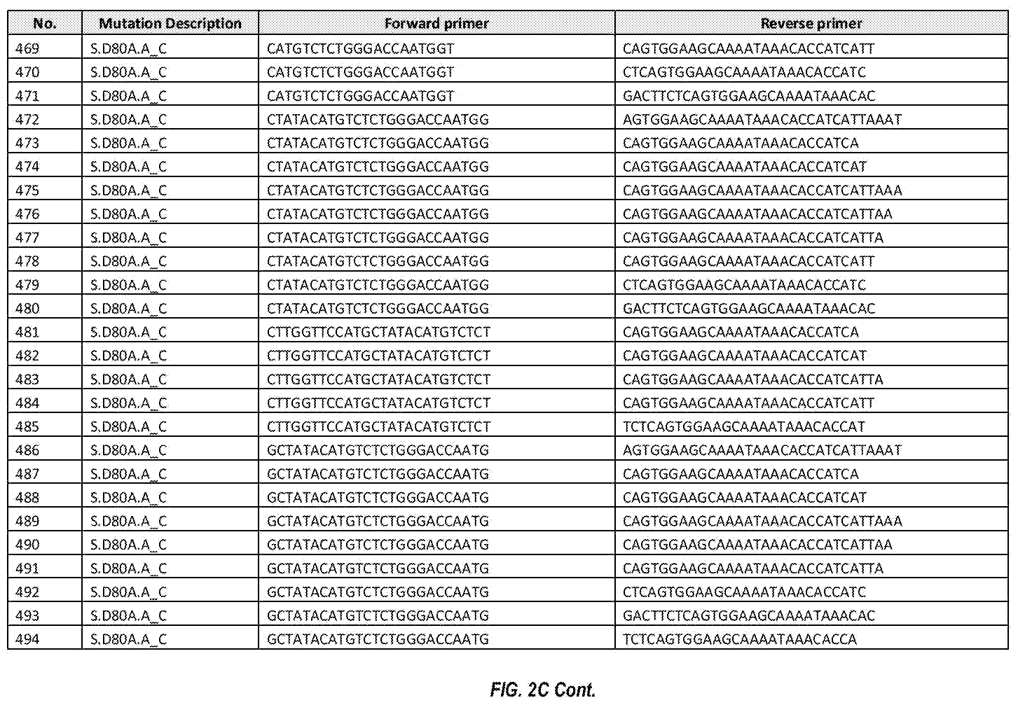

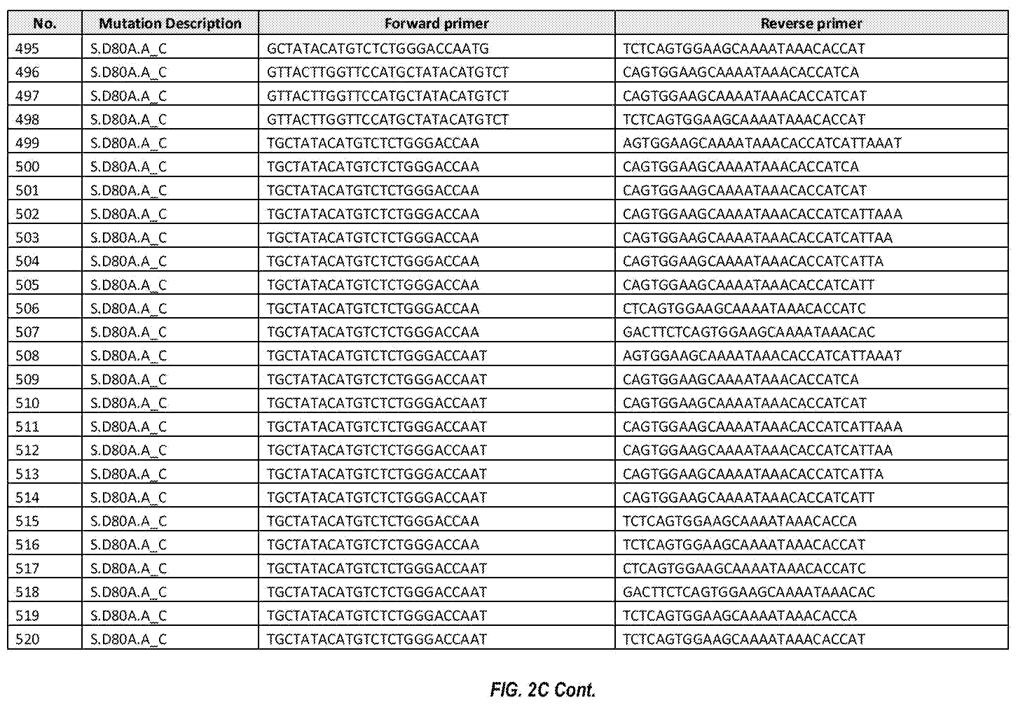

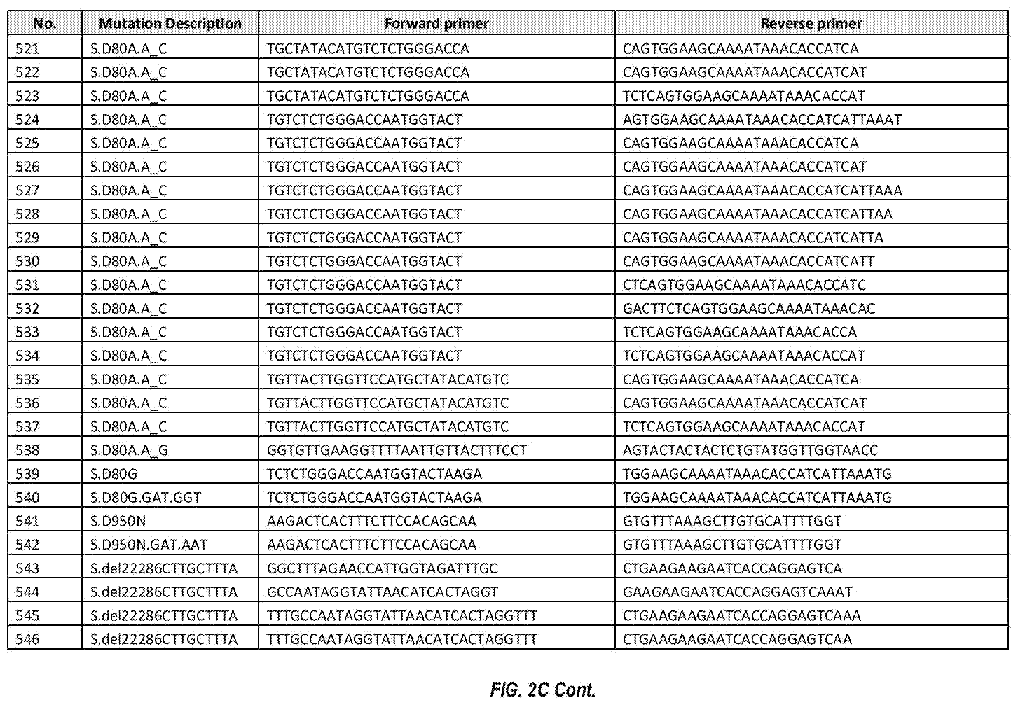

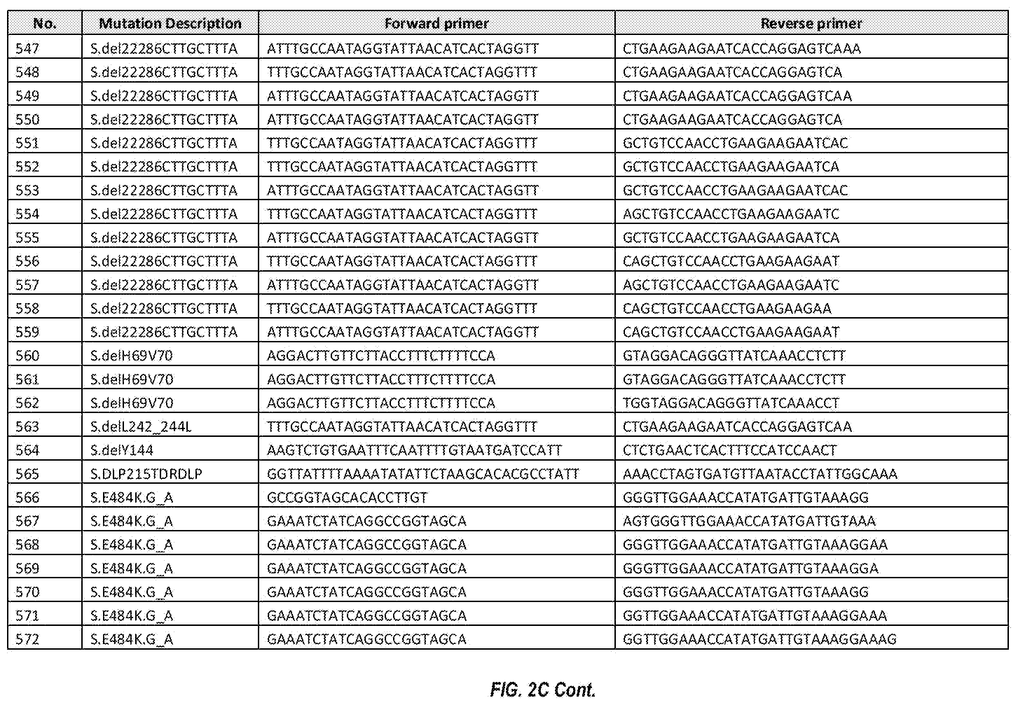

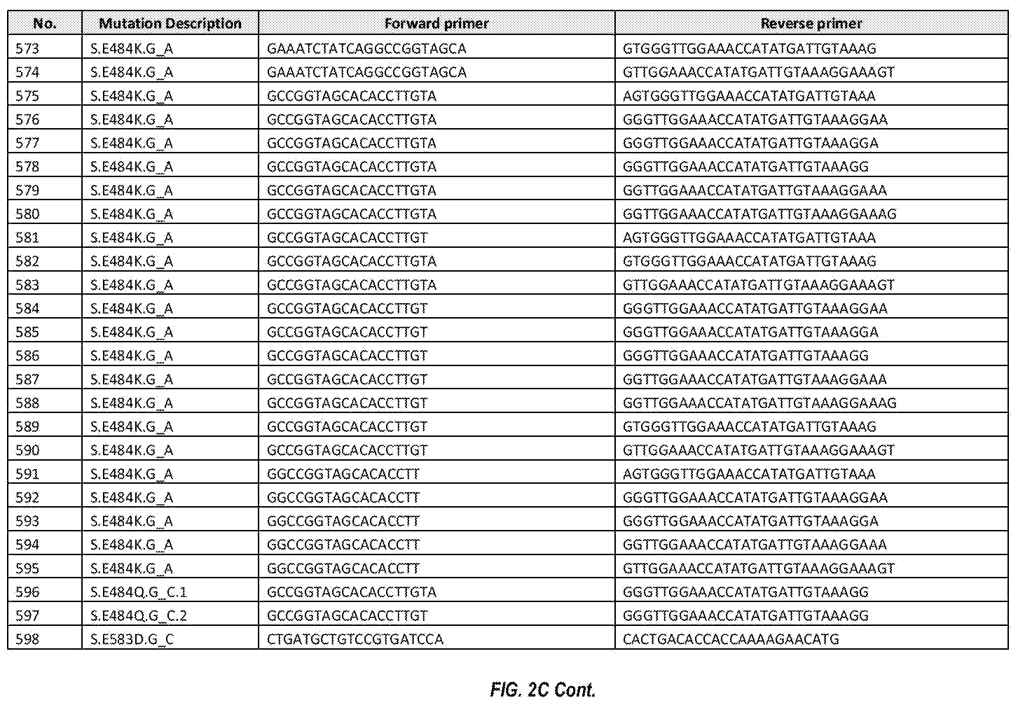

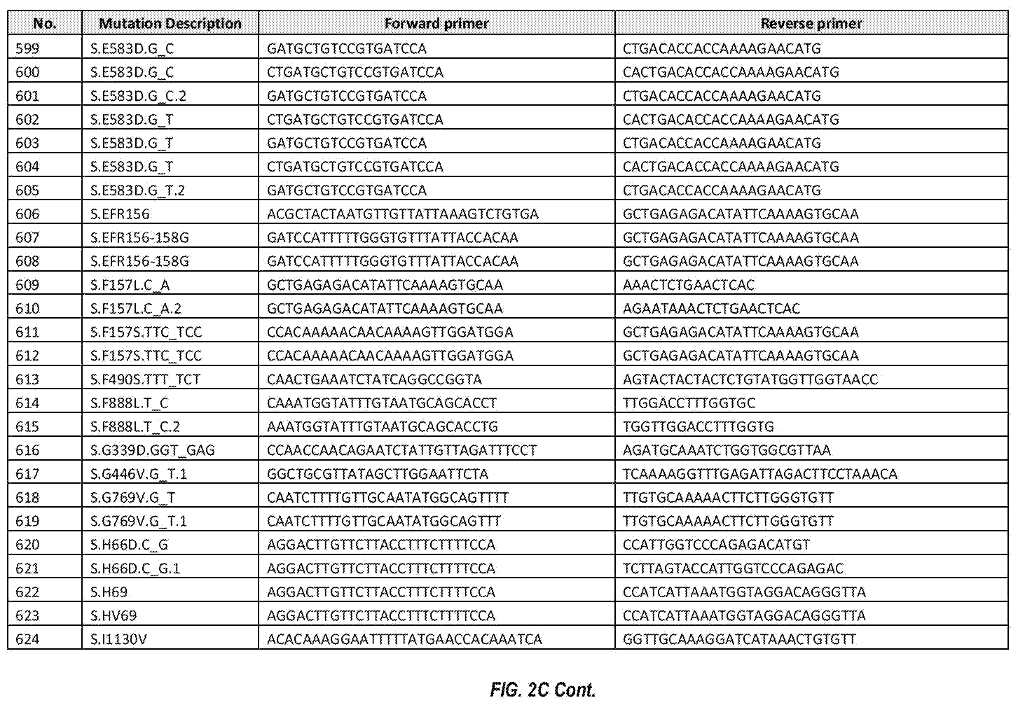

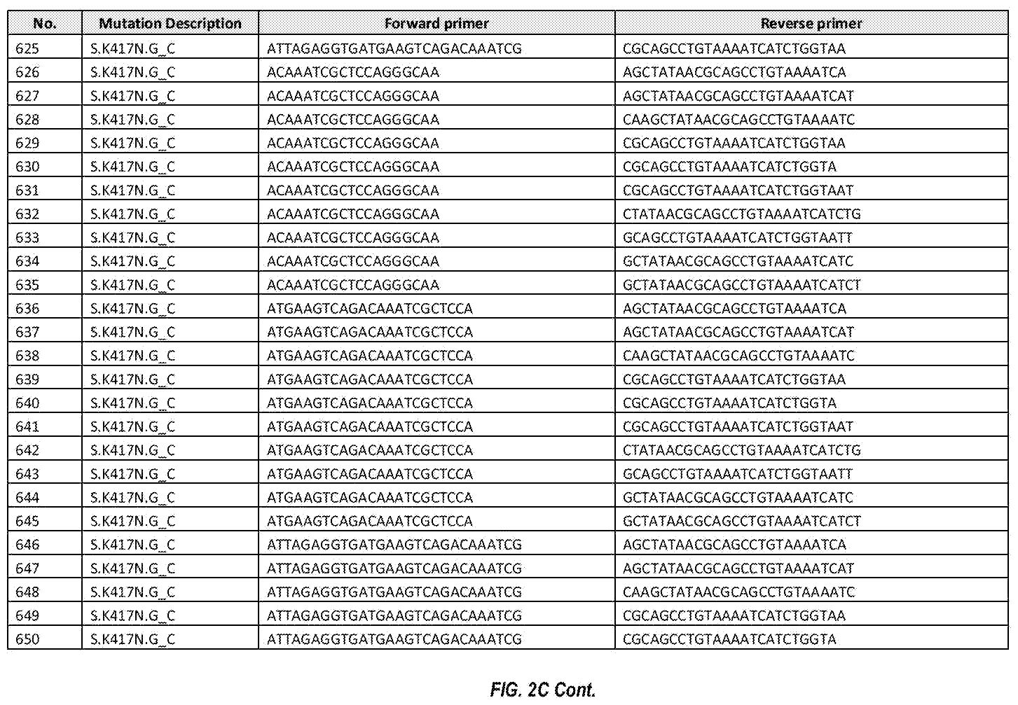

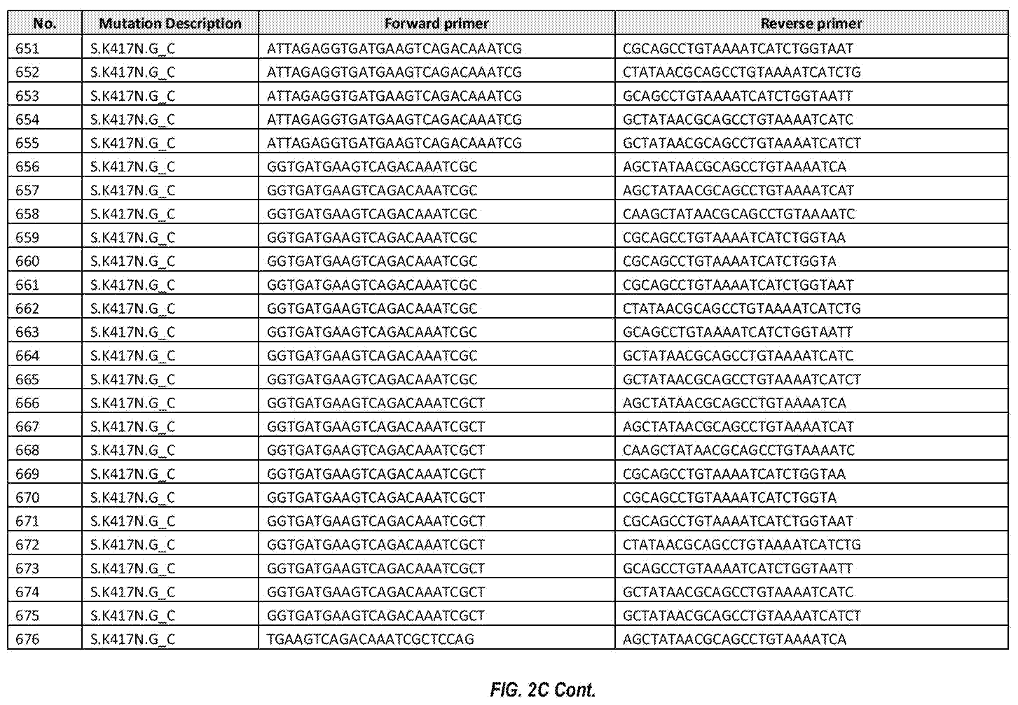

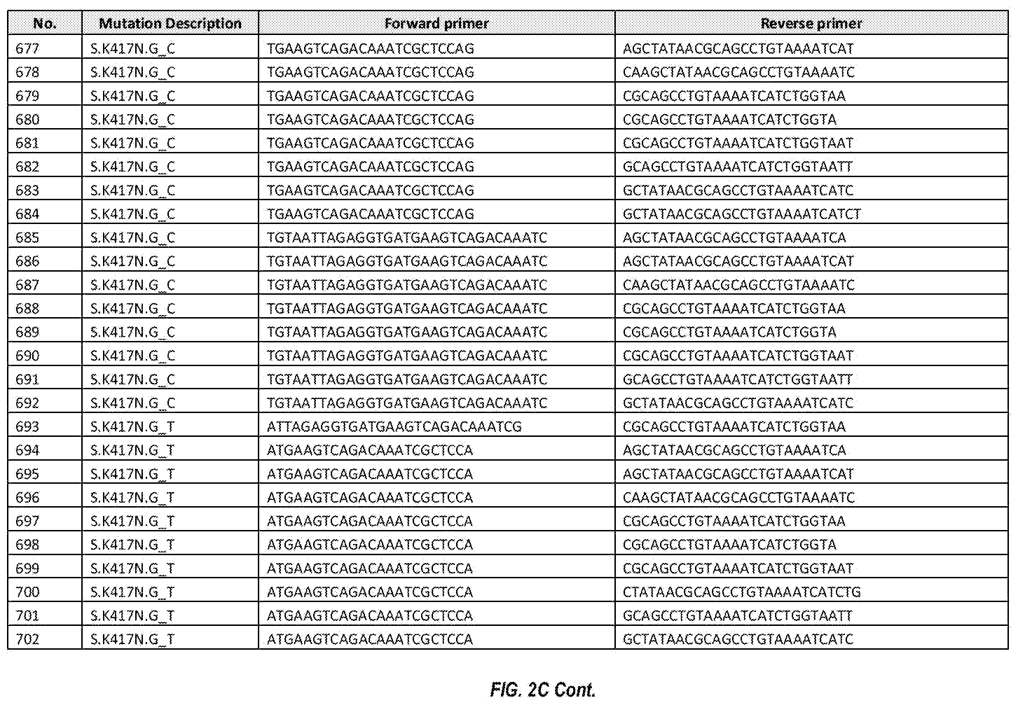

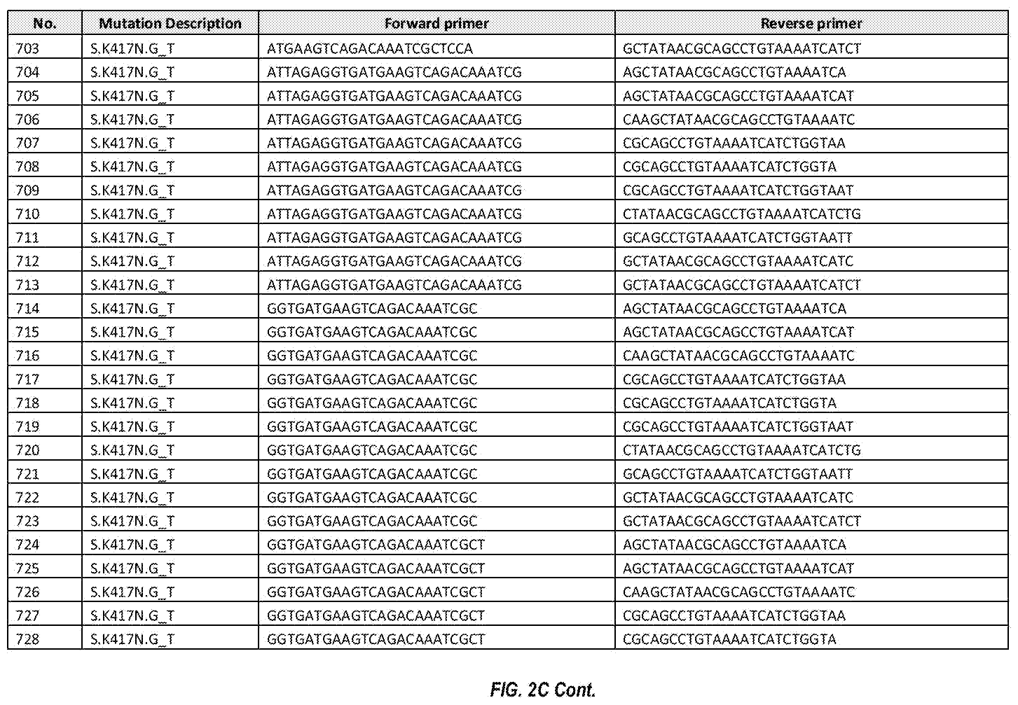

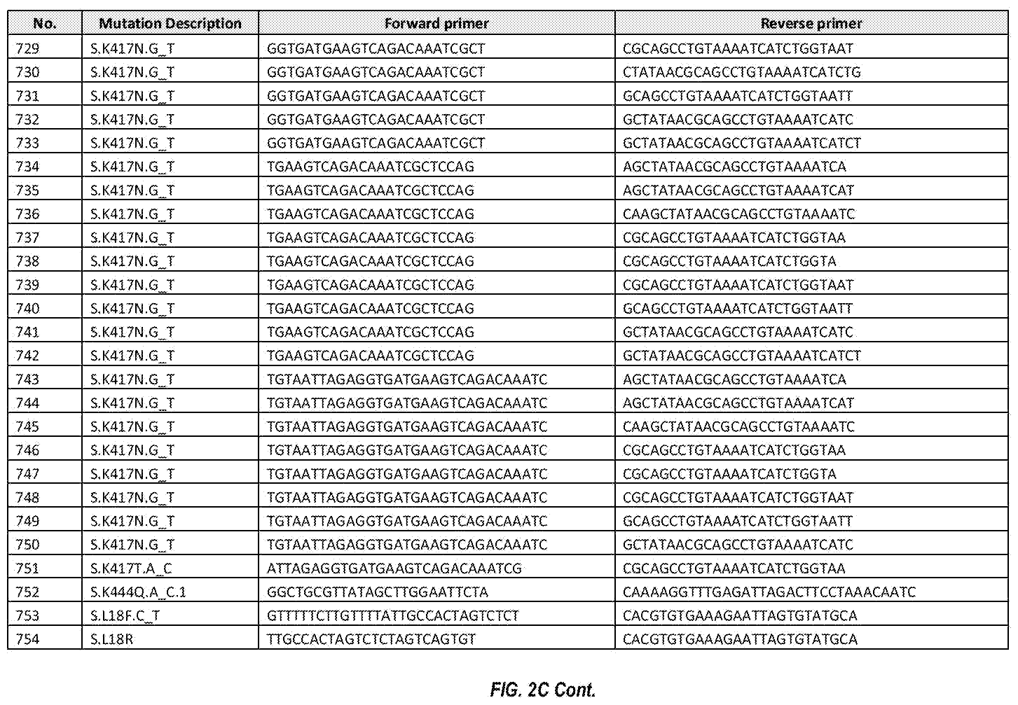

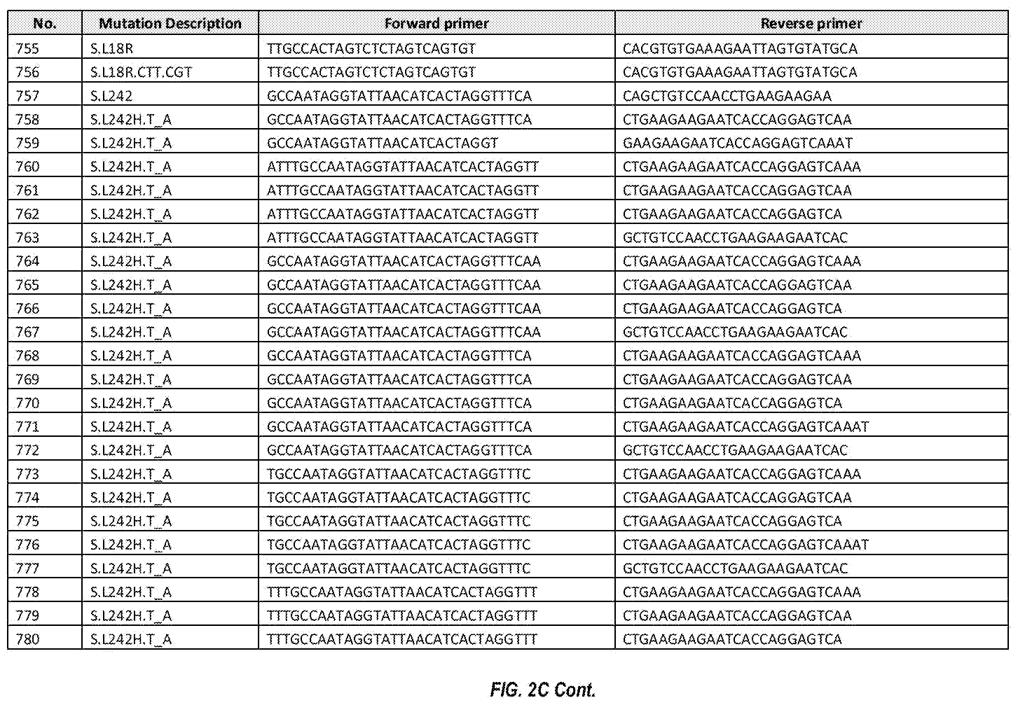

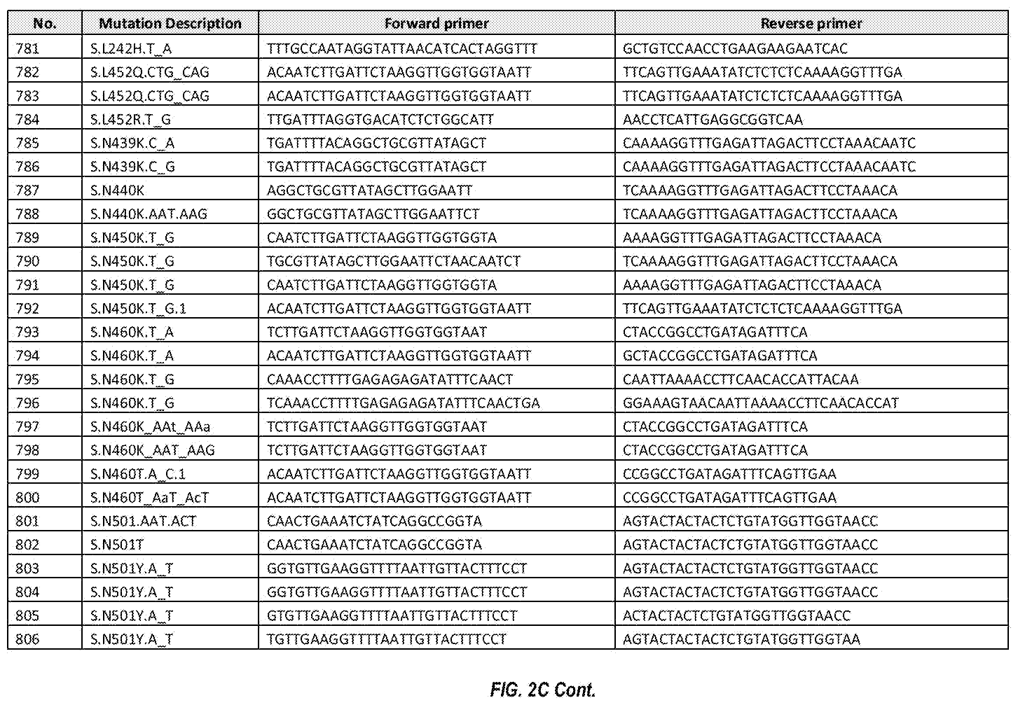

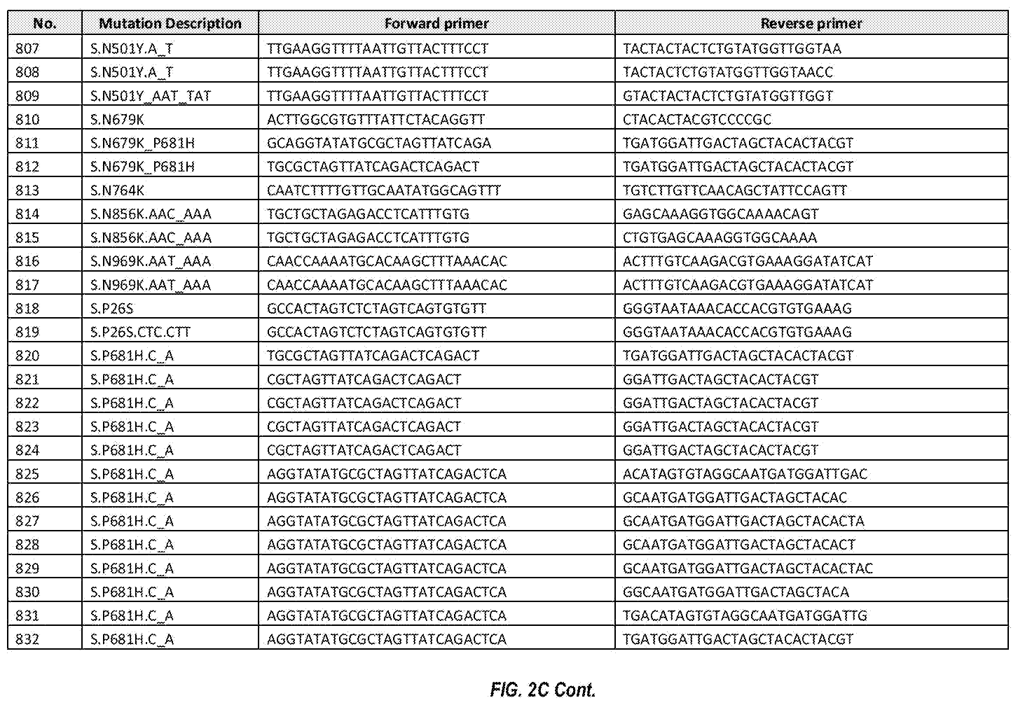

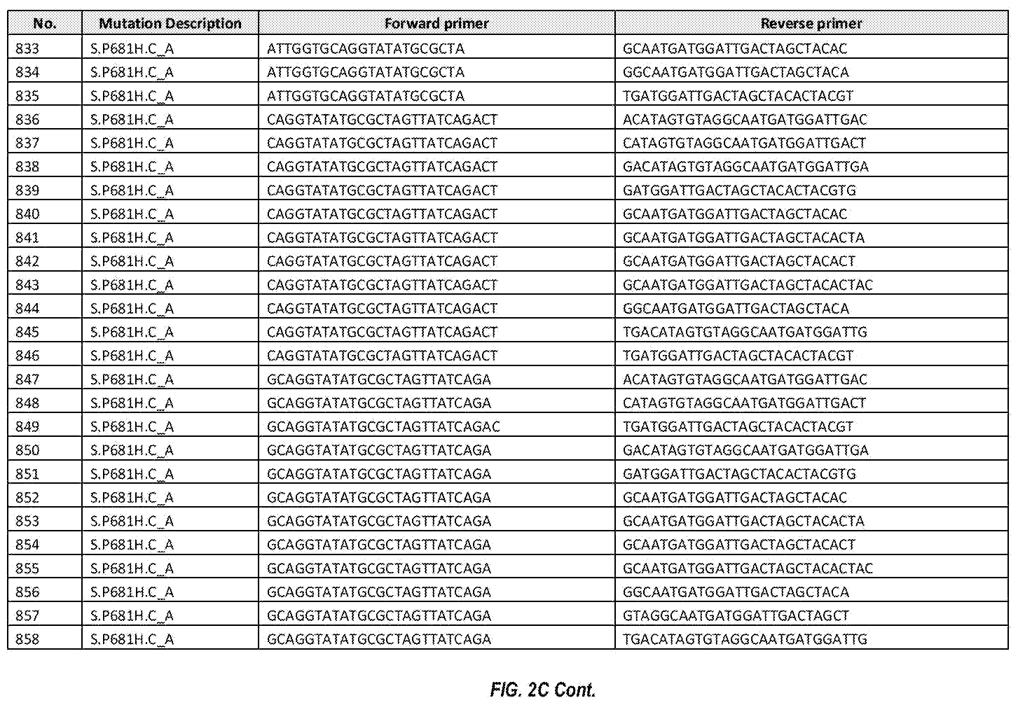

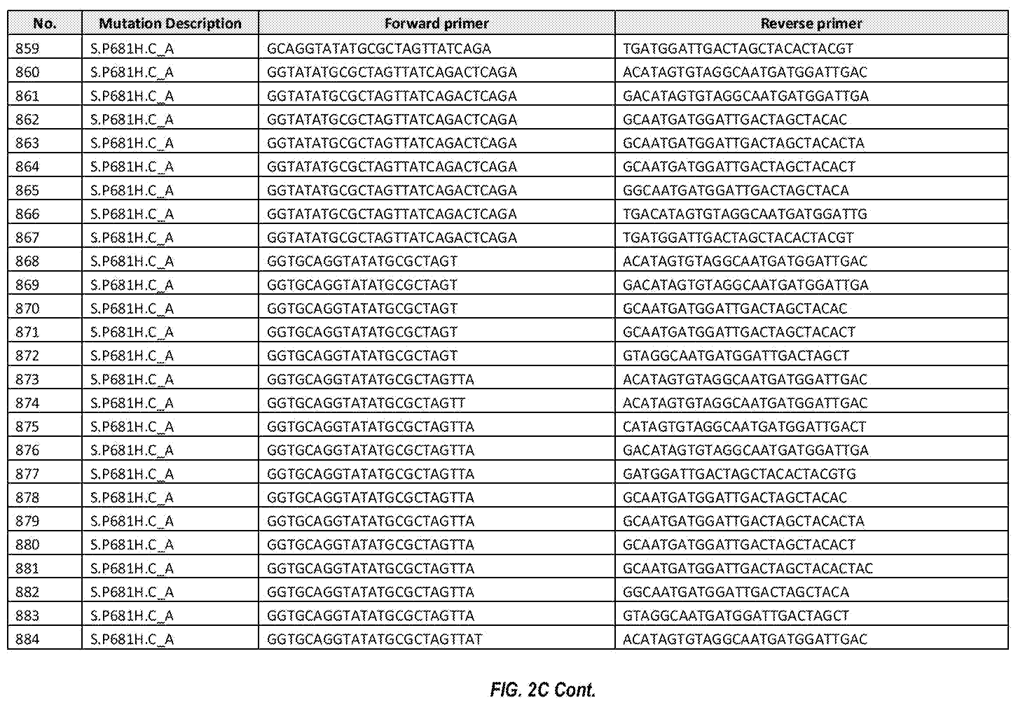

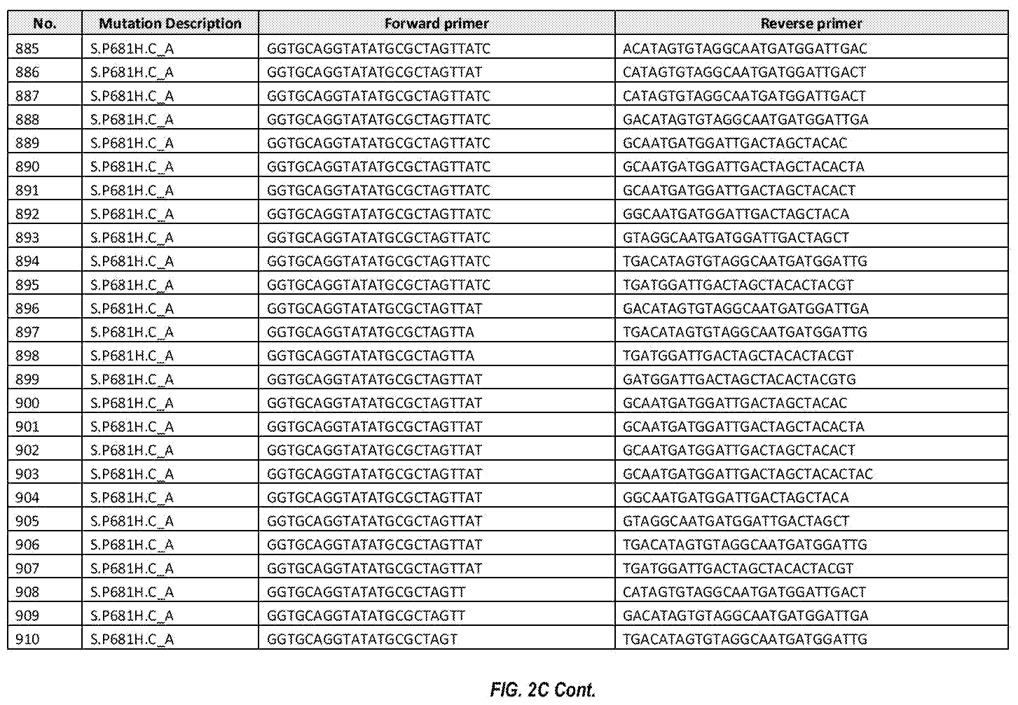

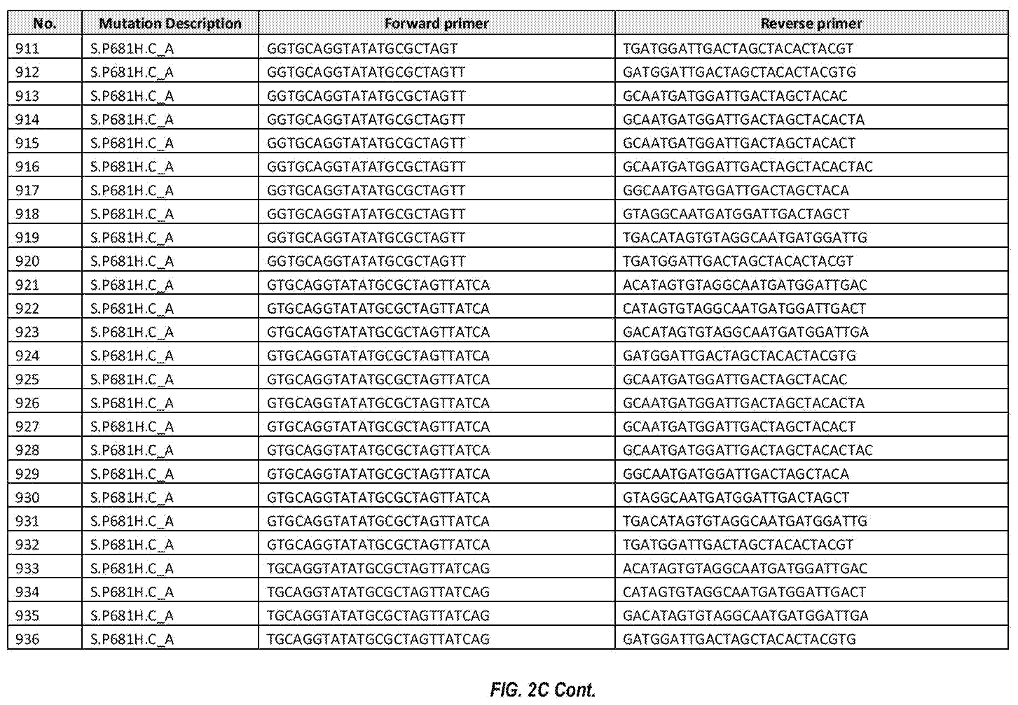

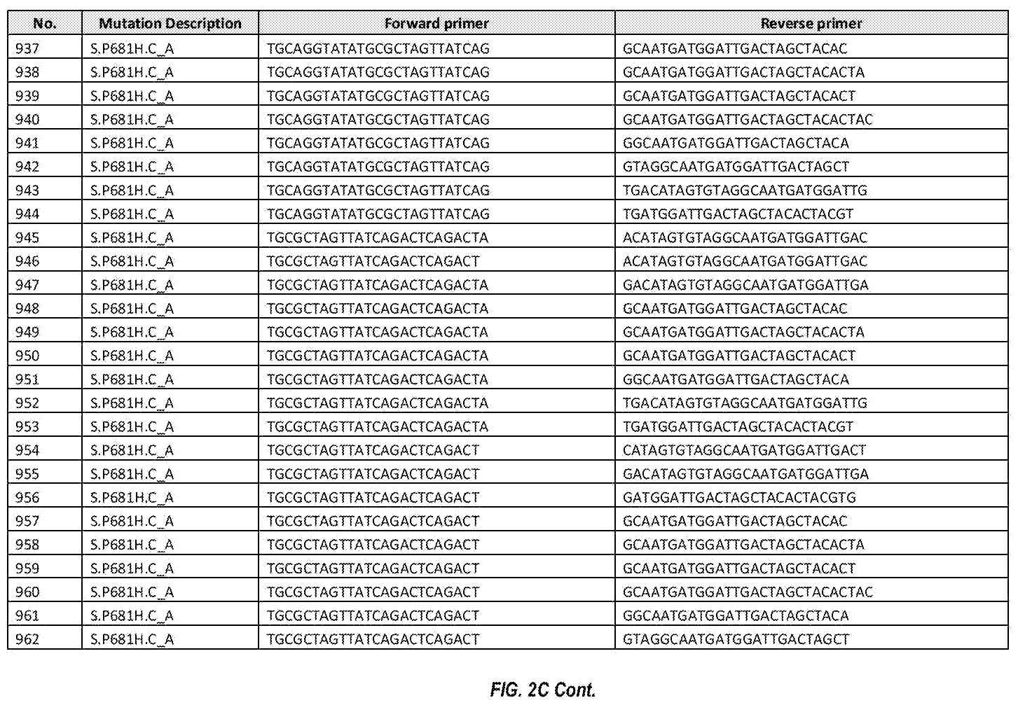

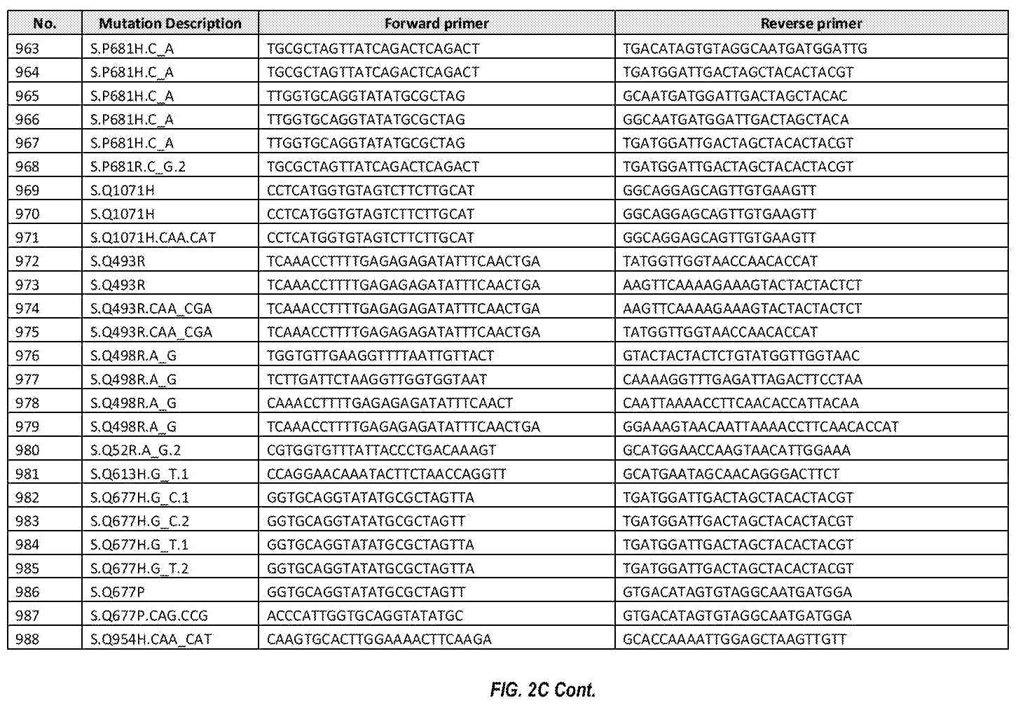

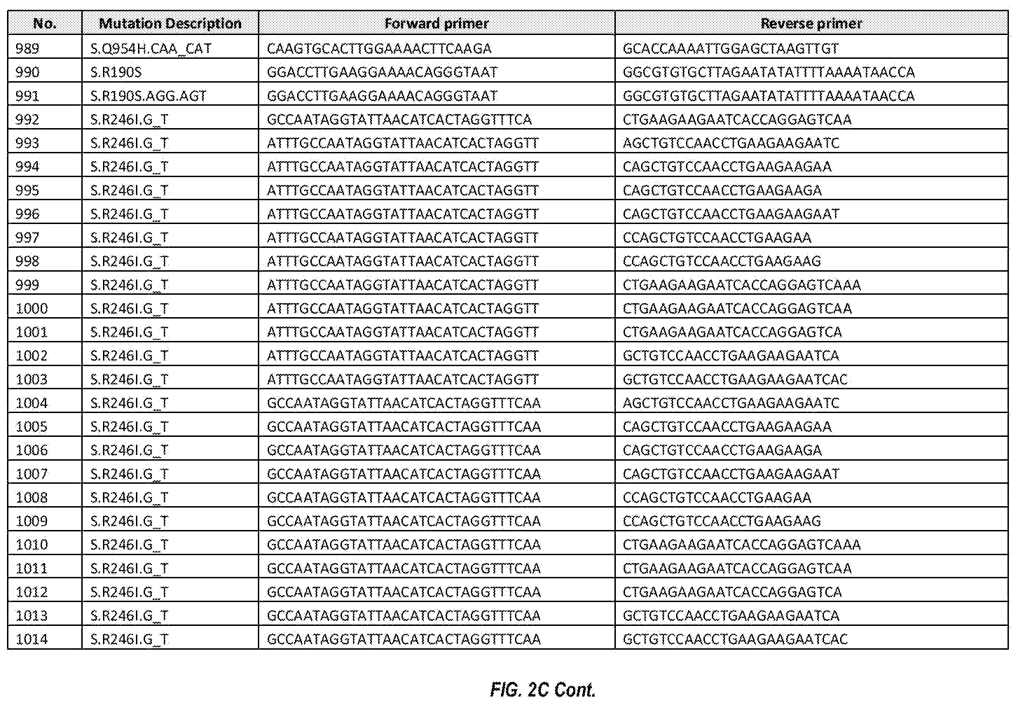

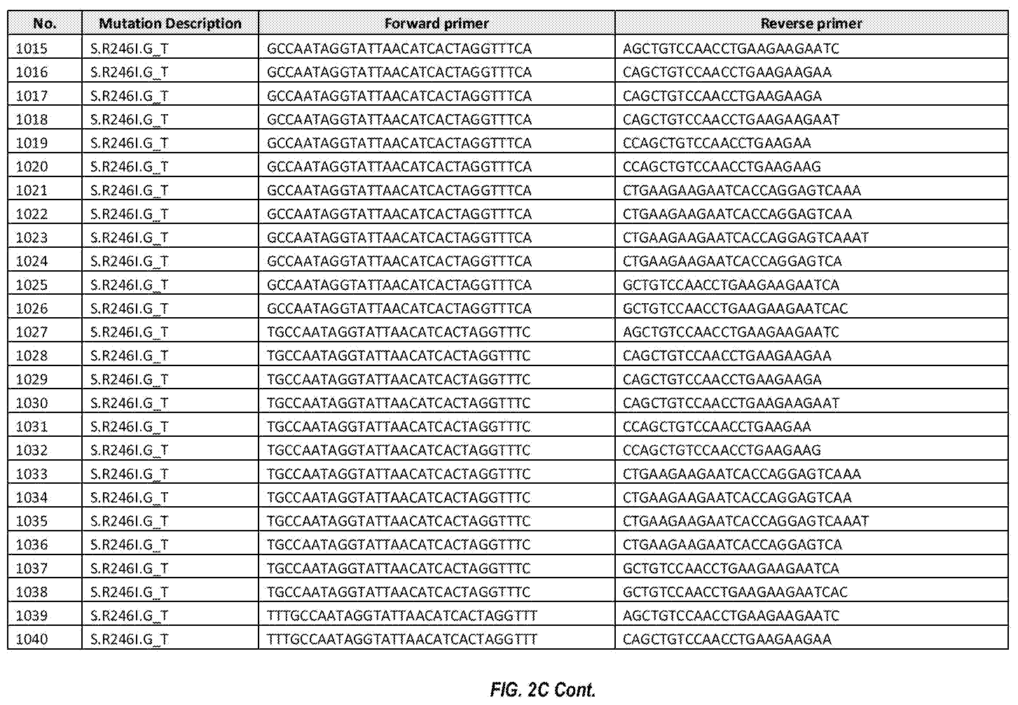

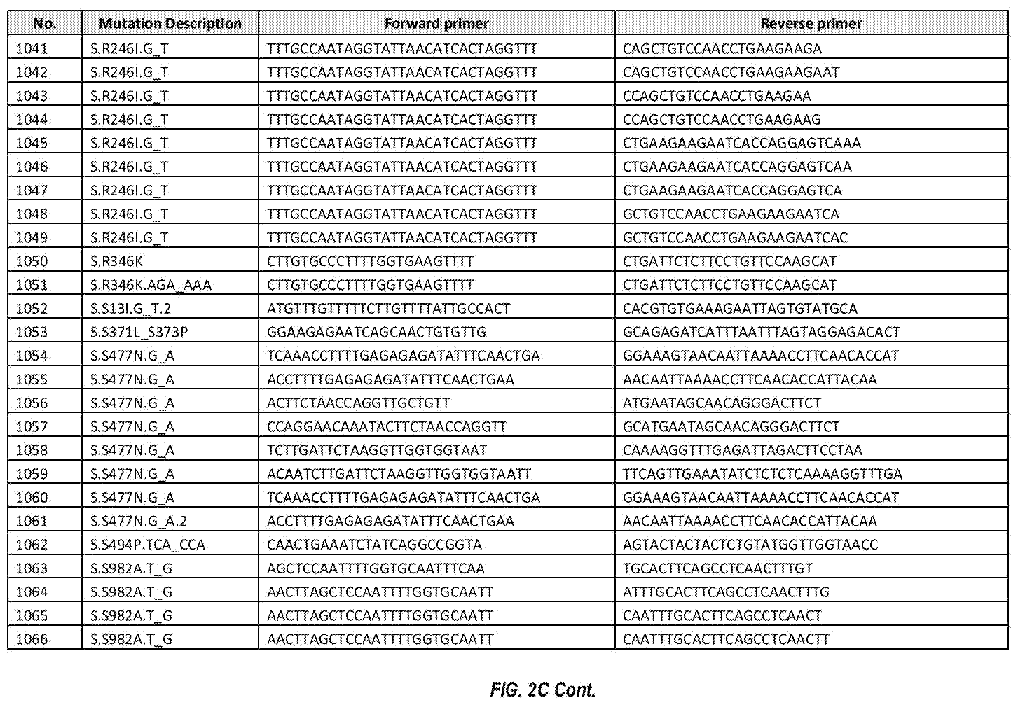

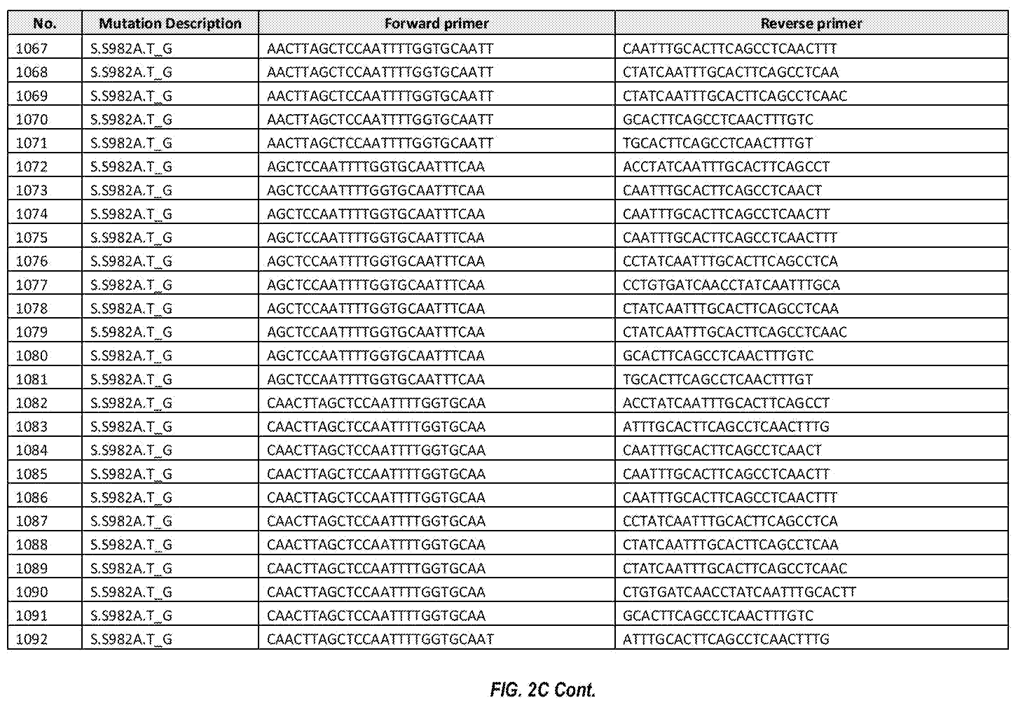

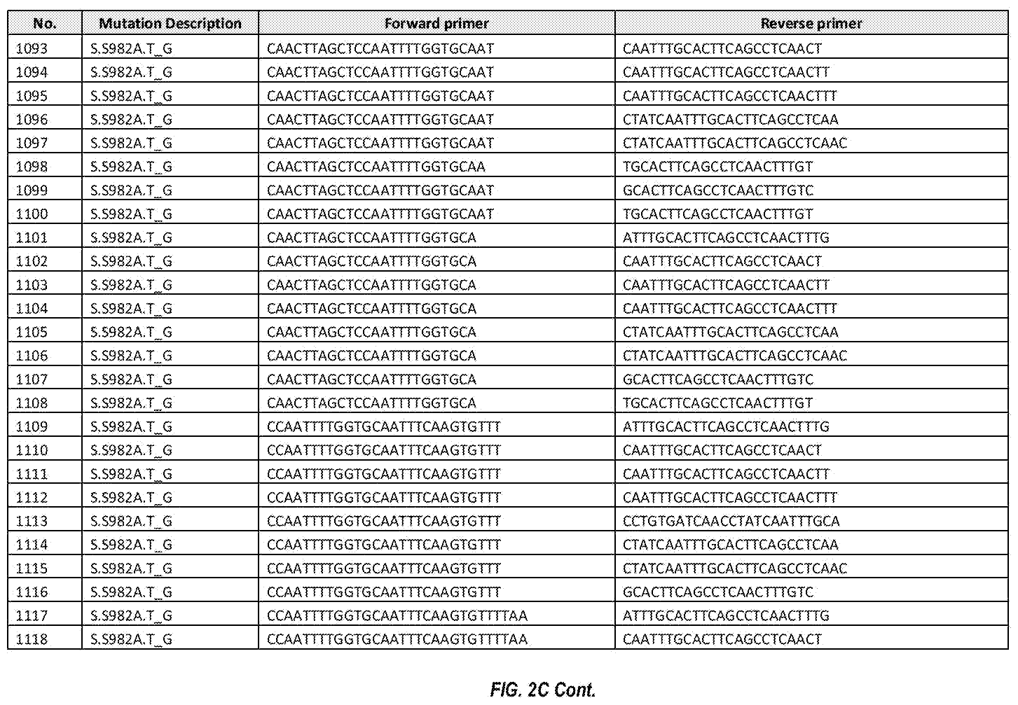

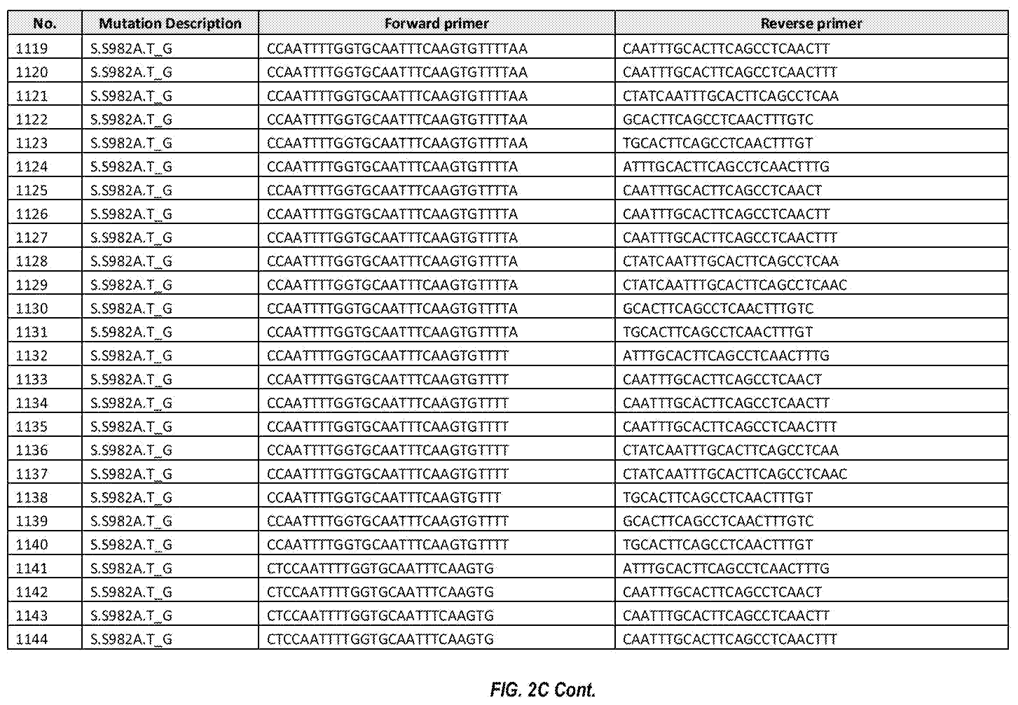

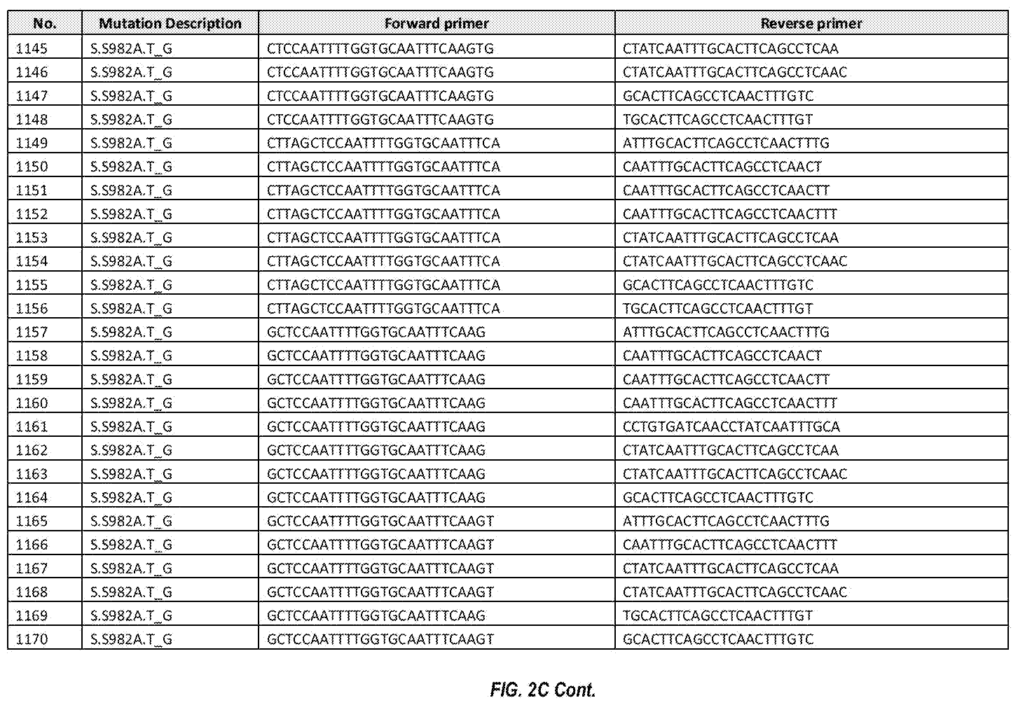

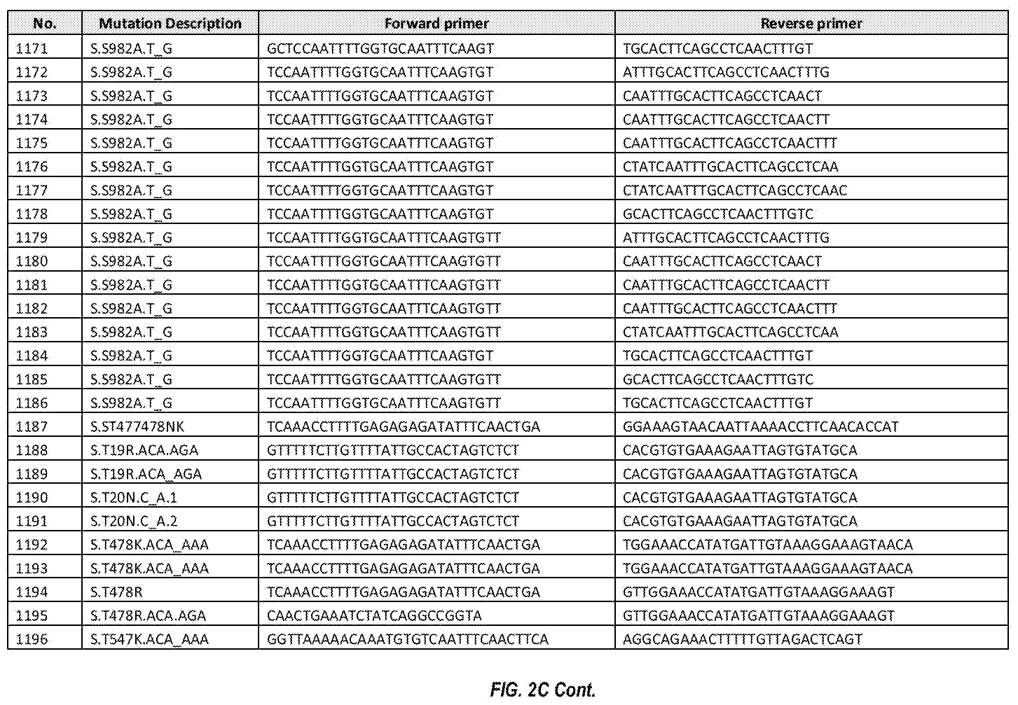

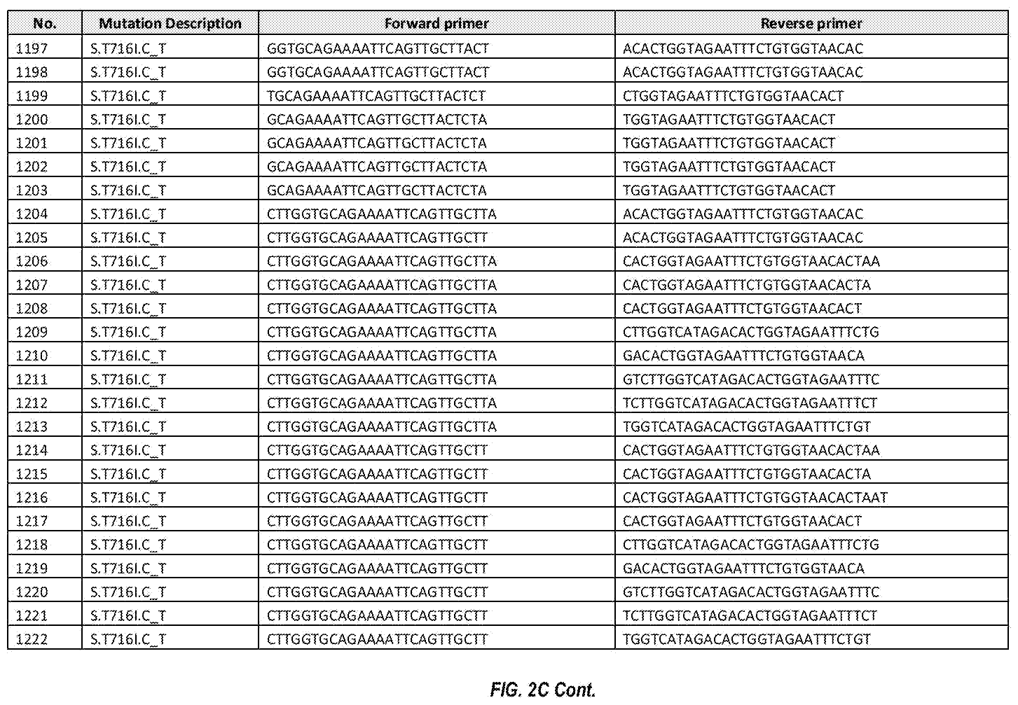

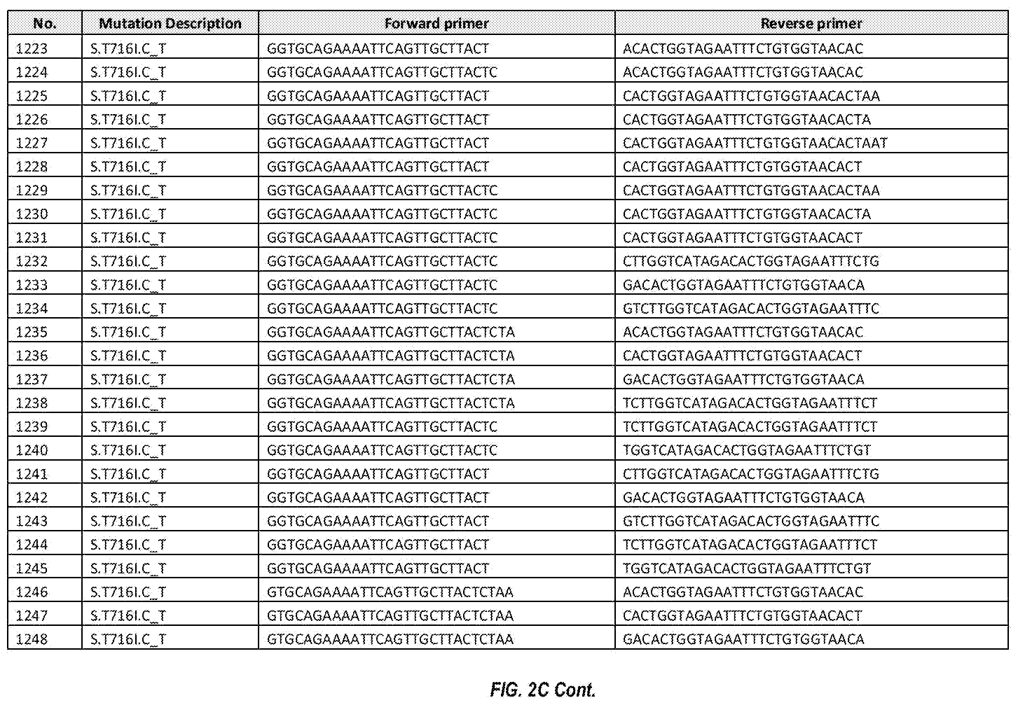

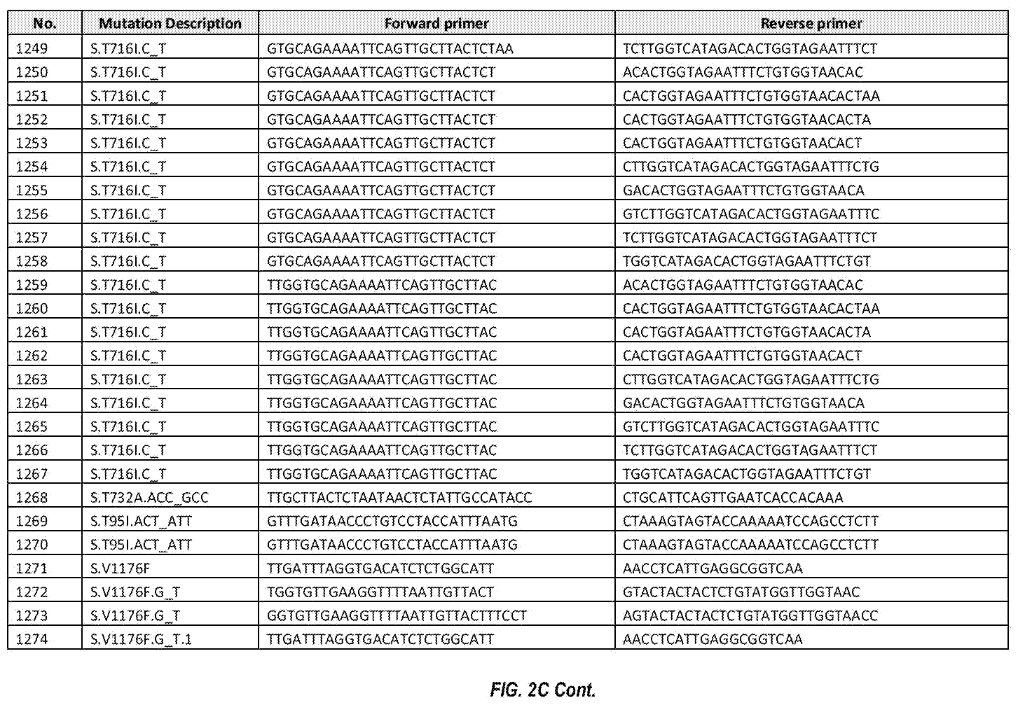

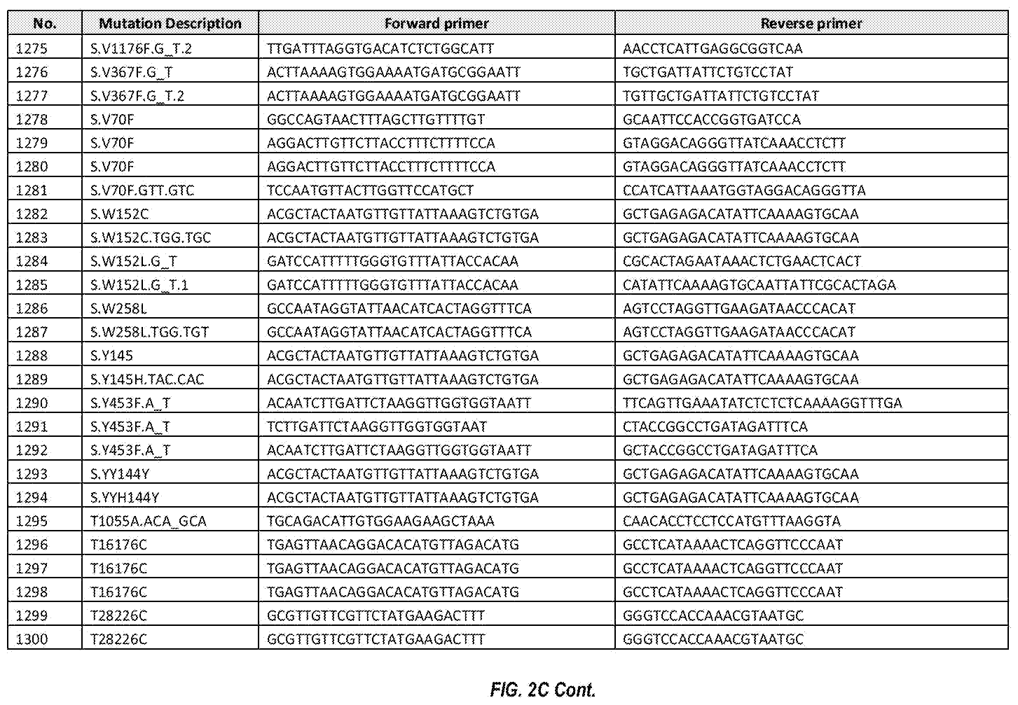

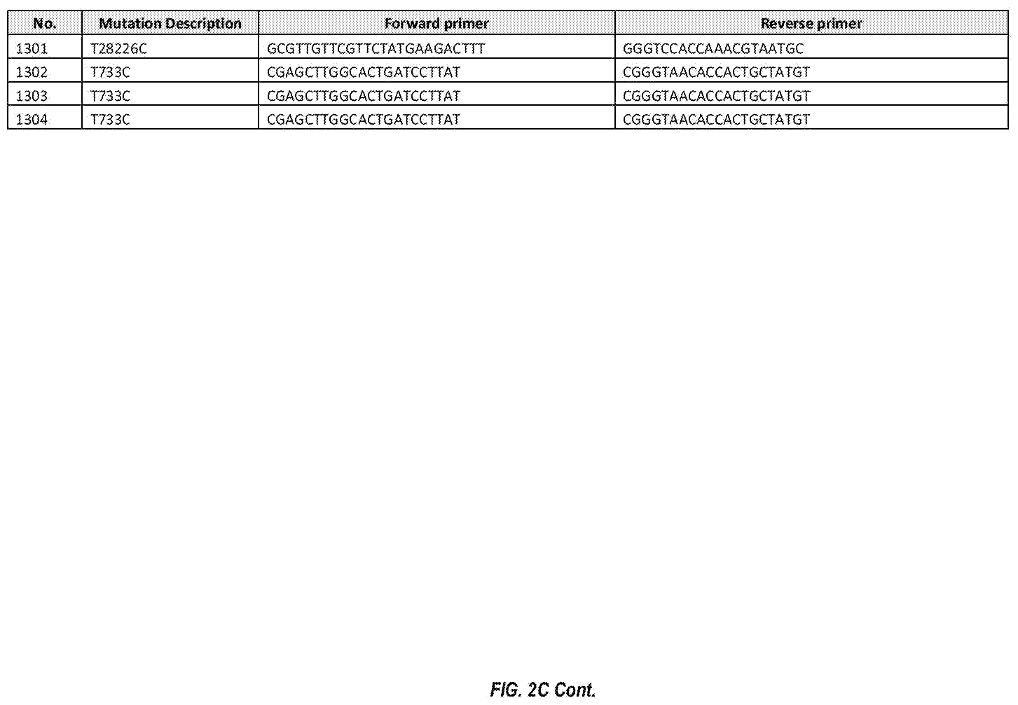

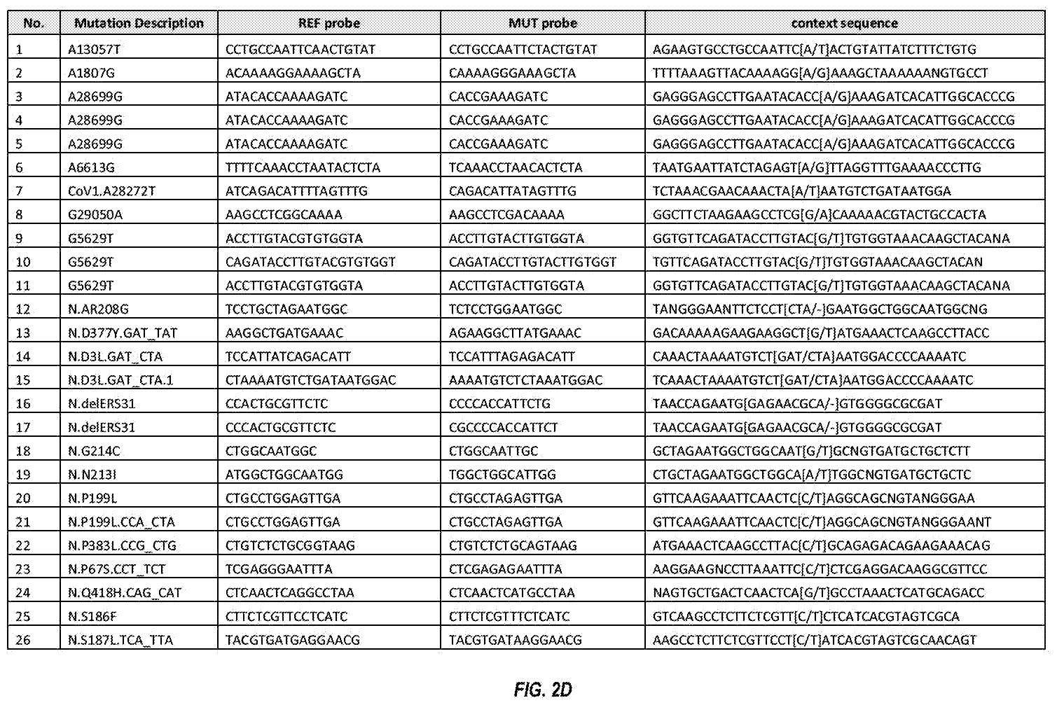

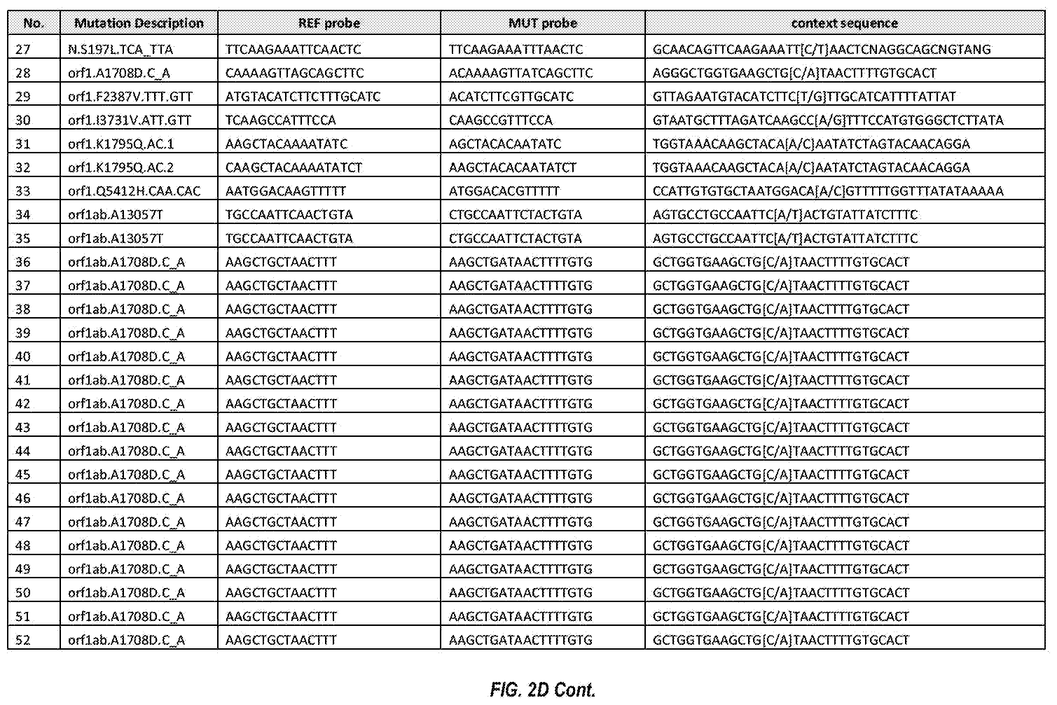

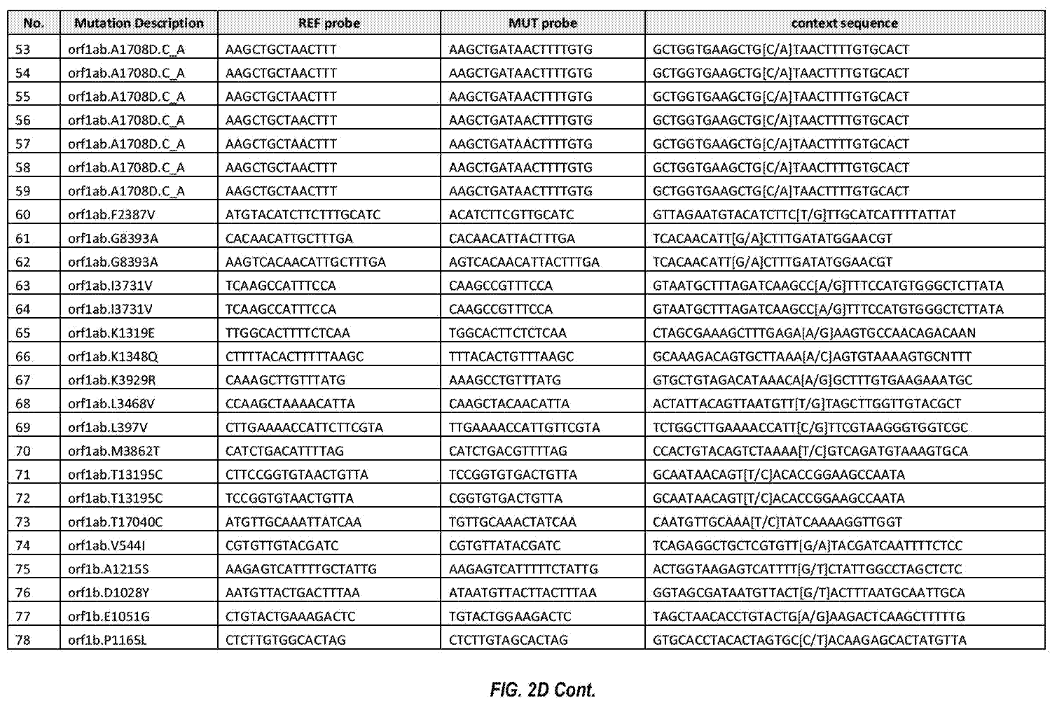

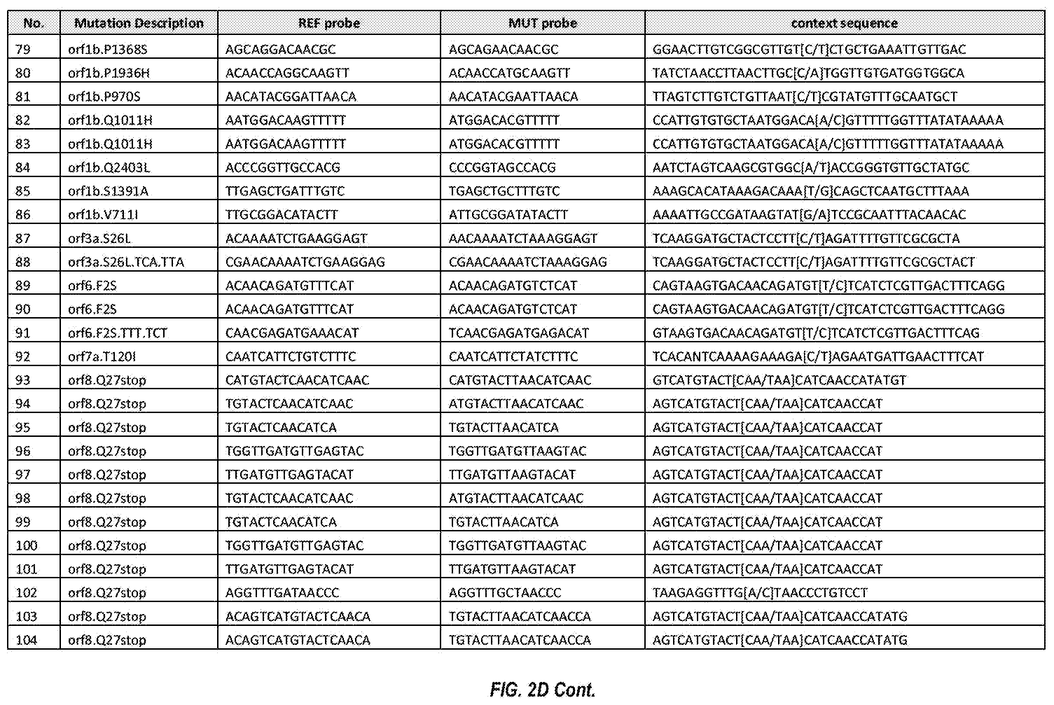

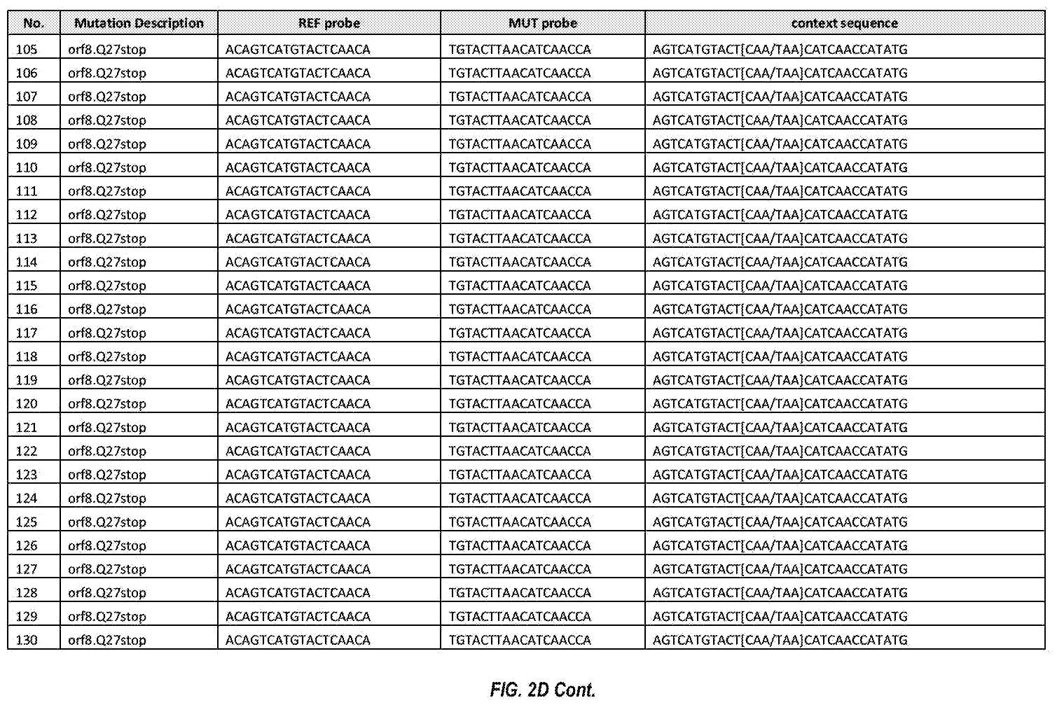

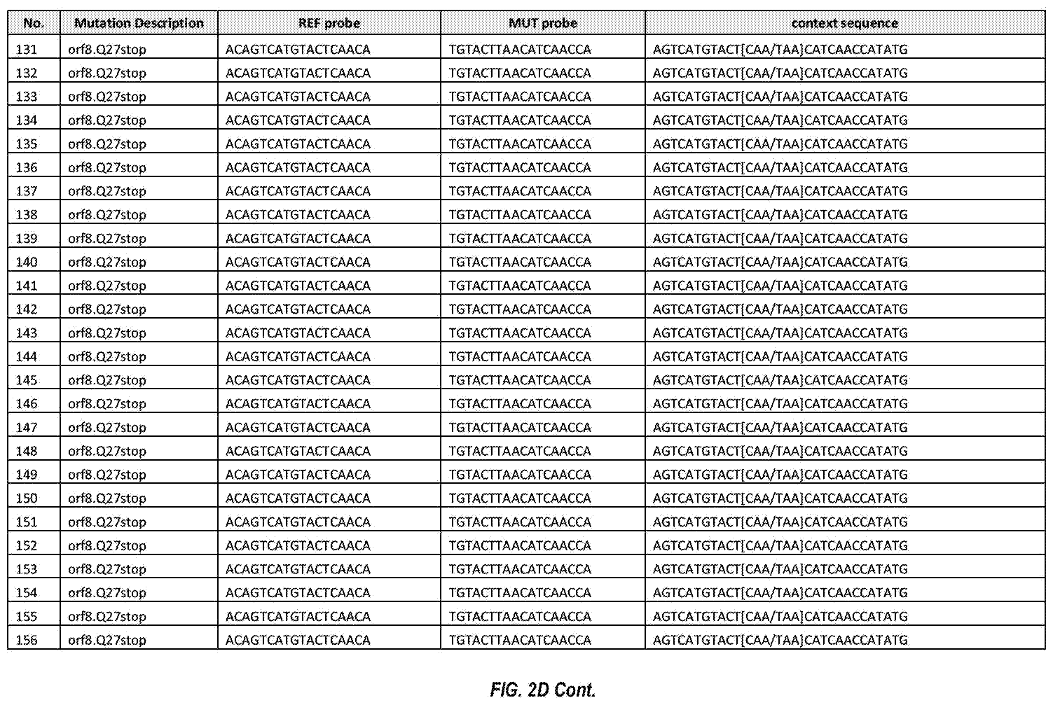

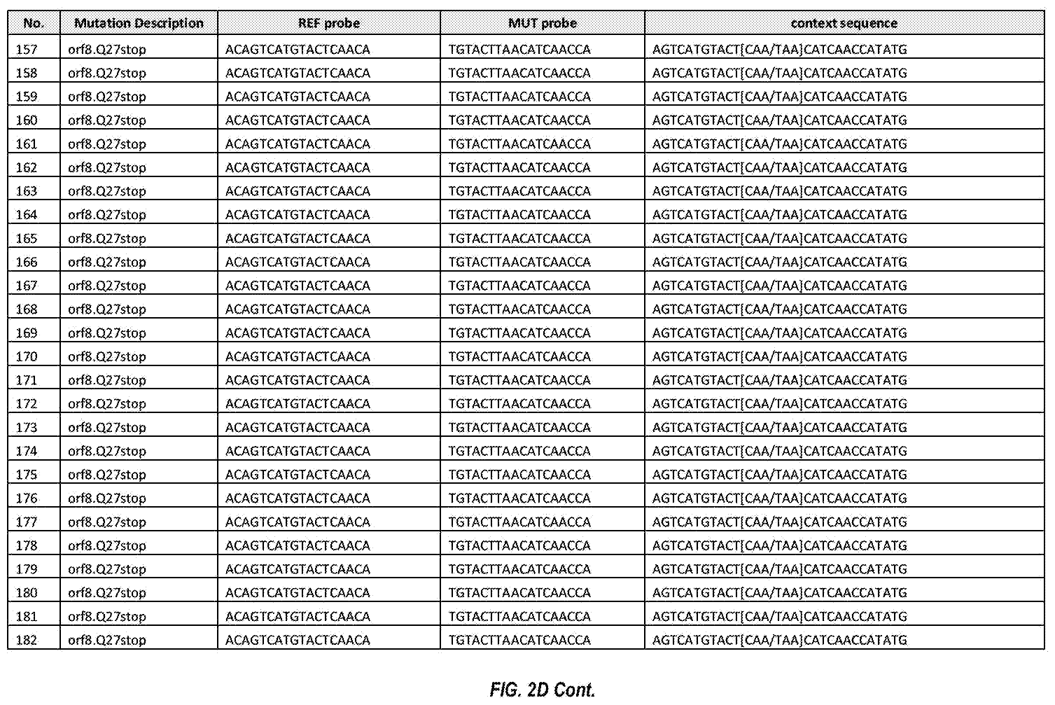

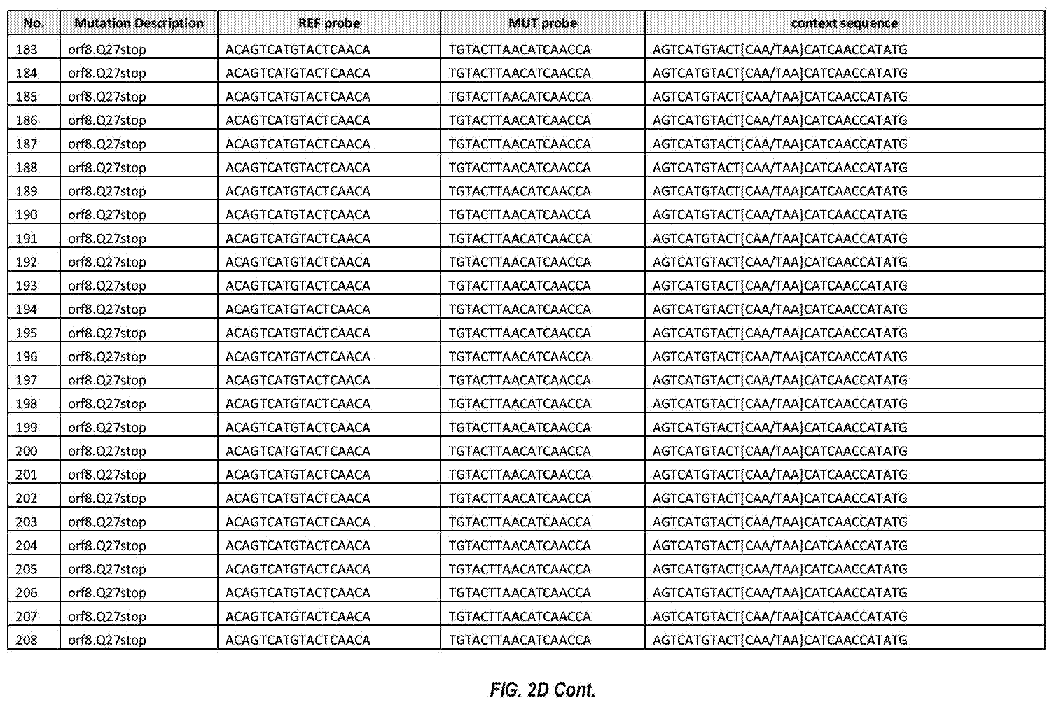

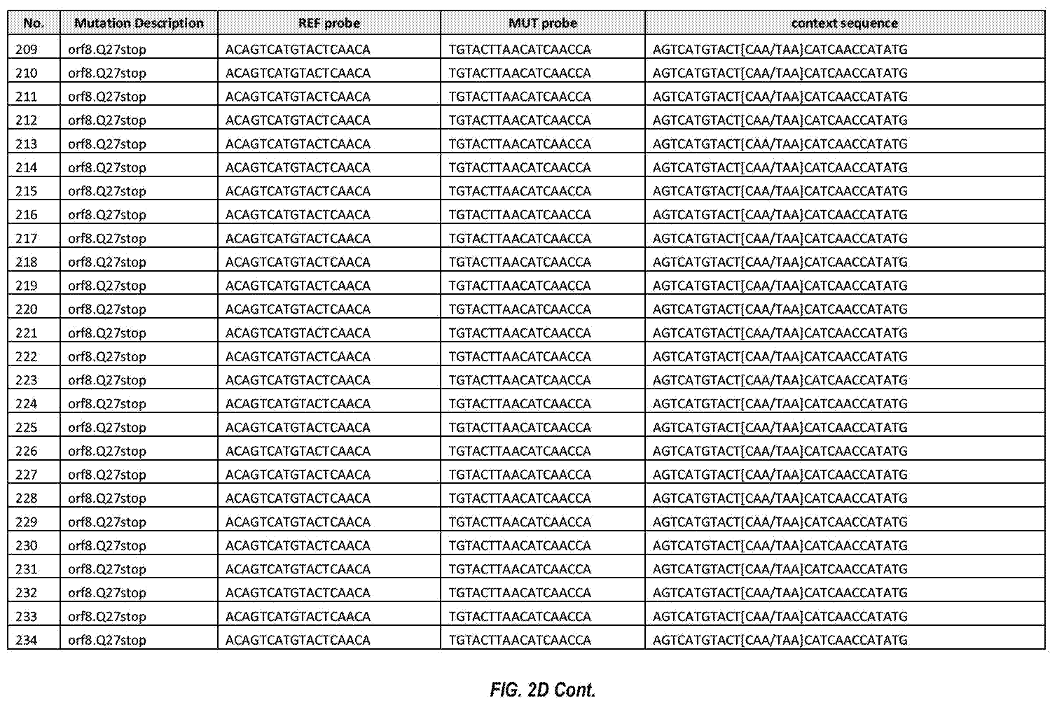

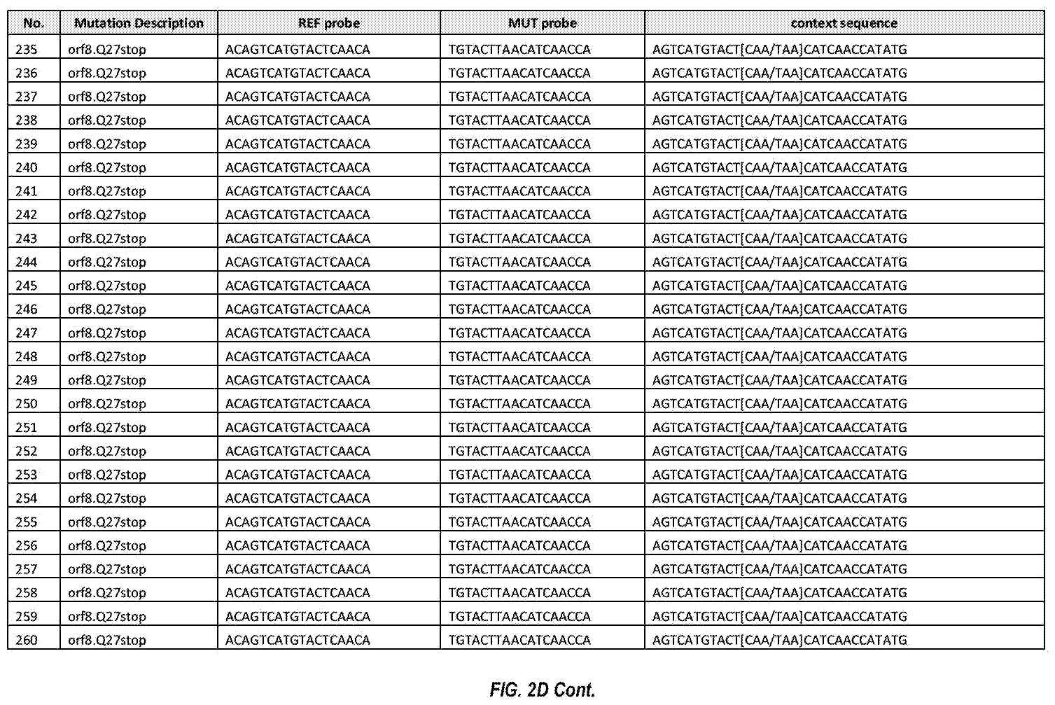

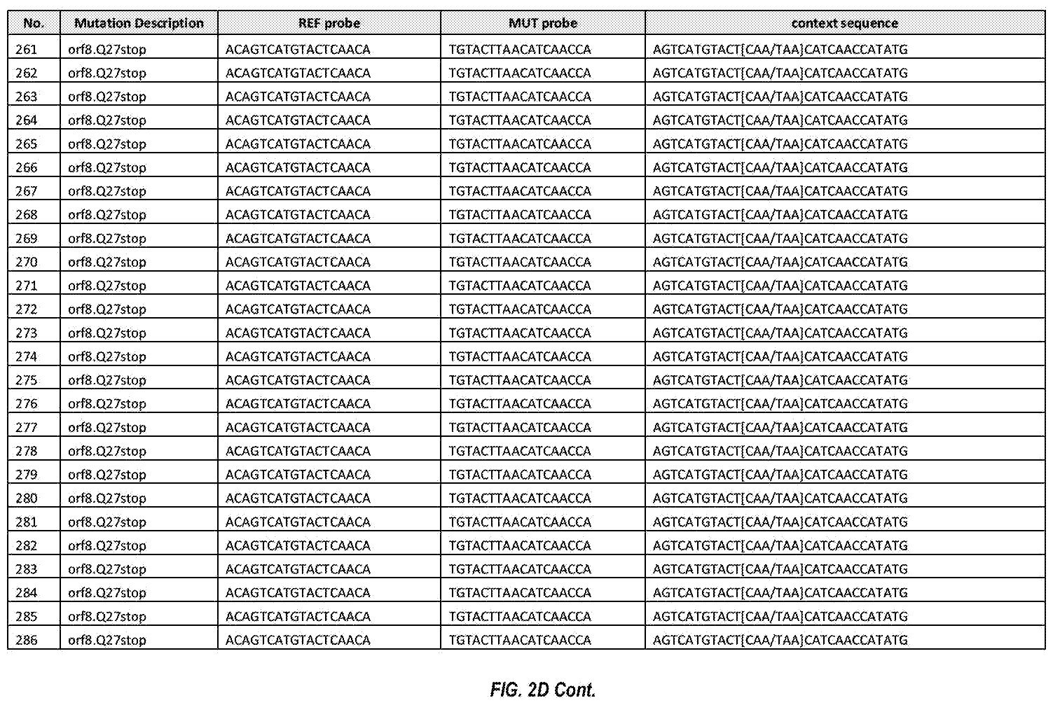

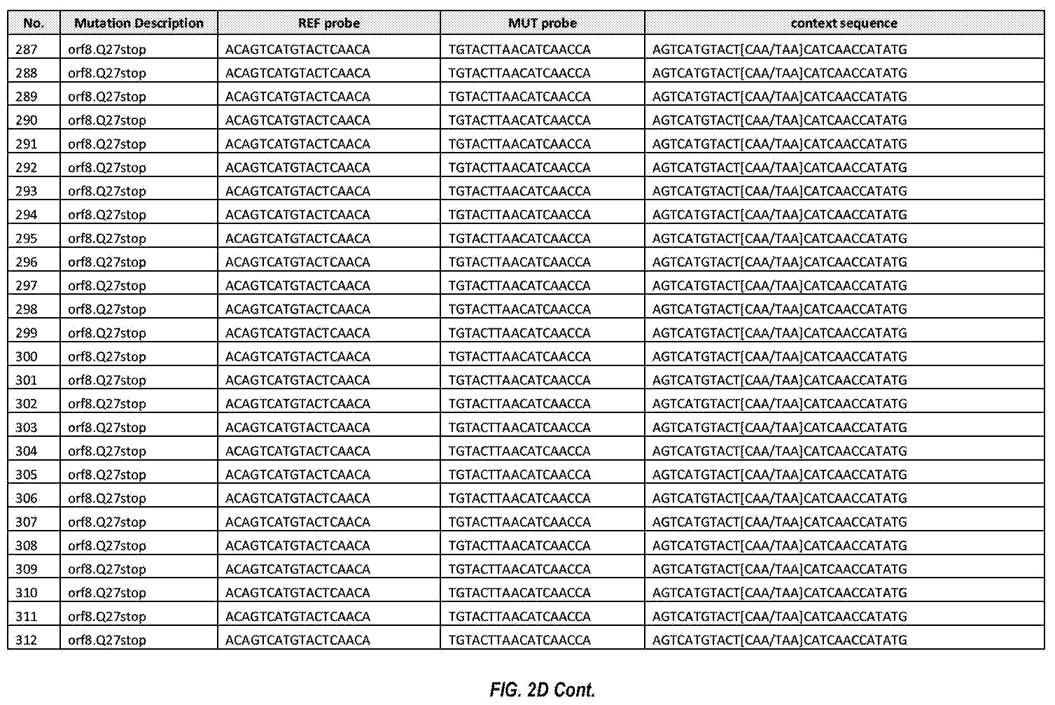

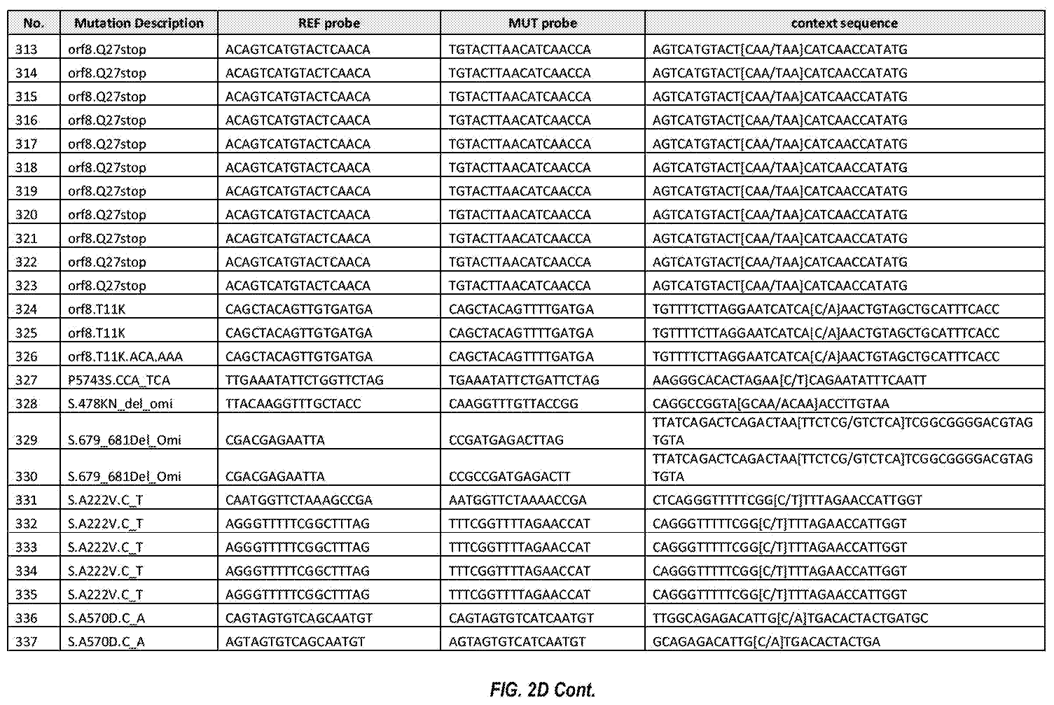

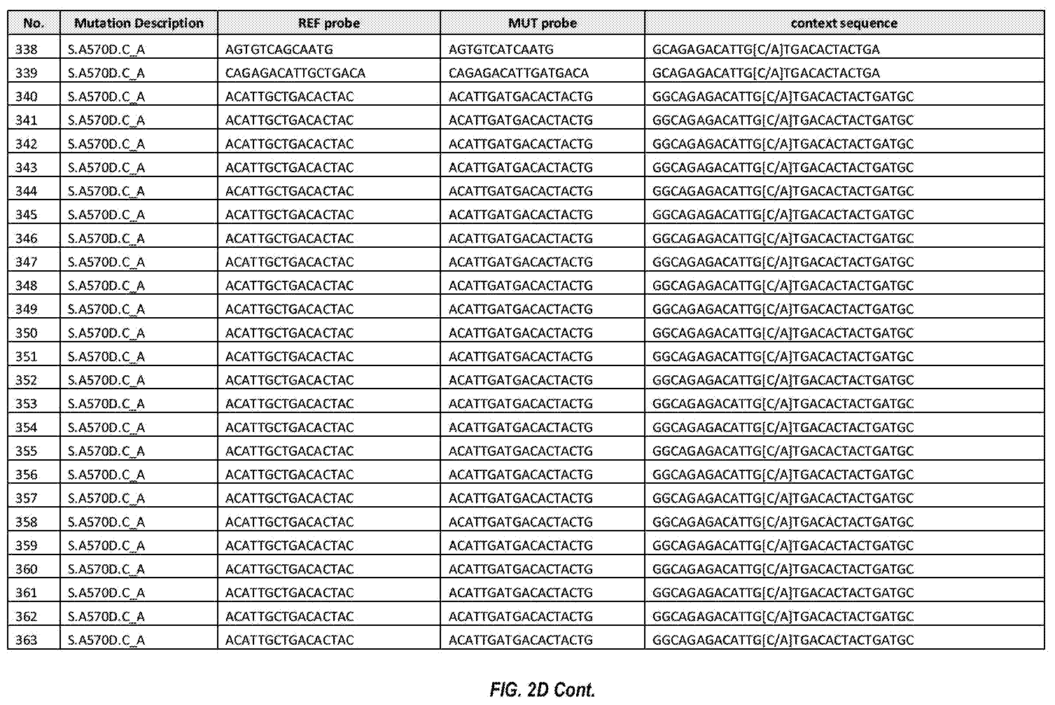

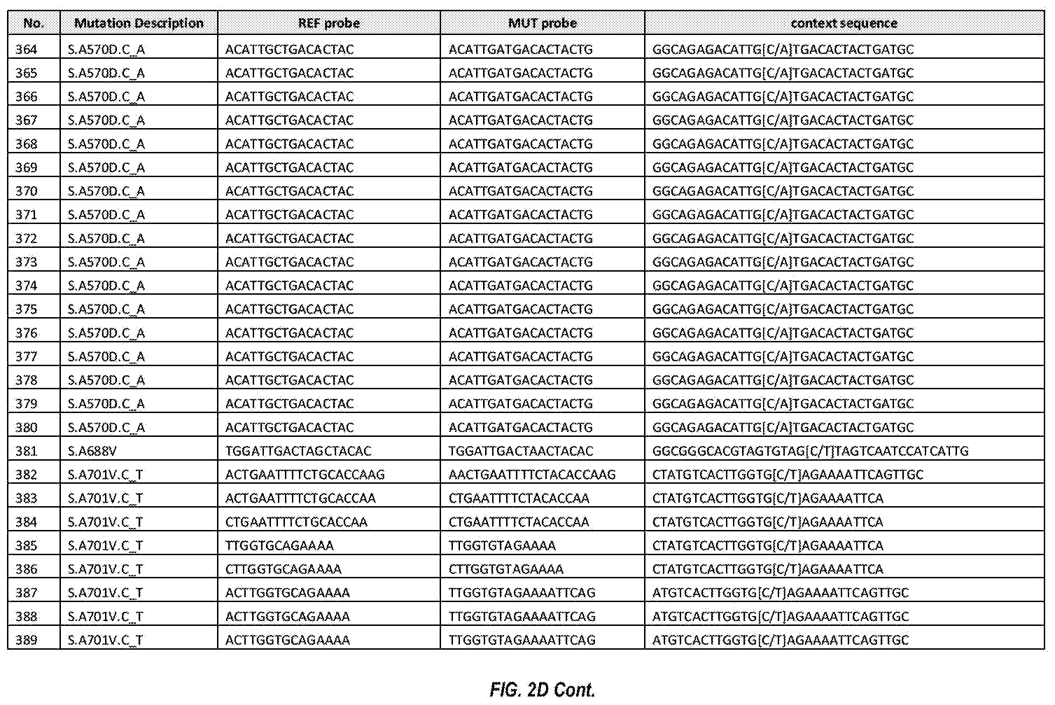

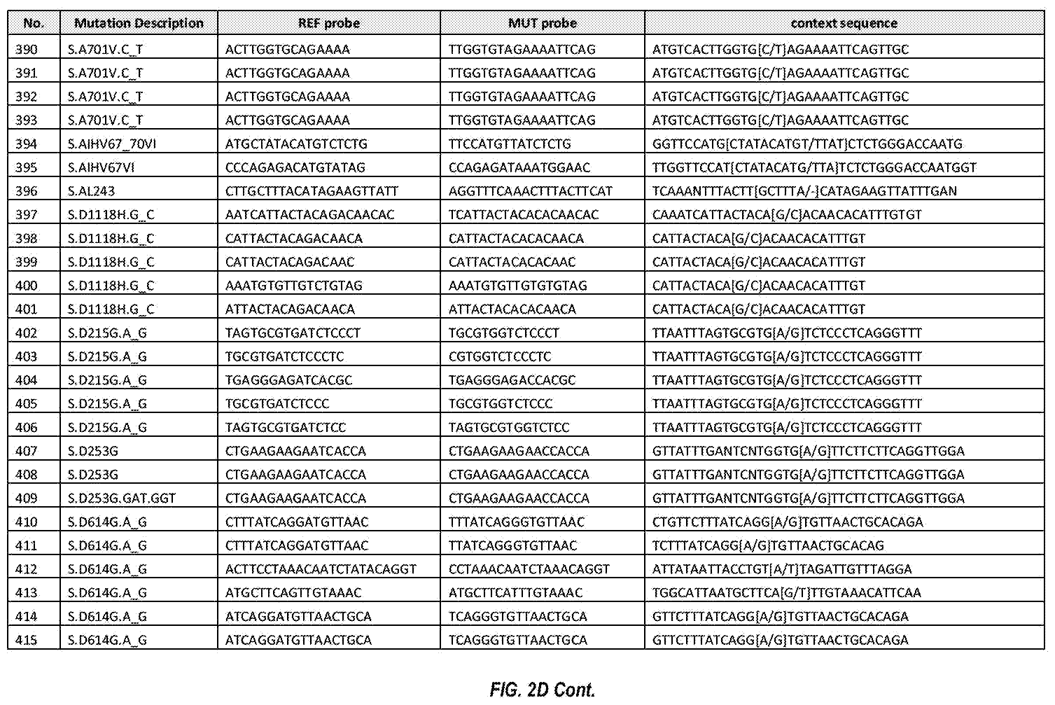

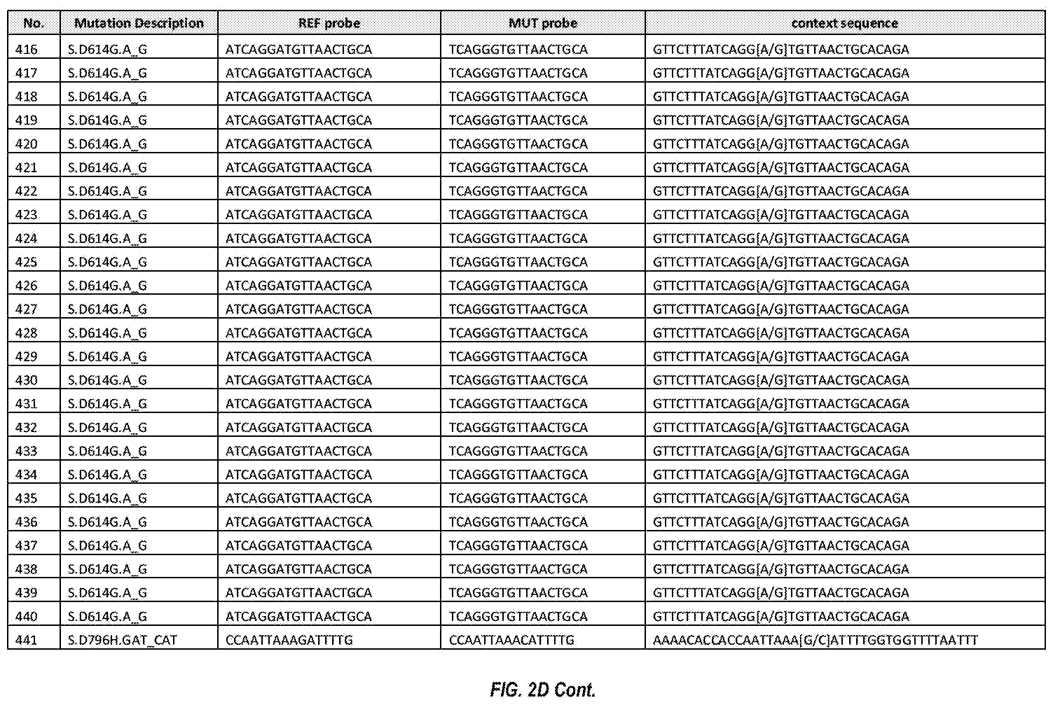

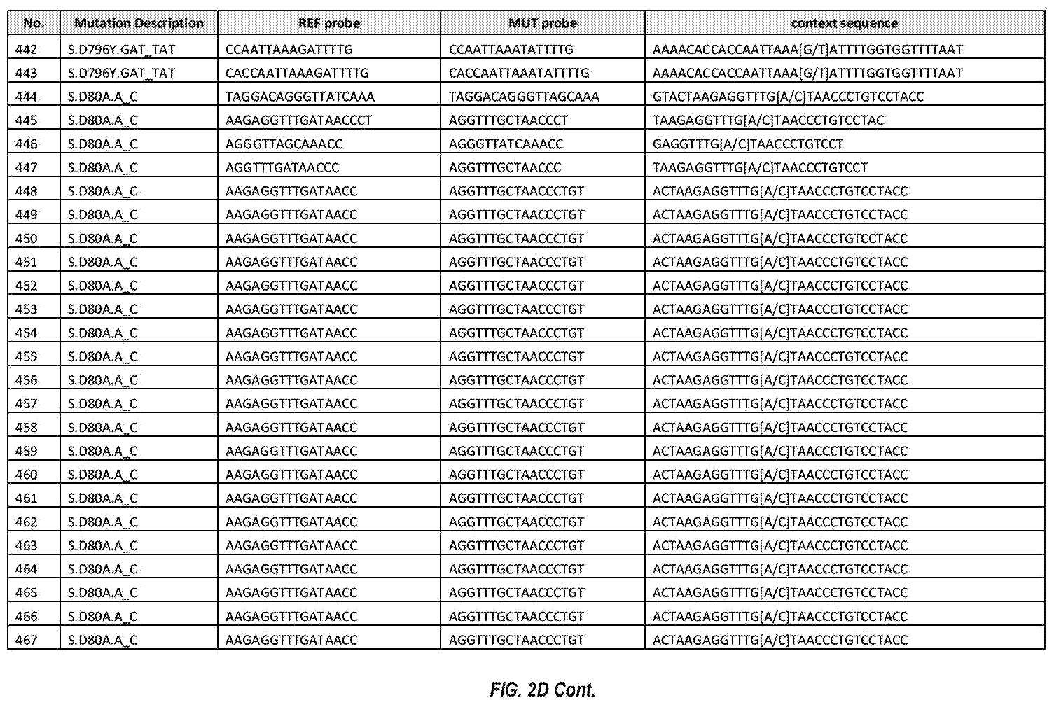

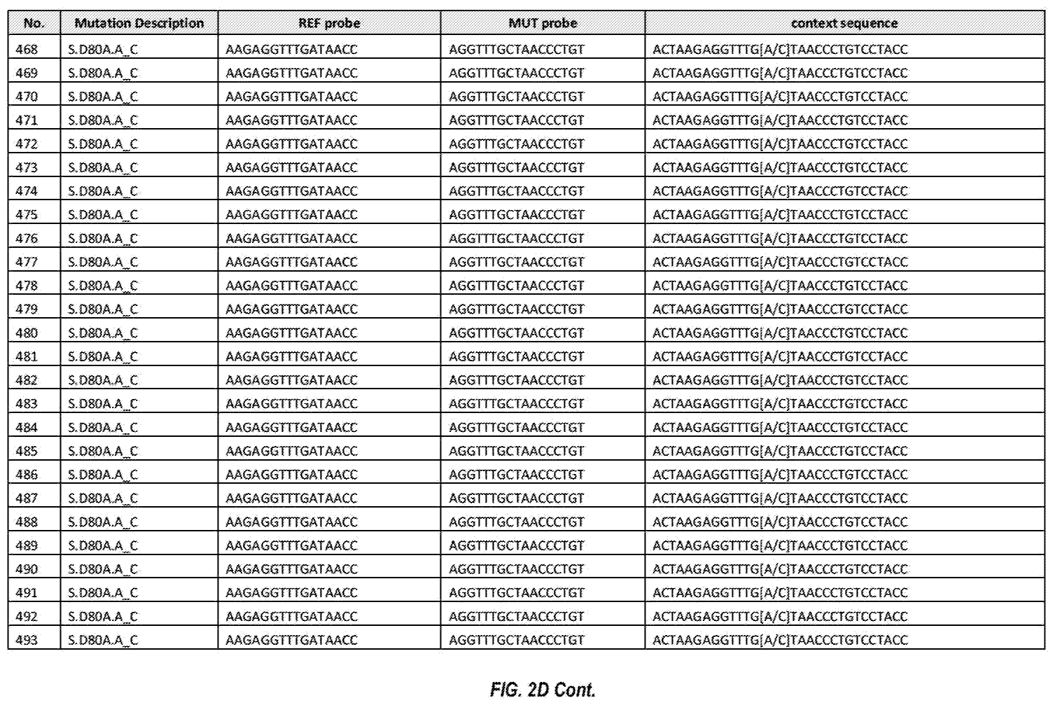

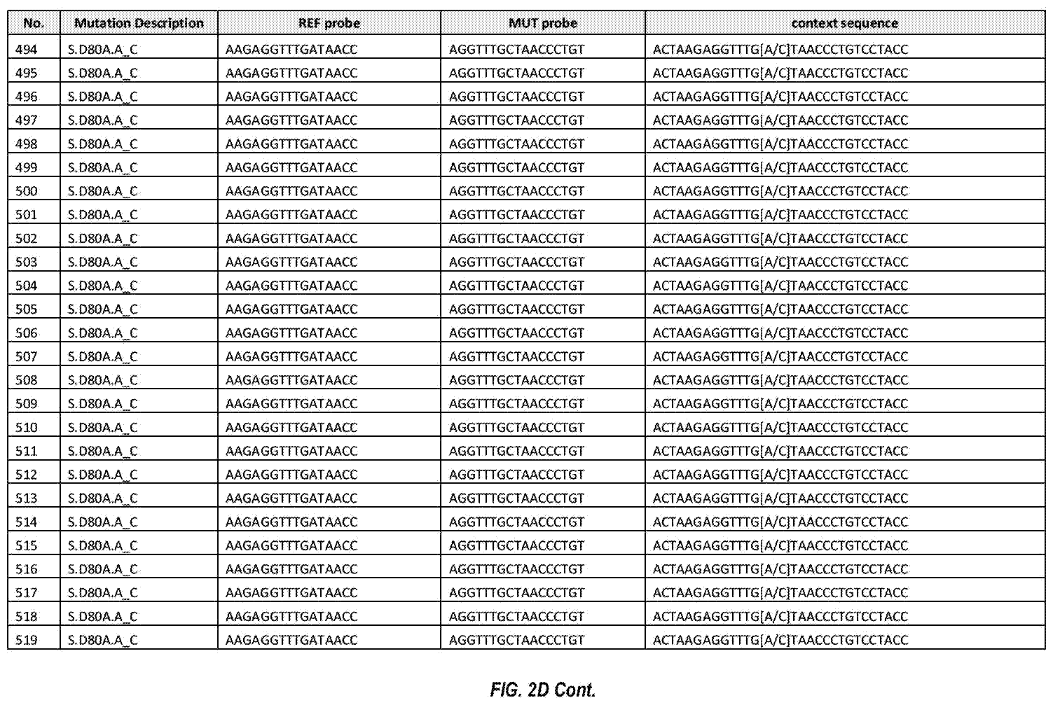

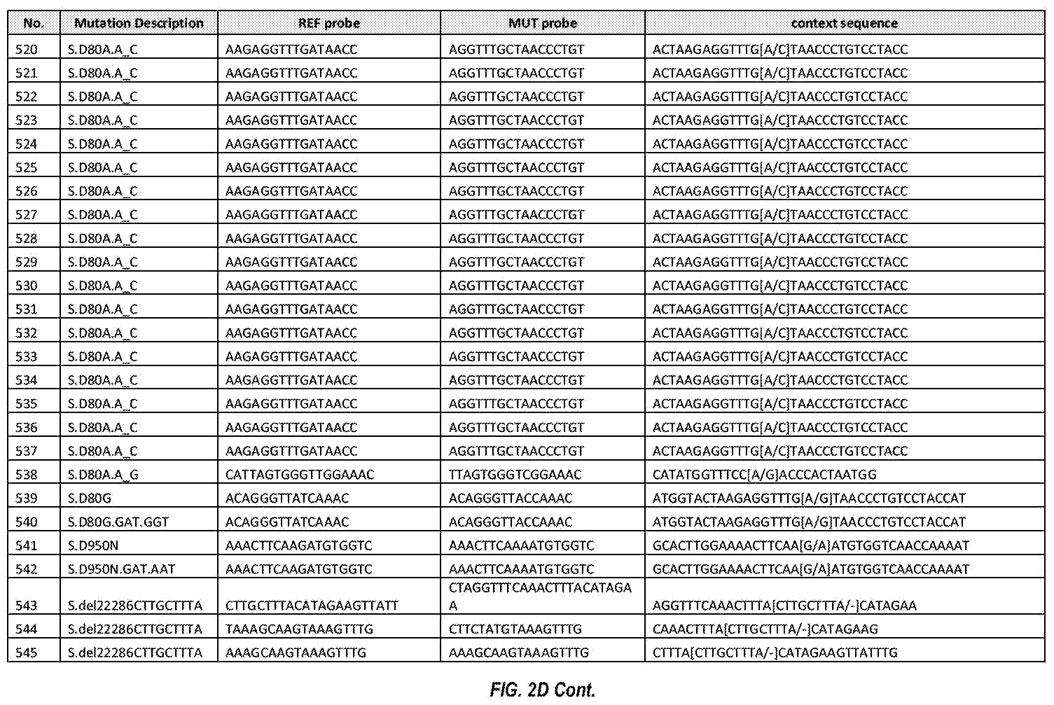

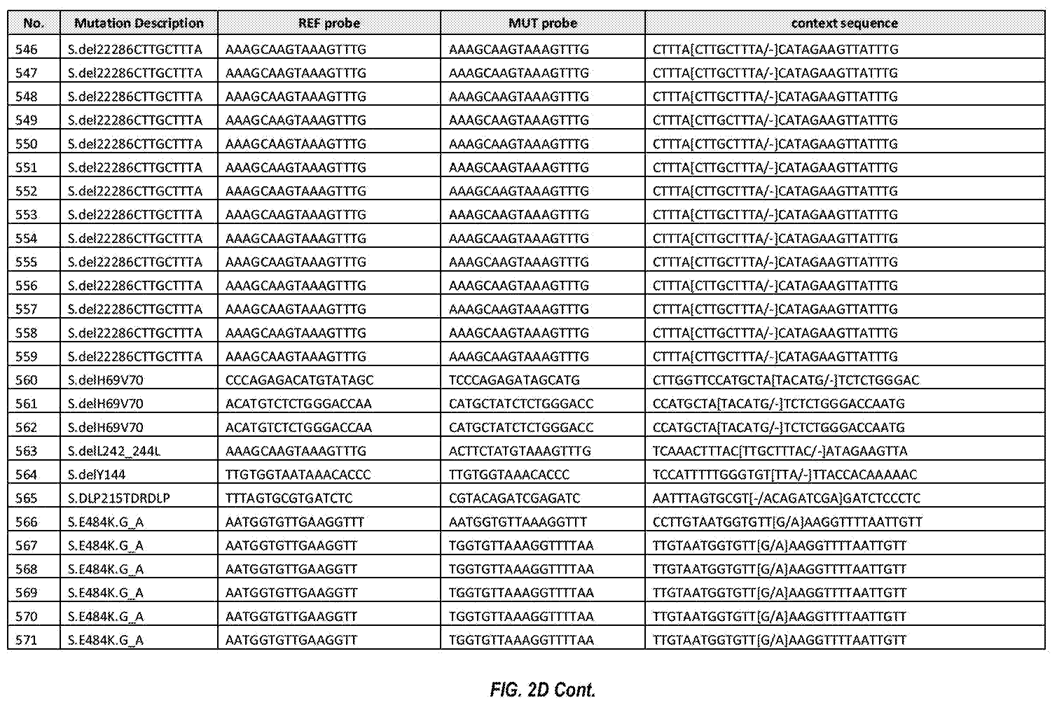

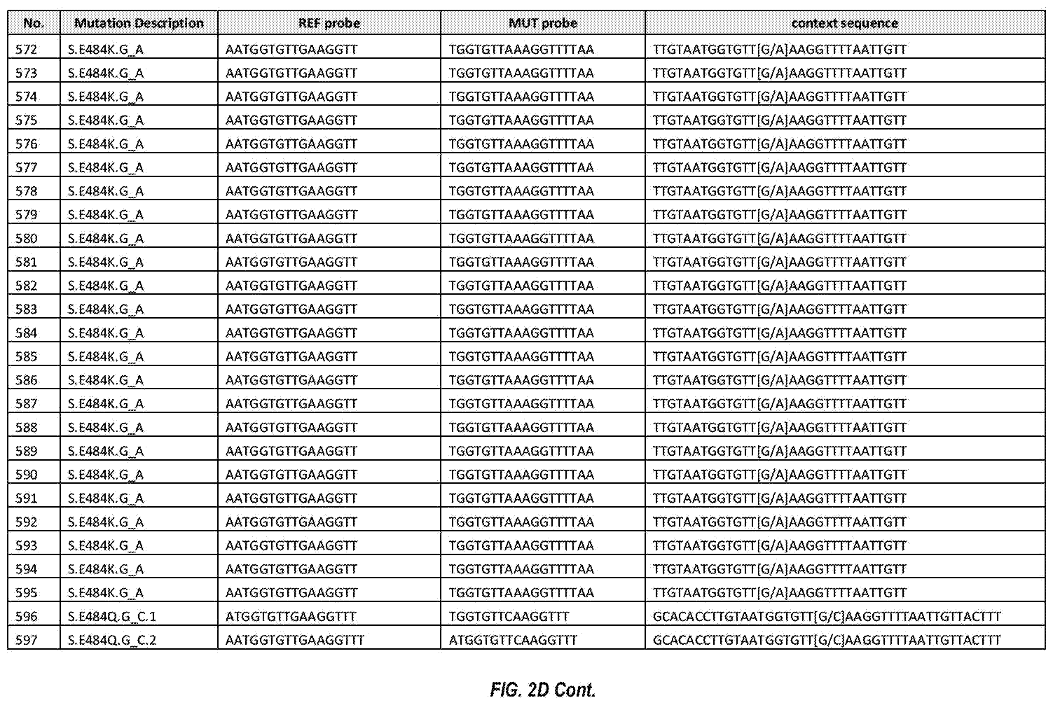

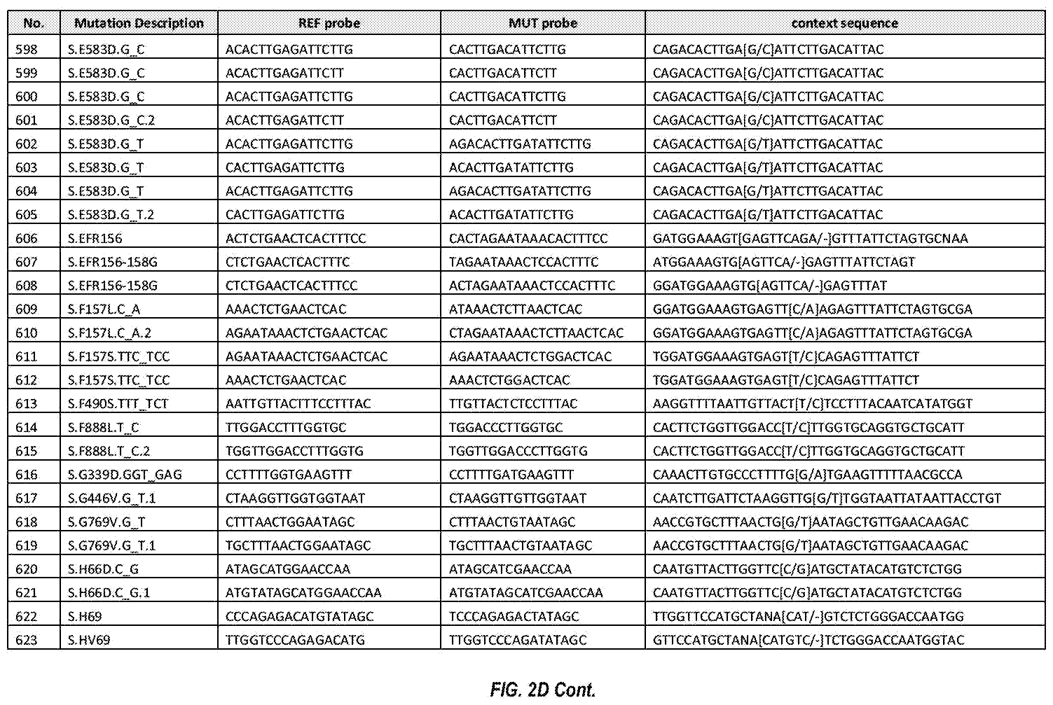

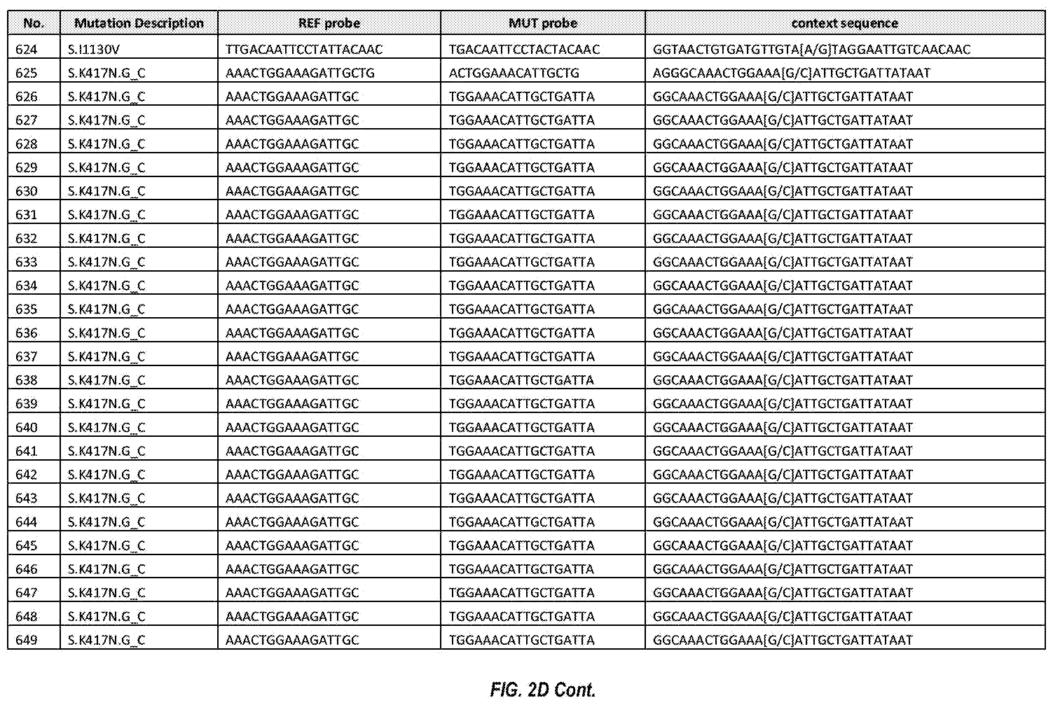

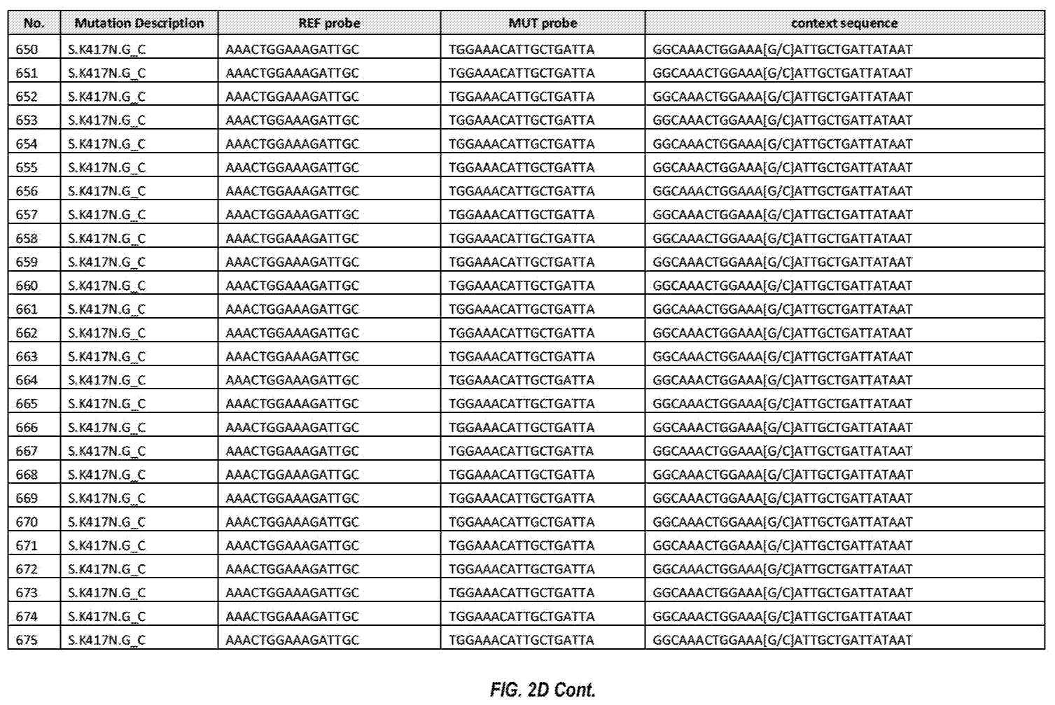

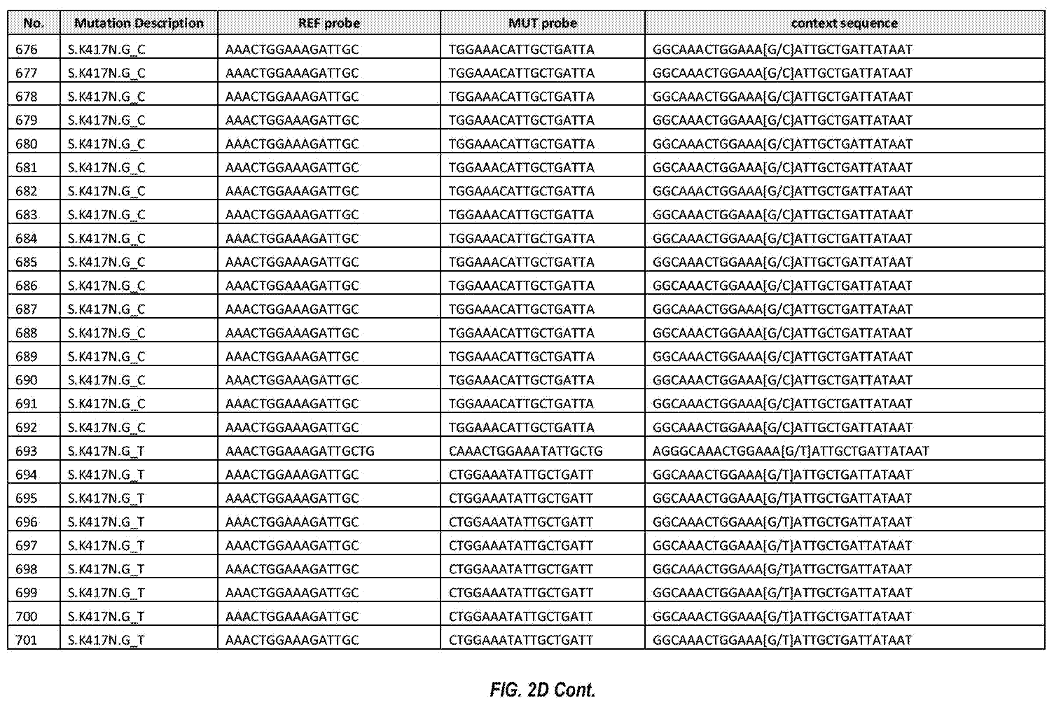

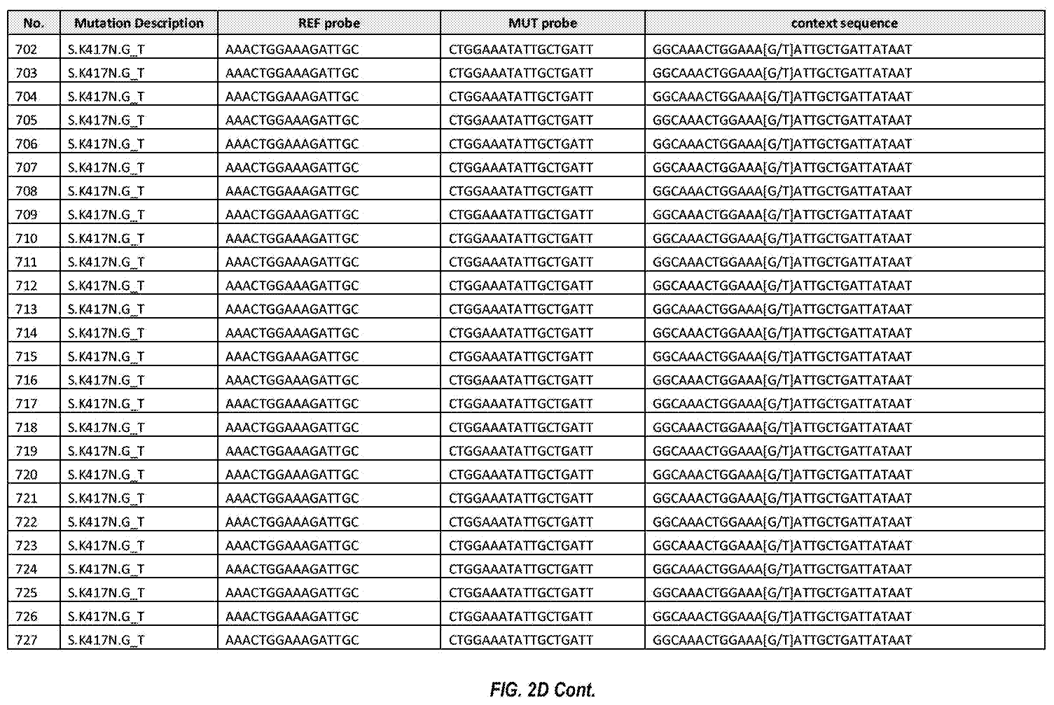

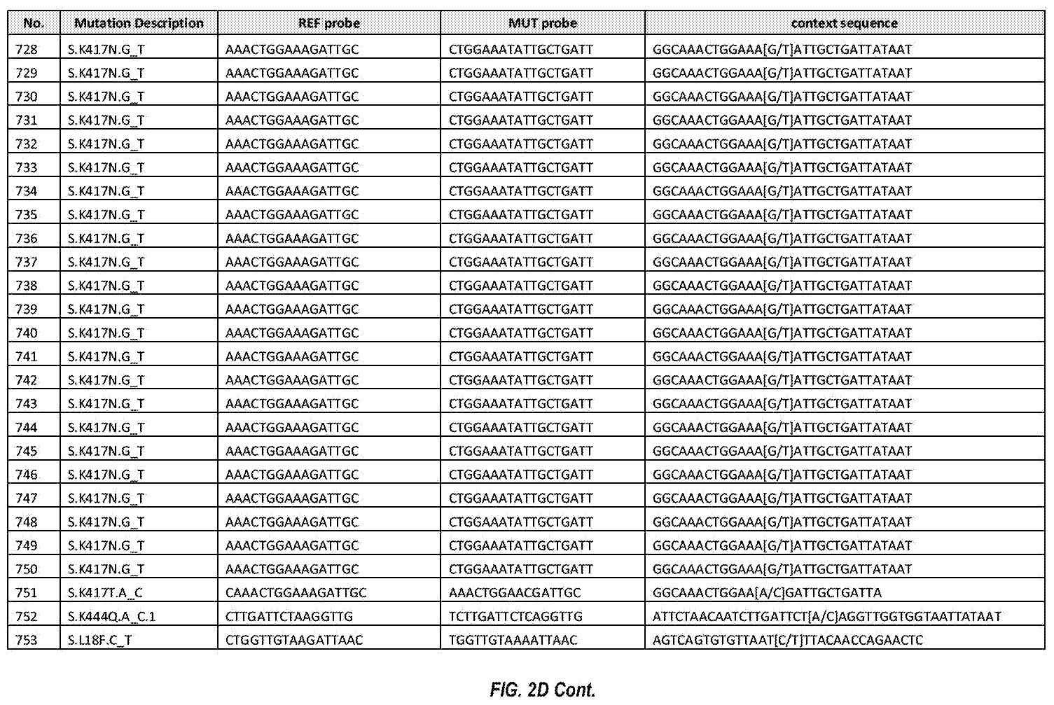

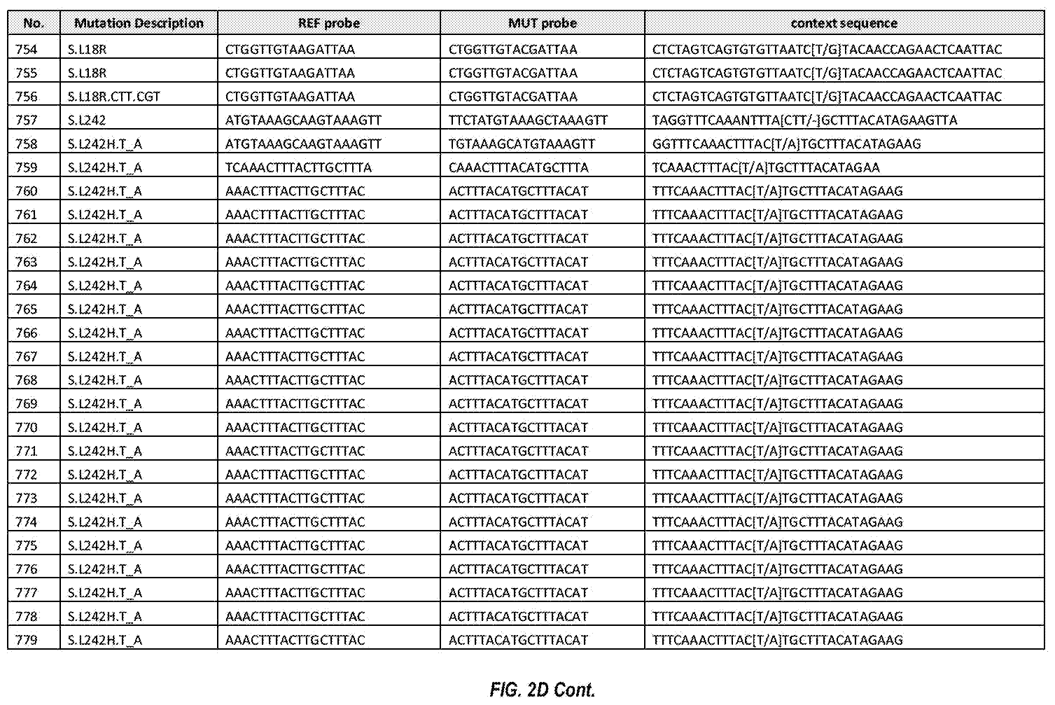

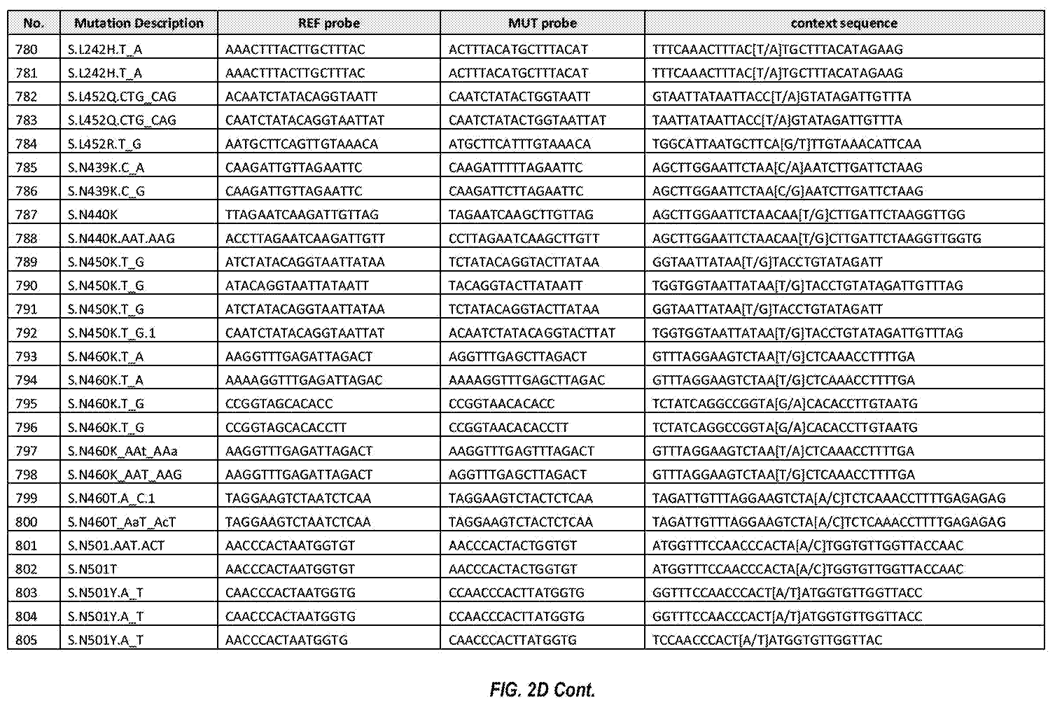

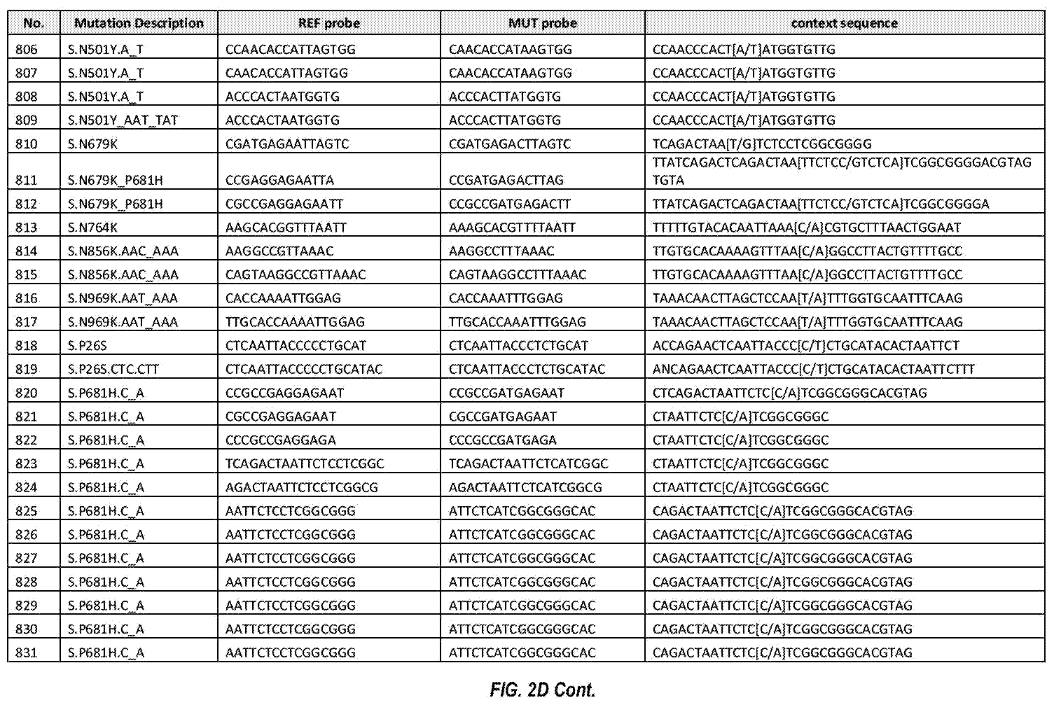

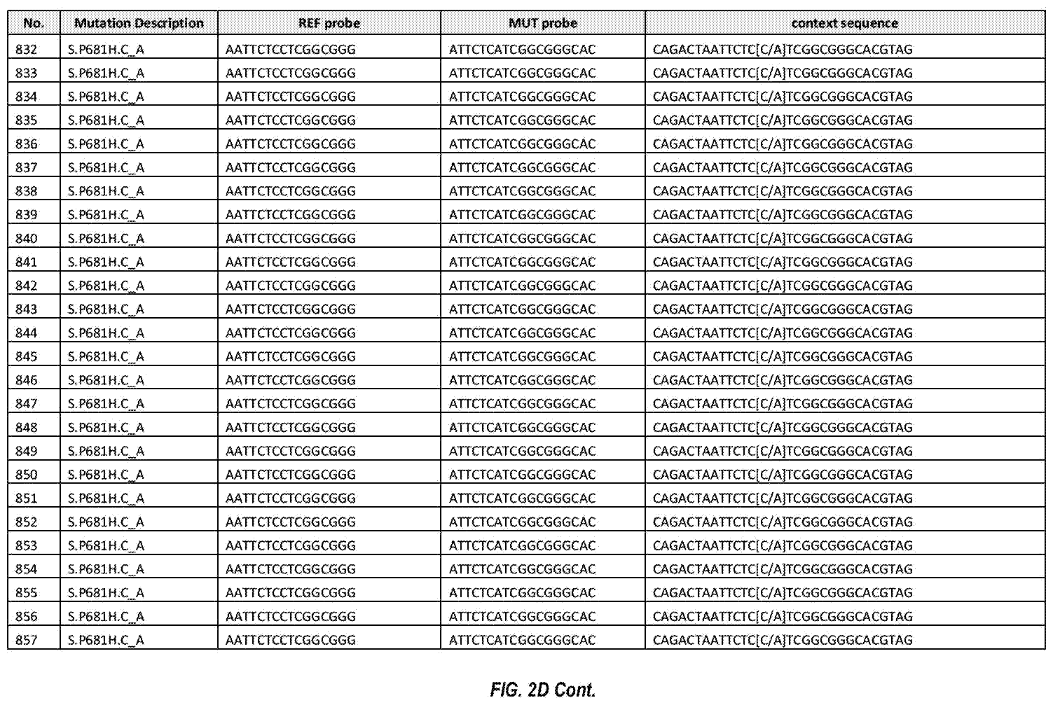

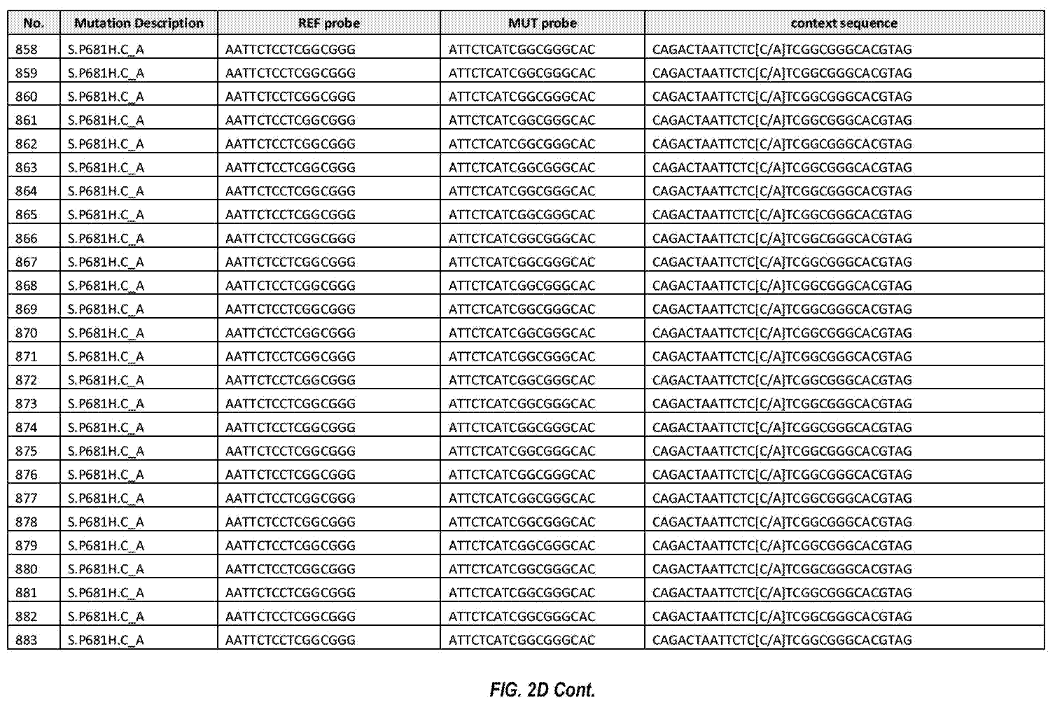

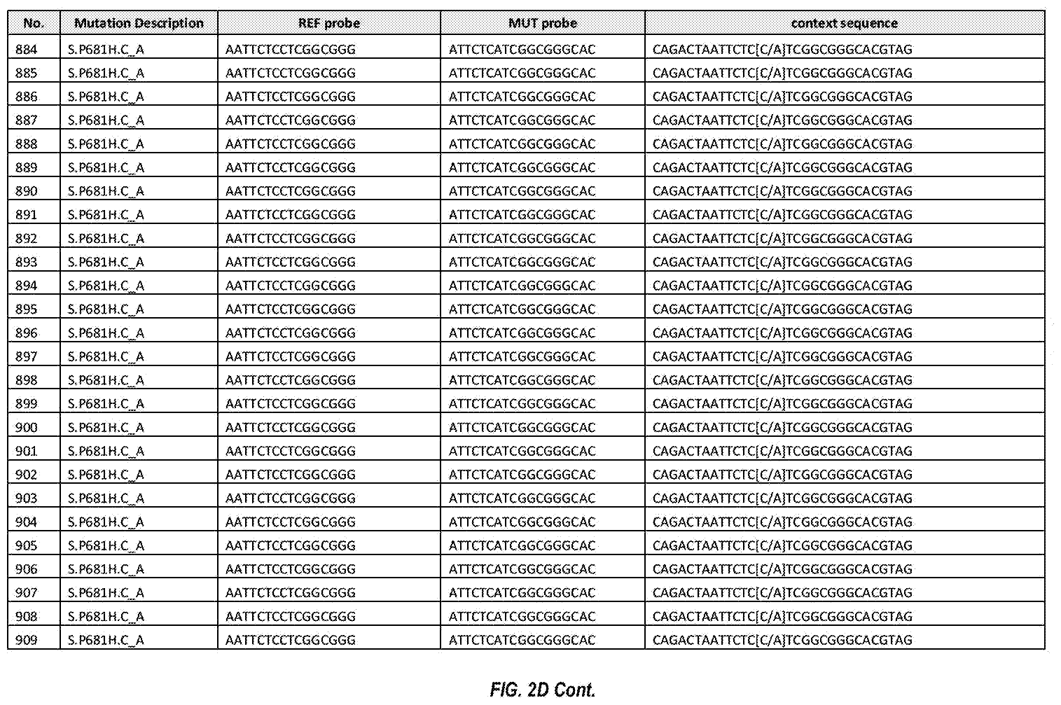

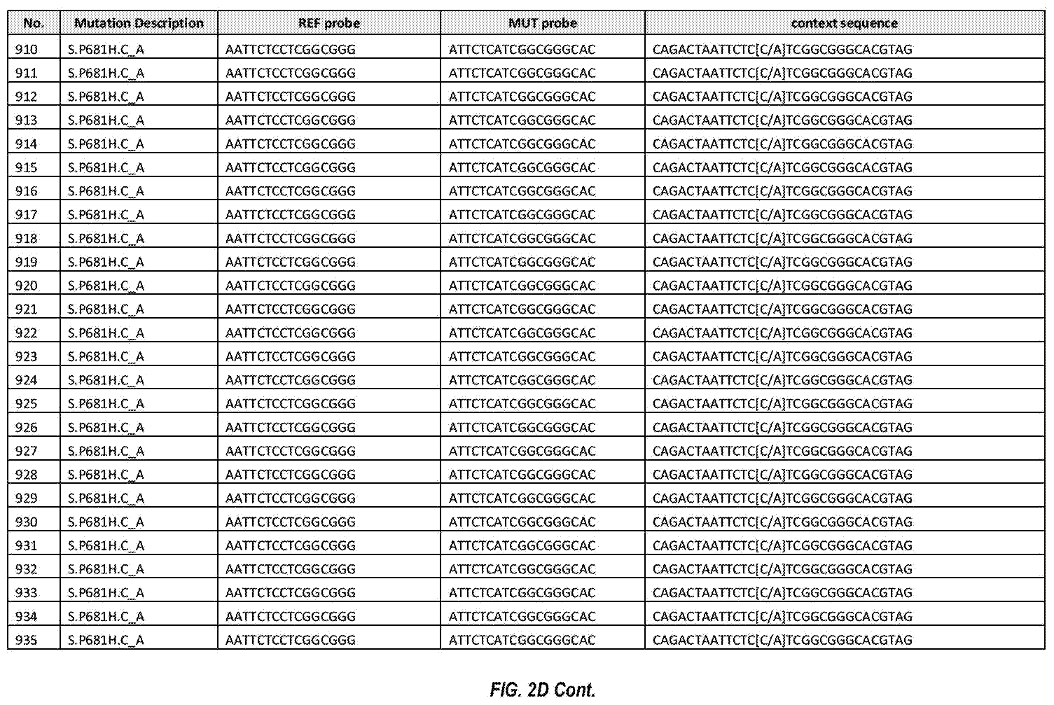

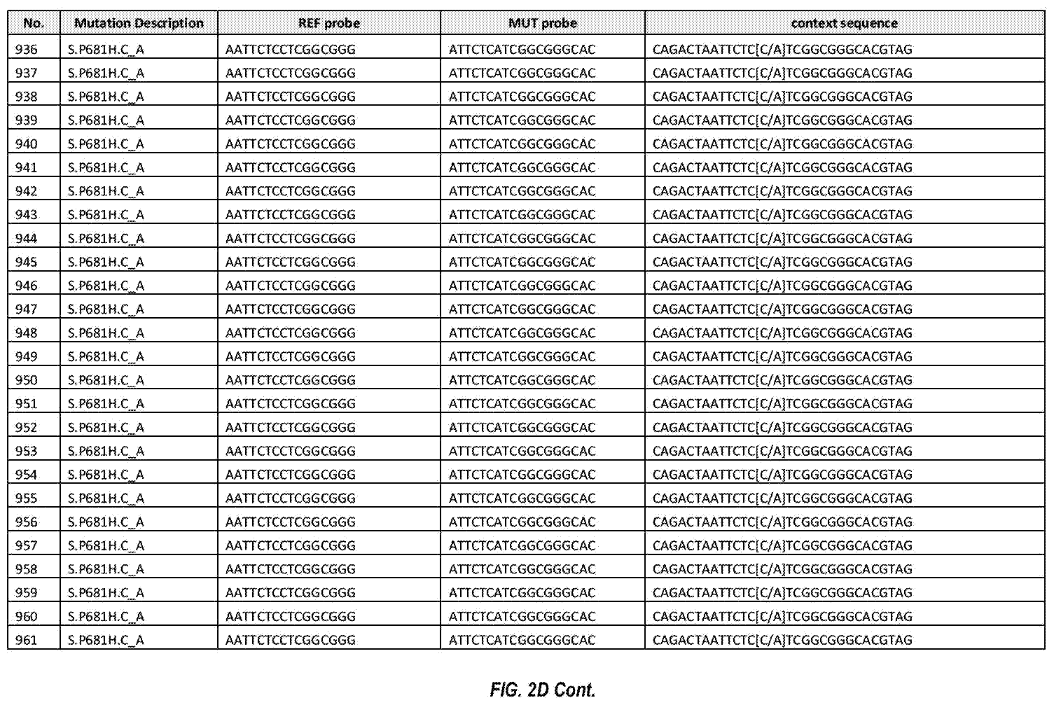

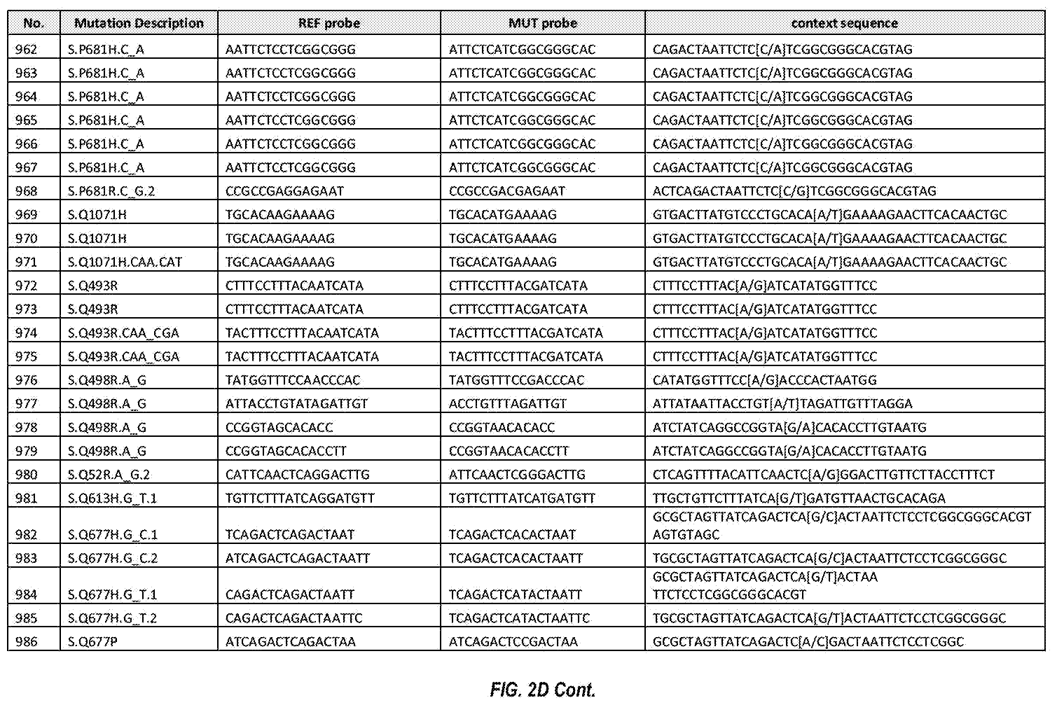

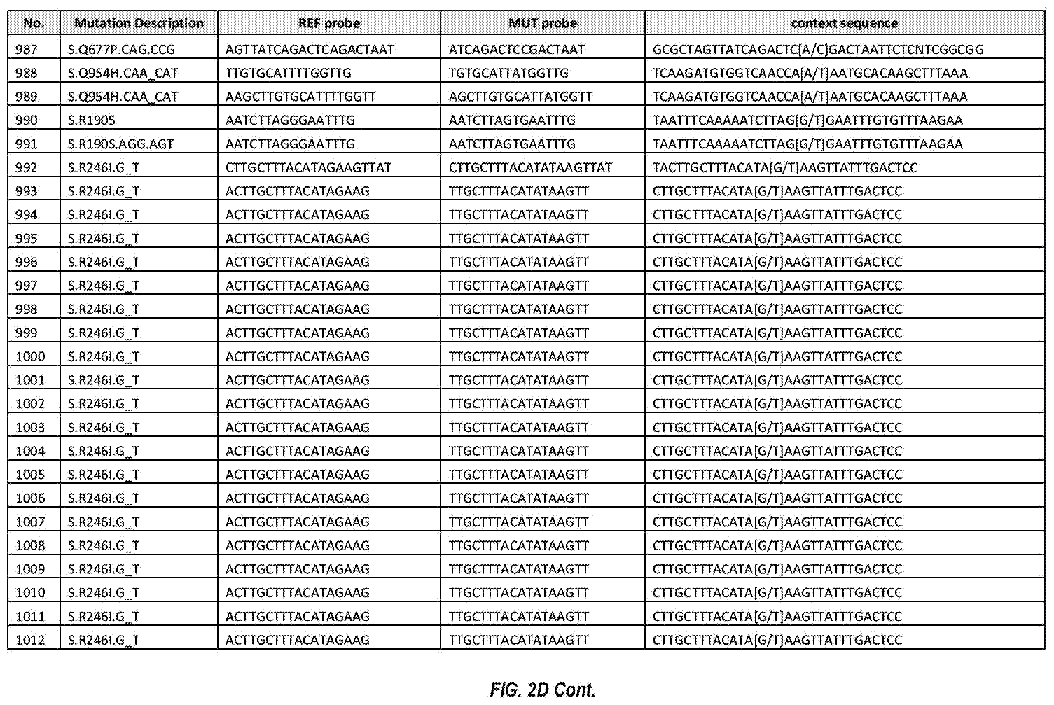

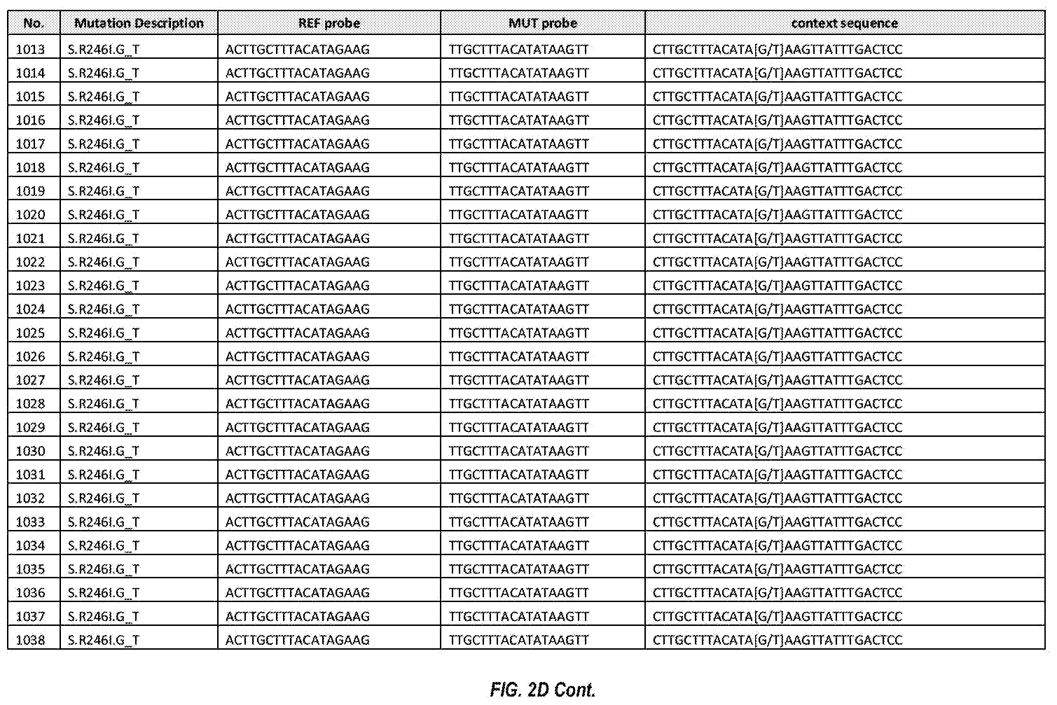

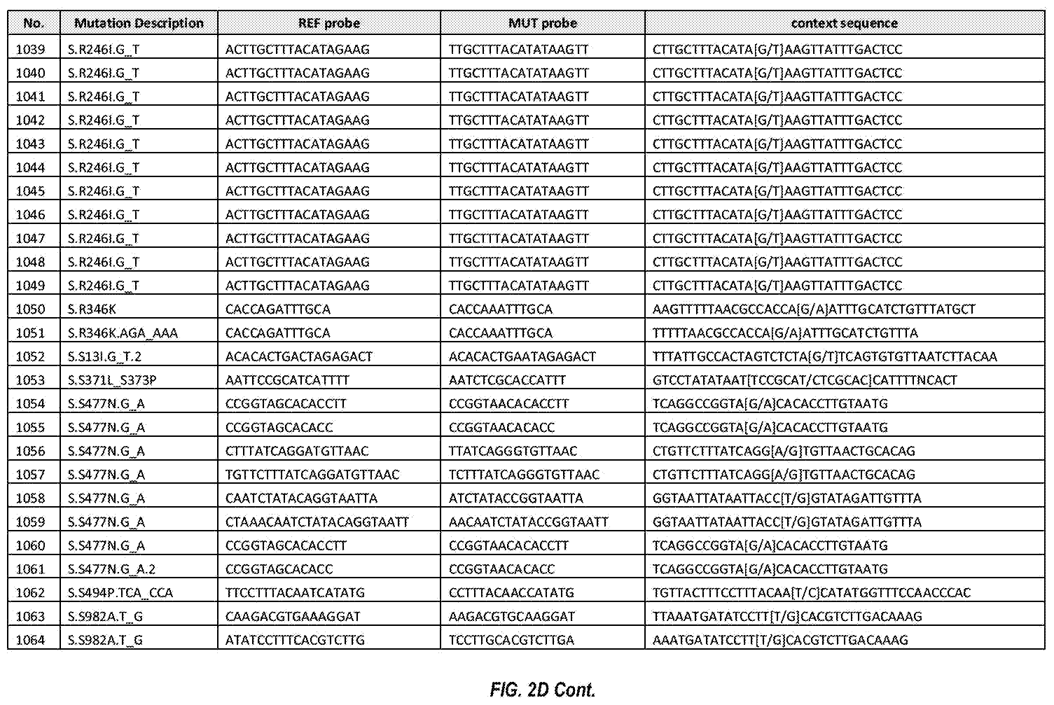

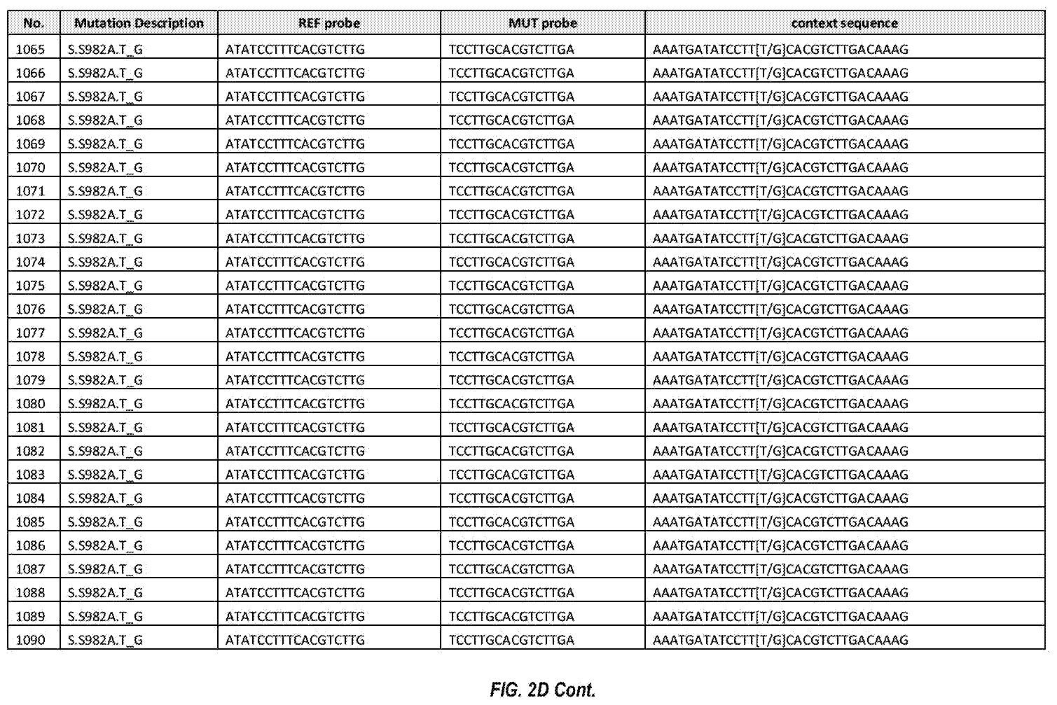

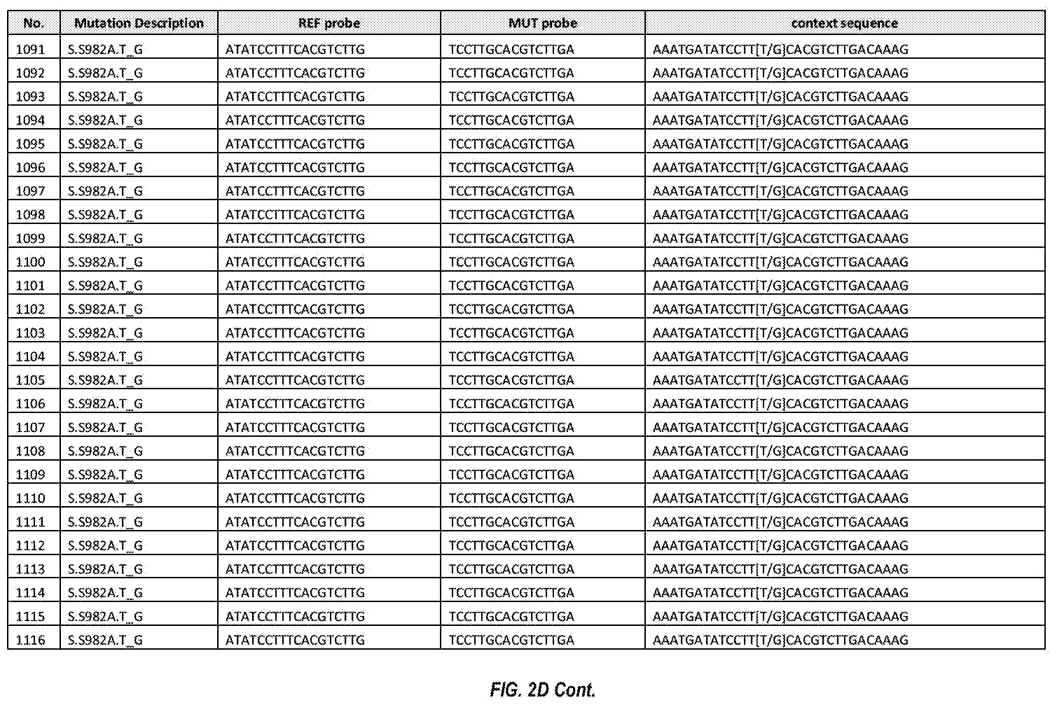

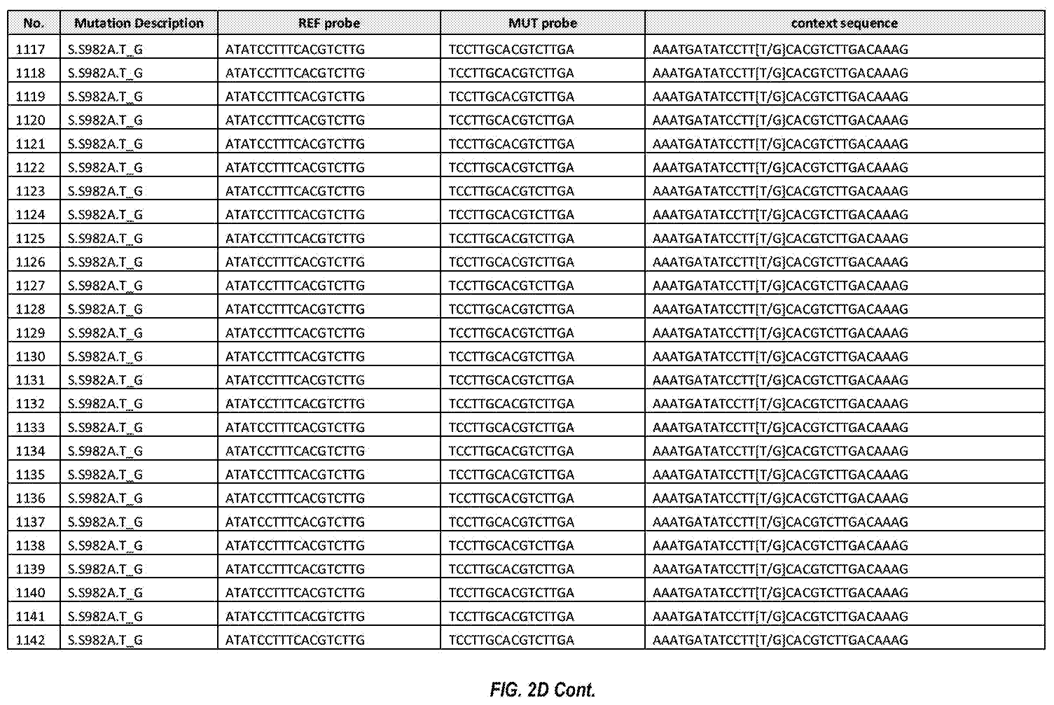

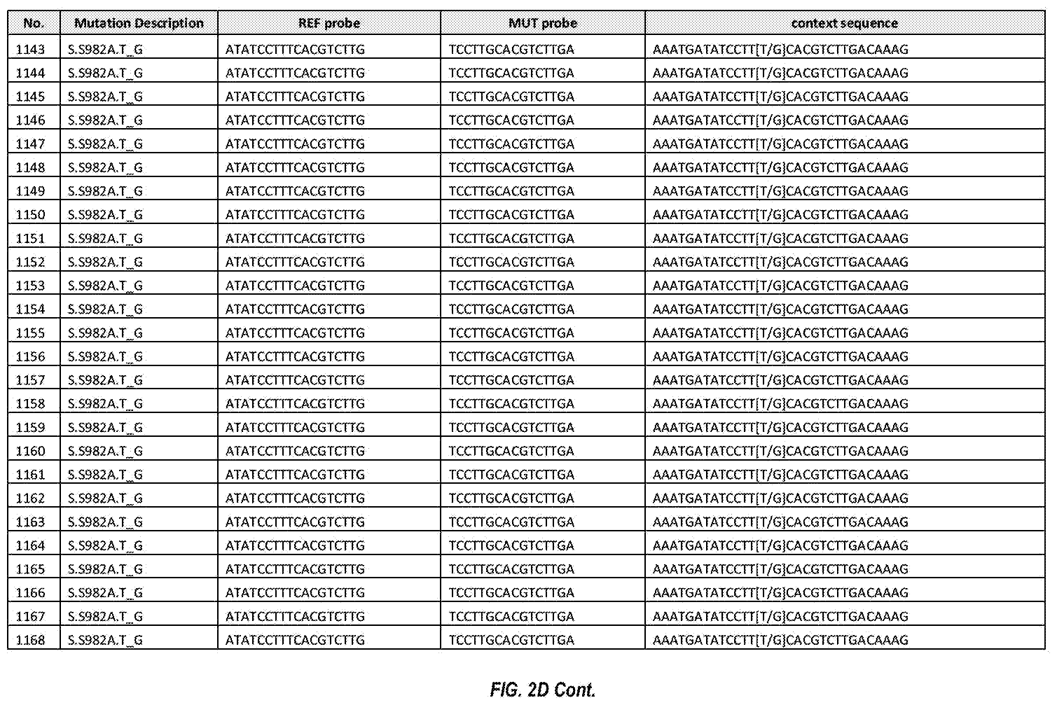

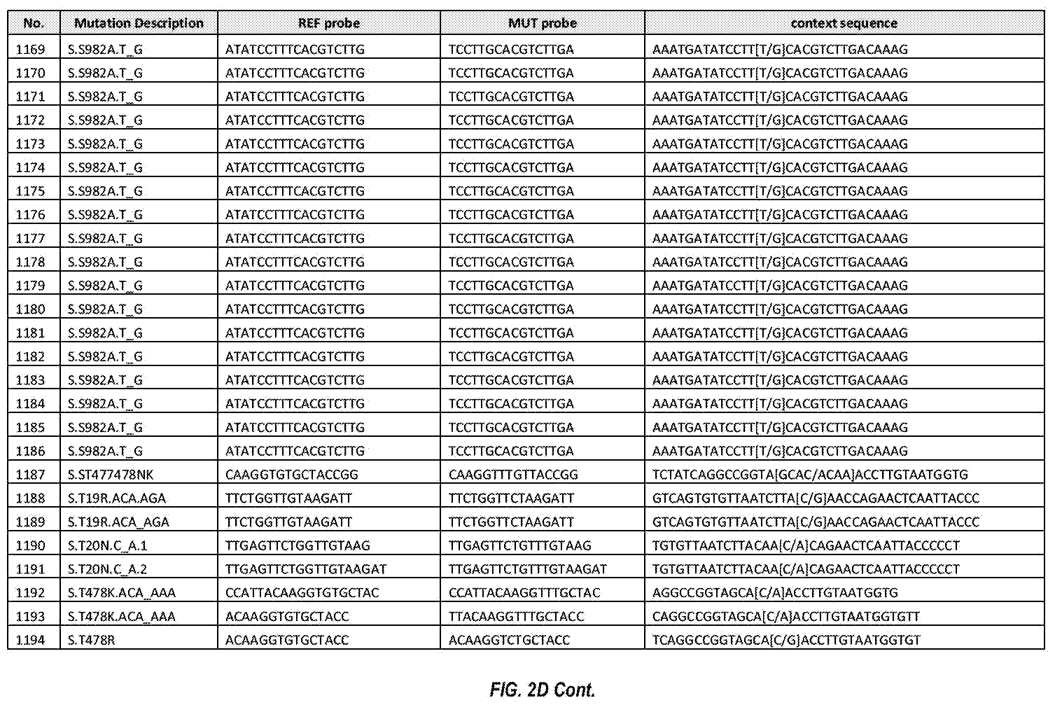

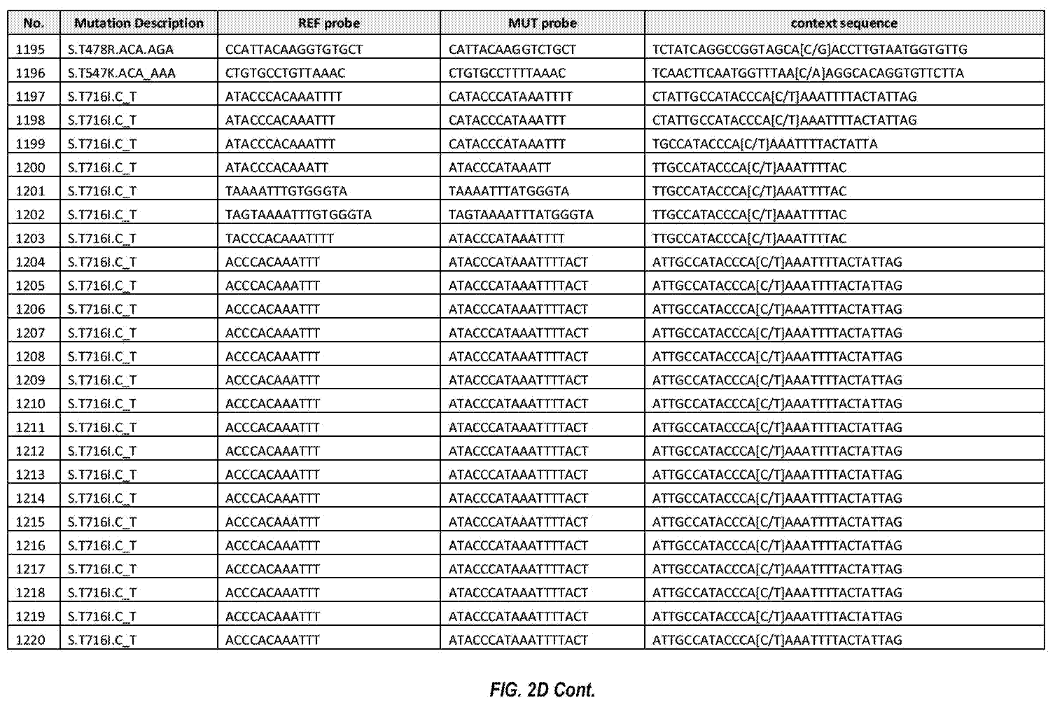

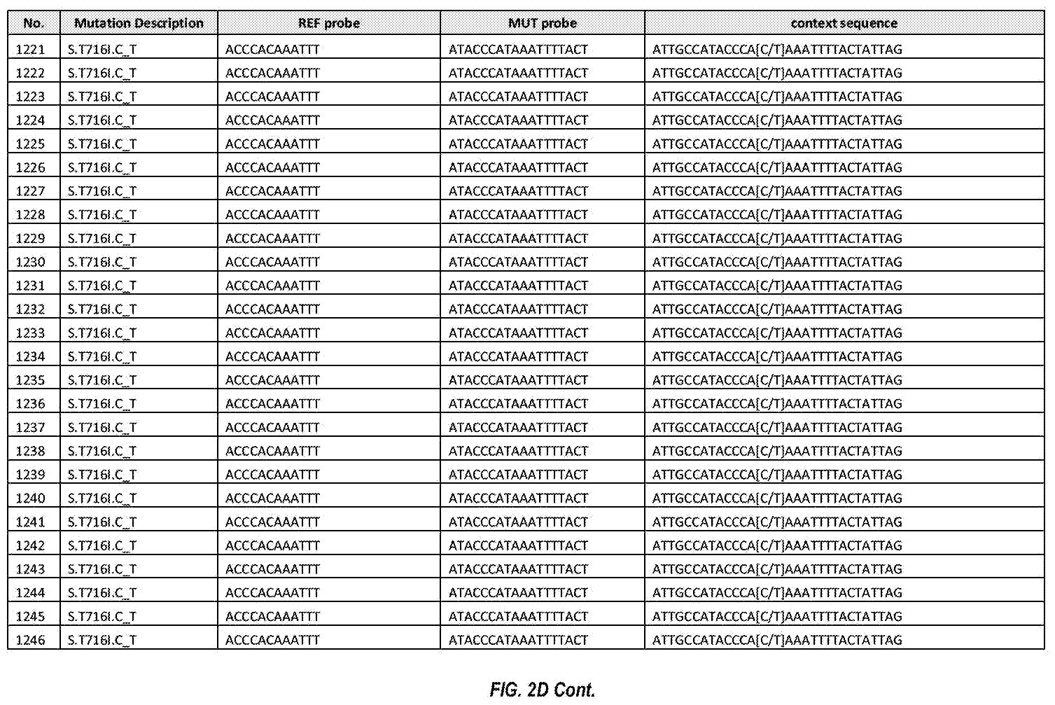

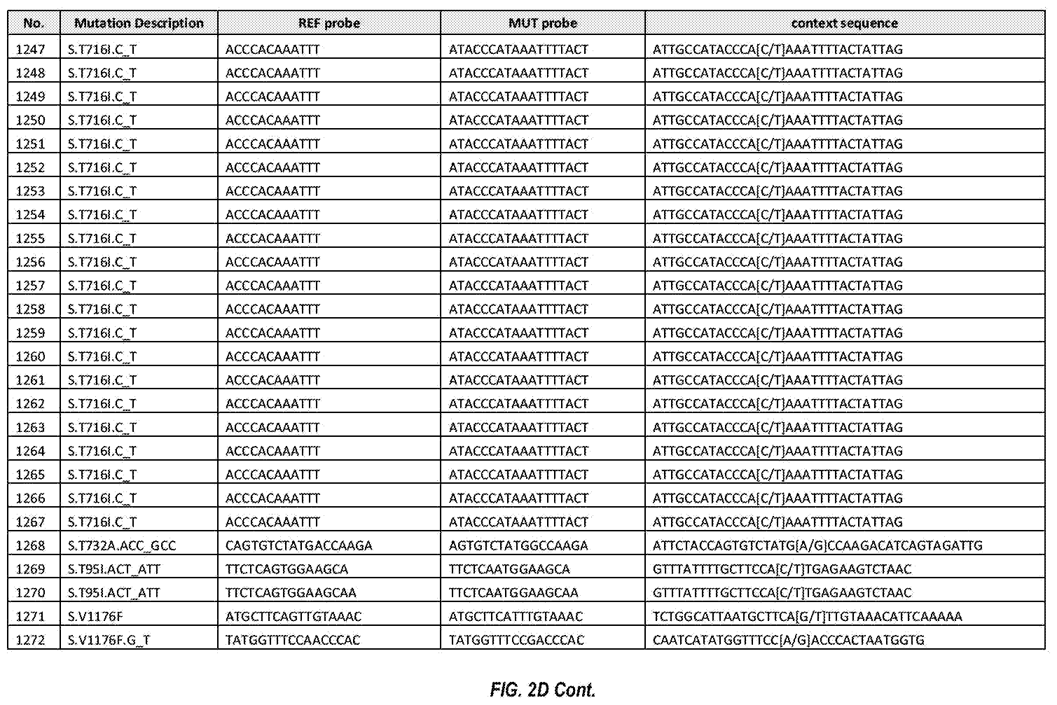

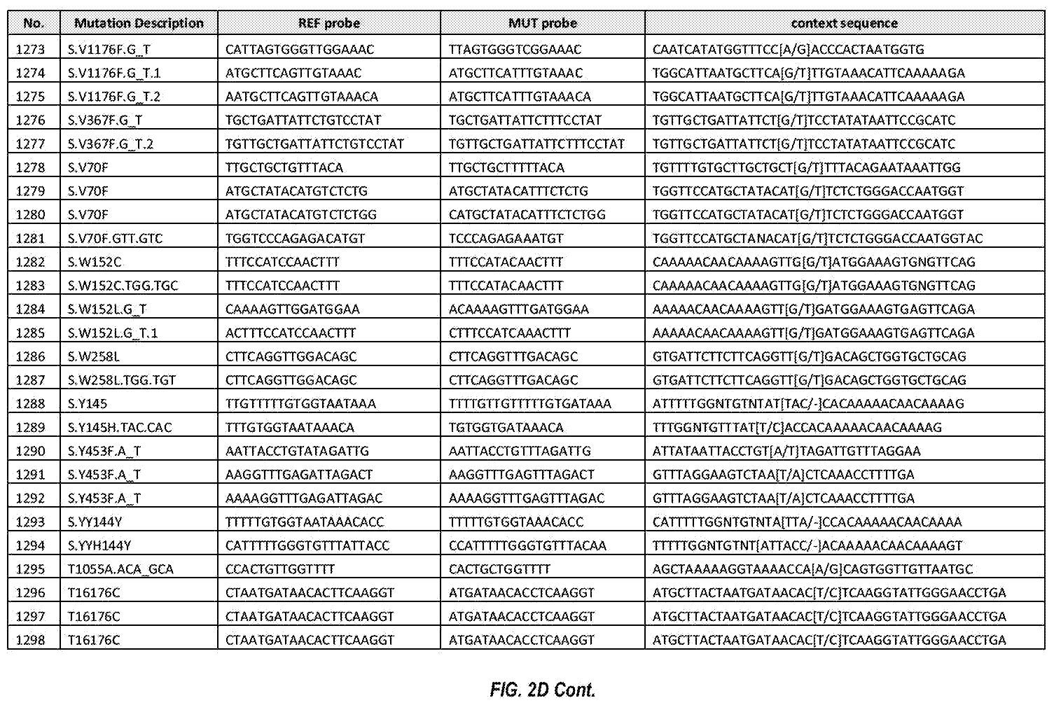

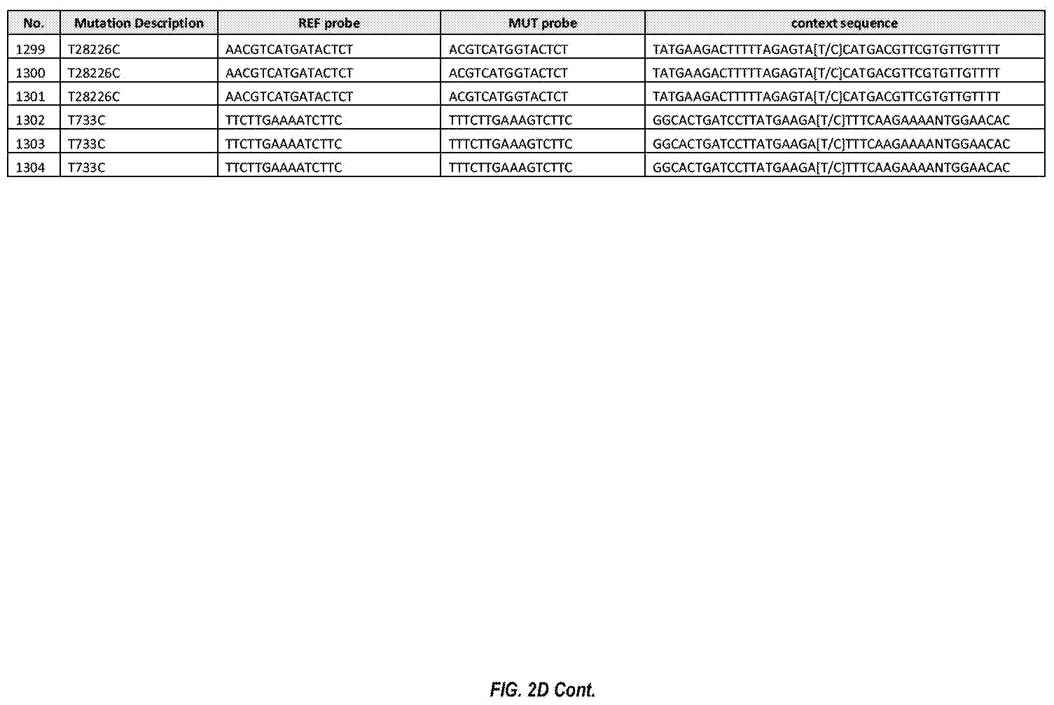

FIGS. 2A and 2B illustrate one exemplary process for detecting SARS-CoV-2 and distinguishing between different forms, such as between forms that share the reference allele and a variant form or between different variant forms. As shown in FIG. 2A, reverse transcription of target RNA is followed by amplification of the resulting cDNA. The reaction mixture includes two or more separate probes each designed to target a different form of the target. In this example, a wild type “W” probe is designed to hybridize to amplicons resulting from the reference form (or from variants that share the reference allele for the targeted region), while a mutant “M” probe is designed to hybridize to amplicons resulting from a variant form having one or more mutations in the targeted region. Each probe type is also associated with a different dye channel to enable differential detection. In this example, the reference “W” probe includes a VIC dye label and the variant “M” probe includes a FAM dye label. The probes may be configured as TaqMan probes, which are known in the art and described in greater detail below. As shown in FIG. 2B, when the probe is able to hybridize to a target downstream from a primer, the exonuclease activity of the polymerase during subsequent primer extension separates the dye label from the quencher to increase the dye signal.

Disclosed herein are primers and probes that correspond to mutations and variants (e.g., mutations and/or variants disclosed in Table 4), and that may be utilized in assays that can beneficially identify particular variants associated with such mutations and/or distinguish such variants from other variants (and/or from reference SARS-CoV-2). FIGS. 2C and 2D illustrate exemplary primers and probes that may be utilized in such assays. FIG. 2C illustrates exemplary forward primers (corresponding to SEQ ID NO:1-SEQ ID NO: 1304) and reverse primers (corresponding to SEQ ID NO:2001-SEQ ID NO:3304), while FIG. 2D illustrates exemplary probes (corresponding to SEQ ID NO:4001-SEQ ID NO:5304 and SEQ ID NO:6001-SEQ ID NO:7304) that may be utilized in conjunction with corresponding forward and reverse primers of FIG. 2C. For example, in some embodiments, an assay can include a forward primer and reverse primer in a particular “No.” row of FIG. 2C and a reference probe and mutant probe in the same “No.” row of FIG. 2D. In other embodiments, an assay can include one or more forward primers selected from SEQ ID NO:1-SEQ ID NO:1304, one or more reverse primers selected from SEQ ID NO:2001-SEQ ID NO:3304, one or more reference sequence probes selected from SEQ ID NO:4001-SEQ ID NO:5304 and/or one or more mutant sequence probes selected from SEQ ID NO:6001-SEQ ID NO:7304. In particular, the probes of SEQ ID NO:4001-SEQ ID NO:5304 are configured for association with a reference SARS-CoV-2 allele at the corresponding target, while the probes of SEQ ID NO:6001-SEQ ID NO:7304 are configured for association with a mutant allele at the corresponding target. The probes of SEQ ID NO:4001-SEQ ID NO:5304 and SEQ ID NO:6001-SEQ ID NO:7304 may therefore be used in conjunction with each other to identify whether a SARS-CoV-2 mutation is present at the target, to distinguish between reference SARS-CoV-2 and one or more mutants/variants, and/or to distinguish between different variants. Thus, in some embodiments, a “singleplex” reaction as described herein may comprise a single forward and a single reverse primer for each target, used in combination with a set of at least two probes, in some embodiments including a first probe specific to a reference SARS-CoV-2 allele and a second probe specific to a mutant/variant allele. In some embodiments, a first probe is specific to a first mutant allele at the target and a second probe is specific to a second, different mutant allele at the target.

In some embodiments, multiple assays each corresponding to a different mutation can be combined to create an assay panel targeted to a specific variant of SARS-CoV-2 and/or to distinguish between different strains of SARS-CoV-2. For example, with reference to Table 4, the B.1.1.7 (Alpha) variant includes the delH69V70, N501Y, P681H, Q27stop, delY144, A570D, T716I, S982A, and D1118H mutations. A selection of one or more assays described herein (e.g., illustrated in FIGS. 2C and 2D) each tailored to a different mutation that characterizes the B.1.1.7 variant can be combined to create an assay panel that is specifically targeted to the B.1.1.7 variant. Such an assay panel can be utilized, for example, to determine that each of the tested mutations are present and are thus indicative of the presence of the variant in the sample.

In another example, the B.1.617.2 (Delta) variant includes the L452R, P681R, T19R, and T478K mutations, among others. A selection of assays tailored to these mutations may be combined to create an assay panel specifically targeted to the B.1.617.2 variant. With reference to FIGS. 2C and 2D, the assay associated with Row No. 784 may be utilized to detect the L452R mutation, the assays associated with Row No. 968 may be utilized to detect the P681R mutation, one or both of the assays associated with Row Nos. 1188 and/or 1189 may be utilized to detect the T19R mutation, one or more of the assays associated with Row Nos. 1192 and/or 1193 may be utilized to detect the T478K mutation, and so on for other mutations for which detection is desired. The assay associated with Row No. 784 corresponds to SEQ ID NO:784 (forward primer), SEQ ID NO:2784 (reverse primer), SEQ ID NO:4784 (reference allele probe), and SEQ ID NO:6784 (mutant allele probe). The other assays correspond to SEQ ID NOs. in the same manner, such that an assay of row “X” includes a forward primer of SEQ ID NO:X, a reverse primer with a SEQ ID NO. of “X” plus 2000, a reference allele probe with a SEQ ID NO. of “X” plus 4000, and a mutant/variant allele probe with a SEQ ID NO. of “X” plus 6000. Other assay panels can be formed for other variants by combining different assays for mutations associated with those variants.

In another example, the B.1.1.529 (Omicron) variant includes the A2710T, G339D, Q493R, T13195C, and T547K mutations, among others. A selection of assays tailored to these mutations may be combined to create an assay panel specifically targeted to the B.1.1.529 variant. With reference to FIGS. 2C and 2D, the assay associated with Row No. 616 may be utilized to detect the G339D mutation, one or more of the assays associated with Row Nos. 972-975 may be utilized to detect the Q493R mutation, one or more of the assays associated with Row Nos. 71-72 may be utilized to detect the T13195C mutation, and the assay associated with Row No. 1196 may be utilized to detect the T547K mutation. As above, the assays numbers correspond to SEQ ID NOs. such that an assay of row “X” includes a forward primer of SEQ ID NO:X, a reverse primer with a SEQ ID NO. of “X” plus 2000, a reference allele probe with a SEQ ID NO. of “X” plus 4000, and a mutant/variant allele probe with a SEQ ID NO. of “X” plus 6000.

In an embodiment, the multiplex assay is designed to differentiate between a first and second organism by assaying for the presence of one or more target sites (also referred to herein as “markers”, e.g., 2, 3, 4, 5, 8, 12, 48, >10, >20, >30, >50, >100, >200, >500) more likely to be associated with the first organism but not the second organism. For example, the one or more markers or target sites are known to be typically present in the first organism and have been found to be typically absent in the second organism. One exemplary multiplex assay is designed to assay for at least one marker (e.g., 2, 3, 4, 5, 8, 12, 24, 48, >10, >20, >30, >50, >100, >200, >500) typically associated with the first organism, and at least one marker (e.g., 2, 3, 4, 5, 8, 12, 24, 48, >10, >20, >30, >50, >100, >200, >500) associated with the second organism. Similarly, the multiplex assay can be designed to distinguish between 3, 4, 5, 8, 10 or more organisms by including marker(s) that are specific to some but not all of the organisms. For example, at least one marker is specific to 2, 3, 4, or 5 (but not all) organisms being assayed.

The first and second organism can be genetically or symptomatically similar and can be difficult to distinguish symptomatically. For example, the first or second organism can be a SARS virus such as SARS-CoV or SARS-CoV-2, MERS-CoV, other viral pathogens such as Influenza Type A and/or Type B, and RSV Type A and/or Type B, bacterial pathogens, and/or fungal pathogens. In one such example, the first organism is SARS-CoV-2 or SARS-CoV or a particular strain of SARS-CoV-2, whereas the second organism is a different species of strain of SARS-CoV, SARS-CoV-2 or Influenza Type A or B. Particularly, the first and second organisms can be different strains of SARS-CoV-2, such as the B.1.1.7 variant and/or the B.1.351 variant. In an embodiment, some or all of the at least one marker typically associated with a first strain of SARS-CoV-2 are typically absent in the second strain of SARs-CoV-2. The first and/or second strain can, for example, be selected from SARS-CoV-2 Alpha, Beta, Gamma, Delta, Epsilon, Eta, Iota, Kappa, Lambda, Mu or Omicron variants. The same multiplex assay can be designed to differentiate between three, four, five, six, seven, eight, 10, 11, or all known strains of SARS-CoV-2 by appropriate selection of a number of target sites. For example, the assay panel can target markers that are associated with one single strain of SARS-CoV-2, or two, three, or four strains of SARS-CoV-2, but are not typically found in all strains of SARS-CoV-2.

One exemplary multiplex assay panel is designed to assay for at least one marker typically associated with a first strain of SARS-CoV-2 (e.g., the B.1.1.7 variant) and at least one marker associated with one or more different strains of SARS-CoV-2, such as the B.1.1.529 (“Omicron”) variant or the B.1.351 variant. Optionally, one or more markers in this assay can be generic (i.e., variant-agnostic) to one or more strains of SARS-CoV-2.

The multiplex assay, by appropriate choice of markers, can be designed to identify one or more organisms with significantly greater than random accuracy, for example greater than 70%, 80%, 90%, 95%, 97%, 98%, 99%, 99.5%, or 99.9% accuracy. In an embodiment, each marker is less than 70%, 80% or 90% accurate in identifying a particular strain of SARS-CoV-2, but a combination of any number of markers, e.g., two, three, four, six, eight or more markers is more than 90%, 95% or 99% accurate in identifying the strain. Optionally, at least one of these markers is typically present in the strain. Optionally, at least one of these markers is typically absent in the strain, but present in at least one other strain of SARS-CoV-2.

In one embodiment, the reference probes are VIC-labelled, while the mutant/variant probes are FAM-labelled. However, these labels may be swapped, or other suitable labels, as known in the art and/or as described elsewhere herein, may be additionally or alternatively be utilized for a reference probe or a mutant/variant probe, including, but not limited to, JUN, ABY, Alexa Fluor dye labels (e.g., AF647 and AF676), and combinations thereof.

### Sample Collection

The disclosed compositions, kits, and methods are configured to detect viral nucleic acid from a sample, preferably a specific and differential detection of SARS-CoV-2 or variant thereof from a sample. The sample may be a veterinary sample, a clinical sample, a food sample, a forensic sample, an environmental sample (e.g., soil, dirt, garbage, sewage, air, or water), including food processing and manufacturing surfaces, or a biological sample. In some embodiments, the sample is a human sample. In some embodiments, the sample is a non-human sample. For instance, the sample may be from a non-human species such as a dog, cat, mink, livestock animal (e.g., pigs, cattle, sheep, goats), etc. In most instances, SARS-CoV-2 or other coronaviruses and respiratory tract pathogens are detected by analysis of swabs or fluid obtained from swabs, such as throat swabs, nasal swabs, nasopharyngeal swabs, cheek swabs, saliva swabs, or other swabs, though it should be appreciated that SARS-CoV-2 or other coronaviruses and/or respiratory tract pathogens may also be detected by analysis of urine samples, saliva samples, or other clinical samples. Such samples may be collected with a collection device such as a tube, a dish, a bag, a plate, or any other appropriate container.

The sample can be collected by a healthcare professional in a healthcare setting, but in some instances, the sample may also be collected by the patient themselves or by an individual assisting the patient in self-collection. For example, a nasopharyngeal swab has heretofore served as the gold standard for obtaining a patient sample to be used in clinical diagnostics. Such swabs are often used by a healthcare professional in a healthcare setting. Other samples, such as a saliva sample, can similarly be obtained in a healthcare setting with the assistance or oversight of a healthcare professional. However, in some instances, self-collection of a sample can be more efficient and can be done outside of a healthcare setting.

In some embodiments, the sample is a raw saliva sample collected—whether by self-collection or assisted/supervised collection—in a sterile tube or specifically-designed saliva collection device. The saliva collection tube/device may be a component of a self-collection kit having instructions for use, such as sample collection instructions, sample preparation or storage instructions, and/or shipping instructions. The raw saliva sample can be collected directly into a sealable container without any preservation solution or other fluid or substance in the container prior to receipt of the saliva sample within the container or because of closing/sealing the container.

Traditionally, a nucleic acid fraction of the sample is extracted or purified from the sample—whether obtained via swab, from raw saliva, or other bodily fluid—prior to any detection of viral nucleic acids therein. Surprisingly, the disclosed embodiments for detecting viral nucleic acid from a sample can be adapted to detect viral nucleic acid directly from a raw saliva sample without a specific nucleic acid purification and/or extraction step prior to its use in downstream detection assays (e.g., RT-qPCR). In some embodiments, the saliva sample is pre-treated prior to use. This can include, for example, heating the saliva sample, such as by placing the raw saliva sample on a heat block/water bath set to a temperature of 95° C. for 30 minutes, followed by combining the heat-treated saliva with a buffer or lysis solution. The buffer or lysis solution can include, for example, any nucleic-acid-amenable buffer such as TBE and may further include a detergent and/or emulsifier such as the polysorbate-type nonionic surfactant, Tween-20.

In some embodiments, a nucleic acid fraction of the sample (e.g., obtained by a swab) can be extracted and used for downstream analysis, such as RT-qPCR. In some embodiments, the sample is a raw saliva sample. As provided above, the raw saliva sample can be self-collected (e.g., within a saliva collection device or sterile tube) or collected from the patient by any other individual in proximity to the patient. In some embodiments, the raw saliva sample is collected directly into a sealable container without any preservation solution or other fluid or substance in the container prior to receipt of the saliva sample or because of closing/sealing the container. The disclosed embodiments for detecting viral nucleic acid from a sample can be adapted to detect viral nucleic acid directly from the saliva sample, or in alternative embodiments, the sample can undergo a specific RNA purification and/or extraction step prior to its use in a detection assay (e.g., RT-qPCR). Thus, it should be appreciated that in some embodiments, a patient sample (e.g., saliva) can directly serve as sample input for subsequent downstream analyses, such as PCR, and this can be accomplished, in some embodiments, with no nucleic acid purification and/or extraction step prior to its use. In some embodiments, the sample used in subsequent downstream analyses is a heat-treated saliva sample as described herein.

In some implementations, viral nucleic acid may be detected directly from a raw or crude sample. For example, a raw saliva sample can be collected from the patient and heat-treated, such as by placing the raw saliva sample on a heat block/water bath set to a temperature of about 95° C. for 30 minutes. The heating step can provide many benefits, including, for example, denaturing nucleases such as RNase within the saliva that may interfere with accurate assessments of viral presence. Heating the raw saliva sample can also break down the mucus, making the sample more amenable to manipulation with laboratory equipment such as pipettes. The high heat can also cause thermal disruption of any prokaryotic and eukaryotic cells present in the sample and can also disrupt enveloped viruses and/or viral capsids present in the sample and thereby increase accessibility to any viral nucleic acid.

The heat-treated sample may also be mixed (e.g., via vortexing the sample for at least 10 seconds) before and/or after equilibrating the heat-treated sample to room temperature. A lysis solution can then be prepared and combined (e.g., in 1:1 proportions) with the heat-treated sample to create a probative template solution for detecting the presence of viral nucleic acid within the sample via nucleic acid amplification reactions (e.g., PCR, RT-PCR, qPCR, RT-qPCR, or the like). The lysis solution can include a nucleic-acid-amenable buffer such as TBE (and/or suitable alternative known in the art) combined with a detergent and/or emulsifier such as Tween-20, the polysorbate-type nonionic surfactant (and/or suitable alternative known in the art). The detergent and/or emulsifier can promote better mixing of the reagents and may also act to increase accessibility to any viral nucleic acid within the sample (e.g., by removing lipid envelopes from virions).

It should be appreciated that in some embodiments, the disclosed compositions can include the sample mixed with a buffer and detergent/emulsifier. The sample can be added to a buffer/detergent mixture or vice versa. In some embodiments, the sample is combined with a buffer and then detergent is added to the saliva/buffer mixture. In other embodiments, the sample is directly combined with a buffer/detergent mixture. As a non-limiting example, a set of patient samples can be prepared as compositions for downstream analysis and detection of viral sequence by adding a volume of heat-treated sample for each patient into one (or a plurality) of wells in a multi-well plate. A volume of a buffer/detergent mixture (e.g., TBE+Tween-20) can then be added to each well containing a patient sample. Alternatively, a multi-well plate can be loaded with a volume of a buffer/detergent mixture into which a volume of heat-treated saliva is added. Once combined, this probative template solution can be used immediately or stored for later analysis. Such probative template solutions can also be combined with PCR reagents (e.g., buffers, dNTPs, master mixes, etc.) prior to or after storage.

In some embodiments, a sample is obtained from multiple organisms (e.g., a plurality of individuals or patients) and the multiples samples are pooled together to make a single pooled sample for testing. In some embodiments, a sample may be obtained from at least two different organisms or individuals for pooling together to form a single sample for testing. In some embodiments, a sample may be obtained from between 2 to 10 different organisms or individuals for pooling together to form a single sample for testing. In some embodiments, a sample may be obtained from 2, 3, 4, 5, 6, 7, 8, 9, or 10 different organisms or individuals for pooling together to form a single sample for testing. In some embodiments, a sample may be obtained from up to and including 6 different organisms or individuals for pooling together to form a single sample for testing. For example, a sample used for testing, according to the methods and compositions described herein, may comprise a multiplicity of samples obtained from different organisms or individuals (e.g., 2, 3, 4, 5, or 6 different individuals) which are combined together to form a single “pooled” sample used for subsequent detection of a pathogen such as SARS-CoV-2.

### Nucleic Acid Amplification & Detection

Amplified products (“amplicons”) resulting from use of one or more embodiments described herein can be generated, detected, and/or analyzed using any suitable method and on any suitable platform. In some embodiments, SARS-CoV-2 or other target organism is detected by analysis of swabs, or fluid obtained from swabs, such as throat swabs, nasal swabs, nasopharyngeal swabs, cheek swabs, saliva swabs, or other swabs. SARS-CoV-2, other coronaviruses, or other target organisms may additionally or alternatively be detected by analysis of saliva samples, buccal samples, nasal samples, nasal pharyngeal samples, blood samples, urine samples, semen samples, or other biological samples.

In some embodiments, the nucleic acid assays as described herein can be used to detect, identify, characterize, quantify, or otherwise measure one or more nucleic acid targets in a sample. In some embodiments, the nucleic acid targets may be single-stranded, double-stranded, or any other nucleic acid molecule of any size or conformation. Optionally, the nucleic acid assays described herein can include polymerase chain reaction (PCR) assays (see, e.g., U.S. Pat. No. 4,683,202), loop-mediated isothermal amplification (“LAMP”) (see, e.g., U.S. Pat. No. 6,410,278), and other methods, including methods discussed below for detecting nucleic acid targets in a sample. In some embodiments, the PCR assays can be real time PCR or quantitative (qPCR) assays. In some other embodiments, the PCR assays can be end point PCR assays. Nucleic acid markers may be detected by any suitable means, including means that include nucleic acid amplification (e.g., thermal cycling amplification methods including PCR, and other nucleic acid amplification methods; isothermal amplification methods, including LAMP, etc.) and any other method that can be used to detect the presence of nucleic acid markers indicative of a disease-causing organism in a sample.

In some embodiments, the primers described herein are used in nucleic acid assays at a concentration from about 100 nM to 1 mM (e.g., 300 nM, 400 nM, 500 nM, etc.), including all concentration amounts and ranges in between. In some embodiments, the probes described herein are used in a nucleic acid assay at a concentration from about 50 nM to 500 nM (e.g., 75 nM, 125 nM, 250 nM, etc.), including all concentration amounts and ranges in between.

The primers and/or probes described herein may further comprise a fluorescent or other detectable label. In some embodiments the primers and/or probes may further comprise a quencher and in other embodiments the probes may further comprise a minor groove binder (MGB) moiety. Suitable fluorescent labels include but are not limited to 6FAM, ABY, VIC, JUN, FAM. Suitable quenchers include but are not limited to QSY (e.g., QSY7 and QSY21), BHQ (Black Hole Quencher) and DFQ (Dark Fluorescent Quencher).

In some multiplex assay embodiments, various SARS-CoV-2 genomic regions are detected, including assays for the SARS-CoV-2 Orf region (e.g., Orf1a, Orf1b, Orf1ab, Orf8), N Protein, S Protein, other genomic regions, and/or combinations thereof. Such multiplex assay embodiments may include multiple different probes for the same target genomic region in order to detect and/or distinguish between SARS-CoV-2 variants. For example, a multiplex assay that includes a target in the S Protein genomic region may include multiple different probes (each differentially labelled) for different variant forms of the targeted S Protein genomic region. Other target regions (including the N Protein and/or Orf regions) may also include multiple probes corresponding to different variant forms of such target regions. Optionally, in some embodiments, control sequence primers and/or probes (e.g., JUN-labeled probes), such as for amplification and/or detection of bacteriophage MS2 or human RNase P control sequences, are included in the multiplex assays using primer/probe sequences disclosed herein (and may be included as singleplex assays as well).

In some embodiments array formatted assays can be run as singleplex assays or as multiplex assays. In some embodiments, a panel of different assays may be formatted onto an array or a multi-well plate. In some embodiments, the panel can include some combination of one or more assays present in the TaqMan Array Respiratory Tract Microbiota Comprehensive Card (Thermo Fisher Scientific, Waltham, MA; Catalog No. A41238), along with one or more assays containing at least one primer or probe of FIGS. 2C-2D present in at least one well of an array or a well of a multi-well plate. In some embodiments, the panel includes assays for other circulating SARS-CoV-2 strains, including but not limited to 229E, KHU1, NL63, and OC43. In some embodiments, the disclosed methods include using the panel to profile respiratory microorganisms present in a sample taken from an organism (e.g., human) and determining the profile of respiratory microbiota present in the organism's sample. Optionally, the disclosed methods can include diagnosing an infection present in an organism (e.g., human) from which a sample is taken.

In some embodiments, a panel of different qPCR assays can be used to test for multiple strains or types of pathogens in addition to SARS-CoV-2, and variants thereof, including, but not limited to, other viral pathogens such as Influenza Type A and/or Type B, and RSV Type A and/or Type B, bacterial pathogens, and/or fungal pathogens. In some embodiments, the panel of qPCR assays can be used simultaneously to test a single patient sample or a single pooled sample comprising multiple patient samples, with each assay run in parallel in array format (“array formatted”). Optionally, different qPCR assays specific for each of the following target assays can be plated into individual wells of a single array or multi-well plate, such as for example a TaqMan Array Card (see, e.g., Thermo Fisher Scientific, Waltham, MA; Catalog Nos. 4346800 and 4342265) or a MicroAmp multi-well (e.g., 96-well, 384-well) reaction plate (see, e.g., Thermo Fisher Scientific, Waltham, MA; Catalog Nos. 4346906, 4366932, 4306737, 4326659 and N8010560). Optionally, the different qPCR assays present in different wells of an array or plate can be dried or freeze-dried in situ and the array or plate can be stored or shipped prior to use.

In some embodiments, the panel of qPCR assays includes at least one qPCR assay for detecting SARS-CoV-2 (including one or more variants described herein). In some other embodiments, the panel of qPCR assays includes at least one qPCR assay for detecting SARS-CoV-2 (including one or more variants described herein), plus at least one qPCR assay for detecting one or more of respiratory microorganisms listed in Table 5, below. Each qPCR assay can include a forward primer and a reverse primer for each target. Optionally, the assay can further include one or more probes.

TABLE 5
Respiratory Microorganisms

| | | | | |
|---|---|---|---|---|
| AdV 1 of 2 | FluB (pan) | CoV HKU1 | Parecho V | M. catarrhalis |
| AdV 2 of 2 | hPIV1 | CoV NL63 | SARS-CoV-2-1 | M. pneumoniae |
| HBoV | hPIV2 | CoV OC43 | Bordetella spp. | S. aureus |
| HHV3 | hPIV3 | Mumps | B. holmesii | S. pneumoniae |
| HHV4 | hPIV4 | MERS-CoV | B. pertussis | P. jirovecii |
| HHV5 | RSVA | SARS-CoV | SARS-CoV-2-2 | SARS-CoV-2-3 |
| HHV6 | RSVB | Entero V (pan) | C. pneumoniae | Internal Control |
| FluA (pan) | hMPV | Entero V D68 | H. influenzae | (IC) - RNase P |
| FluA (H1) | Measles | RhinoV 1of2 | K. pneumoniae | |
| FluA (H3) | CoV 229E | Rhino V 2of2 | L. pneumophila | |

In some embodiments, the multiplex assay detects two or more (e.g., 2, 3, 4, 5, 6, etc.) of the targets of Table 5. In some embodiments, the multiplex assay detects one or more targets within the SARS-CoV-2 genome (e.g., including reference and/or mutant or variant SARS-CoV-2 targets) as wells an internal positive control, such as RNase P.

In some other embodiments, the primers and/or probes provided in FIGS. 2C-2D can be used to amplify one or more specific target sequences present in a SARS-CoV-2 target and to enable identification or differentiation between different SARS-CoV-2 variants, such as those described in Table 4, (including differentiation between a mutant/variant and reference form SARS-CoV-2 target).

The primer and probe sequences described herein need not have 100% homology/identity to their targets to be effective, though in some embodiments, homology is substantially 100% or exactly 100%. In some embodiments, one or more of the disclosed primer and/or probe sequences have a homology to their respective target of at least about 50%, at least about 60%, at least about 70%, at least about 75%, at least about 80%, at least about 85%, at least about 90%, at least about 95%, at least about 97%, at least about 98%, at least about 99%, at least about 99.9%, or up to substantially 100% or exactly 100%. Some combinations of primers and/or probes may include primers and/or probes each with different homologies to their respective targets, and the homologies may be, for example, within a range with endpoints defined by any two of the foregoing values.

PCR and related methods are common methods of nucleic acid amplification. PCR is one, but not the only, example of a nucleic acid polymerase reaction method for amplifying a nucleic acid test sample comprising the use of a known nucleic acid as a primer and a nucleic acid polymerase to amplify or generate a specific target nucleic acid. In general, PCR utilizes a primer pair that consists of a forward primer and a reverse primer configured to amplify a target segment of a nucleic acid template. Typically, but not always, the forward primer hybridizes to the 5′ end of the target sequence and the reverse primer will be identical to a sequence present at the 3′ end of the target sequence. The reverse primer will typically hybridize to a complement of the target sequence, for example an extension product of the forward primer and/or vice versa. PCR methods are typically performed at multiple different temperatures, causing repeated temperature changes during the PCR reaction (“thermal cycling”). Other amplification methods, such as, e.g., LAMP methods, and other isothermal methods, such as those listed in Table 6, may require less or less extensive thermal cycling than does PCR, or require no thermal cycling. Such isothermal amplification methods are also contemplated for use with the assay compositions, kits, and methods described herein.

TABLE 6
Summary of optional isothermal amplification methods.

| | |
|---|---|
| NASBA | Nucleic acid sequence-based amplification (NASBA) is a method |
| | used to amplify RNA. |
| LAMP | Loop-mediated isothermal amplification (LAMP) is a single tube |
| | technique for the amplification of DNA. It typically uses 4-6 primers, which |
| | form loop structures to facilitate subsequent rounds of amplification. |
| HDA | Helicase-dependent amplification (HDA) uses the double-stranded |
| | DNA unwinding activity of a helicase to separate strands for in vitro DNA |
| | amplification at constant temperature. |
| RCA | Rolling circle amplification (RCA) starts from a circular DNA |
| | template and a short DNA or RNA primer to form a long single stranded |
| | molecule. |
| MDA | Multiple displacement amplification (MDA) is a technique that |
| | initiates when multiple random primers anneal to the DNA template and the |
| | polymerase amplifies DNA at constant temperature. |
| WGA | When MDA is used to amplify DNA from a whole genome of a cell it |
| | is called whole genome amplification (WGA). (Other methods of WGA |
| | include MALBAC, LIANTI, DOP-PCR.) |
| RPA | Recombinase polymerase amplification (RPA) is a low temperature |
| | DNA and RNA amplification technique. |

Methods of performing PCR are well known in the art; nevertheless, further discussion of PCR and other methods may be found, for example, in Molecular Cloning: A Laboratory Manual by Green and Sambrook, Cold Spring Harbor Laboratory Press, 4th Edition 2012, which is incorporated by reference herein in its entirety.

SARS-CoV-2 has a single-stranded positive-sense RNA genome. In some embodiments, therefore, the amplification reaction (e.g., LAMP or PCR) can be combined with a reverse transcription (RT) reaction, such as in RT-LAMP or RT-PCR to convert the RNA genome to a cDNA template. The cDNA template is then used to create amplicons of the target sequences in the subsequent amplification reactions. In some embodiments, RT-PCR is performed using samples comprising virus particles or suspected of comprising virus particles. In some embodiments, the viral particles are live particles. In some embodiments, the viral particles are dead or inactivated particles. In some embodiments, the RT-PCR may be a one-step procedure using one or more primers and one or more probes as described herein. In some embodiments, the RT-PCR may be carried out in a single reaction tube, reaction vessel (e.g., “single-tube” or “1-tube” or “single-vessel” reaction). In some embodiments, the RT-PCR may be carried out in a multi-site reaction vessel, such as a multi-well plate or array. In some embodiments, RT and PCR are performed in the same reaction vessel or reaction site, such as in 1-step or 1-tube RT-qPCR. Suitable exemplary RTs can include, for instance, a Moloney Murine Leukemia Virus (M-MLV) Reverse transcriptase, SuperScript Reverse Transcriptases (Thermo Fisher Scientific), SuperScript IV Reverse Transcriptases (Thermo Fisher Scientific), or Maxima Reverse Transcriptases (Thermo Fisher Scientific), or modified forms of any such RTs.

In some embodiments, different assay products (e.g., amplicons from different variants) can be independently detected or at least discriminated from each other. For example, different assay products may be distinguished optically (e.g., using optically different labels for each qPCR assay) or can be discriminated using some other suitable method, including as described in U.S. Patent Publication No. 2019/0002963, which is incorporated herein by reference in its entirety. In some embodiments, specific combinations of labels are used to differentiate between different SARS-CoV-2 variants. For example, different SARS-CoV-2 variants may be differentiated from one another using different labels specific to each variant such that the label is detectable only in the presence—and amplification—of the associated variant sequence.

In some embodiments, the assays disclosed herein are used to create a panel of different assays for use in SNP genotyping methods. In some embodiments, the panel comprises two or more assays selected from FIG. 2C-2D. In some embodiments, the assays disclosed herein are used in methods for identifying the presence or absence of one or more clinically relevant mutations associated with an emergent SARS-CoV-2 variant. In some embodiments, the emergent SARS-CoV-2 variant includes B.1.617.2, B.1.1.7, and/or B.1.351 variants. In some embodiments, the emergent SARS-CoV-2 variant is selected from a variant listed in Table 4. In some embodiments, the one or more clinically relevant mutations are selected from a mutation listed in Table 4 and/or in FIGS. 2C-2D. In some embodiments, clinically relevant mutations are selected from the 69/70 deletion, N501Y, N439K, E484K, K417N, A222V, D614G, Y453F, P681R, and P681H mutations. In some embodiments, the panel of assays can detect up to 20 clinically relevant gene mutations or SNPs. In some embodiments, the assays disclosed herein are used in methods for identification of SARS-CoV-2 variant mutations in symptomatic and/or asymptomatic patients. In some embodiments, the assays disclosed herein are used for identification of patients who are COVID-19 positive. In some embodiments, the assays disclosed herein are used for profiling one or more SARS-CoV-2 variants. In some embodiments, the variant profiling is based on a particular pattern of detected mutations, such as those disclosed in Table 4 and FIGS. 2C-2D.

Each assay embodiment described herein may be used independently to identify a particular SARS-CoV-2 mutation. Alternatively, a panel of multiple assays may be used to identify the presence (or absence) of multiple mutations. A particular SARS-CoV-2 mutation may be characteristic of multiple SARS-CoV-2 variants, and thus while detection of such a mutation may illustrate that a sample includes a SARS-CoV-2 variant, it may not, by itself, allow for complete identification of the particular variant involved. Moreover, many SARS-CoV-2 variants have multiple mutations at multiple genomic regions. Thus, while a single assay can function to identify the presence (or absence) of a particular mutation, multiple assays can function together to identify a set of particular mutations that can together identify a particular variant and/or resolve between different variants that have overlapping mutation profiles. As such, in some embodiments, multiple assays disclosed herein, when used in combination, can be used in methods to provide a SARS-CoV-2 variant profile.

As a particular example, the B.1.1.7 variant and the B.1.351 variant are two notable variants. While each of these variants have the S gene N501Y.A_T mutation, the B.1.1.7 variant has the 69/70 S gene deletion mutation while the B.1.351 variant does not, and the B.1.351 variant has the S gene E484K.G_A mutation while the B.1.1.7 variant does not (see Table 4). Thus, an assay panel configured to test for at least two of the S gene N501Y.A_T mutation, the 69/70 S gene deletion mutation, and the S gene E484k.G_A mutation can aid in identifying these variants and/or resolving between these variants, despite some overlap in each of their respective mutation profiles. Of course, other assays focusing on additional and/or alternative distinguishing target mutation loci may be also be utilized. Various assay panels including two or more of the assays shown in FIGS. 2C and 2D may be designed to identify targeted variants and/or to resolve between variants.

In some embodiments, the amplifying step can include performing qPCR, as that term is defined herein. qPCR is a sensitive and specific method for detecting and optionally quantifying amounts of starting nucleic acid template (e.g., coronaviral nucleic acid) in a sample. Methods of qPCR are well known in the art; one leading method involves the use of a specific hydrolysis probe in conjunction with a primer pair. The hydrolysis probe can include an optical label (e.g., fluorophore) at one end and a quencher that quenches the optical label at the other end. In some embodiments, the label is at the 5′ end of the probe and cleavage of the 5′ label occurs via 5′ hydrolysis of the probe by the nucleic acid polymerase as it extends the forward primer towards the probe binding site within the target sequence. The separation of the probe label from the probe quencher via cleavage (or unfolding) of the probe results in an increase in optical signal which can be detected and optionally quantified. The optical signal can be monitored over time and analyzed to determine the relative or absolute amount of starting nucleic acid template present in the sample. Suitable labels are described herein.

The reaction vessel or volume can optionally include a tube, channel, well, cavity, site or feature on a surface, or alternatively a droplet (e.g., a microdroplet or nanodroplet) that may be deposited onto a surface or into a surface well or cavity, or suspended within (or partially bounded by) a fluid stream. In some embodiments, the reaction volume includes one or more droplets arrayed on a surface or present in an emulsion. The reaction volumes can optionally be formed by fusion of multiple pre-reaction volumes containing different components of an amplification reaction. For example, pre-reaction volumes containing one or more primers can be fused with pre-reaction volumes containing human nucleic acid samples and/or polymerase enzymes, nucleotides, and buffer. In some embodiments involving performing qPCR reactions in array format, a surface contains multiple grooves, channels, wells, cavities, sites, or features defining a reaction volume containing one or more amplification reagents (e.g., primers, probes, buffer, polymerase, nucleotides, and the like). In some array-formatted singleplex embodiments, the reaction volume within the selected tubes, grooves, channels, wells, cavities, sites, or features contains only a single forward primer sequence and a single reverse primer sequence. Optionally, one or more probe sequences are also included in the singleplex reaction volume.

In some array-formatted multiplex embodiments, the reaction volume within the selected tubes, grooves, channels, wells, cavities, sites, or features contains multiple (e.g., 2, 3, 4, 5, 6, etc.) forward and reverse primer sequences and/or multiple probe sequences. For instance, exemplary methods for polymerizing and/or amplifying and detecting nucleic acids suitable for use as described herein are commercially available as TaqMan assays (see, e.g., U.S. Pat. Nos. 4,889,818; 5,079,352; 5,210,015; 5,436,134; 5,487,972; 5,658,751; 5,210,015; 5,487,972; 5,538,848; 5,618,711; 5,677,152; 5,723,591; 5,773,258; 5,789,224; 5,801,155; 5,804,375; 5,876,930; 5,994,056; 6,030,787; 6,084,102; 6,127,155; 6,171,785; 6,214,979; 6,258,569; 6,814,934; 6,821,727; 7,141,377; and/or 7,445,900, all of which are hereby incorporated herein by reference in their entirety).

TaqMan assays are typically carried out by performing nucleic acid amplification on a target polynucleotide using a nucleic acid polymerase having 5′-to-3′ nuclease activity, a primer capable of hybridizing to the target polynucleotide, and an oligonucleotide probe capable of hybridizing to said target polynucleotide 3′ relative to the primer. The oligonucleotide probe typically includes a detectable label (e.g., a fluorescent reporter molecule) and a quencher molecule capable of quenching the fluorescence of the reporter molecule. Typically, the detectable label and quencher molecule are part of a single probe. As amplification proceeds, the polymerase digests the probe to separate the detectable label from the quencher molecule. The detectable label is monitored during the reaction, where detection of the label corresponds to the occurrence of nucleic acid amplification (e.g., the higher the signal the greater the amount of amplification). Variations of TaqMan assays are known in the art and would be suitable for use in the methods described herein.

For example, a singleplex or multiplex qPCR can include a single TaqMan assay associated with a locus-specific sequence or multiple TaqMan assays respectively associated with a plurality of loci in a multiplex format. As a non-limiting example, a 4-plex reaction can include FAM (emission peak ˜517 nm), VIC (emission peak ˜551 nm), ABY (emission peak ˜580 nm), and JUN (emission peak ˜617 nm) dyes. In some embodiments, each dye is associated with one or more target sequences. In some embodiments, one or more dyes are quenched by a QSY quencher (e.g., QSY21). In some embodiments, each multiplex reaction allows up to 12 targets to be amplified and tracked real-time within a single reaction vessel. In some embodiments, up to 2, 4, 6, 8, 10, or 12 targets are amplified and tracked real-time within a single reaction vessel, using any combination of detectable labels disclosed herein or otherwise known to those of skill in the art. The reporter dyes are optimized to work together with minimal spectral overlap for improved performance. Any combination of dyes described herein can additionally be combined with other dyes (e.g., Mustang Purple (emission peak ˜654 nm) or one or more Alexa Fluors (e.g., AF647 and AF676)), for use in monitoring fluorescence of a control or for use in a non-emission-spectrum-overlapping 5-plex assay. In addition, the QSY quencher is fully compatible with probes that have minor-groove binder quenchers.

Where multiple detection channels are utilized, it is desirable to minimize crosstalk between fluorescence reporters and select reporters that avoid excessive spectral overlap. One example of an assay that includes 5 detection channels incorporates the dyes FAM, ABY, VIC, and JUN, along with Mustang Purple (emission peak ˜654 nm) or an appropriate Alexa Fluor, for example. The dyes may be associated with a corresponding primer and/or with a probe of the assay, as described herein. Other embodiments may utilize other combinations of dyes to define different sets of detection channels (including in assays with more than 5 detection channels) according to particular preferences or application needs. Additional examples of multiplex assays (including related dye compounds, compositions, methods, and kits) are described in U.S. Provisional Patent Application No. 62/705,935, filed Jul. 23, 2020 and titled “Compositions, Systems and Methods for Biological Analysis Involving Energy Transfer Dye Conjugates and Analytes Comprising the Same”, which is incorporated herein by this reference in its entirety.

Detector probes may be associated with alternative quenchers, including without limitation, dark fluorescent quencher (DFQ), black hole quenchers (BHQ), Iowa Black, QSY quencher, and Dabsyl and Dabcel sulfonate/carboxylate Quenchers. Detector probes may also include two probes, wherein, for example, a fluorophore is associated with one probe and a quencher is associated with a complementary probe such that hybridization of the two probes on a target quenches the fluorescent signal or hybridization on the target alters the signal signature via a change in fluorescence. Detector probes may also include sulfonate derivatives of fluorescein dyes with SO₃ instead of the carboxylate group, phosphoramidite forms of fluorescein, phosphoramidite forms of Cy5.

It should be appreciated that when using more than one detectable label, particularly in a multiplex format, each detectable label preferably differs in its spectral properties from the other detectable labels used therewith such that the labels may be distinguished from each other, or such that together the detectable labels emit a signal that is not emitted by either detectable label alone. Exemplary detectable labels include, for instance, a fluorescent dye or fluorophore (e.g., a chemical group that can be excited by light to emit fluorescence or phosphorescence), “acceptor dyes” capable of quenching a fluorescent signal from a fluorescent donor dye, and the like, as described above. Suitable detectable labels may include, for example, fluoresceins (e.g., 5-carboxy-2,7-dichlorofluorescein; 5-Carboxyfluorescein (5-FAM); 5-Hydroxy Tryptamine (5-HAT); 6-JOE; 6-carboxyfluorescein (6-FAM); Mustang Purple, VIC, ABY, JUN; FITC; 6-carboxy-4′,5′-dichloro-2′,7′-dimethoxy¬fluorescein (JOE)); 6-carboxy-1,4-dichloro-2′,7′-dichloro-fluorescein (TET); 6-carboxy-1,4-dichloro-2′,4′,5′,7′-tetra-chlorofluorescein (HEX); Alexa Fluor fluorophores (e.g., 350, 405, 430, 488, 500, 514, 532, 546, 555, 568, 594, 610, 633, 635, 647, 660, 680, 700, 750); BODIPY fluorophores (e.g., 492/515, 493/503, 500/510, 505/515, 530/550, 542/563, 558/568, 564/570, 576/589, 581/591, 630/650-X, 650/665-X, 665/676, FL, FL ATP, FI-Ceramide, R6G SE, TMR, TMR-X conjugate, TMR-X, SE, TR, TR ATP, TR-X SE), Cascade Blue, Cascade Yellow; CyM dyes (e.g., 3, 3.18, 3.5, 5, 5.18, 5.5, 7), cyan GFP, cyclic AMP Fluorosensor (FiCRhR), fluorescent proteins (e.g., green fluorescent protein (e.g., GFP. EGFP), blue fluorescent protein (e.g., BFP, EBFP, EBFP2, Azurite, mKalamal), cyan fluorescent protein (e.g., ECFP, Cerulean, CyPet), yellow fluorescent protein (e.g., YFP, Citrine, Venus, YPet), FRET donor/acceptor pairs (e.g., fluorescein/fluorescein, fluorescein/tetramethylrhodamine, IAEDANS/fluorescein, EDANS/dabcyl, BODIPY FL/BODIPY FL, Fluorescein/QSY7 and QSY9), LysoTracker and LysoSensor (e.g., LysoTracker Blue DND-22, LysoTracker Blue-White DPX, LysoTracker Yellow HCK-123, LysoTracker Green DND-26, LysoTracker Red DND-99, LysoSensor Blue DND-167, LysoSensor Green DND-189, LysoSensor Green DND-153, LysoSensor Yellow/Blue DND-160, LysoSensor Yellow/Blue 10,000 MW dextran), Oregon Green (e.g., 488, 488-X, 500, 514); rhodamines (e.g., 110, 123, B, B 200, BB, BG, B extra, 5-carboxytetramethylrhodamine (5-TAMRA), 5 GLD, 6-Carboxyrhodamine 6G, Lissamine, Lissamine Rhodamine B, Phallicidine, Phalloidine, Red, Rhod-2, ROX (6-carboxy-X-rhodamine), 5-ROX (carboxy-X-rhodamine), Sulphorhodamine B can C, Sulphorhodamine G Extra, TAMRA (6-carboxytetramethyl,rhodamine), Tetramethylrhodamine (TRITC), WT), Texas Red, Texas Red-X, among others as would be known to those of skill in the art.

Other detectable labels may be used in addition to or as an alternative to labelled probes. For example, primers can be labeled and used to both generate amplicons and to detect the presence (or concentration) of amplicons generated in the reaction, and such may be used in addition to or as an alternative to labeled probes described herein. As a further example, primers may be labeled and utilized as described in Nazarenko et al. (Nucleic Acids Res. 2002 May 1; 30(9): e37), Hayashi et al. (Nucleic Acids Res. 1989 May 11; 17(9): 3605), and/or Neilan et al. (Nucleic Acids Res. Vol. 25, Issue 14, 1 Jul. 1997, pp. 2938-39). Those of skill in the art will also understand and be capable of utilizing the PCR processes (and associated probe and primer design techniques) described in Zhu et al. (Biotechniques. 2020 July: 10.2144/btn-2020-0057).

Any of these systems and detectable labels, as well as many others, may be used to detect amplified target nucleic acids. In some embodiments, intercalating labels can be used such as ethidium bromide, SYBR Green I, SYBR GreenER, and PicoGreen (Life Technologies Corp., Carlsbad, CA), thereby allowing visualization in real-time, or end point, of an amplification product in the absence of a detector probe. In some embodiments, real-time visualization may include both an intercalating detector probe and a sequence-based detector probe. In some embodiments, the detector probe is at least partially quenched when not hybridized to a complementary sequence in the amplification reaction and is at least partially unquenched when hybridized to a complementary sequence in the amplification reaction. In some embodiments, probes may further comprise various modifications such as a minor groove binder to further provide desirable thermodynamic characteristics.

In some embodiments, the amplicon is labeled by incorporation of or hybridization to labeled primer. In some embodiments, the amplicon is labeled by hybridization to a labeled probe. In some embodiments, the amplicon is labeled by binding of a DNA-binding dye. In some embodiments, the dye may be a single-strand DNA binding dye. In other embodiments, the dye may be a double-stranded DNA binding dye. In other embodiments, the amplicon is labeled via polymerization or incorporation of labeled nucleotides in a template-dependent (or template-independent) polymerization reaction. This can be part of the amplifying step or alternatively the labeled nucleotide can be added after amplifying is completed. The labeled amplicon (or labeled derivative thereof) can be detected using any suitable method such as, for example, electrophoresis, hybridization-based detection (e.g., microarray, molecular beacons, and the like), chromatography, NMR, and the like.

In one exemplary embodiment, the labeled amplicon is detected using capillary electrophoresis. In another embodiment, the labeled amplicon is detected using qPCR. In some embodiments, a plurality of different amplicons is formed, and optionally labeled, within a single reaction volume via a single amplification reaction. For example, a multiplex reaction (e.g., 2-plex, 3-plex, 4-plex, 5-plex, 6-plex) carried out in a single tube or reaction vessel (e.g., “single-tube” or “1-tube” or “single-vessel” reaction) can produce a plurality of amplicons that are labeled. In some embodiments, the plurality of amplicons can be differentially labeled. In some embodiments, each of the plurality of amplicons produced during amplification is labeled with a different label.

Optionally, in some embodiments, a control template and/or assay, such as bacteriophage MS2 or RNase P control, is included in the kit. If the positive control sequence is an endogenously-derived control, such as RNase P, the presence of patient-derived nucleic acid (e.g., genomic DNA coding for RNase P, RNase P RNA, and/or reverse transcribed RNase P transcript), can be used as the template for an RNase P qPCR assay. Exemplary primers and probes for such an RNase P and MS2 positive controls can include sequences of SEQ ID NO:7305-SEQ ID NO:7310, although those having skill in the art should appreciate that other RNase-P-specific primers and/or probes could be used. If the positive control sequence is an exogenously derived control, such as a component of the MS2 bacteriophage, a known or predetermined concentration of template nucleic acid is added to the reaction volume to serve as the requisite template for an MS2 qPCR assay.

In some embodiments, the nucleic acid amplification assays as described herein are performed using a Real-time PCR (qPCR) instrument, including for example a QuantStudio Real-Time PCR system, such as the QuantStudio 5 RealTime PCR System (QS5), QuantStudio 7 RealTime PCR System (QS7), and/or QuantStudio 12K Flex System (QS12K), or a 7500 Real-Time PCR system, such as the 7500 Fast Dx system, from Thermo Fisher Scientific.

In some embodiments, the systems, compositions, methods, and devices used for nucleic acid amplification comprise a “point-of-service” (POS) system. In some embodiments, samples may be collected and/or analyzed at a “point-of-care” (POC) location. In some embodiments, analysis at a POC location typically does not require specialized equipment and has rapid and easy-to-read visual results. In some embodiments, analysis can be performed in the field, in a home setting, and/or by a lay person not having specialized skills. In certain embodiments, for example, the analysis of a small-volume clinical sample may be completed using a POS system in a short period of time (e.g., within hours or minutes).

Optionally, a POS system is utilized at a location that is capable of providing a service (e.g., testing, monitoring, treatment, diagnosis, guidance, sample collection, verification of identity (ID verification), and other services) at or near the site or location of the subject. A service may be a medical service or it may be a non-medical service. In some situations, a POS system provides a service at a predetermined location, such as a subject's home, school, or work, or at a grocery store, a drug store, a community center, a clinic, a doctor's office, a hospital, an outdoor triage tent, a makeshift hospital, a border check point, etc. A POS system can include one or more point of service devices, such as a portable virus/pathogen detector. In some embodiments, a POS system is a point of care system. In some embodiments, the POS system is suitable for use by non-specialized workers or personnel, such as nurses, police officers, civilian volunteers, or the patient.

In certain embodiments, a POC system is utilized at a location at which medical-related care (e.g., treatment, testing, monitoring, diagnosis, counseling, etc.) is provided. A POC may be, e.g., at a subject's home, work, or school, or at a grocery store, a community center, a drug store, a doctor's office, a clinic, a hospital, an outdoor triage tent, a makeshift hospital, a border check point, etc. A POC system is a system which may aid in, or may be used in, providing such medical-related care, and may be located at or near the site or location of the subject or the subject's health care provider (e.g., subject's home, work, or school, or at a grocery store, a community center, a drug store, a doctor's office, a clinic, a hospital, etc.).

In embodiments, a POS system is configured to accept a clinical sample obtained from a subject at the associated POS location. In embodiments, a POS system is further configured to analyze the clinical sample at the POS location. In embodiments, the clinical sample is a small volume clinical sample. In embodiments, the clinical sample is analyzed in a short period of time. In embodiments, the short period of time is determined with respect to the time at which sample analysis began. In embodiments, the short period of time is determined with respect to the time at which the sample was inserted into a device for the analysis of the sample. In embodiments, the short period of time is determined with respect to the time at which the sample was obtained from the subject.

In some embodiments, a POS system or a POC system can include the amplification-based methods, compositions and kits disclosed herein, including any of the described assays and/or assay panels. Such assays are contemplated for use with both thermal cycling amplification workflows and protocols, such as in PCR, as well as isothermal amplification workflows and protocols, such as in LAMP.

In some embodiments, a POS or a POC system comprises self-collection of a biological sample, such as a nasal swab or a saliva sample. In some embodiments, the self-collection may comprise the use of a self-collection kit and/or device, such as a swab or a tube (e.g., a saliva collection tube or similar saliva collection device). In some embodiments, the self-collection kit comprises instructions for use, including collection instructions, sample preparation or storage instructions, and/or shipping instructions. For example, the self-collection kit and/or device may be used by an individual, such as lay person, not having specialized skills or medical expertise. In some embodiments, self-collection may be performed by the patient themselves or by any other individual in proximity to the patient, such as but not limited to a parent, a care giver, a teacher, a friend, or other family member.

Notably, in some embodiments, the nucleic acid amplification protocol can be configured for rapid processing (e.g., in less than about 45 minutes) and high throughput, allowing for a minimally invasive method to quickly screen large numbers of individuals in a scalable way. This can be particularly useful to perform asymptomatic testing (e.g., high frequency/widespread testing at schools, workplaces, conventions, sporting events, large social gatherings, etc.) or for epidemiological purposes. The disclosed embodiments can also beneficially provide a lower cost sample collection system and method that enables self-collection (reducing health care professional staffing needs) using a low-cost collection device. This eliminates the requirements for swabs, buffers, virus transmission media (or other specialized transport medium), and the like. The disclosed embodiments also allow for a reduction in Personal Protective Equipment (PPE) requirements and costs. Because the reagents and methods are streamlined (e.g., no precursor nucleic acid purification and/or extraction step), there is a reduced use of nucleic acid preparation plastics which brings a coincident reduction in reagent costs and inventory costs. There is also a beneficial reduced dependence on supply-constrained items, and the compatibility of these methods and kit components with existing equipment improves the flexibility and simplicity of their implementation to the masses. Overall, such embodiments allow for a less expensive assay that can be accomplished more quickly from sample collection through result generation.

Some embodiments relate to kits containing one or more of the primers and probes disclosed in FIGS. 2C-2D. Optionally, the kit can further include a master mix. In some embodiments, the master mix is TaqMan Fast Virus 1-Step Master Mix (Thermo Fisher Scientific, Waltham, MA, Catalog No. 44444432). In some embodiments, the master mix is TaqPath 1-Step RT-qPCR Master Mix, CG (Thermo Fisher Scientific, Waltham, MA, Catalog No. A15299). In other embodiments the master mix is TaqPath™ 1 Step Multiplex Master Mix (No ROX™) (Thermo Fisher Scientific, Waltham, MA, Catalog No. A48111, A28521). In some embodiments, the kit includes primers, probes and master mix sufficient to constitute a reaction mixture supporting amplification of one or more target regions from SARS-CoV-2 and/or variant thereof (e.g., reference and one or more mutants/variants). Target regions can include the genes encoding the N protein, the S protein, and/or Orf proteins.

In some array-based embodiments, two or more different qPCR assays (each containing a forward primer, a reverse primer and optionally a probe) are used in a single well, cavity, site or feature of the array and products of each assay can be independently detected. For example, different assay products may be discriminated optically (e.g., using different labels present in components each assay) or using some other suitable method, including as described in U.S. Patent Publication No. 2019/0002963, incorporated by reference herein. In some embodiments, at least one primer of each assay contains an optically detectable label that can be discriminated from the optical label of at least one other assay.

In some embodiments, at least one of the qPCR assays targets a sequence within a gene encoding the N protein, the S protein, and/or an Orf protein (e.g., ORF1a, ORF1b, Orf1ab, Orf8). In some embodiments, the target sequence within N protein, S protein, and/or the Orf genes (e.g., Orf1a, Orf1b, Orf1ab, Orf8) is a reference form sequence. In some embodiments, the target sequence within N protein, S protein, and/or the Orf genes (e.g., Orf1a, Orf1b, Orf1ab, Orf8) is a variant or mutant sequence. In some embodiments, the reaction volume further includes a second qPCR assay that targets a different gene of the group from the first. In some embodiments, the reaction volume further includes a third qPCR assay that targets the third gene from the group, such that when the reaction volume is subjected to amplification conditions and if the sample includes SARS-CoV-2 genomic RNA, at least one amplicon is produced from genetic sequence encoding the S protein, at least one amplicon from genetic sequence encoding the N protein and at least one amplicon from the genetic sequence encoding the Orf genes (e.g., Orf1a, Orf1b, Orf1ab, Orf8). In other embodiments, the reaction volume further includes a fourth qPCR assay that targets the exogenous positive control sequence, such that when the reaction volume is subjected to amplification conditions and if the sample includes SARS-CoV-2 genomic RNA, at least one amplicon is produced from genetic sequence encoding the S protein, at least one amplicon from genetic sequence encoding the N protein, at least one amplicon from the genetic sequence encoding the Orf genes (e.g., Orf1a, Orf1b, Orf1ab, Orf8) and at least one amplicon from the exogenous positive control sequence. In some embodiments, the reaction volume further includes a fifth qPCR assay that targets two separate exogenous positive control sequences, such that when the reaction volume is subjected to amplification conditions and if the sample includes SARS-CoV-2 genomic RNA, at least one amplicon is produced from genetic sequence encoding the S protein, at least one amplicon from genetic sequence encoding the N protein, at least one amplicon from the genetic sequence encoding an Orf protein (e.g., Orf1a, Orf1b, Orf1ab, Orf8) and at least two amplicons from the two exogenous positive control sequences.

In some embodiments, optimal amplification and detectability for viral genomes is achieved by adding a master mix to the reaction volume prior to amplification. The master mix optionally includes a polymerase, nucleotides, buffers, and salts. In some embodiments (particularly multiplex assays), the reaction volume includes TaqMan Fast Virus 1-Step Master Mix (Thermo Fisher Scientific, Waltham, MA, Catalog No. 44444432). In some embodiments, the reaction volume includes TaqPath 1-Step RT-qPCR Master Mix, CG (Thermo Fisher Scientific, Waltham, MA, Catalog No. A15299). In other embodiments the master mix is TaqPath™ 1 Step Multiplex Master Mix (No ROX™) (Thermo Fisher Scientific, Waltham, MA, Catalog No. A48111, A28521).

### EXAMPLES

The following Examples may reference specific target nucleic acids, compositions, formulations, and/or process steps. It will be understood, however, that these Examples may be modified by using any of the components described elsewhere herein, including by using any of the primers and/or probes described herein.

### Example 1: Singleplex Assay for Detecting SARS-CoV-2

An exemplary protocol for detecting SARS-CoV-2 from a biological sample via a singleplex assay was performed using the TaqMan 2019-nCoV Assay Kit (Thermo Fisher Scientific, Catalog No. A47532). The assay kit included primers and FAM-labeled probes for detecting the Orf1ab, S protein, and N protein coding sequences for SARS-CoV-2. An optional VIC-labeled internal control directed to RNase P was also included. In a separate kit, the same primers/probes were included and used as positive controls to detect the target sequences from a synthetic DNA construct encoding the target sequences for Orf1ab, S protein, N protein, and RNase P.

The total nucleic acid content was isolated from samples collected via nasopharyngeal swab, nasopharyngeal aspirate, or bronchoalveolar lavage using the MagMAX Viral/Pathogen Nucleic Acid Isolation Kit (Thermo Fisher Scientific, Catalog No. A42356) in accordance with the instructions provided therewith.

For each assay, the components in Table 7 were combined for the number of reactions, plus 10% overage:

TABLE 7
RT-qPCR Reaction Mix

| Component | Volume/reaction | |
|---|---|---|
| RT-qPCR Master Mix, CG (4X) | 6.25 | μL |
| 2019 nCoV TaqMan Assay (20X) | 1.25 | μL |
| TaqMan RNase P Assay, VIC dye/QSY assay (20X) | 1.25 | μL |
| Nuclease-free water | 11.25 | μL |
| Total Reaction Mix Volume | 20.00 | μL |

The “Master Mix” referenced in Table 7 was one of TaqPath™ 1-Step RT-qPCR Master Mix, CG (Thermo Fisher Scientific, Catalog Nos. A15299 and A15300) or TaqMan™ Fast Virus 1-Step Master Mix (Thermo Fisher Scientific, Catalog Nos. 4444432, 4444434, or 4444436)

The reaction mixes were vortexed for about 10-30 seconds and centrifuged briefly. For each reaction, the components in Table 8, below, were combined in a MicroAmp™ Optical 96-Well Reaction Plate (0.2 mL/well) (Thermo Fisher Scientific, Catalog No. N8010560):

TABLE 8
RT-qPCR Reaction

| Component | Volume/reaction | |
|---|---|---|
| Reaction Mix (see Table 7) | 20.0 | μL |
| Nucleic acid sample (from extraction) or | 5.0 | μL |
| 1 μL 2019-nCoV Control construct + 4 μL | | |
| PCR-grade water or | | |
| No template control (5 μL PCR-grade water) | | |
| Total Reaction Volume | 25.0 | μL |

The plate was sealed with a MicroAmp Optical Adhesive Film (Thermo Fisher Scientific, Catalog No. 4306311) and vortexed briefly to mix the contents. The plate was centrifuged briefly to collect the contents at the bottom of the wells. The plate was loaded into a 7500 Real-Time PCR Instrument (Thermo Fisher Scientific, Catalog Nos. 4351104-4351107) and the protocol in either Table 9 or Table 10 was run, depending on the respective RT-qPCR Master Mix used to create the reaction mix.

TABLE 9
RT-qPCR Protocol using TaqPath 1-Step RT-qPCR Master Mix
RT-qPCR Protocol
TaqPath 1-Step RT-qPCR Master Mix

| Step | Stage | # of cycles | Temp. | | Time | |
|---|---|---|---|---|---|---|
| UNG incubation | 1 | 1 | 25° | C. | 2 | min |
| Reverse transcription^{†} | 2 | 1 | 50° | C.^{†} | 15 | min |
| Polymerase activation^{‡} | 3 | 1 | 95° | C.^{‡} | 2 | min |
| Amplification | 4 | 40 | 95° | C. | 3 | sec |
| | | | 60° | C. | 30 | sec |

^{†}Preferably any temperature between 48° C.-55° C.
^{‡}RT inactivation, initial denaturation, and activation of DNAP.

TABLE 10
RT-qPCR Protocol using TaqMan Fast Virus 1-Step Master Mix
RT-qPCR Protocol
TaqMan Fast Virus 1-Step Master Mix

| Step | Stage | # of cycles | Temp. | | Time | |
|---|---|---|---|---|---|---|
| Reverse transcription | 1 | 1 | 50° | C.^{†} | 5 | min |
| RT inactivation/initial | 2 | 1 | 95° | C. | 20 | sec |
| denaturation | | | | | | |
| Amplification | 3 | 40 | 95° | C. | 3 | sec |
| | | | 60° | C. | 30 | sec |

^{†}Preferably any temperature between 48° C.-55° C.

The resulting data were analyzed using the included 7500 Software v2.3. The analysis was performed using the Auto Baseline and Auto Threshold analysis settings of the software. For each plate, the control reactions were confirmed to perform as expected (i.e., the no template control had an undetermined Cₜ value and the positive control had a Cₜ value less than or equal to 30).

The Cₜ value for each individual assay was also analyzed in accordance with Table 11.

TABLE 11
Individual assay results guide

| 2019-nCOV | RNaseP | |
|---|---|---|
| assay (FAM) | assay (VIC) | Interpreted Result |
| Cₜ < 37 | Any Value | Positive. |
| 37 ≤ Cₜ < 40 | Any Value | Inconclusive. Repeat the test. |
| Cₜ = 40 or | Cₜ < 40 | Negative. |
| undetermined | | |

The results for each tested sample was interpreted to have SARS-CoV-2 RNA present if either (i) any two of the three 2019-nCoV assays were positive or (ii) any one of the 2019-nCoV assays were positive in two different samples collected from the same subject. SARS-CoV-2 RNA was not present in the sample if all three of the 2019-nCoV assays were negative.

### Example 2: Multiplex Assay for Detecting SARS-CoV-2

An exemplary protocol for detecting SARS-CoV-2 from a biological sample via a multiplex assay was performed using the TaqPath™ COVID-19 Combo Kit (Thermo Fisher Scientific, Catalog No. A47813) or the TaqPath™ COVID-19 Combo Kit Advanced (Thermo Fisher Scientific, Catalog No. A47814). The kits are similar but with some different reagent volumes for workflows of different sample volumes. The assay kit included a “COVID-19 Real Time PCR Assay Multiplex” component that included primers and FAM-labeled probes for detecting Orf1ab, primers and ABY-labeled probes for detecting S protein, and primers and VIC-labeled probes for detecting N protein coding sequences for SARS-CoV-2, as well as a JUN-labeled internal positive control directed to either endogenous RNase P or an exogenous MS2 RNA template. The assay kit also included a synthetic DNA construct COVID-19 Control (1×104 copies/μL) encoding the target sequences for Orf1ab, S protein, and N protein.

The total nucleic acid content was isolated from samples collected via nasopharyngeal swab, nasopharyngeal aspirate, or bronchoalveolar lavage using the MagMAX Viral/Pathogen Nucleic Acid Isolation Kit (Thermo Fisher Scientific, Catalog No. A42356) in accordance with the instructions provided therewith.

For each assay, the components in Table 12 were combined for the number of reactions, plus 10% overage:

TABLE 12
RT-qPCR Reaction Mix

| Component | Volume/reaction | |
|---|---|---|
| Master Mix (4X) | 6.25 | Ml |
| COVID-19 Real Time PCR | 1.25 | μL |
| Assay Multiplex | | |
| Nuclease-free water | 12.50 | μL |
| Total Reaction Mix Volume | 20.00 | μL |

The “Master Mix” referenced in Table 12 was a TaqPath™ 1-Step Multiplex Master Mix (No ROX™) (Thermo Fisher Scientific, Catalog Nos. A28521, A28522, A28523).

The COVID-19 Control was diluted to a working stock of 25 copies/μL. The reaction mixes were vortexed for about 10-30 seconds) and centrifuged briefly. For each reaction, the components in Table 13, below, were combined in a MicroAmp™ Optical 96-Well Reaction Plate (0.2 mL/well) (Thermo Fisher Scientific, Catalog No. N8010560):

TABLE 13
RT-qPCR Reactions

| Component | Volume/reaction | |
|---|---|---|
| Reaction Mix (see Table 12) | 20.0 | μL |
| Nucleic acid sample (from RNA extraction) or | 5.00 | μL |
| 2 μL COVID-19 Control + 3 μL PCR-grade | | |
| water or | | |
| Purified Negative Control (from RNA | | |
| extraction) | | |
| Total Reaction Volume | 25.00 | μL |

The plate was sealed with a MicroAmp Optical Adhesive Film (Thermo Fisher Scientific, Catalog No. 4306311) and vortexed briefly to mix the contents. The plate was centrifuged briefly to collect the contents at the bottom of the wells. The plate was loaded into a QuantStudio 5 Real-Time PCR System (Thermo Fisher Scientific, Catalog No. A28139) and the protocol in Table 14 was run.

TABLE 14
RT-qPCR Protocol for Multiplex Assay

| Step | Stage | # of cycles | Temp. | | Time | |
|---|---|---|---|---|---|---|
| UNG incubation | 1 | 1 | 25° | C. | 2 | min |
| Reverse transcription^{†} | 2 | 1 | 53° | C.^{†} | 10 | min |
| Polymerase activation^{‡} | 3 | 1 | 95° | C. | 2 | min |
| Amplification | 4 | 40 | 95° | C. | 3 | sec |
| | | | 60° | C. | 30 | sec |

^{†}Preferably any temperature between 48° C.-55° C.
^{‡}RT inactivation, initial denaturation, and activation of DNAP.

The resulting data were analyzed using the QuantStudio Design and Analysis Software v1.5.1 included with the QuantStudio 5 Real-Time PCR System. For each plate, the control reactions were confirmed to perform as expected (i.e., the no template control had an undetermined Cₜ value and the positive control had a Cₜ value less than or equal to 30).

The Cₜ value for each individual assay was also analyzed in accordance with Table 15.

TABLE 15
Multiplex Assay Results Guide

| | | | Ct = 40 or |
|---|---|---|---|
| | Ct < 37 | 37 ≤ Ct < 40 | undetermined |
| Orflab (FAM) | Positive | Repeat test | Negative |
| N protein (VIC) | Positive | Repeat test | Negative |
| S protein (ABY) | Positive | Repeat test | Negative |
| Pos. Control (JUN) | Positive | Positive | Negative |

The results for each tested sample was interpreted to have SARS-CoV-2 RNA present if either (i) any two of Orf1ab, S protein, or N protein were positive or (ii) any one of Orf1ab, S protein, or N protein were positive in two different samples collected from the same subject. SARS-CoV-2 RNA was not present in the sample if all three of Orf1ab, S protein, and N protein were negative.

### Example 3: Detection of SARS-CoV-2 Mutant Variants

An exemplary protocol for discriminating reference SARS-CoV-2 from mutant variant SARS-CoV-2 was performed using the TaqMan™ SARS-CoV-2 Mutation Panel (Thermo Fisher Scientific, Catalog Nos. 4332077, 4332075). Each exemplary mutation assay included primers, VIC-labelled probes for detecting reference SARS-CoV-2, FAM-labelled probes for detecting a targeted mutation of a mutant variant, and optionally an internal control such as an in vitro transcribed (IVT) RNA control.

Each assay utilized the components of the reaction mix shown in Table 16. The illustrated volumes were for each well of a well plate with 0.2 ml wells and can be halved where 0.1 ml wells are used.

TABLE 16
RT-qPCR Reaction Mix

| Component | Volume | |
|---|---|---|
| RT-qPCR Master Mix, CG (4X) | 5 | μL |
| Mutation Assay (40x) | 0.5 | μL |
| RNA Sample (or nuclease-free water for | 5.0 | μL |
| control) | | |
| Nuclease-free water | 9.5 | μL |
| Total Reaction Mix Volume | 20 | μL |

The “RT-qPCR Master Mix, CG” referenced in Table 16 is available from Thermo Fisher Scientific, Catalog Nos. A15299 and A15300. Reaction mixes were vortexed for about 10-30 seconds and centrifuged briefly. The plate was loaded into a QuantStudio Real-Time PCR System utilizing QuantStudio Design and Analysis Software v2.5, and the protocol shown in Table 17 was run.

TABLE 17
RT-qPCR Protocol

| Step | Temp. | Time | | No. of cycles |
|---|---|---|---|---|
| Pre-read | 60° C. | 30 | sec | 1 |
| Reverse transcription | 50° C. | 10 | min | 1 |
| DNA Polymerase | 95° C. | 2 | min | 1 |
| activation | | | | |
| Denaturation | 95° C. | 3 | sec | 45 |
| Anneal/extend | 60° C. | 30 | sec | |
| Post-read | 60° C. | 30 | sec | 1 |

Data was generated using IVT-RNA controls for reference and mutant alleles at four different input concentrations: 100,000 copies, 10,000 copies, 1,000 copies, and 250 copies per 20 μL reaction.

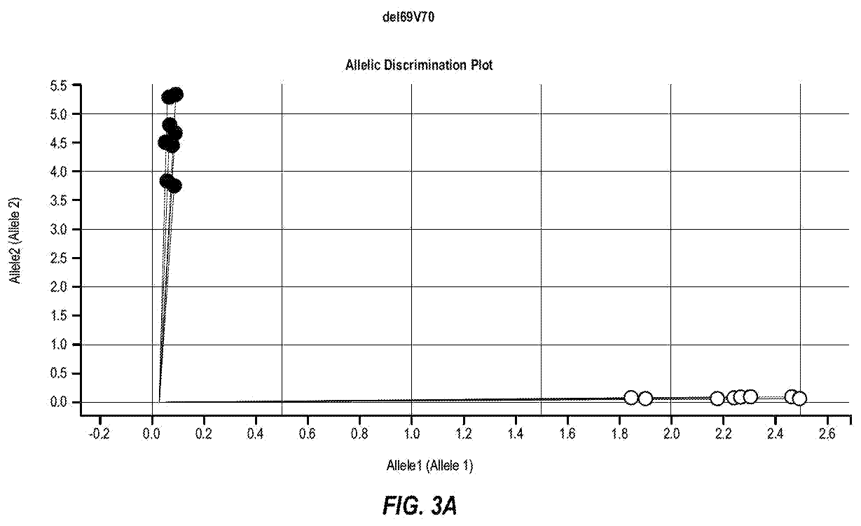

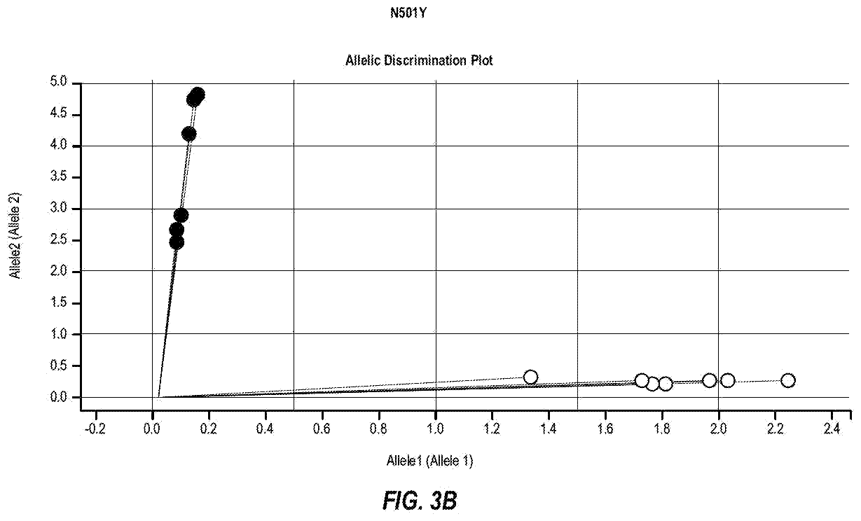

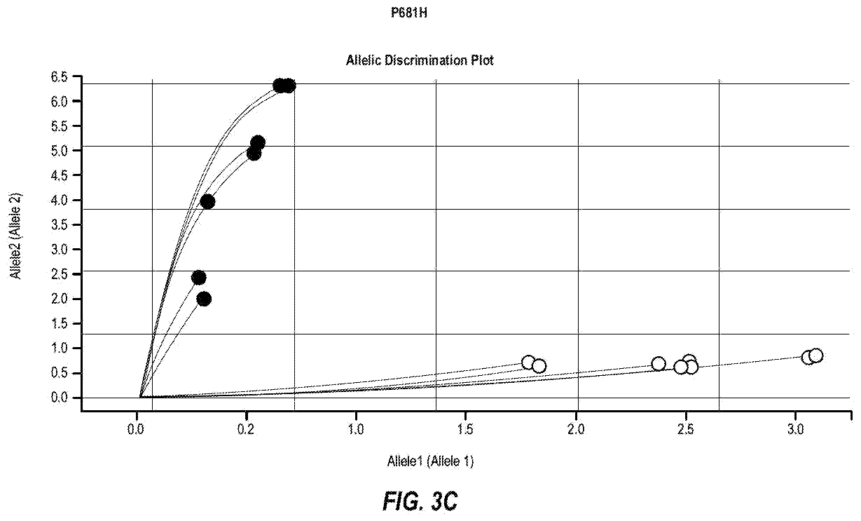

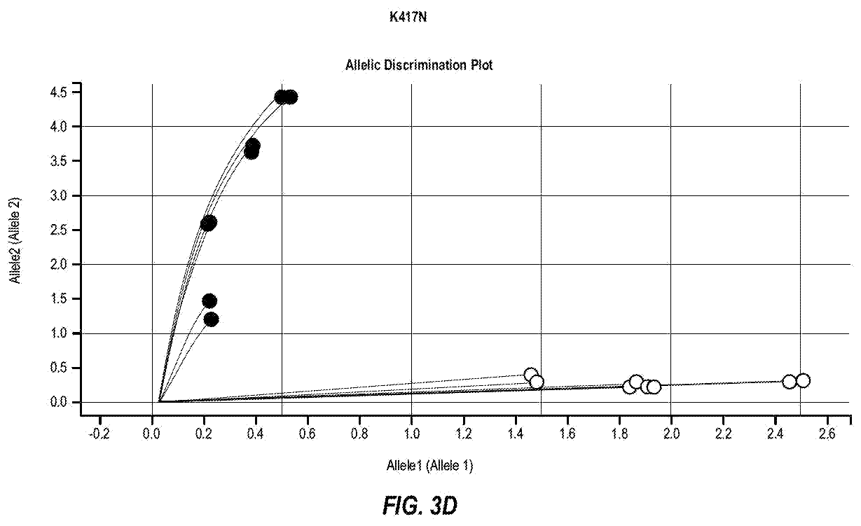

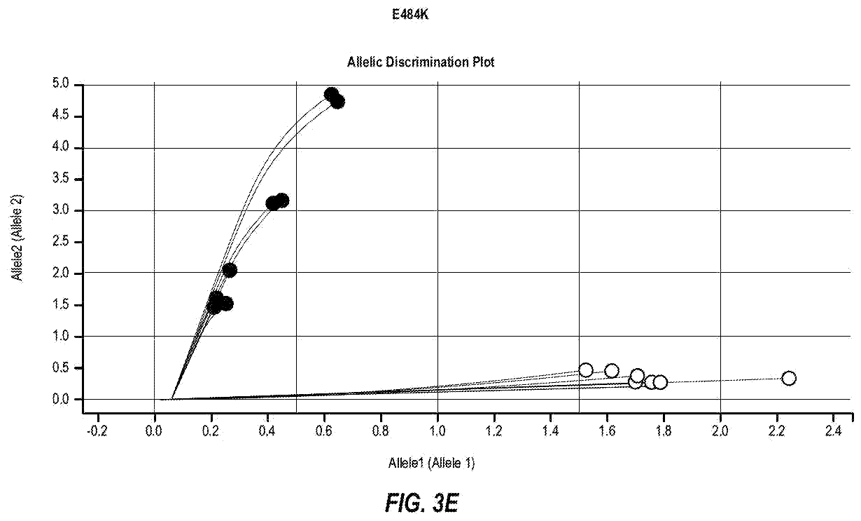

Resulting allelic discrimination plots are shown in FIGS. 3A-3E. The Figures illustrate that assays targeting del69V70, N501Y, P681H, K417N, and E484K mutations are able to effectively discriminate between these mutations and the co-mixed sequences with reference alleles.

### Example 4: Multiplex Assay Panels

A panel of assays was designed to differentiate between a first strain of SARS-CoV-2 and a second, different strain of SARS-CoV-2. The panel included assays for two or more SNP markers selected to enable differentiation between different SARS-CoV-2 variants.

Marker Selection—Data analysis for identifying SARS-CoV-2 markers was performed using the Variant Analysis for Diagnostic Monitoring (DxM) system (ROSALIND). Genome sequences and metadata used for the selection of markers in this study were obtained through a Direct Connectivity Agreement for complete daily worldwide downloads from the GISAID EpiCov database. Sequences not tagged with the “is_complete’ and sequences with “n_content” of more than 0.05 were excluded. Pairwise whole-genome alignments of all sequences were performed using LASTZ v1.04.02 with NCBI Reference Sequence: NC_045512.2 as the SARS-CoV-2 reference genome. The Bioconductor package for genetic variants, VariantAnnotation v1.20.2, was then used for the translation into amino acids in R v3.3.2, and the identification of amino acid substitutions or frameshifts were used to call a unique mutation incident.

Selection of the lineages considered for the marker panel was performed by combining the top 100 most frequent lineages reported worldwide for the 120-day period between May 12, 2021 and Sep. 11, 2021 (data not shown). 1,200,791 sequences representing 393 lineages were analyzed. The top 10 most unique mutations for each World Health Organization (WHO) label were then identified, and multiple combinations of these unique mutations were evaluated to classify a viral sequence into a WHO label with at least 90% overall accuracy. Additional mutations were added to ensure coverage for the Centers for Disease Control and Prevention (CDC) Variants Being Monitored (VBM), Variants of Interest (VOI), Variants of Concern (VOC), and Variants of High Consequence (VOHC) classifications.

The positive percent agreement (PPA) and negative percent agreement (NPA) for each marker set compared to NGS was calculated according to the Clinical and Laboratory Standards Institute (CLSI) EP12-A2: User Protocol for Evaluation of Qualitative Test Performance. A classifier algorithm was developed to measure the presence, absence, and combination of mutations to accurately assign the WHO label classification. A dedicated system was established to host the classifier algorithm and provide a web application with Application Programmer Interface (API) capabilities for standardized data submission and processing. This system was established on a secure virtual private cloud instance on the Google Cloud Platform (GCP) with the ability to process thousands of specimens per minute.

Samples—SARS-CoV-2 positive subject samples used in this investigation were collected in November 2021 and December 2021 by Helix OpCo and The University of Washington (UW), Clinical Laboratory Improvement Amendments (CLIA)-certified labs participating in the CDC National SARS-CoV-2 Strain Surveillance (NS3) sequencing program to monitor variant distribution in the United States. The Helix OpCo samples were de-identified remnants of clinical testing that were banked and used pursuant to an Institutional Research Board (IRB)-approved research registry and biobanking protocol. Use of the UW de-identified excess clinical specimens was approved with a consent waiver by the UW RB.

Genotyping Assay—Primers were selected based on mapping to genome regions with a mutation frequency of less than one percent (1%), ensuring no major polymorphisms interfere with the primers. Primer sets were designed such that amplicon sizes were below 150 base pairs (bp). Minor groove binder (MGB) probes were designed to achieve optimal discrimination between the two (2) alleles by taking the position, nucleotide composition, melting temperature (Tm), and the type of allele into consideration. The Tm of the primers ranged from 59-62° C. and the Tm of the probes ranged from 59-65° C. Viral RNA was extracted using the MagMAX Viral/Pathogen II Nucleic Acid Isolation Kit (Thermo Fisher Scientific). Real-time reverse transcription PCR using the selected panel was performed using the TaqPath™ 1-Step RT-qPCR Master Mix, CG (Thermo Fisher Scientific) on a QuantStudio™ 7 Real-Time PCR System or ProFlex™ 2×384-well PCR System (Thermo Fisher Scientific) followed by endpoint data collection using the QuantStudio™ 7 Real-Time PCR System. Data were analyzed using the TaqMan™ Genotyper v1.6 software (Thermo Fisher Scientific). Normalized reported emission of (Rn) VIC (x-axis) versus Rn FAM (y-axis) from amplification of the reference and mutant alleles was used by the software algorithm to obtain genotype calls. The specific assays for each of the markers are available commercially.

Marker Panel—An assay panel can be designed using two or more of the markers shown in Table 18. The set of assays shown in Table 18 included 45 lineage specific markers and 3 generic (variant-agnostic) markers.

TABLE 18
Example 48-, 24-, 16-, 12-, and 8-marker panels

| | | Marker Set | | | | | |
|---|---|---|---|---|---|---|---|
| Nucleotide Mutation | AA Mutation | 48 | 24 | 16 | 12 | 8 | Classification Outcome |
| nsp 10 gene (position | None | + | + | + | + | + | SARS-CoV-2 detected |
| 13025-13441) | | | | | | | |
| A23403G | S:D614G | + | + | + | + | | |
| N gene SC2 (position | None | + | | | | | |
| 29461-29482) | | | | | | | |
| T16176C | None | + | + | + | + | + | Alpha |
| A21801C | S:DB0A | + | + | + | + | + | Beta |
| A22812C | S:K417T | + | + | + | + | + | Gamma |
| C21618G | S:T19R | + | + | + | + | + | Delta |
| C22995A | S:T478K | + | + | + | + | + | Delta |
| T7424G | orf1ab:F2387V | + | + | + | + | + | Lambda |
| A13057T | None | + | + | + | + | + | Mu |
| G22018T | S:W152C | + | + | + | + | | Epsilon |
| A16500C | orf1b:Q1011H | + | + | + | + | | Iota |
| T22917A | S:L452Q | + | + | + | + | | Lambda |
| A11456G | orf1ab:I3731V | + | + | + | | | Delta |
| A28699G | None | + | + | + | | | Eta |
| G23593C | S:Q677H | + | + | + | | | Eta |
| A24775T | S:Q1071H | + | + | + | | | Kappa |
| TACATG21765---- | S:HV69-- | + | + | | | | Alpha |
| TTA21991- | S:Y144- | + | + | | | | Alpha (when combined |
| | | | | | | | with T16176C) |
| CTTTACTTG22281------ | S:LLA241-- | + | + | | | | Beta (when combined |
| | | | | | | | with A21801C) |
| T733C | None | + | + | | | | Gamma |
| T22917G | S:L452R | + | + | | | | Delta (or Epsilon when |
| | | | | | | | combined with G22018T) |
| A22320G | S:D253G | + | + | | | | Iota (when combined with |
| | | | | | | | A16500C) |
| G23012C | S:E484Q | + | + | | | | Kappa (when combined |
| | | | | | | | with A24775T) |
| C27925A | ORF8:T11K | + | + | | | | Mu |
| G22132T | S:R190S | + | | | | | Gamma |
| C23604G | S:P681R | + | | | | | Delta |
| C25469T | ORF3a:S26L | + | | | | | Delta |
| G5629T | None | + | | | | | Centers for Disease |
| C21614T | S:L18F | + | | | | | Control and Prevention |
| T21615G | S:L18R | + | | | | | (CDC) Variant Being |
| C21638T | S:P26S | + | | | | | Monitored (VBM), |
| G21770T | S:V70F | + | | | | | Variant of Interest (VOI), |
| A21801G | S:DB0G | + | | | | | Variant of Concern |
| G22335T | S:W258L | + | | | | | (VOC), or Variant of |
| G22813T | S:K417N | + | | | | | High Consequence |
| T22882G | S:N440K | + | | | | | (VOHC) |
| C22995G | S:T478R | + | | | | | |
| G23012A | S:E484K | + | | | | | |
| A23063T | S:N501Y | + | | | | | |
| A23064C | S:N501T | + | | | | | |
| A23592C | S:Q677P | + | | | | | |
| G23593T | S:Q677H | + | | | | | |
| C23604A | S:P681H | + | | | | | |
| C23664T | S:A701V | + | | | | | |
| G24410A | S:D950N | + | | | | | |
| T27206C | ORF6:F2S | + | | | | | |
| T28226C | None | + | | | | | |

Variant-Agnostic Positivity Markers—The variant-agnostic markers include 1) the S Gene: D614G (S:A23403G) mutation-a nonsynonymous mutation resulting in the replacement of aspartic acid with glycine at position 614 of the viral spike protein; 2) a conserved sequence in nsp10 (nucleotides 13025-13441); and 3) a conserved sequence identified by the CDC in the N Gene SC2 region (nucleotides 29461-29482).

A total of 1,128 retrospective samples (1,031 SARS-CoV-2 positive and 97 SARS-CoV-2 negative) were evaluated using the variant agnostic positivity markers (Table 19). The combined markers were detected in all but seven (7) of the 1,031 SARS-CoV-2 positive samples. The positive percent agreement (PPA) using any combination of two (2) or more markers is greater than or equal to 98.9% with the criteria being that one (1) marker detected is enough to make a positive call. Additionally, the PPA using one (1) marker is greater than or equal to 96%. There were no false positive results (data not shown).

TABLE 19
Variant agnostic positivity markers in vitro performance

| | Variant-Agnostic Marker | | | | |
|---|---|---|---|---|---|
| No. of | | | N gene | Positive | |
| Markers | S: D614G | nsp10 gene | SC2 | Calls | PPA (%) |
| 3 | + | + | + | 1024 | 99.3 |
| 2 | + | + | | 1020 | 98.9 |
| | + | | + | 1021 | 99 |
| | | + | + | 1023 | 99.2 |
| 1 | + | | | 993 | 96.3 |
| | | + | | 1018 | 98.7 |
| | | | + | 990 | 96 |
| 0 | | | | 7 | 0.7 |

| | |
|---|---|
| Total SARS-COV-2 positive samples: | 1031 |

Lineage Assignment—The performance of the genotyping assay panel and the associated classifier was determined by in silico and in vitro studies with retrospectively collected SARS-CoV-2 specimens. A bioinformatics simulation was performed using GISAID SARS-CoV-2 sequence data from the first week of each month beginning November 2020 through October 2021. 323,148 GISAID sequences were analyzed. With the 48-marker set, simulated PPA ranged from 80.7% to 99.9% and simulated NPA ranged from 98.1% to 100% for the top 10 WHO lineages. The performance for the Kappa variant was impacted by reporting from Asia and Oceania where many Kappa positive samples were misclassified as Delta.

TABLE 20
48-marker set in silico classifier performance

| | Simulated PPA (%) | Simulated NPA (%) |
|---|---|---|
| Alpha | 99.9 | 98.8 |
| Beta | 97.7 | 100 |
| Gamma | 99 | 100 |
| Delta | 99.8 | 98.1 |
| Epsilon | 96.1 | 100 |
| Eta | 96.2 | 100 |
| Iota | 98.4 | 100 |
| Kappa | 80.7 | 100 |
| Lambda | 98.1 | 100 |
| Mu | 98.8 | 100 |

The 1,031 SARS-CoV-2 positive samples were genotyped and classified with the 48 markers shown in Table 18. The classifications were then compared to the Phylogenetic Assignment of Named Global Outbreak Lineages (Pango) lineage assignment based on the whole-genome sequences in the GISAID database (Table 21). The PPA ranged from 96.312 to 200 and the NPA ranged from 99.2% to 100% for the top 10 WHO lineages. The classifier categorized an additional 78 samples as undetermined (data not shown). Pango assigned 77 of these samples to 14 lineages for which the genotyping assay does not include specific markers (Zeta, B.1, B.1.1.507, B.1.2, B.1.221, B.1.241, B.1.517, B.1.596, B.1.609, B.1.625, B.1.628, B.1.634, B.1.637, and C.36.3), and did not classify one (1) of these samples.

TABLE 21
48-marker set in vitro classifier performance

| | | Classifier Call | | | |
|---|---|---|---|---|---|
| Pango Assignment | | Positive | Negative | PPA/PPN (%) | |
| Alpha | Positive | 122 | 1 | 99.2 | PPA |
| | Negative | 7 | 908 | 99.2 | PPN |
| Beta | Positive | 13 | 0 | 100 | PPA |
| | Negative | 0 | 1018 | 100 | PPN |
| Gamma | Positive | 109 | 0 | 100 | PPA |
| | Negative | 0 | 922 | 100 | PPN |
| Delta | Positive | 456 | 5 | 98.9 | PPA |
| | Negative | 1 | 570 | 99.8 | PPN |
| Epsilon | Positive | 78 | 3 | 96.3 | PPA |
| | Negative | 3 | 950 | 99.7 | PPN |
| Eta | Positive | 27 | 0 | 100 | PPA |
| | Negative | 0 | 1004 | 100 | PPN |
| Iota | Positive | 82 | 0 | 100 | PPA |
| | Negative | 1 | 949 | 99.9 | PPN |
| Kappa | Positive | 1 | 0 | 100 | PPA |
| | Negative | 0 | 1030 | 100 | PPN |
| Lambda | Positive | 2 | 0 | 100 | PPA |
| | Negative | 0 | 1029 | 100 | PPN |
| Mu | Positive | 54 | 0 | 100 | PPA |
| | Negative | 0 | 977 | 100 | PPN |

Marker Reduction—To optimize assay performance in terms of sample input, reductions of the 48-marker panel were explored. We assessed the performance of 24-, 16-, 12-, and 8-marker sets that were defined based on mutation combination performance and targeted lineage prevalence during the 120-day period between May 12, 2021 and Sep. 11, 2021 (Table 22). Each of the panels also included two (2) of the variant agnostic positivity markers (nsp10 gene and S:D614G), which were used as assay internal controls. The 48-, 24-, and 16-marker sets identified the top 10 most prevalent WHO lineages as of Sep. 11, 2021 (Alpha, Beta, Gamma, Delta, Epsilon, Eta, Iota, Kappa, Lambda, and Mu), while the 12- and eight (8)-marker sets identified eight (8) and six (6) of the top 10 WHO lineages, respectively.

TABLE 22
48-, 24-, 16-, 12-, and 8-marker sets in vitro classifier performance

| | Markers: | | | | | | | | | |
|---|---|---|---|---|---|---|---|---|---|---|
| | 48 | | 24 | | 16 | | 12 | | 8 | |
| | PPA | NPA | PPA | NPA | PPA | NPA | PPA | NPA | PPA | NPA |
| | (%) | (%) | (%) | (%) | (%) | (%) | (%) | (%) | (%) | (%) |
| Alpha | 99.2 | 99.2 | 99.2 | 99.2 | 98.4 | 99.1 | 98.4 | 99.1 | 98.4 | 99.1 |
| Beta | 100 | 100 | 100 | 100 | 100 | 100 | 100 | 100 | 100 | 100 |
| Gamma | 100 | 100 | 100 | 100 | 100 | 100 | 100 | 100 | 100 | 100 |
| Delta | 98.9 | 99.8 | 98.7 | 99.6 | 98.7 | 99.6 | 98.7 | 99.6 | 98.9 | 99.8 |
| Epsilon | 96.3 | 99.7 | 96.3 | 99.7 | 96.3 | 99.7 | 96.3 | 99.7 | * | 91.5 |
| Eta | 100 | 100 | 100 | 100 | 100 | 100 | * | 97.3 | * | 97.3 |
| Iota | 100 | 99.9 | 100 | 99.9 | 100 | 99.9 | 100 | 99.9 | * | 91.3 |
| Kappa | 100 | 100 | 100 | 100 | 100 | 100 | * | 99.9 | * | 99.9 |
| Lambda | 100 | 100 | 100 | 100 | 100 | 100 | 100 | 100 | 100 | 100 |
| Mu | 100 | 100 | 100 | 100 | 100 | 100 | 100 | 100 | 100 | 100 |

*Could not call

Increase in Undetermined Calls as an Indicator of New Variant—An increase in the number of undetermined calls by the classifier provides a signal for focused sequencing of those samples, potentially allowing early detection of new variants. To test this hypothesis, a bioinformatics simulation was performed using a modification of the 12-marker panel. The two Delta-specific markers were removed to simulate what would have been observed before and during the emergence of the Delta variant. The 10-marker set was able to assign lineages to all positive samples in GISAID for North America in November 2020 and December 2020 (data not shown). The number of undetermined calls was 5, 7, and 1 respectively in January 2021, February 2021, and March 2021. In April 2021, the number increased to 51 followed by a rapid increase over the following three months to 12,825 undetermined calls in July 2021. We then compared these results to the average daily Delta prevalence in the United States from March 2021 to July 2021 as reported by the CDC. The prevalence data for the emerging Delta variant mirrors the rate of increase in undetermined calls over the same period.

### Example 5: Distinguishing Between Delta and Omicron Variants

Sequence analysis of the first 132 Omicron sequences revealed three (3) markers-ORFlab:A2710T, ORFlab:T13195C, and S:T547K-found in high percentages of these sequences. Based on in silico modeling, there was greater than 99% concurrence between the Pango assignment based on the GISAID sequence and the combined three markers (data not shown). Subsequently, we developed a genotyping assay consisting of the three Omicron-specific markers and one Delta-specific marker (S:T19R).

A total of 1,631 SARS-CoV-2 positive samples were collected and genotyped (Table 23). Sequencing confirmed that these samples consisted of 615 Omicron, 992 Delta, and three B.1 variants, as well as 21 samples that were not classified by Pango. The four-marker panel for Omicron genotyping correctly identified all 615 Omicron samples and 902 of the 992 Delta samples. The 90 Delta samples that were not detected were identified as Delta subtypes by Pango. The four-marker panel classified the three B.1 samples as undetermined, and the 21 samples not classified by Pango as 10 Omicron and 11 Delta.

TABLE 23
Four-marker set in vitro classifier performance

| | | Classifier Call | | | |
|---|---|---|---|---|---|
| Pango Assignment | | Positive | Negative | PPA/NPA (%) | |
| Omicron | Positive | 615 | 0 | 100 | PPA |
| | Negative | 10 | 1016 | 99 | NPA |
| Delta | Positive | 902 | 90 | 90.9 | PPA |
| | Negative | 11 | 639 | 98.3 | NPA |

As one example of such a four-marker set, a panel of assays may include: (1) assay no. 1188, (2) assay no. 1196, (3) assay no. 62 (or alternatively assay no. 61), and (4) assay no. 71 (or alternatively assay no. 72), as those assays are illustrated in FIGS. 2C and 2D. In some embodiments, the reference strain probes are optional in each of such assays that make up the panel.

We next deployed the four-marker panel in two CLIA-certified labs and genotyped 5,372 SARS-CoV-2 positive samples collected mainly in the States of Washington, California, and New York in December 2021. Using the four-marker panel, we determined that the relative prevalence of the Omicron variant grew from approximately 15% on Dec. 9, 2021 to approximately 80% on Dec. 21, 2021, while Delta decreased from 80% to 20% over the same period.

### EXAMPLE EMBODIMENTS

Provided below is a non-exhaustive list of numbered items reciting certain preferred embodiments:
1. A composition for detecting the presence of SARS-CoV-2 in a biological sample, comprising a nucleic acid primer and/or probe with a sequence having at least 75%, at least 80%, at least 85%, at least 90%, at least 95%, at least 97%, at least 98%, at least 99%, or 100% homology to: SEQ ID NO:1 to SEQ ID NO:1304; SEQ ID NO:2001 to SEQ ID NO:3304; SEQ ID NO:4001 to SEQ ID NO:5304; and/or SEQ ID NO:6001 to SEQ ID NO:7304.
2. The composition of item 1, comprising:
   a forward primer having at least 75%, at least 80%, at least 85%, at least 90%, at least 95%, at least 97%, at least 98%, at least 99%, or 100% homology to a sequence selected from SEQ ID NO:1 to SEQ ID NO:1304; and
   a reverse primer having at least 75%, at least 80%, at least 85%, at least 90%, at least 95%, at least 97%, at least 98%, at least 99%, or 100% homology to a sequence selected from SEQ ID NO:2001 to SEQ ID NO:3304.
3. The composition of item 2, wherein the composition further comprises one or both of:
   a probe configured for association with a reference SARS-CoV-2 allele, the probe having at least 75%, at least 80%, at least 85%, at least 90%, at least 95%, at least 97%, at least 98%, at least 99%, or 100% homology to SEQ ID NO:4001 to SEQ ID NO:5304;
   a probe configured for association with a mutant SARS-CoV-2 allele, the probe having at least 75%, at least 80%, at least 85%, at least 90%, at least 95%, at least 97%, at least 98%, at least 99%, or 100% homology to SEQ ID NO:6001 to SEQ ID NO:7304.
4. The composition of item 3, comprising:
   a sequence having at least 75%, at least 80%, at least 85%, at least 90%, at least 95%, at least 97%, at least 98%, at least 99%, or 100% homology to SEQ ID NO:X as a forward primer;
   a sequence having at least 75%, at least 80%, at least 85%, at least 90%, at least 95%, at least 97%, at least 98%, at least 99%, or 100% homology to SEQ ID NO:(X+2000) as a reverse primer;
   optionally, a sequence having at least 75%, at least 80%, at least 85%, at least 90%, at least 95%, at least 97%, at least 98%, at least 99%, or 100% homology to SEQ ID NO:(X+4000) as a probe configured for detecting a reference SARS-CoV-2 allele; and
   a sequence having at least 75%, at least 80%, at least 85%, at least 90%, at least 95%, at least 97%, at least 98%, at least 99%, or 100% homology to SEQ ID NO:(X+6000) as a probe configured for detecting a mutant SARS-CoV-2 allele, wherein X is from 1 to 1304.
5. The composition of item 3 or item 4, comprising:
   SEQ ID NO:X as a forward primer;
   SEQ ID NO:(X+2000) as a reverse primer;
   optionally SEQ ID NO:(X+4000) as a probe configured for detecting a reference SARS-CoV-2 allele; and
   SEQ ID NO:(X+6000) as a probe configured for detecting a mutant SARS-CoV-2 allele,
   wherein X is from 1 to 1304.
6. The composition of any one of items 3-5, wherein a first probe is selected from SEQ ID NO:4001 to SEQ ID NO:5304 and a second probe is selected from SEQ ID NO:6001 to SEQ ID NO:7304.
7. The composition of item 6, wherein the first probe and the second probe are differentially labeled.
8. The composition of any one of items 3-7, formulated to target one or more of an Alpha, Delta, or Omicron variant of SARS-CoV-2.
9. The composition of item 8, formulated to target the Alpha variant of SARS-CoV-2.
10. The composition of item 9, comprising two or more of:
   an assay including one of SEQ ID NO:560 through SEQ ID NO:562 as a forward primer, one of SEQ ID NO:2560 through SEQ ID NO:2562 as a reverse primer, one of SEQ ID NO:4560 through SEQ ID NO:4562 as a probe configured to target a reference SARS-CoV-2 allele, and one of SEQ ID NO:6560 through SEQ ID NO:6562 as a probe configured to target a mutant SARS-CoV-2 allele;
   an assay including SEQ ID NO:564 as a forward primer, SEQ ID NO:2564 as a reverse primer, SEQ ID NO:4564 as a probe configured to target a reference SARS-CoV-2 allele, and SEQ ID NO:6564 as a probe configured to target a mutant SARS-CoV-2 allele;
   an assay including one of SEQ ID NO:803 through SEQ ID NO:809 as a forward primer, one of SEQ ID NO:2803 through SEQ ID NO:2809 as a reverse primer, one of SEQ ID NO:4803 through SEQ ID NO:4809 as a probe configured to target a reference SARS-CoV-2 allele, and one of SEQ ID NO:6803 through SEQ ID NO:6809 as a probe configured to target a mutant SARS-CoV-2 allele;
   an assay including one of SEQ ID NO:820 through SEQ ID NO:967 as a forward primer, one of SEQ ID NO:2820 through SEQ ID NO:2967 as a reverse primer, one of SEQ ID NO:4820 through SEQ ID NO:4967 as a probe configured to target a reference SARS-CoV-2 allele, and one of SEQ ID NO:6820 through SEQ ID NO:6967 as a probe configured to target a mutant SARS-CoV-2 allele;
   an assay including SEQ ID NO:28 as a forward primer, SEQ ID NO:2028 as a reverse primer, SEQ ID NO:4028 as a probe configured to target a reference SARS-CoV-2 allele, and SEQ ID NO:6028 as a probe configured to target a mutant SARS-CoV-2 allele;
   an assay including one of SEQ ID NO:336 through SEQ ID NO:380 as a forward primer, one of SEQ ID NO:2336 through SEQ ID NO:2380 as a reverse primer, one of SEQ ID NO:4336 through SEQ ID NO:4380 as a probe configured to target a reference SARS-CoV-2 allele, and one of SEQ ID NO:6336 through SEQ ID NO:6380 as a probe configured to target a mutant SARS-CoV-2 allele;
   an assay including one of SEQ ID NO:93 through SEQ ID NO:323 as a forward primer, one of SEQ ID NO:2093 through SEQ ID NO:2323 as a reverse primer, one of SEQ ID NO:4093 through SEQ ID NO:4323 as a probe configured to target a reference SARS-CoV-2 allele, and one of SEQ ID NO:6093 through SEQ ID NO:6323 as a probe configured to target a mutant SARS-CoV-2 allele;
   an assay including one of SEQ ID NO:1063 through SEQ ID NO:1186 as a forward primer, one of SEQ ID NO:3063 through SEQ ID NO:3186 as a reverse primer, one of SEQ ID NO:5063 through SEQ ID NO:5186 as a probe configured to target a reference SARS-CoV-2 allele, and one of SEQ ID NO:7063 through SEQ ID NO:7186 as a probe configured to target a mutant SARS-CoV-2 allele;
   an assay including one of SEQ ID NO:1197 through SEQ ID NO:1267 as a forward primer, one of SEQ ID NO:3197 through SEQ ID NO:3267 as a reverse primer, one of SEQ ID NO:5197 through SEQ ID NO:5267 as a probe configured to target a reference SARS-CoV-2 allele, and one of SEQ ID NO:7197 through SEQ ID NO:7267 as a probe configured to target a mutant SARS-CoV-2 allele. 11. The composition of item 9, comprising two or more of:
   an assay including one of SEQ ID NO:560 through SEQ ID NO:562 as a forward primer, one of SEQ ID NO:2560 through SEQ ID NO:2562 as a reverse primer, one of SEQ ID NO:4560 through SEQ ID NO:4562 as a probe configured to target a reference SARS-CoV-2 allele, and one of SEQ ID NO:6560 through SEQ ID NO:6562 as a probe configured to target a mutant SARS-CoV-2 allele;
   an assay including SEQ ID NO:564 as a forward primer, SEQ ID NO:2564 as a reverse primer, SEQ ID NO:4564 as a probe configured to target a reference SARS-CoV-2 allele, and SEQ ID NO:6564 as a probe configured to target a mutant SARS-CoV-2 allele;
   an assay including one of SEQ ID NO:803 through SEQ ID NO:809 as a forward primer, one of SEQ ID NO:2803 through SEQ ID NO:2809 as a reverse primer, one of SEQ ID NO:4803 through SEQ ID NO:4809 as a probe configured to target a reference SARS-CoV-2 allele, and one of SEQ ID NO:6803 through SEQ ID NO:6809 as a probe configured to target a mutant SARS-CoV-2 allele;
   an assay including one of SEQ ID NO:820 through SEQ ID NO:824 as a forward primer, one of SEQ ID NO:2820 through SEQ ID NO:2824 as a reverse primer, one of SEQ ID NO:4820 through SEQ ID NO:4824 as a probe configured to target a reference SARS-CoV-2 allele, and one of SEQ ID NO:6820 through SEQ ID NO:6824 as a probe configured to target a mutant SARS-CoV-2 allele;
   an assay including SEQ ID NO:28 as a forward primer, SEQ ID NO:2028 as a reverse primer, SEQ ID NO:4028 as a probe configured to target a reference SARS-CoV-2 allele, and SEQ ID NO:6028 as a probe configured to target a mutant SARS-CoV-2 allele;
   an assay including one of SEQ ID NO:336 through SEQ ID NO:339 as a forward primer, one of SEQ ID NO:2336 through SEQ ID NO:2339 as a reverse primer, one of SEQ ID NO:4336 through SEQ ID NO:4339 as a probe configured to target a reference SARS-CoV-2 allele, and one of SEQ ID NO:6336 through SEQ ID NO:6339 as a probe configured to target a mutant SARS-CoV-2 allele;
   an assay including one of SEQ ID NO:93 through SEQ ID NO:102 as a forward primer, one of SEQ ID NO:2093 through SEQ ID NO:2102 as a reverse primer, one of SEQ ID NO:4093 through SEQ ID NO:4102 as a probe configured to target a reference SARS-CoV-2 allele, and one of SEQ ID NO:6093 through SEQ ID NO:6102 as a probe configured to target a mutant SARS-CoV-2 allele;
   an assay including SEQ ID NO:1063 as a forward primer, SEQ ID NO:3063 as a reverse primer, SEQ ID NO:5063 as a probe configured to target a reference SARS-CoV-2 allele, and SEQ ID NO:7063 as a probe configured to target a mutant SARS-CoV-2 allele;
   an assay including one of SEQ ID NO:1197 through SEQ ID NO:1203 as a forward primer, one of SEQ ID NO:3197 through SEQ ID NO:3203 as a reverse primer, one of SEQ ID NO:5197 through SEQ ID NO:5203 as a probe configured to target a reference SARS-CoV-2 allele, and one of SEQ ID NO:7197 through SEQ ID NO:7203 as a probe configured to target a mutant SARS-CoV-2 allele. 12. The composition of item 8, formulated to target the Delta variant of SARS-CoV-2.
13. The composition of item 12, comprising two or more of:
   an assay including SEQ ID NO:784 as a forward primer, SEQ ID NO:2784 as a reverse primer, SEQ ID NO:4784 as a probe configured to target a reference SARS-CoV-2 allele, and SEQ ID NO:6784 as a probe configured to target a mutant SARS-CoV-2 allele;
   an assay including SEQ ID NO:968 as a forward primer, SEQ ID NO:2968 as a reverse primer, SEQ ID NO:4968 as a probe configured to target a reference SARS-CoV-2 allele, and SEQ ID NO:6968 as a probe configured to target a mutant SARS-CoV-2 allele;
   an assay including one of SEQ ID NO:607 through SEQ ID NO:608 as a forward primer, one of SEQ ID NO:2607 through SEQ ID NO:2608 as a reverse primer, one of SEQ ID NO:4607 through SEQ ID NO:4608 as a probe configured to target a reference SARS-CoV-2 allele, and one of SEQ ID NO:6607 through SEQ ID NO:6608 as a probe configured to target a mutant SARS-CoV-2 allele;
   an assay including one of SEQ ID NO:1188 through SEQ ID NO:1189 as a forward primer, one of SEQ ID NO:3188 through SEQ ID NO:3189 as a reverse primer, one of SEQ ID NO:5188 through SEQ ID NO:5189 as a probe configured to target a reference SARS-CoV-2 allele, and one of SEQ ID NO:7188 through SEQ ID NO:7189 as a probe configured to target a mutant SARS-CoV-2 allele;
   an assay including one of SEQ ID NO:1192 through SEQ ID NO:1193 as a forward primer, one of SEQ ID NO:3192 through SEQ ID NO:3193 as a reverse primer, one of SEQ ID NO:5192 through SEQ ID NO:5193 as a probe configured to target a reference SARS-CoV-2 allele, and one of SEQ ID NO:7192 through SEQ ID NO:7193 as a probe configured to target a mutant SARS-CoV-2 allele.
14. The composition of item 8, formulated to target the Omicron variant of SARS-CoV-2.
15. The composition of item 14, comprising two or more of:
   an assay including one of SEQ ID NO:61 through SEQ ID NO:62 as a forward primer, one of SEQ ID NO:2061 through SEQ ID NO:2062 as a reverse primer, one of SEQ ID NO:4061 through SEQ ID NO:4062 as a probe configured to target a reference SARS-CoV-2 allele, and one of SEQ ID NO:6061 through SEQ ID NO:6062 as a probe configured to target a mutant SARS-CoV-2 allele;
   an assay including SEQ ID NO:616 as a forward primer, SEQ ID NO:2616 as a reverse primer, SEQ ID NO:4616 as a probe configured to target a reference SARS-CoV-2 allele, and SEQ ID NO:6616 as a probe configured to target a mutant SARS-CoV-2 allele;
   an assay including one of SEQ ID NO:972 through SEQ ID NO:975 as a forward primer, one of SEQ ID NO:2972 through SEQ ID NO:2975 as a reverse primer, one of SEQ ID NO:4972 through SEQ ID NO:4975 as a probe configured to target a reference SARS-CoV-2 allele, and one of SEQ ID NO:6972 through SEQ ID NO:6975 as a probe configured to target a mutant SARS-CoV-2 allele;
   an assay including one of SEQ ID NO:71 through SEQ ID NO:72 as a forward primer, one of SEQ ID NO:2071 through SEQ ID NO:2072 as a reverse primer, one of SEQ ID NO:4071 through SEQ ID NO:4072 as a probe configured to target a reference SARS-CoV-2 allele, and one of SEQ ID NO:6071 through SEQ ID NO:6072 as a probe configured to target a mutant SARS-CoV-2 allele;
   an assay including SEQ ID NO:1196 as a forward primer, SEQ ID NO:3196 as a reverse primer, SEQ ID NO:5196 as a probe configured to target a reference SARS-CoV-2 allele, and SEQ ID NO:7196 as a probe configured to target a mutant SARS-CoV-2 allele.
16. The composition of item 8, formulated to target and/or distinguish between the Alpha and Delta variants of SARS-CoV-2.
17. The composition of item 16, comprising at least one assay formulated for targeting the Alpha variant selected from:
   an assay including one of SEQ ID NO:560 through SEQ ID NO:562 as a forward primer, one of SEQ ID NO:2560 through SEQ ID NO:2562 as a reverse primer, one of SEQ ID NO:4560 through SEQ ID NO:4562 as a probe configured to target a reference SARS-CoV-2 allele, and one of SEQ ID NO:6560 through SEQ ID NO:6562 as a probe configured to target a mutant SARS-CoV-2 allele;
   an assay including SEQ ID NO:564 as a forward primer, SEQ ID NO:2564 as a reverse primer, SEQ ID NO:4564 as a probe configured to target a reference SARS-CoV-2 allele, and SEQ ID NO:6564 as a probe configured to target a mutant SARS-CoV-2 allele;
   an assay including one of SEQ ID NO:803 through SEQ ID NO:809 as a forward primer, one of SEQ ID NO:2803 through SEQ ID NO:2809 as a reverse primer, one of SEQ ID NO:4803 through SEQ ID NO:4809 as a probe configured to target a reference SARS-CoV-2 allele, and one of SEQ ID NO:6803 through SEQ ID NO:6809 as a probe configured to target a mutant SARS-CoV-2 allele;
   an assay including one of SEQ ID NO:820 through SEQ ID NO:967 as a forward primer, one of SEQ ID NO:2820 through SEQ ID NO:2967 as a reverse primer, one of SEQ ID NO:4820 through SEQ ID NO:4967 as a probe configured to target a reference SARS-CoV-2 allele, and one of SEQ ID NO:6820 through SEQ ID NO:6967 as a probe configured to target a mutant SARS-CoV-2 allele;
   an assay including SEQ ID NO:28 as a forward primer, SEQ ID NO:2028 as a reverse primer, SEQ ID NO:4028 as a probe configured to target a reference SARS-CoV-2 allele, and SEQ ID NO:6028 as a probe configured to target a mutant SARS-CoV-2 allele;
   an assay including one of SEQ ID NO:336 through SEQ ID NO:380 as a forward primer, one of SEQ ID NO:2336 through SEQ ID NO:2380 as a reverse primer, one of SEQ ID NO:4336 through SEQ ID NO:4380 as a probe configured to target a reference SARS-CoV-2 allele, and one of SEQ ID NO:6336 through SEQ ID NO:6380 as a probe configured to target a mutant SARS-CoV-2 allele;
   an assay including one of SEQ ID NO:93 through SEQ ID NO:323 as a forward primer, one of SEQ ID NO:2093 through SEQ ID NO:2323 as a reverse primer, one of SEQ ID NO:4093 through SEQ ID NO:4323 as a probe configured to target a reference SARS-CoV-2 allele, and one of SEQ ID NO:6093 through SEQ ID NO:6323 as a probe configured to target a mutant SARS-CoV-2 allele;
   an assay including one of SEQ ID NO:1063 through SEQ ID NO:1186 as a forward primer, one of SEQ ID NO:3063 through SEQ ID NO:3186 as a reverse primer, one of SEQ ID NO:5063 through SEQ ID NO:5186 as a probe configured to target a reference SARS-CoV-2 allele, and one of SEQ ID NO:7063 through SEQ ID NO:7186 as a probe configured to target a mutant SARS-CoV-2 allele;
   an assay including one of SEQ ID NO:1197 through SEQ ID NO:1267 as a forward primer, one of SEQ ID NO:3197 through SEQ ID NO:3267 as a reverse primer, one of SEQ ID NO:5197 through SEQ ID NO:5267 as a probe configured to target a reference SARS-CoV-2 allele, and one of SEQ ID NO:7197 through SEQ ID NO:7267 as a probe configured to target a mutant SARS-CoV-2 allele; and at least one assay formulated for targeting the Delta variant selected from:
   an assay including SEQ ID NO:784 as a forward primer, SEQ ID NO:2784 as a reverse primer, SEQ ID NO:4784 as a probe configured to target a reference SARS-CoV-2 allele, and SEQ ID NO:6784 as a probe configured to target a mutant SARS-CoV-2 allele;
   an assay including SEQ ID NO:968 as a forward primer, SEQ ID NO:2968 as a reverse primer, SEQ ID NO:4968 as a probe configured to target a reference SARS-CoV-2 allele, and SEQ ID NO:6968 as a probe configured to target a mutant SARS-CoV-2 allele;
   an assay including one of SEQ ID NO:607 through SEQ ID NO:608 as a forward primer, one of SEQ ID NO:2607 through SEQ ID NO:2608 as a reverse primer, one of SEQ ID NO:4607 through SEQ ID NO:4608 as a probe configured to target a reference SARS-CoV-2 allele, and one of SEQ ID NO:6607 through SEQ ID NO:6608 as a probe configured to target a mutant SARS-CoV-2 allele;
   an assay including one of SEQ ID NO:1188 through SEQ ID NO:1189 as a forward primer, one of SEQ ID NO:3188 through SEQ ID NO:3189 as a reverse primer, one of SEQ ID NO:5188 through SEQ ID NO:5189 as a probe configured to target a reference SARS-CoV-2 allele, and one of SEQ ID NO:7188 through SEQ ID NO:7189 as a probe configured to target a mutant SARS-CoV-2 allele;
   an assay including one of SEQ ID NO:1192 through SEQ ID NO:1193 as a forward primer, one of SEQ ID NO:3192 through SEQ ID NO:3193 as a reverse primer, one of SEQ ID NO:5192 through SEQ ID NO:5193 as a probe configured to target a reference SARS-CoV-2 allele, and one of SEQ ID NO:7192 through SEQ ID NO:7193 as a probe configured to target a mutant SARS-CoV-2 allele.
18. The composition of item 8, formulated to target and/or distinguish between the Alpha and Omicron variants of SARS-CoV-2.
19. The composition of item 18, comprising at least one assay formulated for targeting the Alpha variant selected from:
   an assay including one of SEQ ID NO:560 through SEQ ID NO:562 as a forward primer, one of SEQ ID NO:2560 through SEQ ID NO:2562 as a reverse primer, one of SEQ ID NO:4560 through SEQ ID NO:4562 as a probe configured to target a reference SARS-CoV-2 allele, and one of SEQ ID NO:6560 through SEQ ID NO:6562 as a probe configured to target a mutant SARS-CoV-2 allele;
   an assay including SEQ ID NO:564 as a forward primer, SEQ ID NO:2564 as a reverse primer, SEQ ID NO:4564 as a probe configured to target a reference SARS-CoV-2 allele, and SEQ ID NO:6564 as a probe configured to target a mutant SARS-CoV-2 allele;
   an assay including one of SEQ ID NO:803 through SEQ ID NO:809 as a forward primer, one of SEQ ID NO:2803 through SEQ ID NO:2809 as a reverse primer, one of SEQ ID NO:4803 through SEQ ID NO:4809 as a probe configured to target a reference SARS-CoV-2 allele, and one of SEQ ID NO:6803 through SEQ ID NO:6809 as a probe configured to target a mutant SARS-CoV-2 allele;
   an assay including one of SEQ ID NO:820 through SEQ ID NO:967 as a forward primer, one of SEQ ID NO:2820 through SEQ ID NO:2967 as a reverse primer, one of SEQ ID NO:4820 through SEQ ID NO:4967 as a probe configured to target a reference SARS-CoV-2 allele, and one of SEQ ID NO:6820 through SEQ ID NO:6967 as a probe configured to target a mutant SARS-CoV-2 allele;
   an assay including SEQ ID NO:28 as a forward primer, SEQ ID NO:2028 as a reverse primer, SEQ ID NO:4028 as a probe configured to target a reference SARS-CoV-2 allele, and SEQ ID NO:6028 as a probe configured to target a mutant SARS-CoV-2 allele;
   an assay including one of SEQ ID NO:336 through SEQ ID NO:380 as a forward primer, one of SEQ ID NO:2336 through SEQ ID NO:2380 as a reverse primer, one of SEQ ID NO:4336 through SEQ ID NO:4380 as a probe configured to target a reference SARS-CoV-2 allele, and one of SEQ ID NO:6336 through SEQ ID NO:6380 as a probe configured to target a mutant SARS-CoV-2 allele;
   an assay including one of SEQ ID NO:93 through SEQ ID NO:323 as a forward primer, one of SEQ ID NO:2093 through SEQ ID NO:2323 as a reverse primer, one of SEQ ID NO:4093 through SEQ ID NO:4323 as a probe configured to target a reference SARS-CoV-2 allele, and one of SEQ ID NO:6093 through SEQ ID NO:6323 as a probe configured to target a mutant SARS-CoV-2 allele;
   an assay including one of SEQ ID NO:1063 through SEQ ID NO:1186 as a forward primer, one of SEQ ID NO:3063 through SEQ ID NO:3186 as a reverse primer, one of SEQ ID NO:5063 through SEQ ID NO:5186 as a probe configured to target a reference SARS-CoV-2 allele, and one of SEQ ID NO:7063 through SEQ ID NO:7186 as a probe configured to target a mutant SARS-CoV-2 allele;
   an assay including one of SEQ ID NO:1197 through SEQ ID NO:1267 as a forward primer, one of SEQ ID NO:3197 through SEQ ID NO:3267 as a reverse primer, one of SEQ ID NO:5197 through SEQ ID NO:5267 as a probe configured to target a reference SARS-CoV-2 allele, and one of SEQ ID NO:7197 through SEQ ID NO:7267 as a probe configured to target a mutant SARS-CoV-2 allele; and at least one assay formulated for targeting the Omicron variant selected from:
   an assay including one of SEQ ID NO:61 through SEQ ID NO:62 as a forward primer, one of SEQ ID NO:2061 through SEQ ID NO:2062 as a reverse primer, one of SEQ ID NO:4061 through SEQ ID NO:4062 as a probe configured to target a reference SARS-CoV-2 allele, and one of SEQ ID NO:6061 through SEQ ID NO:6062 as a probe configured to target a mutant SARS-CoV-2 allele;
   an assay including SEQ ID NO:616 as a forward primer, SEQ ID NO:2616 as a reverse primer, SEQ ID NO:4616 as a probe configured to target a reference SARS-CoV-2 allele, and SEQ ID NO:6616 as a probe configured to target a mutant SARS-CoV-2 allele;
   an assay including one of SEQ ID NO:972 through SEQ ID NO:975 as a forward primer, one of SEQ ID NO:2972 through SEQ ID NO:2975 as a reverse primer, one of SEQ ID NO:4972 through SEQ ID NO:4975 as a probe configured to target a reference SARS-CoV-2 allele, and one of SEQ ID NO:6972 through SEQ ID NO:6975 as a probe configured to target a mutant SARS-CoV-2 allele;
   an assay including one of SEQ ID NO:71 through SEQ ID NO:72 as a forward primer, one of SEQ ID NO:2071 through SEQ ID NO:2072 as a reverse primer, one of SEQ ID NO:4071 through SEQ ID NO:4072 as a probe configured to target a reference SARS-CoV-2 allele, and one of SEQ ID NO:6071 through SEQ ID NO:6072 as a probe configured to target a mutant SARS-CoV-2 allele;
   an assay including SEQ ID NO:1196 as a forward primer, SEQ ID NO:3196 as a reverse primer, SEQ ID NO:5196 as a probe configured to target a reference SARS-CoV-2 allele, and SEQ ID NO:7196 as a probe configured to target a mutant SARS-CoV-2 allele.
20. The composition of item 8, formulated to target and/or distinguish between the Delta and Omicron variants of SARS-CoV-2.
21. The composition of item 20, comprising at least one assay formulated for targeting the Delta variant selected from:
   an assay including SEQ ID NO:784 as a forward primer, SEQ ID NO:2784 as a reverse primer, SEQ ID NO:4784 as a probe configured to target a reference SARS-CoV-2 allele, and SEQ ID NO:6784 as a probe configured to target a mutant SARS-CoV-2 allele;
   an assay including SEQ ID NO:968 as a forward primer, SEQ ID NO:2968 as a reverse primer, SEQ ID NO:4968 as a probe configured to target a reference SARS-CoV-2 allele, and SEQ ID NO:6968 as a probe configured to target a mutant SARS-CoV-2 allele;
   an assay including one of SEQ ID NO:607 through SEQ ID NO:608 as a forward primer, one of SEQ ID NO:2607 through SEQ ID NO:2608 as a reverse primer, one of SEQ ID NO:4607 through SEQ ID NO:4608 as a probe configured to target a reference SARS-CoV-2 allele, and one of SEQ ID NO:6607 through SEQ ID NO:6608 as a probe configured to target a mutant SARS-CoV-2 allele;
   an assay including one of SEQ ID NO:1188 through SEQ ID NO:1189 as a forward primer, one of SEQ ID NO:3188 through SEQ ID NO:3189 as a reverse primer, one of SEQ ID NO:5188 through SEQ ID NO:5189 as a probe configured to target a reference SARS-CoV-2 allele, and one of SEQ ID NO:7188 through SEQ ID NO:7189 as a probe configured to target a mutant SARS-CoV-2 allele;
   an assay including one of SEQ ID NO:1192 through SEQ ID NO:1193 as a forward primer, one of SEQ ID NO:3192 through SEQ ID NO:3193 as a reverse primer, one of SEQ ID NO:5192 through SEQ ID NO:5193 as a probe configured to target a reference SARS-CoV-2 allele, and one of SEQ ID NO:7192 through SEQ ID NO:7193 as a probe configured to target a mutant SARS-CoV-2 allele; and at least one assay formulated for targeting the Omicron variant selected from:
   an assay including one of SEQ ID NO:61 through SEQ ID NO:62 as a forward primer, one of SEQ ID NO:2061 through SEQ ID NO:2062 as a reverse primer, one of SEQ ID NO:4061 through SEQ ID NO:4062 as a probe configured to target a reference SARS-CoV-2 allele, and one of SEQ ID NO:6061 through SEQ ID NO:6062 as a probe configured to target a mutant SARS-CoV-2 allele;
   an assay including SEQ ID NO:616 as a forward primer, SEQ ID NO:2616 as a reverse primer, SEQ ID NO:4616 as a probe configured to target a reference SARS-CoV-2 allele, and SEQ ID NO:6616 as a probe configured to target a mutant SARS-CoV-2 allele;
   an assay including one of SEQ ID NO:972 through SEQ ID NO:975 as a forward primer, one of SEQ ID NO:2972 through SEQ ID NO:2975 as a reverse primer, one of SEQ ID NO:4972 through SEQ ID NO:4975 as a probe configured to target a reference SARS-CoV-2 allele, and one of SEQ ID NO:6972 through SEQ ID NO:6975 as a probe configured to target a mutant SARS-CoV-2 allele;
   an assay including one of SEQ ID NO:71 through SEQ ID NO:72 as a forward primer, one of SEQ ID NO:2071 through SEQ ID NO:2072 as a reverse primer, one of SEQ ID NO:4071 through SEQ ID NO:4072 as a probe configured to target a reference SARS-CoV-2 allele, and one of SEQ ID NO:6071 through SEQ ID NO:6072 as a probe configured to target a mutant SARS-CoV-2 allele;
   an assay including SEQ ID NO:1196 as a forward primer, SEQ ID NO:3196 as a reverse primer, SEQ ID NO:5196 as a probe configured to target a reference SARS-CoV-2 allele, and SEQ ID NO:7196 as a probe configured to target a mutant SARS-CoV-2 allele.
22. The composition of item 8, formulated to target and/or distinguish between the Alpha, Delta, and Omicron variants of SARS-CoV-2.
23. The composition of item 22, comprising at least one assay formulated for targeting the Alpha variant selected from:
   an assay including one of SEQ ID NO:560 through SEQ ID NO:562 as a forward primer, one of SEQ ID NO:2560 through SEQ ID NO:2562 as a reverse primer, one of SEQ ID NO:4560 through SEQ ID NO:4562 as a probe configured to target a reference SARS-CoV-2 allele, and one of SEQ ID NO:6560 through SEQ ID NO:6562 as a probe configured to target a mutant SARS-CoV-2 allele;
   an assay including SEQ ID NO:564 as a forward primer, SEQ ID NO:2564 as a reverse primer, SEQ ID NO:4564 as a probe configured to target a reference SARS-CoV-2 allele, and SEQ ID NO:6564 as a probe configured to target a mutant SARS-CoV-2 allele;
   an assay including one of SEQ ID NO:803 through SEQ ID NO:809 as a forward primer, one of SEQ ID NO:2803 through SEQ ID NO:2809 as a reverse primer, one of SEQ ID NO:4803 through SEQ ID NO:4809 as a probe configured to target a reference SARS-CoV-2 allele, and one of SEQ ID NO:6803 through SEQ ID NO:6809 as a probe configured to target a mutant SARS-CoV-2 allele;
   an assay including one of SEQ ID NO:820 through SEQ ID NO:967 as a forward primer, one of SEQ ID NO:2820 through SEQ ID NO:2967 as a reverse primer, one of SEQ ID NO:4820 through SEQ ID NO:4967 as a probe configured to target a reference SARS-CoV-2 allele, and one of SEQ ID NO:6820 through SEQ ID NO:6967 as a probe configured to target a mutant SARS-CoV-2 allele;
   an assay including SEQ ID NO:28 as a forward primer, SEQ ID NO:2028 as a reverse primer, SEQ ID NO:4028 as a probe configured to target a reference SARS-CoV-2 allele, and SEQ ID NO:6028 as a probe configured to target a mutant SARS-CoV-2 allele;
   an assay including one of SEQ ID NO:336 through SEQ ID NO:380 as a forward primer, one of SEQ ID NO:2336 through SEQ ID NO:2380 as a reverse primer, one of SEQ ID NO:4336 through SEQ ID NO:4380 as a probe configured to target a reference SARS-CoV-2 allele, and one of SEQ ID NO:6336 through SEQ ID NO:6380 as a probe configured to target a mutant SARS-CoV-2 allele;
   an assay including one of SEQ ID NO:93 through SEQ ID NO:323 as a forward primer, one of SEQ ID NO:2093 through SEQ ID NO:2323 as a reverse primer, one of SEQ ID NO:4093 through SEQ ID NO:4323 as a probe configured to target a reference SARS-CoV-2 allele, and one of SEQ ID NO:6093 through SEQ ID NO:6323 as a probe configured to target a mutant SARS-CoV-2 allele;
   an assay including one of SEQ ID NO:1063 through SEQ ID NO:1186 as a forward primer, one of SEQ ID NO:3063 through SEQ ID NO:3186 as a reverse primer, one of SEQ ID NO:5063 through SEQ ID NO:5186 as a probe configured to target a reference SARS-CoV-2 allele, and one of SEQ ID NO:7063 through SEQ ID NO:7186 as a probe configured to target a mutant SARS-CoV-2 allele;
   an assay including one of SEQ ID NO:1197 through SEQ ID NO:1267 as a forward primer, one of SEQ ID NO:3197 through SEQ ID NO:3267 as a reverse primer, one of SEQ ID NO:5197 through SEQ ID NO:5267 as a probe configured to target a reference SARS-CoV-2 allele, and one of SEQ ID NO:7197 through SEQ ID NO:7267 as a probe configured to target a mutant SARS-CoV-2 allele;
   at least one assay formulated for targeting the Delta variant selected from: an assay including SEQ ID NO:784 as a forward primer, SEQ ID NO:2784 as a reverse primer, SEQ ID NO:4784 as a probe configured to target a reference SARS-CoV-2 allele, and SEQ ID NO:6784 as a probe configured to target a mutant SARS-CoV-2 allele;
   an assay including SEQ ID NO:968 as a forward primer, SEQ ID NO:2968 as a reverse primer, SEQ ID NO:4968 as a probe configured to target a reference SARS-CoV-2 allele, and SEQ ID NO:6968 as a probe configured to target a mutant SARS-CoV-2 allele;
   an assay including one of SEQ ID NO:607 through SEQ ID NO:608 as a forward primer, one of SEQ ID NO:2607 through SEQ ID NO:2608 as a reverse primer, one of SEQ ID NO:4607 through SEQ ID NO:4608 as a probe configured to target a reference SARS-CoV-2 allele, and one of SEQ ID NO:6607 through SEQ ID NO:6608 as a probe configured to target a mutant SARS-CoV-2 allele;
   an assay including one of SEQ ID NO:1188 through SEQ ID NO:1189 as a forward primer, one of SEQ ID NO:3188 through SEQ ID NO:3189 as a reverse primer, one of SEQ ID NO:5188 through SEQ ID NO:5189 as a probe configured to target a reference SARS-CoV-2 allele, and one of SEQ ID NO:7188 through SEQ ID NO:7189 as a probe configured to target a mutant SARS-CoV-2 allele;
   an assay including one of SEQ ID NO:1192 through SEQ ID NO:1193 as a forward primer, one of SEQ ID NO:3192 through SEQ ID NO:3193 as a reverse primer, one of SEQ ID NO:5192 through SEQ ID NO:5193 as a probe configured to target a reference SARS-CoV-2 allele, and one of SEQ ID NO:7192 through SEQ ID NO:7193 as a probe configured to target a mutant SARS-CoV-2 allele; and at least one assay formulated for targeting the Omicron variant selected from:
   an assay including one of SEQ ID NO:61 through SEQ ID NO:62 as a forward primer, one of SEQ ID NO:2061 through SEQ ID NO:2062 as a reverse primer, one of SEQ ID NO:4061 through SEQ ID NO:4062 as a probe configured to target a reference SARS-CoV-2 allele, and one of SEQ ID NO:6061 through SEQ ID NO:6062 as a probe configured to target a mutant SARS-CoV-2 allele;
   an assay including SEQ ID NO:616 as a forward primer, SEQ ID NO:2616 as a reverse primer, SEQ ID NO:4616 as a probe configured to target a reference SARS-CoV-2 allele, and SEQ ID NO:6616 as a probe configured to target a mutant SARS-CoV-2 allele;
   an assay including one of SEQ ID NO:972 through SEQ ID NO:975 as a forward primer, one of SEQ ID NO:2972 through SEQ ID NO:2975 as a reverse primer, one of SEQ ID NO:4972 through SEQ ID NO:4975 as a probe configured to target a reference SARS-CoV-2 allele, and one of SEQ ID NO:6972 through SEQ ID NO:6975 as a probe configured to target a mutant SARS-CoV-2 allele;
   an assay including one of SEQ ID NO:71 through SEQ ID NO:72 as a forward primer, one of SEQ ID NO:2071 through SEQ ID NO:2072 as a reverse primer, one of SEQ ID NO:4071 through SEQ ID NO:4072 as a probe configured to target a reference SARS-CoV-2 allele, and one of SEQ ID NO:6071 through SEQ ID NO:6072 as a probe configured to target a mutant SARS-CoV-2 allele;
   an assay including SEQ ID NO:1196 as a forward primer, SEQ ID NO:3196 as a reverse primer, SEQ ID NO:5196 as a probe configured to target a reference SARS-CoV-2 allele, and SEQ ID NO:7196 as a probe configured to target a mutant SARS-CoV-2 allele.
24. The composition of item 23, comprising:
   an assay including one of SEQ ID NO:61 through SEQ ID NO:62 as a forward primer, one of SEQ ID NO:2061 through SEQ ID NO:2062 as a reverse primer, one of SEQ ID NO:4061 through SEQ ID NO:4062 as a probe configured to target a reference SARS-CoV-2 allele, and one of SEQ ID NO:6061 through SEQ ID NO:6062 as a probe configured to target a mutant SARS-CoV-2 allele;
   an assay including one of SEQ ID NO:71 through SEQ ID NO:72 as a forward primer, one of SEQ ID NO:2071 through SEQ ID NO:2072 as a reverse primer, one of SEQ ID NO:4071 through SEQ ID NO:4072 as a probe configured to target a reference SARS-CoV-2 allele, and one of SEQ ID NO:6071 through SEQ ID NO:6072 as a probe configured to target a mutant SARS-CoV-2 allele;
   an assay including SEQ ID NO:1188 as a forward primer, SEQ ID NO:3188 as a reverse primer, SEQ ID NO:5188 as a probe configured to target a reference SARS-CoV-2 allele, and SEQ ID NO:7188 as a probe configured to target a mutant SARS-CoV-2 allele;
   an assay including SEQ ID NO:1196 as a forward primer, SEQ ID NO:3196 as a reverse primer, SEQ ID NO:5196 as a probe configured to target a reference SARS-CoV-2 allele, and SEQ ID NO:7196 as a probe configured to target a mutant SARS-CoV-2 allele.
25. The composition of any one of items 1-24, further including one or more of a nucleic acid template, a polymerase, a buffer, and/or nucleotides.
26. The composition of item 25, wherein the nucleic acid template is an RNA template.
27. The composition of item 25, wherein the nucleic acid template is a DNA template.
28. The composition of item 25, wherein the nucleic acid template is a cDNA template.
29. The composition of item 25, wherein the nucleic acid template is a genomic DNA (gDNA) template.
30. The composition of any one of items 1-29, wherein a first probe includes a fluorescent or other detectable label.
31. The composition of item 30, wherein the label of the first probe is a fluorescent label.
32. The composition of item 31, wherein the probe further includes a quencher that quenches the fluorescent label.
33. The composition of any one of items 1-32, wherein at least one of the primers or probes of the composition are configured to hybridize with a target region within SEQ ID NO:7311, SEQ ID NO:7312, or SEQ ID NO:7313.
34. The composition of any one of items 1-33 comprising a plurality of forward primers and a plurality of reverse primers to enable multiplex analysis of multiple target regions.
35. The composition of item 34, wherein the multiple target regions are mutation sites that are together associated with a particular variant.
36. The composition of item 35, comprising a plurality of different mutant allele probes each configured to associate with amplicons of a respective target region if the target region includes the mutation.
37. The composition of item 36, comprising a plurality of different reference probes each configured to associate with amplicons of a respective target region if the target region does not include the mutation.
38. The composition of any one of items 1-37, wherein the biological sample is a saliva sample, buccal sample, nasal sample, nasal pharyngeal sample, blood sample, urine sample, or semen sample.
39. The composition of any one of items 1-38 wherein the biological sample is a human sample.
40. The composition of any one of items 1-38, wherein the biological sample is a non-human sample.
41. The composition of item 40, wherein the non-human sample is a mammalian sample.
42. The composition of item 41, wherein the mammalian sample is from a mink, cat, dog, or livestock animal.
43. A kit for detecting SARS-CoV-2 nucleic acid in a biological sample, comprising a composition of any one of items 1-42.
44. The kit of item 43, further including a master mix.
45. The kit of item 44, wherein the master mix is a TaqMan Fast Virus 1-Step Master Mix or a TaqPath 1-Step RT-qPCR Master Mix.
46. The kit of any one of items 43-45, wherein at least one of the components is dried or freeze dried.
47. The kit of any one of items 43-46, further including an array of PCR assays, each PCR assay situated in a different locus of the array.
48. The kit of item 47, wherein the PCR assays are qPCR assays.
49. A method for the detection of one or more SARS-CoV-2 coronaviruses in a biological sample comprising:
   (a) providing a reaction mixture containing the biological sample and a composition or kit as in any one of items 1-48; and
   (b) subjecting the reaction mixture to reaction conditions suitable to amplify a targeted SARS-CoV-2 nucleic acid.
50. The method of item 49, further including generating one or more amplicons via PCR.
51. The method of item 49 or 50, further including monitoring fluorescence produced during the PCR.
52. The method of any one of items 49-51, further including determining the amount of nucleic acid present in the biological sample.
53. The method of any one of items 49-52, wherein a positive control and/or a negative control are analyzed in conjunction with the sample.
54. The method of item 53, where in the positive control is a synthetic plasmid comprising targets from the coronavirus Orf genes (e.g., Orf1a, Orf1b, Orf1ab, Orf8), the S protein gene, the N protein gene and/or RNase P.
55. The method of any one of items 49-54, further comprising identifying the SARS-CoV-2 as a variant or as reference form.
56. The method of any one of items 49-55, wherein the biological sample is a saliva sample, buccal sample, nasal sample, nasal pharyngeal sample, blood sample, urine sample, or semen sample.
57. The method of any one of items 49-56, wherein the biological sample is a human sample.
58. The method of any one of items 49-56, wherein the biological sample is a non-human sample.
59. The method of item 58, wherein the non-human sample is a mammalian sample.
60. The method of item 59, wherein the mammalian sample is from a mink, cat, dog, or livestock animal.
61. A method of detecting SARS-CoV-2 viral nucleic acid present in a biological sample, comprising:
   (a) providing a composition or a kit as in any one of items 1-48;
   (b) forming a reaction volume by contacting the composition, in any order or combination, with a polymerase, nucleotides, and nucleic acid obtained from a biological sample; and
   (c) forming one or more amplification products containing amplified coronaviral sequences in the reaction volume, wherein the forming includes subjecting the reaction volume to amplification conditions suitable to amplify target coronaviral sequences from coronaviral nucleic acid, wherein the coronaviral nucleic acid are present in the sample prior to amplification.
62. The method of item 61, further including detecting at least one of the amplification products during and/or after the forming step.
63. The method of item 62, further including diagnosing a coronaviral infection in the organism.
64. The method of any one of items 61-63, wherein the forming includes amplifying coronaviral target sequences from the coronaviral nucleic acid derived from the N gene, the S gene, the Orf genes (e.g., Orf1a, Orf1b, Orf1ab, Orf8), or combinations thereof.
65. The method of any one of items 61-64, further comprising identifying the presence of a SARS-CoV-2 variant.
66. The method of any one of items 61-65, further comprising determining whether detected SARS-CoV-2 nucleic acid is associated with a variant or a reference type.
67. The method of item 66, wherein the variant is selected from Alpha, Delta, and Omicron.
68. The method of any one of items 61-67, wherein the biological sample is a saliva sample, buccal sample, nasal sample, nasal pharyngeal sample, blood sample, urine sample, or semen sample.
69. The method of any one of items 61-68, wherein the biological sample is a human sample.
70. The method of any one of items 61-68, wherein the biological sample is a non-human sample.
71. The method of item 70, wherein the non-human sample is a mammalian sample.
72. The method of item 71, wherein the mammalian sample is from a mink, cat, dog, or livestock animal.

1. A composition for detecting the presence of SARS-CoV-2 in a biological sample, comprising a nucleic acid primer and/or probe with a sequence having at least 75%, at least 80%, at least 85%, at least 90%, at least 95%, at least 97%, at least 98%, at least 99%, or 100% homology to: SEQ ID NO:1 to SEQ ID NO:1304; SEQ ID NO:2001 to SEQ ID NO:3304; SEQ ID NO:4001 to SEQ ID NO:5304; and/or SEQ ID NO:6001 to SEQ ID NO:7304.

2. The composition of claim 1, comprising:
a forward primer having at least 75%, at least 80%, at least 85%, at least 90%, at least 95%, at least 97%, at least 98%, at least 99%, or 100% homology to a sequence selected from SEQ ID NO:1 to SEQ ID NO:1304; and
a reverse primer having at least 75%, at least 80%, at least 85%, at least 90%, at least 95%, at least 97%, at least 98%, at least 99%, or 100% homology to a sequence selected from SEQ ID NO:2001 to SEQ ID NO:3304.

3. The composition of claim 2, wherein the composition further comprises one or both of:
a probe configured for association with a reference SARS-CoV-2 allele, the probe having at least 75%, at least 80%, at least 85%, at least 90%, at least 95%, at least 97%, at least 98%, at least 99%, or 100% homology to SEQ ID NO:4001 to SEQ ID NO:5304;
a probe configured for association with a mutant SARS-CoV-2 allele, the probe having at least 75%, at least 80%, at least 85%, at least 90%, at least 95%, at least 97%, at least 98%, at least 99%, or 100% homology to SEQ ID NO:6001 to SEQ ID NO:7304.

4. (canceled)

5. The composition of claim 3, comprising:
SEQ ID NO:X as a forward primer;
SEQ ID NO:(X+2000) as a reverse primer;
optionally SEQ ID NO:(X+4000) as a probe configured for detecting a reference SARS-CoV-2 allele; and
SEQ ID NO:(X+6000) as a probe configured for detecting a mutant SARS-CoV-2 allele, wherein X is from 1 to 1304.

6. (canceled)

7. (canceled)

8. The composition of claim 3, formulated to target one or more of an Alpha, Delta, or Omicron variant of SARS-CoV-2.

9. The composition of claim 8, formulated to target the Alpha variant of SARS-CoV-2.

10. The composition of claim 9, comprising two or more of:
an assay including one of SEQ ID NO:560 through SEQ ID NO:562 as a forward primer, one of SEQ ID NO:2560 through SEQ ID NO:2562 as a reverse primer, one of SEQ ID NO:4560 through SEQ ID NO:4562 as a probe configured to target a reference SARS-CoV-2 allele, and one of SEQ ID NO:6560 through SEQ ID NO:6562 as a probe configured to target a mutant SARS-CoV-2 allele;
an assay including SEQ ID NO:564 as a forward primer, SEQ ID NO:2564 as a reverse primer, SEQ ID NO:4564 as a probe configured to target a reference SARS-CoV-2 allele, and SEQ ID NO:6564 as a probe configured to target a mutant SARS-CoV-2 allele;
an assay including one of SEQ ID NO:803 through SEQ ID NO:809 as a forward primer, one of SEQ ID NO:2803 through SEQ ID NO:2809 as a reverse primer, one of SEQ ID NO:4803 through SEQ ID NO:4809 as a probe configured to target a reference SARS-CoV-2 allele, and one of SEQ ID NO:6803 through SEQ ID NO:6809 as a probe configured to target a mutant SARS-CoV-2 allele;
an assay including one of SEQ ID NO:820 through SEQ ID NO:967 as a forward primer, one of SEQ ID NO:2820 through SEQ ID NO:2967 as a reverse primer, one of SEQ ID NO:4820 through SEQ ID NO:4967 as a probe configured to target a reference SARS-CoV-2 allele, and one of SEQ ID NO:6820 through SEQ ID NO:6967 as a probe configured to target a mutant SARS-CoV-2 allele;
an assay including SEQ ID NO:28 as a forward primer, SEQ ID NO:2028 as a reverse primer, SEQ ID NO:4028 as a probe configured to target a reference SARS-CoV-2 allele, and SEQ ID NO:6028 as a probe configured to target a mutant SARS-CoV-2 allele;
an assay including one of SEQ ID NO:336 through SEQ ID NO:380 as a forward primer, one of SEQ ID NO:2336 through SEQ ID NO:2380 as a reverse primer, one of SEQ ID NO:4336 through SEQ ID NO:4380 as a probe configured to target a reference SARS-CoV-2 allele, and one of SEQ ID NO:6336 through SEQ ID NO:6380 as a probe configured to target a mutant SARS-CoV-2 allele;
an assay including one of SEQ ID NO:93 through SEQ ID NO:323 as a forward primer, one of SEQ ID NO:2093 through SEQ ID NO:2323 as a reverse primer, one of SEQ ID NO:4093 through SEQ ID NO:4323 as a probe configured to target a reference SARS-CoV-2 allele, and one of SEQ ID NO:6093 through SEQ ID NO:6323 as a probe configured to target a mutant SARS-CoV-2 allele;
an assay including one of SEQ ID NO:1063 through SEQ ID NO:1186 as a forward primer, one of SEQ ID NO:3063 through SEQ ID NO:3186 as a reverse primer, one of SEQ ID NO:5063 through SEQ ID NO:5186 as a probe configured to target a reference SARS-CoV-2 allele, and one of SEQ ID NO:7063 through SEQ ID NO:7186 as a probe configured to target a mutant SARS-CoV-2 allele;
an assay including one of SEQ ID NO:1197 through SEQ ID NO:1267 as a forward primer, one of SEQ ID NO:3197 through SEQ ID NO:3267 as a reverse primer, one of SEQ ID NO:5197 through SEQ ID NO:5267 as a probe configured to target a reference SARS-CoV-2 allele, and one of SEQ ID NO:7197 through SEQ ID NO:7267 as a probe configured to target a mutant SARS-CoV-2 allele.

11. The composition of claim 9, comprising two or more of:
an assay including one of SEQ ID NO:560 through SEQ ID NO:562 as a forward primer, one of SEQ ID NO:2560 through SEQ ID NO:2562 as a reverse primer, one of SEQ ID NO:4560 through SEQ ID NO:4562 as a probe configured to target a reference SARS-CoV-2 allele, and one of SEQ ID NO:6560 through SEQ ID NO:6562 as a probe configured to target a mutant SARS-CoV-2 allele;
an assay including SEQ ID NO:564 as a forward primer, SEQ ID NO:2564 as a reverse primer, SEQ ID NO:4564 as a probe configured to target a reference SARS-CoV-2 allele, and SEQ ID NO:6564 as a probe configured to target a mutant SARS-CoV-2 allele;
an assay including one of SEQ ID NO:803 through SEQ ID NO:809 as a forward primer, one of SEQ ID NO:2803 through SEQ ID NO:2809 as a reverse primer, one of SEQ ID NO:4803 through SEQ ID NO:4809 as a probe configured to target a reference SARS-CoV-2 allele, and one of SEQ ID NO:6803 through SEQ ID NO:6809 as a probe configured to target a mutant SARS-CoV-2 allele;
an assay including one of SEQ ID NO:820 through SEQ ID NO:824 as a forward primer, one of SEQ ID NO:2820 through SEQ ID NO:2824 as a reverse primer, one of SEQ ID NO:4820 through SEQ ID NO:4824 as a probe configured to target a reference SARS-CoV-2 allele, and one of SEQ ID NO:6820 through SEQ ID NO:6824 as a probe configured to target a mutant SARS-CoV-2 allele;
an assay including SEQ ID NO:28 as a forward primer, SEQ ID NO:2028 as a reverse primer, SEQ ID NO:4028 as a probe configured to target a reference SARS-CoV-2 allele, and SEQ ID NO:6028 as a probe configured to target a mutant SARS-CoV-2 allele;
an assay including one of SEQ ID NO:336 through SEQ ID NO:339 as a forward primer, one of SEQ ID NO:2336 through SEQ ID NO:2339 as a reverse primer, one of SEQ ID NO:4336 through SEQ ID NO:4339 as a probe configured to target a reference SARS-CoV-2 allele, and one of SEQ ID NO:6336 through SEQ ID NO:6339 as a probe configured to target a mutant SARS-CoV-2 allele;
an assay including one of SEQ ID NO:93 through SEQ ID NO:102 as a forward primer, one of SEQ ID NO:2093 through SEQ ID NO:2102 as a reverse primer, one of SEQ ID NO:4093 through SEQ ID NO:4102 as a probe configured to target a reference SARS-CoV-2 allele, and one of SEQ ID NO:6093 through SEQ ID NO:6102 as a probe configured to target a mutant SARS-CoV-2 allele;
an assay including SEQ ID NO:1063 as a forward primer, SEQ ID NO:3063 as a reverse primer, SEQ ID NO:5063 as a probe configured to target a reference SARS-CoV-2 allele, and SEQ ID NO:7063 as a probe configured to target a mutant SARS-CoV-2 allele;
an assay including one of SEQ ID NO:1197 through SEQ ID NO:1203 as a forward primer, one of SEQ ID NO:3197 through SEQ ID NO:3203 as a reverse primer, one of SEQ ID NO:5197 through SEQ ID NO:5203 as a probe configured to target a reference SARS-CoV-2 allele, and one of SEQ ID NO:7197 through SEQ ID NO:7203 as a probe configured to target a mutant SARS-CoV-2 allele.

12. The composition of claim 8, formulated to target the Delta variant of SARS-CoV-2.

13. The composition of claim 12, comprising two or more of:
an assay including SEQ ID NO:784 as a forward primer, SEQ ID NO:2784 as a reverse primer, SEQ ID NO:4784 as a probe configured to target a reference SARS-CoV-2 allele, and SEQ ID NO:6784 as a probe configured to target a mutant SARS-CoV-2 allele;
an assay including SEQ ID NO:968 as a forward primer, SEQ ID NO:2968 as a reverse primer, SEQ ID NO:4968 as a probe configured to target a reference SARS-CoV-2 allele, and SEQ ID NO:6968 as a probe configured to target a mutant SARS-CoV-2 allele;
an assay including one of SEQ ID NO:607 through SEQ ID NO:608 as a forward primer, one of SEQ ID NO:2607 through SEQ ID NO:2608 as a reverse primer, one of SEQ ID NO:4607 through SEQ ID NO:4608 as a probe configured to target a reference SARS-CoV-2 allele, and one of SEQ ID NO:6607 through SEQ ID NO:6608 as a probe configured to target a mutant SARS-CoV-2 allele;
an assay including one of SEQ ID NO:1188 through SEQ ID NO:1189 as a forward primer, one of SEQ ID NO:3188 through SEQ ID NO:3189 as a reverse primer, one of SEQ ID NO:5188 through SEQ ID NO:5189 as a probe configured to target a reference SARS-CoV-2 allele, and one of SEQ ID NO:7188 through SEQ ID NO:7189 as a probe configured to target a mutant SARS-CoV-2 allele;
an assay including one of SEQ ID NO:1192 through SEQ ID NO:1193 as a forward primer, one of SEQ ID NO:3192 through SEQ ID NO:3193 as a reverse primer, one of SEQ ID NO:5192 through SEQ ID NO:5193 as a probe configured to target a reference SARS-CoV-2 allele, and one of SEQ ID NO:7192 through SEQ ID NO:7193 as a probe configured to target a mutant SARS-CoV-2 allele.

14. The composition of claim 8, formulated to target the Omicron variant of SARS-CoV-2.

15. The composition of claim 14, comprising two or more of:
an assay including one of SEQ ID NO:61 through SEQ ID NO:62 as a forward primer, one of SEQ ID NO:2061 through SEQ ID NO:2062 as a reverse primer, one of SEQ ID NO:4061 through SEQ ID NO:4062 as a probe configured to target a reference SARS-CoV-2 allele, and one of SEQ ID NO:6061 through SEQ ID NO:6062 as a probe configured to target a mutant SARS-CoV-2 allele;
an assay including SEQ ID NO:616 as a forward primer, SEQ ID NO:2616 as a reverse primer, SEQ ID NO:4616 as a probe configured to target a reference SARS-CoV-2 allele, and SEQ ID NO:6616 as a probe configured to target a mutant SARS-CoV-2 allele;
an assay including one of SEQ ID NO:972 through SEQ ID NO:975 as a forward primer, one of SEQ ID NO:2972 through SEQ ID NO:2975 as a reverse primer, one of SEQ ID NO:4972 through SEQ ID NO:4975 as a probe configured to target a reference SARS-CoV-2 allele, and one of SEQ ID NO:6972 through SEQ ID NO:6975 as a probe configured to target a mutant SARS-CoV-2 allele;
an assay including one of SEQ ID NO:71 through SEQ ID NO:72 as a forward primer, one of SEQ ID NO:2071 through SEQ ID NO:2072 as a reverse primer, one of SEQ ID NO:4071 through SEQ ID NO:4072 as a probe configured to target a reference SARS-CoV-2 allele, and one of SEQ ID NO:6071 through SEQ ID NO:6072 as a probe configured to target a mutant SARS-CoV-2 allele;
an assay including SEQ ID NO:1196 as a forward primer, SEQ ID NO:3196 as a reverse primer, SEQ ID NO:5196 as a probe configured to target a reference SARS-CoV-2 allele, and SEQ ID NO:7196 as a probe configured to target a mutant SARS-CoV-2 allele.

16. The composition of claim 8, formulated to target and/or distinguish between the Alpha and Delta variants of SARS-CoV-2.

17. The composition of claim 16, comprising at least one assay formulated for targeting the Alpha variant selected from:
an assay including one of SEQ ID NO:560 through SEQ ID NO:562 as a forward primer, one of SEQ ID NO:2560 through SEQ ID NO:2562 as a reverse primer, one of SEQ ID NO:4560 through SEQ ID NO:4562 as a probe configured to target a reference SARS-CoV-2 allele, and one of SEQ ID NO:6560 through SEQ ID NO:6562 as a probe configured to target a mutant SARS-CoV-2 allele;
an assay including SEQ ID NO:564 as a forward primer, SEQ ID NO:2564 as a reverse primer, SEQ ID NO:4564 as a probe configured to target a reference SARS-CoV-2 allele, and SEQ ID NO:6564 as a probe configured to target a mutant SARS-CoV-2 allele;
an assay including one of SEQ ID NO:803 through SEQ ID NO:809 as a forward primer, one of SEQ ID NO:2803 through SEQ ID NO:2809 as a reverse primer, one of SEQ ID NO:4803 through SEQ ID NO:4809 as a probe configured to target a reference SARS-CoV-2 allele, and one of SEQ ID NO:6803 through SEQ ID NO:6809 as a probe configured to target a mutant SARS-CoV-2 allele;
an assay including one of SEQ ID NO:820 through SEQ ID NO:967 as a forward primer, one of SEQ ID NO:2820 through SEQ ID NO:2967 as a reverse primer, one of SEQ ID NO:4820 through SEQ ID NO:4967 as a probe configured to target a reference SARS-CoV-2 allele, and one of SEQ ID NO:6820 through SEQ ID NO:6967 as a probe configured to target a mutant SARS-CoV-2 allele;
an assay including SEQ ID NO:28 as a forward primer, SEQ ID NO:2028 as a reverse primer, SEQ ID NO:4028 as a probe configured to target a reference SARS-CoV-2 allele, and SEQ ID NO:6028 as a probe configured to target a mutant SARS-CoV-2 allele;
an assay including one of SEQ ID NO:336 through SEQ ID NO:380 as a forward primer, one of SEQ ID NO:2336 through SEQ ID NO:2380 as a reverse primer, one of SEQ ID NO:4336 through SEQ ID NO:4380 as a probe configured to target a reference SARS-CoV-2 allele, and one of SEQ ID NO:6336 through SEQ ID NO:6380 as a probe configured to target a mutant SARS-CoV-2 allele;
an assay including one of SEQ ID NO:93 through SEQ ID NO:323 as a forward primer, one of SEQ ID NO:2093 through SEQ ID NO:2323 as a reverse primer, one of SEQ ID NO:4093 through SEQ ID NO:4323 as a probe configured to target a reference SARS-CoV-2 allele, and one of SEQ ID NO:6093 through SEQ ID NO:6323 as a probe configured to target a mutant SARS-CoV-2 allele;
an assay including one of SEQ ID NO:1063 through SEQ ID NO:1186 as a forward primer, one of SEQ ID NO:3063 through SEQ ID NO:3186 as a reverse primer, one of SEQ ID NO:5063 through SEQ ID NO:5186 as a probe configured to target a reference SARS-CoV-2 allele, and one of SEQ ID NO:7063 through SEQ ID NO:7186 as a probe configured to target a mutant SARS-CoV-2 allele;
an assay including one of SEQ ID NO:1197 through SEQ ID NO:1267 as a forward primer, one of SEQ ID NO:3197 through SEQ ID NO:3267 as a reverse primer, one of SEQ ID NO:5197 through SEQ ID NO:5267 as a probe configured to target a reference SARS-CoV-2 allele, and one of SEQ ID NO:7197 through SEQ ID NO:7267 as a probe configured to target a mutant SARS-CoV-2 allele; and
at least one assay formulated for targeting the Delta variant selected from:
an assay including SEQ ID NO:784 as a forward primer, SEQ ID NO:2784 as a reverse primer, SEQ ID NO:4784 as a probe configured to target a reference SARS-CoV-2 allele, and SEQ ID NO:6784 as a probe configured to target a mutant SARS-CoV-2 allele;
an assay including SEQ ID NO:968 as a forward primer, SEQ ID NO:2968 as a reverse primer, SEQ ID NO:4968 as a probe configured to target a reference SARS-CoV-2 allele, and SEQ ID NO:6968 as a probe configured to target a mutant SARS-CoV-2 allele;
an assay including one of SEQ ID NO:607 through SEQ ID NO:608 as a forward primer, one of SEQ ID NO:2607 through SEQ ID NO:2608 as a reverse primer, one of SEQ ID NO:4607 through SEQ ID NO:4608 as a probe configured to target a reference SARS-CoV-2 allele, and one of SEQ ID NO:6607 through SEQ ID NO:6608 as a probe configured to target a mutant SARS-CoV-2 allele;
an assay including one of SEQ ID NO:1188 through SEQ ID NO:1189 as a forward primer, one of SEQ ID NO:3188 through SEQ ID NO:3189 as a reverse primer, one of SEQ ID NO:5188 through SEQ ID NO:5189 as a probe configured to target a reference SARS-CoV-2 allele, and one of SEQ ID NO:7188 through SEQ ID NO:7189 as a probe configured to target a mutant SARS-CoV-2 allele;
an assay including one of SEQ ID NO:1192 through SEQ ID NO:1193 as a forward primer, one of SEQ ID NO:3192 through SEQ ID NO:3193 as a reverse primer, one of SEQ ID NO:5192 through SEQ ID NO:5193 as a probe configured to target a reference SARS-CoV-2 allele, and one of SEQ ID NO:7192 through SEQ ID NO:7193 as a probe configured to target a mutant SARS-CoV-2 allele.

18. The composition of claim 8, formulated to target and/or distinguish between the Alpha and Omicron variants of SARS-CoV-2.

19. The composition of claim 18, comprising at least one assay formulated for targeting the Alpha variant selected from:
an assay including one of SEQ ID NO:560 through SEQ ID NO:562 as a forward primer, one of SEQ ID NO:2560 through SEQ ID NO:2562 as a reverse primer, one of SEQ ID NO:4560 through SEQ ID NO:4562 as a probe configured to target a reference SARS-CoV-2 allele, and one of SEQ ID NO:6560 through SEQ ID NO:6562 as a probe configured to target a mutant SARS-CoV-2 allele;
an assay including SEQ ID NO:564 as a forward primer, SEQ ID NO:2564 as a reverse primer, SEQ ID NO:4564 as a probe configured to target a reference SARS-CoV-2 allele, and SEQ ID NO:6564 as a probe configured to target a mutant SARS-CoV-2 allele;
an assay including one of SEQ ID NO:803 through SEQ ID NO:809 as a forward primer, one of SEQ ID NO:2803 through SEQ ID NO:2809 as a reverse primer, one of SEQ ID NO:4803 through SEQ ID NO:4809 as a probe configured to target a reference SARS-CoV-2 allele, and one of SEQ ID NO:6803 through SEQ ID NO:6809 as a probe configured to target a mutant SARS-CoV-2 allele;
an assay including one of SEQ ID NO:820 through SEQ ID NO:967 as a forward primer, one of SEQ ID NO:2820 through SEQ ID NO:2967 as a reverse primer, one of SEQ ID NO:4820 through SEQ ID NO:4967 as a probe configured to target a reference SARS-CoV-2 allele, and one of SEQ ID NO:6820 through SEQ ID NO:6967 as a probe configured to target a mutant SARS-CoV-2 allele;
an assay including SEQ ID NO:28 as a forward primer, SEQ ID NO:2028 as a reverse primer, SEQ ID NO:4028 as a probe configured to target a reference SARS-CoV-2 allele, and SEQ ID NO:6028 as a probe configured to target a mutant SARS-CoV-2 allele;
an assay including one of SEQ ID NO:336 through SEQ ID NO:380 as a forward primer, one of SEQ ID NO:2336 through SEQ ID NO:2380 as a reverse primer, one of SEQ ID NO:4336 through SEQ ID NO:4380 as a probe configured to target a reference SARS-CoV-2 allele, and one of SEQ ID NO:6336 through SEQ ID NO:6380 as a probe configured to target a mutant SARS-CoV-2 allele;
an assay including one of SEQ ID NO:93 through SEQ ID NO:323 as a forward primer, one of SEQ ID NO:2093 through SEQ ID NO:2323 as a reverse primer, one of SEQ ID NO:4093 through SEQ ID NO:4323 as a probe configured to target a reference SARS-CoV-2 allele, and one of SEQ ID NO:6093 through SEQ ID NO:6323 as a probe configured to target a mutant SARS-CoV-2 allele;
an assay including one of SEQ ID NO:1063 through SEQ ID NO:1186 as a forward primer, one of SEQ ID NO:3063 through SEQ ID NO:3186 as a reverse primer, one of SEQ ID NO:5063 through SEQ ID NO:5186 as a probe configured to target a reference SARS-CoV-2 allele, and one of SEQ ID NO:7063 through SEQ ID NO:7186 as a probe configured to target a mutant SARS-CoV-2 allele;
an assay including one of SEQ ID NO:1197 through SEQ ID NO:1267 as a forward primer, one of SEQ ID NO:3197 through SEQ ID NO:3267 as a reverse primer, one of SEQ ID NO:5197 through SEQ ID NO:5267 as a probe configured to target a reference SARS-CoV-2 allele, and one of SEQ ID NO:7197 through SEQ ID NO:7267 as a probe configured to target a mutant SARS-CoV-2 allele; and
at least one assay formulated for targeting the Omicron variant selected from:
an assay including one of SEQ ID NO:61 through SEQ ID NO:62 as a forward primer, one of SEQ ID NO:2061 through SEQ ID NO:2062 as a reverse primer, one of SEQ ID NO:4061 through SEQ ID NO:4062 as a probe configured to target a reference SARS-CoV-2 allele, and one of SEQ ID NO:6061 through SEQ ID NO:6062 as a probe configured to target a mutant SARS-CoV-2 allele;
an assay including SEQ ID NO:616 as a forward primer, SEQ ID NO:2616 as a reverse primer, SEQ ID NO:4616 as a probe configured to target a reference SARS-CoV-2 allele, and SEQ ID NO:6616 as a probe configured to target a mutant SARS-CoV-2 allele;
an assay including one of SEQ ID NO:972 through SEQ ID NO:975 as a forward primer, one of SEQ ID NO:2972 through SEQ ID NO:2975 as a reverse primer, one of SEQ ID NO:4972 through SEQ ID NO:4975 as a probe configured to target a reference SARS-CoV-2 allele, and one of SEQ ID NO:6972 through SEQ ID NO:6975 as a probe configured to target a mutant SARS-CoV-2 allele;
an assay including one of SEQ ID NO:71 through SEQ ID NO:72 as a forward primer, one of SEQ ID NO:2071 through SEQ ID NO:2072 as a reverse primer, one of SEQ ID NO:4071 through SEQ ID NO:4072 as a probe configured to target a reference SARS-CoV-2 allele, and one of SEQ ID NO:6071 through SEQ ID NO:6072 as a probe configured to target a mutant SARS-CoV-2 allele;
an assay including SEQ ID NO:1196 as a forward primer, SEQ ID NO:3196 as a reverse primer, SEQ ID NO:5196 as a probe configured to target a reference SARS-CoV-2 allele, and SEQ ID NO:7196 as a probe configured to target a mutant SARS-CoV-2 allele.

20. The composition of claim 8, formulated to target and/or distinguish between the Delta and Omicron variants of SARS-CoV-2.

21. The composition of claim 20, comprising at least one assay formulated for targeting the Delta variant selected from:
an assay including SEQ ID NO:784 as a forward primer, SEQ ID NO:2784 as a reverse primer, SEQ ID NO:4784 as a probe configured to target a reference SARS-CoV-2 allele, and SEQ ID NO:6784 as a probe configured to target a mutant SARS-CoV-2 allele;
an assay including SEQ ID NO:968 as a forward primer, SEQ ID NO:2968 as a reverse primer, SEQ ID NO:4968 as a probe configured to target a reference SARS-CoV-2 allele, and SEQ ID NO:6968 as a probe configured to target a mutant SARS-CoV-2 allele;
an assay including one of SEQ ID NO:607 through SEQ ID NO:608 as a forward primer, one of SEQ ID NO:2607 through SEQ ID NO:2608 as a reverse primer, one of SEQ ID NO:4607 through SEQ ID NO:4608 as a probe configured to target a reference SARS-CoV-2 allele, and one of SEQ ID NO:6607 through SEQ ID NO:6608 as a probe configured to target a mutant SARS-CoV-2 allele;
an assay including one of SEQ ID NO:1188 through SEQ ID NO:1189 as a forward primer, one of SEQ ID NO:3188 through SEQ ID NO:3189 as a reverse primer, one of SEQ ID NO:5188 through SEQ ID NO:5189 as a probe configured to target a reference SARS-CoV-2 allele, and one of SEQ ID NO:7188 through SEQ ID NO:7189 as a probe configured to target a mutant SARS-CoV-2 allele;
an assay including one of SEQ ID NO:1192 through SEQ ID NO:1193 as a forward primer, one of SEQ ID NO:3192 through SEQ ID NO:3193 as a reverse primer, one of SEQ ID NO:5192 through SEQ ID NO:5193 as a probe configured to target a reference SARS-CoV-2 allele, and one of SEQ ID NO:7192 through SEQ ID NO:7193 as a probe configured to target a mutant SARS-CoV-2 allele; and
at least one assay formulated for targeting the Omicron variant selected from:
an assay including one of SEQ ID NO:61 through SEQ ID NO:62 as a forward primer, one of SEQ ID NO:2061 through SEQ ID NO:2062 as a reverse primer, one of SEQ ID NO:4061 through SEQ ID NO:4062 as a probe configured to target a reference SARS-CoV-2 allele, and one of SEQ ID NO:6061 through SEQ ID NO:6062 as a probe configured to target a mutant SARS-CoV-2 allele;
an assay including SEQ ID NO:616 as a forward primer, SEQ ID NO:2616 as a reverse primer, SEQ ID NO:4616 as a probe configured to target a reference SARS-CoV-2 allele, and SEQ ID NO:6616 as a probe configured to target a mutant SARS-CoV-2 allele;
an assay including one of SEQ ID NO:972 through SEQ ID NO:975 as a forward primer, one of SEQ ID NO:2972 through SEQ ID NO:2975 as a reverse primer, one of SEQ ID NO:4972 through SEQ ID NO:4975 as a probe configured to target a reference SARS-CoV-2 allele, and one of SEQ ID NO:6972 through SEQ ID NO:6975 as a probe configured to target a mutant SARS-CoV-2 allele;
an assay including one of SEQ ID NO:71 through SEQ ID NO:72 as a forward primer, one of SEQ ID NO:2071 through SEQ ID NO:2072 as a reverse primer, one of SEQ ID NO:4071 through SEQ ID NO:4072 as a probe configured to target a reference SARS-CoV-2 allele, and one of SEQ ID NO:6071 through SEQ ID NO:6072 as a probe configured to target a mutant SARS-CoV-2 allele;
an assay including SEQ ID NO:1196 as a forward primer, SEQ ID NO:3196 as a reverse primer, SEQ ID NO:5196 as a probe configured to target a reference SARS-CoV-2 allele, and SEQ ID NO:7196 as a probe configured to target a mutant SARS-CoV-2 allele.

22. The composition of claim 8, formulated to target and/or distinguish between the Alpha, Delta, and Omicron variants of SARS-CoV-2.

23. The composition of claim 22, comprising at least one assay formulated for targeting the Alpha variant selected from:
an assay including one of SEQ ID NO:560 through SEQ ID NO:562 as a forward primer, one of SEQ ID NO:2560 through SEQ ID NO:2562 as a reverse primer, one of SEQ ID NO:4560 through SEQ ID NO:4562 as a probe configured to target a reference SARS-CoV-2 allele, and one of SEQ ID NO:6560 through SEQ ID NO:6562 as a probe configured to target a mutant SARS-CoV-2 allele;
an assay including SEQ ID NO:564 as a forward primer, SEQ ID NO:2564 as a reverse primer, SEQ ID NO:4564 as a probe configured to target a reference SARS-CoV-2 allele, and SEQ ID NO:6564 as a probe configured to target a mutant SARS-CoV-2 allele;
an assay including one of SEQ ID NO:803 through SEQ ID NO:809 as a forward primer, one of SEQ ID NO:2803 through SEQ ID NO:2809 as a reverse primer, one of SEQ ID NO:4803 through SEQ ID NO:4809 as a probe configured to target a reference SARS-CoV-2 allele, and one of SEQ ID NO:6803 through SEQ ID NO:6809 as a probe configured to target a mutant SARS-CoV-2 allele;
an assay including one of SEQ ID NO:820 through SEQ ID NO:967 as a forward primer, one of SEQ ID NO:2820 through SEQ ID NO:2967 as a reverse primer, one of SEQ ID NO:4820 through SEQ ID NO:4967 as a probe configured to target a reference SARS-CoV-2 allele, and one of SEQ ID NO:6820 through SEQ ID NO:6967 as a probe configured to target a mutant SARS-CoV-2 allele;
an assay including SEQ ID NO:28 as a forward primer, SEQ ID NO:2028 as a reverse primer, SEQ ID NO:4028 as a probe configured to target a reference SARS-CoV-2 allele, and SEQ ID NO:6028 as a probe configured to target a mutant SARS-CoV-2 allele;
an assay including one of SEQ ID NO:336 through SEQ ID NO:380 as a forward primer, one of SEQ ID NO:2336 through SEQ ID NO:2380 as a reverse primer, one of SEQ ID NO:4336 through SEQ ID NO:4380 as a probe configured to target a reference SARS-CoV-2 allele, and one of SEQ ID NO:6336 through SEQ ID NO:6380 as a probe configured to target a mutant SARS-CoV-2 allele;
an assay including one of SEQ ID NO:93 through SEQ ID NO:323 as a forward primer, one of SEQ ID NO:2093 through SEQ ID NO:2323 as a reverse primer, one of SEQ ID NO:4093 through SEQ ID NO:4323 as a probe configured to target a reference SARS-CoV-2 allele, and one of SEQ ID NO:6093 through SEQ ID NO:6323 as a probe configured to target a mutant SARS-CoV-2 allele;
an assay including one of SEQ ID NO:1063 through SEQ ID NO:1186 as a forward primer, one of SEQ ID NO:3063 through SEQ ID NO:3186 as a reverse primer, one of SEQ ID NO:5063 through SEQ ID NO:5186 as a probe configured to target a reference SARS-CoV-2 allele, and one of SEQ ID NO:7063 through SEQ ID NO:7186 as a probe configured to target a mutant SARS-CoV-2 allele;
an assay including one of SEQ ID NO:1197 through SEQ ID NO:1267 as a forward primer, one of SEQ ID NO:3197 through SEQ ID NO:3267 as a reverse primer, one of SEQ ID NO:5197 through SEQ ID NO:5267 as a probe configured to target a reference SARS-CoV-2 allele, and one of SEQ ID NO:7197 through SEQ ID NO:7267 as a probe configured to target a mutant SARS-CoV-2 allele;
at least one assay formulated for targeting the Delta variant selected from:
an assay including SEQ ID NO:784 as a forward primer, SEQ ID NO:2784 as a reverse primer, SEQ ID NO:4784 as a probe configured to target a reference SARS-CoV-2 allele, and SEQ ID NO:6784 as a probe configured to target a mutant SARS-CoV-2 allele;
an assay including SEQ ID NO:968 as a forward primer, SEQ ID NO:2968 as a reverse primer, SEQ ID NO:4968 as a probe configured to target a reference SARS-CoV-2 allele, and SEQ ID NO:6968 as a probe configured to target a mutant SARS-CoV-2 allele;
an assay including one of SEQ ID NO:607 through SEQ ID NO:608 as a forward primer, one of SEQ ID NO:2607 through SEQ ID NO:2608 as a reverse primer, one of SEQ ID NO:4607 through SEQ ID NO:4608 as a probe configured to target a reference SARS-CoV-2 allele, and one of SEQ ID NO:6607 through SEQ ID NO:6608 as a probe configured to target a mutant SARS-CoV-2 allele;
an assay including one of SEQ ID NO:1188 through SEQ ID NO:1189 as a forward primer, one of SEQ ID NO:3188 through SEQ ID NO:3189 as a reverse primer, one of SEQ ID NO:5188 through SEQ ID NO:5189 as a probe configured to target a reference SARS-CoV-2 allele, and one of SEQ ID NO:7188 through SEQ ID NO:7189 as a probe configured to target a mutant SARS-CoV-2 allele;
an assay including one of SEQ ID NO:1192 through SEQ ID NO:1193 as a forward primer, one of SEQ ID NO:3192 through SEQ ID NO:3193 as a reverse primer, one of SEQ ID NO:5192 through SEQ ID NO:5193 as a probe configured to target a reference SARS-CoV-2 allele, and one of SEQ ID NO:7192 through SEQ ID NO:7193 as a probe configured to target a mutant SARS-CoV-2 allele; and
at least one assay formulated for targeting the Omicron variant selected from:
an assay including one of SEQ ID NO:61 through SEQ ID NO:62 as a forward primer, one of SEQ ID NO:2061 through SEQ ID NO:2062 as a reverse primer, one of SEQ ID NO:4061 through SEQ ID NO:4062 as a probe configured to target a reference SARS-CoV-2 allele, and one of SEQ ID NO:6061 through SEQ ID NO:6062 as a probe configured to target a mutant SARS-CoV-2 allele;
an assay including SEQ ID NO:616 as a forward primer, SEQ ID NO:2616 as a reverse primer, SEQ ID NO:4616 as a probe configured to target a reference SARS-CoV-2 allele, and SEQ ID NO:6616 as a probe configured to target a mutant SARS-CoV-2 allele;
an assay including one of SEQ ID NO:972 through SEQ ID NO:975 as a forward primer, one of SEQ ID NO:2972 through SEQ ID NO:2975 as a reverse primer, one of SEQ ID NO:4972 through SEQ ID NO:4975 as a probe configured to target a reference SARS-CoV-2 allele, and one of SEQ ID NO:6972 through SEQ ID NO:6975 as a probe configured to target a mutant SARS-CoV-2 allele;
an assay including one of SEQ ID NO:71 through SEQ ID NO:72 as a forward primer, one of SEQ ID NO:2071 through SEQ ID NO:2072 as a reverse primer, one of SEQ ID NO:4071 through SEQ ID NO:4072 as a probe configured to target a reference SARS-CoV-2 allele, and one of SEQ ID NO:6071 through SEQ ID NO:6072 as a probe configured to target a mutant SARS-CoV-2 allele;
an assay including SEQ ID NO:1196 as a forward primer, SEQ ID NO:3196 as a reverse primer, SEQ ID NO:5196 as a probe configured to target a reference SARS-CoV-2 allele, and SEQ ID NO:7196 as a probe configured to target a mutant SARS-CoV-2 allele.

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)
